(19) United States
(12) Patent Application Publication
(10) Pub. No.: US 2026/0234628 A1
(43) Pub. Date: Aug. 13, 2026

(54) Methods for Reducing HTT Expression

(71) Applicant: Ionis Pharmaceuticals, Inc., Carlsbad, CA (US)

(72) Inventors: C. Frank Bennett, Carlsbad, CA (US); Daniel A. Norris, San Marcos, CA (US); Anne V. Smith, Temecula, CA (US); Roger Lane, Encintas, CA (US)

(73) Assignee: Ionis Pharmaceuticals, Inc., Carlsbad, CA (US)

(21) Appl. No.: 19/659,031

(22) Filed: Apr. 27, 2026

Related U.S. Application Data
(63) Continuation of application No. 17/800,785, filed on Feb. 19, 2021, now abandoned.
(60) Provisional application No. 62/980,010, filed on Feb. 21, 2020.

(51) Int. Cl. C12N 15/113; A61P 25/14

(52) U.S. Cl. CPC C12N 15/113; A61P 25/14; C12N 2310/14; C12N 2310/315; C12N 2310/321; C12N 2310/3341; C12N 2320/35

(57) ABSTRACT

Provided herein are methods of administering ISIS 443139 for ameliorating Huntington's disease, reducing HTT RNA, reducing mHTT RNA, reducing HTT protein, or reducing mHTT protein in a human subject in need thereof. In certain instances, methods are useful for ameliorating at least one symptom of Huntington's disease. Such symptoms of Huntington's disease include, but are not limited to, brain atrophy, muscle atrophy, nerve degeneration, uncontrolled movement, difficulty swallowing, difficulty speaking, anxiety and depression.

### CROSS-REFERENCE TO RELATED APPLICATIONS

This application is a continuation of U.S. patent application Ser. No. 17/800,785, filed Aug. 18, 2022, which is a national phase entry pursuant to 35 U.S.C. § 371 of International Application No. PCT/US2021/018681, filed Feb. 19, 2021, which claims the benefit of priority to U.S. Provisional Patent Application No. 62/980,010, filed Feb. 21, 2020, each of which is incorporated by reference herein in its entirety for any purpose.

### SEQUENCE LISTING

The present application contains a Sequence Listing which has been submitted electronically in XML format. Said XML copy, created on Apr. 14, 2026, is named “01347-0001-01US-ST26.xml” and is 196,128 bytes in size. The information in the electronic format of the sequence listing is incorporated herein by reference in its entirety.

### FIELD

Provided herein are methods of administering ISIS 443139 for ameliorating Huntington's disease, reducing HTT RNA, reducing mHTT RNA, reducing HTT protein, or reducing mHTT protein in a human subject in need thereof. In certain instances, methods are useful for ameliorating at least one symptom of Huntington's disease. Such symptoms of Huntington's disease include, but are not limited to, brain atrophy, muscle atrophy, nerve degeneration, uncontrolled movement, difficulty swallowing, difficulty speaking, anxiety and depression.

### BACKGROUND

Huntington's disease (HD) is a devastating autosomal dominant, neurodegenerative disease characterized by progressive chorea, psychiatric changes, and intellectual decline. HD affects males and females equally, and occurs in all races (Gusella and MacDonald, Curr. Opin. Neurobiol. 1995 5:656-62). Selective cell loss and fibrillary astrocytosis is observed in the brains of HD patients, particularly in the caudate and putamen of the striatum and in the cerebral cortex of HD patients (Vonsattel, J-P. et al., Neuropathol. Exp. Neurol. 1985, 44:559-577), and, to a lesser extent, in the hippocampus (Spargo, E. et al., J. Neurol. Neurosurg. Psychiatry 1993, 56:487-491) and the subthalamus (Byers, R. K. et al., Neurology 1973, 23:561-569). Symptoms of HD are due to the death of neurons in many brain regions, but is most apparent in the striatum, particularly in the caudate nucleus, which suffers a progressive gradient of cell loss that ultimately decimates the entire structure.

HD is caused by the expansion of a cytosine-adenine-guanine (CAG) trinucleotide repeat region in IT15, the gene that encodes huntingtin protein (HTT protein). The resulting expanded CAG repeat region encodes an abnormally long polyglutamine (PolyQ) tract in HTT protein, resulting in the expression of a mutant HTT (mHTT) protein. As a result of excessive polyglutamine length, mHTT protein forms aggregates in the cytoplasm and nucleus of CNS neurons (Davies et al., Cell 1997, 90:537-548). Due to its genomic instability, the expanded CAG repeat region can further expand with age and during meiotic transmission to include additional CAG repeats. Individuals with 27 to 35 CAG repeats typically do not develop HD, but their children are at risk of developing HD. Individuals with 35 to 60 CAG repeats typically experience adult-onset HD. Individuals with greater than 60 CAG repeats generally develop juvenile HD, experiencing symptoms of HD before the age of 20 years. Individuals with a normal number of CAG repeats (<27) are not considered to be at risk of developing HD.

### BRIEF DESCRIPTION OF THE DRAWINGS

FIG. 1A shows mean reduction in cerebrospinal fluid (CSF) mHTT protein trough concentration as a percentage of baseline at multiple time points in human subjects treated with modified oligonucleotide ISIS 443139 every four weeks.

FIG. 1B shows mean reduction in cerebrospinal fluid (CSF) mHTT protein trough concentration as a percentage of baseline at multiple time points in human subjects treated with modified oligonucleotide ISIS 443139 every eight weeks.

### SUMMARY OF THE INVENTION

Provided herein are methods for ameliorating Huntington's disease (HD), and methods of reducing HTT RNA and/or HTT protein in a human subject in need thereof. In certain embodiments, the HTT RNA is mHTT RNA. In certain embodiments, the HTT protein is mHTT protein In certain embodiments, methods comprise administering a therapeutically effective amount of a modified oligonucleotide. In certain embodiments, the modified oligonucleotide is ISIS 443139. In certain embodiments, the therapeutically effective amount is within the range of about 40 mg to about 200 mg. In certain embodiments, the therapeutically effective amount is about 120 mg. In certain embodiments, the therapeutically effective amount is administered once about every 4 weeks. In certain embodiments, the therapeutically effective amount is administered once about every 8 weeks. In certain embodiments, the therapeutically effective amount is administered once about every 16 weeks. In certain embodiments, methods comprise administering a loading dose of about 120 mg of ISIS 443139 once about every 4 weeks, and subsequently administering a maintenance dose of 120 mg of ISIS 443139 once about every 8 weeks or once about every 16 weeks.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxyribonucleoside” means a nucleoside comprising a 2′-H(H) deoxyribosyl sugar moiety. In certain embodiments, a 2′-deoxyribonucleoside is a 2′-β-D deoxyribonucleoside and comprises a 2′-β-D-deoxyribosyl sugar moiety, which has the β-D configuration as found in naturally occurring deoxyribonucleic acids (DNA). In certain embodiments, a 2′-deoxyribonucleoside may comprise a modified nucleobase or may comprise an RNA nucleobase (uracil).

As used herein, “2′-MOE” means a 2′—OCH2CH₂OCH3 group in place of the 2′—OH group of a ribosyl sugar moiety. A “2′-MOE sugar moiety” is a sugar moiety with a 2′—OCH2CH₂OCH3 group in place of the 2′—OH group of a ribosyl sugar moiety. Unless otherwise indicated, a 2′-MOE sugar moiety is in the β-D configuration. “MOE” means O-methoxyethyl.

As used herein, “2′-MOE nucleoside” means a nucleoside comprising a 2′-MOE sugar moiety.

As used herein, “5-methyl cytosine” means a cytosine modified with a methyl group attached to the 5 position. A 5-methyl cytosine is a modified nucleobase.

As used herein, “about” means plus or minus 7% of the provided value.

As used herein, “administering” means providing a pharmaceutical agent to a human subject.

As used herein, “ameliorate” in reference to a treatment means improvement in at least one symptom relative to the same symptom in the absence of the treatment. In certain embodiments, amelioration is the reduction in the severity or frequency of a symptom, or the delayed onset or slowing of progression in the severity or frequency of a symptom.

As used herein, “CAG repeat” means one of multiple contiguous trinucleotide units, wherein each trinucleotide unit consists of three contiguous nucleosides having a nucleobase sequence from 5′ to 3′ of cytosine (C), adenine (A), and guanine (G).

As used herein, “dose” means a quantity of a pharmaceutical agent administered.

As used herein, “HTT RNA” is the RNA expression product of the human gene, IT15. “mHTT RNA” is the RNA expression product of the human gene, IT15, that contains 27 or more contiguous CAG repeats.

As used herein, “HTT protein” is the protein expression product of HTT RNA. “mHTT protein,” is the protein expression product of mHTT.

As used herein, the term “internucleoside linkage” means the covalent linkage between contiguous nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “IT15 gene” refers to a genomic sequence encoding an HTT RNA. In general, a human has two IT15 genes which may have the same or different nucleobase sequences.

As used herein, “loading dose” means a therapeutically effective amount of a pharmaceutical agent administered during an initial dosing phase during which steady state concentration of the pharmaceutical agent is achieved. “Initial loading dose” means the first loading dose administered. “Last loading dose” means the loading dose administered most recently prior to administering a first maintenance dose.

As used herein, “maintenance dose” means a therapeutically effective amount of a pharmaceutical agent administered during a dosing phase after steady state concentration of the pharmaceutical agent has been achieved.

As used herein, “nucleobase” means an unmodified nucleobase or modified nucleobase. An “unmodified nucleobase” is adenine (A), thymine (T), cytosine (C), uracil (U), or guanine (G). A “modified nucleobase” is group of atoms other than unmodified A, T, C, U, or G capable of pairing with at least one unmodified nucleobase. A “5-methyl cytosine” is a modified nucleobase. As used herein, “nucleobase sequence” means the order of contiguous nucleobases in a target nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. “Linked nucleosides” are nucleosides that are connected in a contiguous sequence (i.e., no additional nucleosides are presented between those that are linked). As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified.

As used herein, “pharmaceutically acceptable carrier or diluent” means any substance suitable for use in administering to a human subject. Certain such carriers enable pharmaceutical compositions to be formulated as, for example, tablets, pills, dragees, capsules, liquids, gels, syrups, slurries, suspension, and lozenges for the oral ingestion by a human subject. In certain embodiments, a pharmaceutically acceptable carrier or diluent is sterile water, sterile saline, sterile buffer solution, or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “potassium salt” means a salt of a modified oligonucleotide, wherein the cation of the salt is potassium.

As used herein, “RNA” means an RNA transcript and includes pre-mRNA and mature mRNA unless otherwise specified.

As used herein, “sodium salt” means a salt of a modified oligonucleotide, wherein the cation of the salt is sodium.

As used herein, “subject” means a human or non-human animal. In certain embodiments, the subject is a human subject. A “subject in need thereof,” is a subject who would benefit from administration of a modified oligonucleotide disclosed herein. In certain embodiments, the subject in need thereof has HD.

As used herein, “sugar moiety” means an unmodified sugar moiety or a modified sugar moiety. “Unmodified sugar moiety” means a 2′-OH(H) β-D ribosyl moiety, as found in RNA (an “unmodified RNA sugar moiety”), or a 2′-H(H) β-D deoxyribosyl moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate.

As used herein, “symptom” means any physical feature or test result that indicates the existence or extent of a disease or disorder. In certain embodiments, a symptom is apparent to a subject or to a medical professional examining or testing the subject.

As used herein, “therapeutically effective amount” means an amount of a pharmaceutical agent that provides a therapeutic benefit to a human subject. For example, a therapeutically effective amount improves a symptom of a disease.

As used herein, “trough concentration” means the concentration of an analyte (e.g., mHTT) in a biological sample taken from a dosed human subject immediately prior to the human subject receiving a subsequent dose or the concentration of an analyte on the last study day.

As used herein, “week” means 7 days.

### CERTAIN EMBODIMENTS

Embodiment 1. A method of ameliorating Huntington's disease (HD) in a human subject in need thereof, the method comprising administering to the human subject a therapeutically effective amount of a modified oligonucleotide according to the following chemical structure:

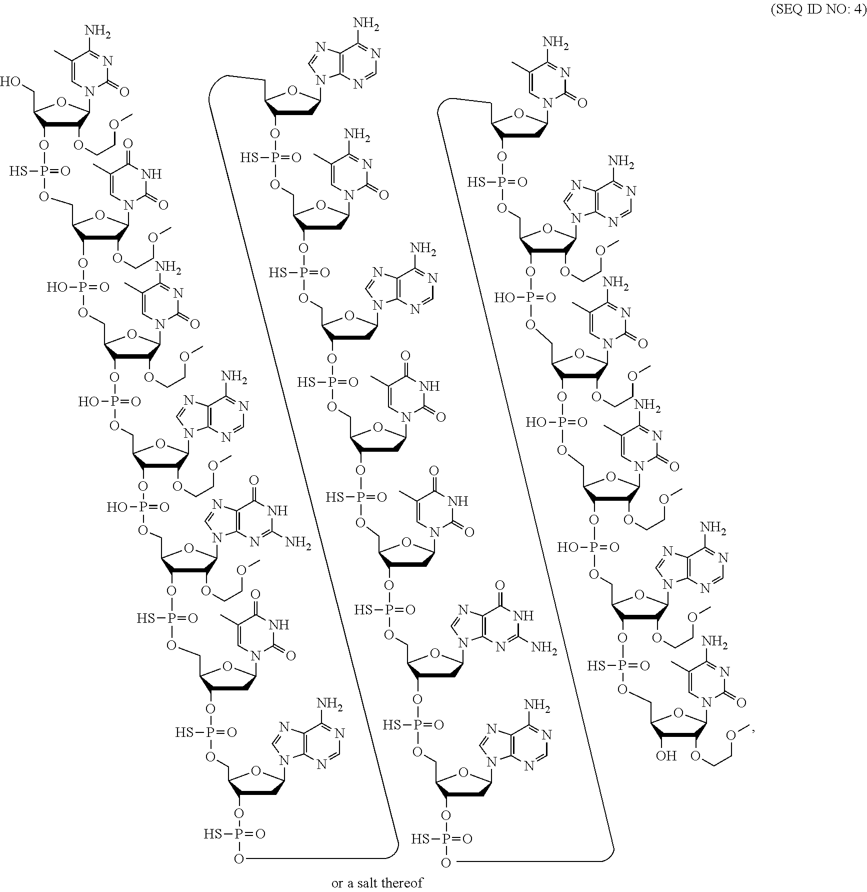

Embodiment 2. The method of embodiment 1, wherein the modified oligonucleotide is the sodium salt or the potassium salt.

Embodiment 3. A method of ameliorating HD in a human subject in need thereof, the method comprising administering to the human subject a therapeutically effective amount of a modified oligonucleotide according to the following chemical structure:

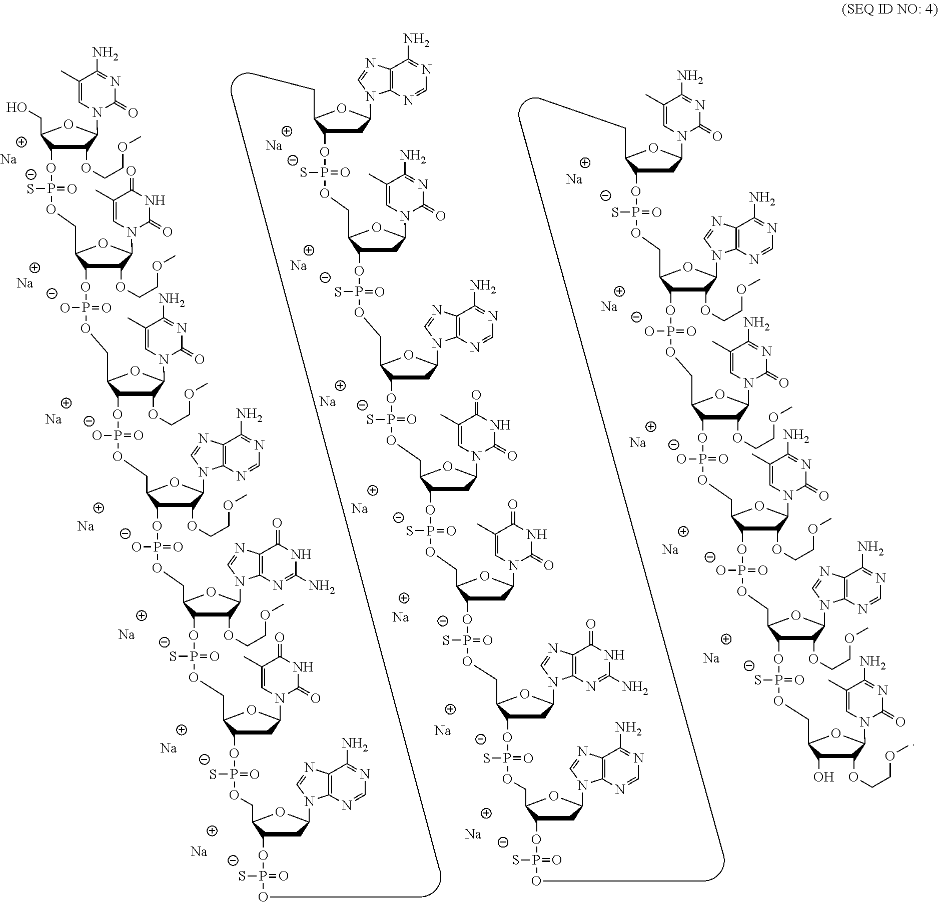

Embodiment 4. A method of ameliorating HD in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 5. The method of any one of embodiments 1-4, wherein at least one symptom of HD is ameliorated.

Embodiment 6. The method of embodiment 5, wherein the at least one symptom comprises brain atrophy, reduced brain activity, reduced brain connectivity, muscle atrophy, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

Embodiment 7. A method of reducing HTT RNA in a human subject in need thereof, the method comprising administering to the human subject a therapeutically effective amount of a modified oligonucleotide according to the following chemical structure:

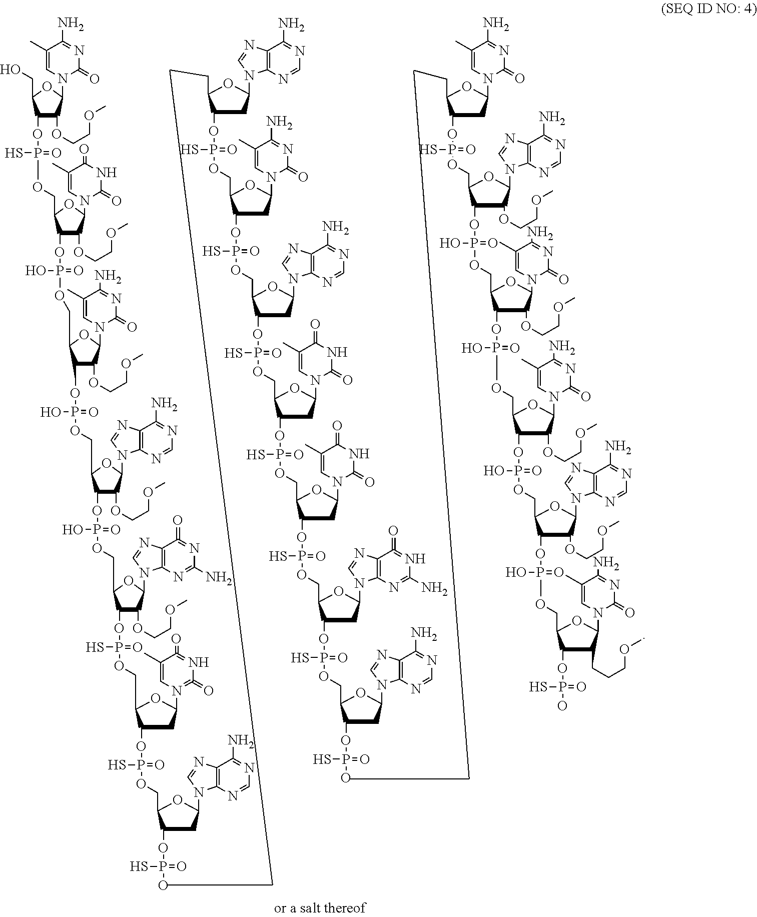

Embodiment 8. The method of embodiment 7, wherein the modified oligonucleotide is the sodium salt or the potassium salt.

Embodiment 9. A method of reducing HTT RNA in a human subject in need thereof, the method comprising administering to the human subject a therapeutically effective amount of a modified oligonucleotide according to the following chemical structure:

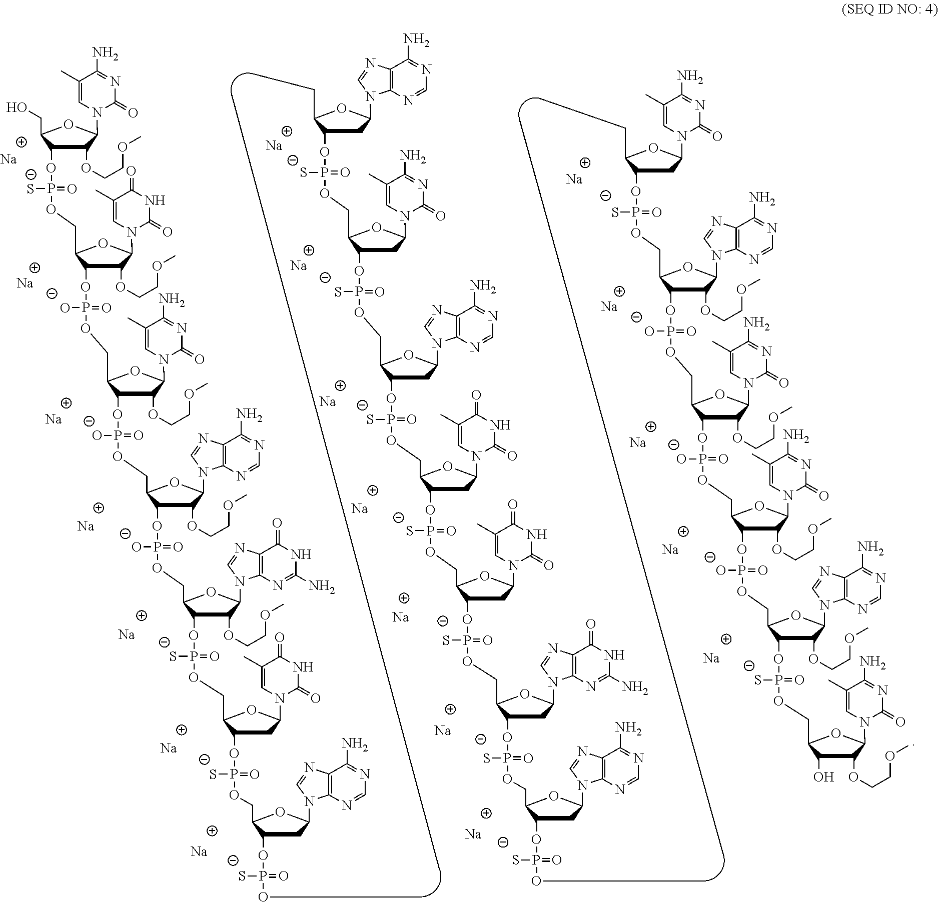

Embodiment 10. A method of reducing HTT RNA in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 11. A method of reducing HTT protein in a human subject in need thereof, the method comprising administering to the human subject a therapeutically effective amount of a modified oligonucleotide according to the following chemical structure:

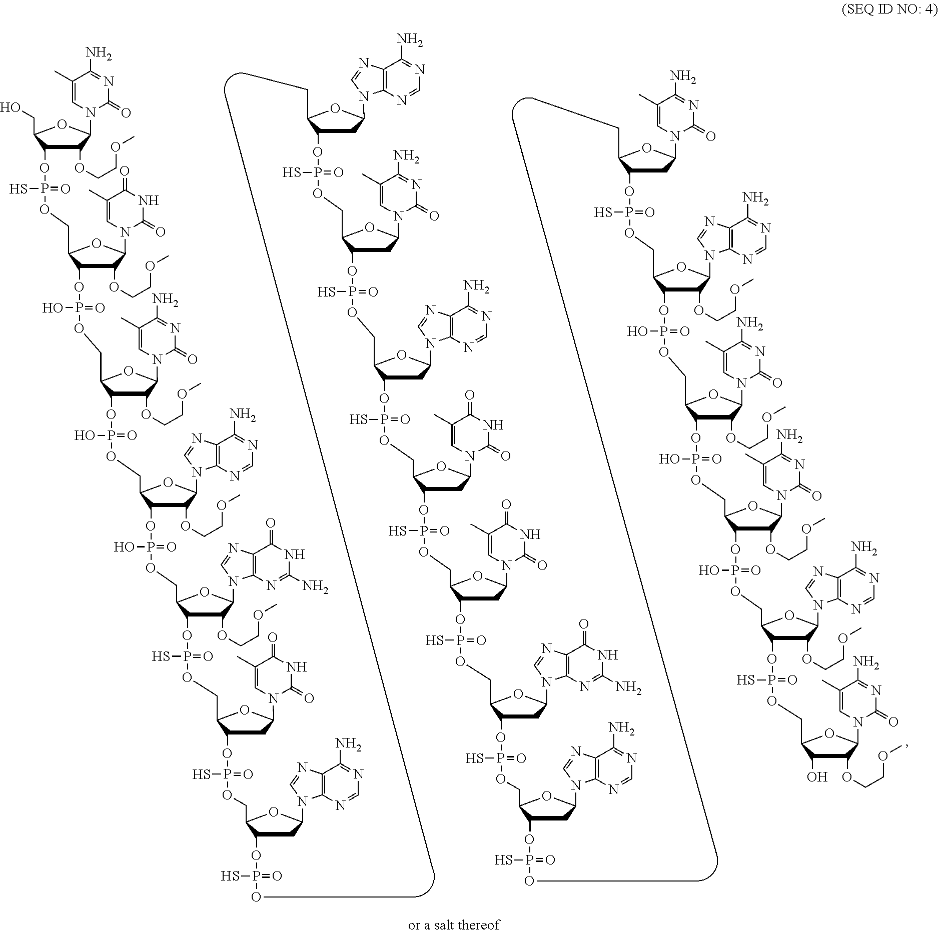

Embodiment 12. The method of embodiment 11, wherein the modified oligonucleotide is the sodium salt or the potassium salt.

Embodiment 13. A method of reducing HTT protein in a human subject in need thereof, the method comprising administering to the human subject a therapeutically effective amount of a modified oligonucleotide according to the following chemical structure:

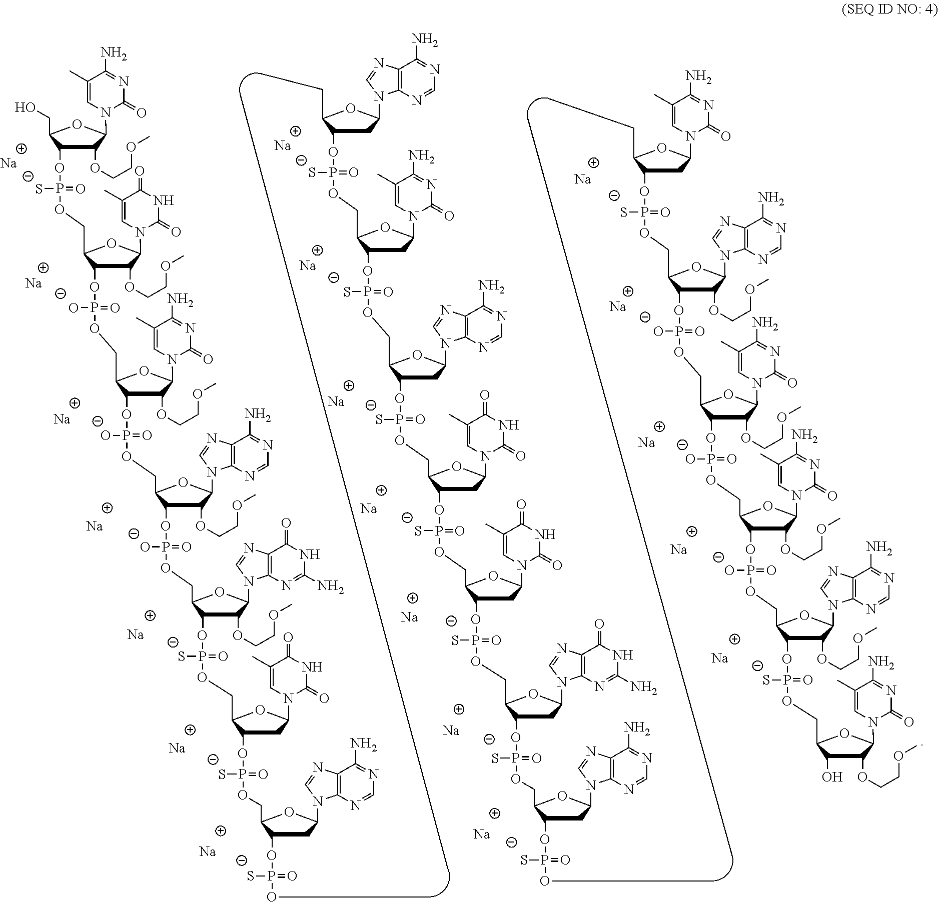

Embodiment 14. A method of reducing HTT protein in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 15. The method of any one of embodiments 1-14, wherein the therapeutically effective amount is 10 mg.

Embodiment 16. The method of any one of embodiments 1-14, wherein the therapeutically effective amount is 30 mg.

Embodiment 17. The method of any one of embodiments 1-14, wherein the therapeutically effective amount is 60 mg.

Embodiment 18. The method of any one of embodiments 1-14, wherein the therapeutically effective amount is 90 mg.

Embodiment 19. The method of any one of embodiments 1-14, wherein the therapeutically effective amount is 120 mg.

Embodiment 20. The method of any one of embodiments 1-14, wherein the therapeutically effective amount is about 10 mg.

Embodiment 21. The method of any one of embodiments 1-14, wherein the therapeutically effective amount is about 30 mg.

Embodiment 22. The method of any one of embodiments 1-14, wherein the therapeutically effective amount is about 60 mg.

Embodiment 23. The method of any one of embodiments 1-14, wherein the therapeutically effective amount is about 90 mg.

Embodiment 24. The method of any one of embodiments 1-14, wherein the therapeutically effective amount is about 120 mg.

Embodiment 25. The method of any one of embodiments 1-14, wherein the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

Embodiment 26. The method of any one of embodiments 1-14, wherein the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, and about 300 mg.

Embodiment 27. The method of any one of embodiments 1-14, wherein the therapeutically effective amount is any of 115.0 mg, 115.1 mg, 115.2 mg, 115.3 mg, 115.4 mg, 115.5 mg, 115.6 mg, 115.7 mg, 115.8 mg, 115.9 mg, 116.0 mg, 116.1 mg, 116.2 mg, 116.3 mg, 116.4 mg, 116.5 mg, 116.6 mg, 116.7 mg, 116.8 mg, 116.9 mg, 117.0 mg, 117.1 mg, 117.2 mg, 117.3 mg, 117.4 mg, 117.5 mg, 117.6 mg, 117.7 mg, 117.8 mg, 117.9 mg, 118.0 mg, 118.1 mg, 118.2 mg, 118.3 mg, 118.4 mg, 118.5 mg, 118.6 mg, 118.7 mg, 118.8 mg, 118.9 mg, 119.0 mg, 119.1 mg, 119.2 mg, 119.3 mg, 119.4 mg, 119.5 mg, 119.6 mg, 119.7 mg, 119.8 mg, 119.9 mg, 120.0 mg, 120.1 mg, 120.2 mg, 120.3 mg, 120.4 mg, 120.5 mg, 120.6 mg, 120.7 mg, 120.8 mg, 120.9 mg, 121.0 mg, 121.1 mg, 121.2 mg, 121.3 mg, 121.4 mg, 121.5 mg, 121.6 mg, 121.7 mg, 121.8 mg, 121.9 mg, 122.0 mg, 122.1 mg, 122.2 mg, 122.3 mg, 122.4 mg, 122.5 mg, 122.6 mg, 122.7 mg, 122.8 mg, 122.9 mg, 123.0 mg, 123.1 mg, 123.2 mg, 123.3 mg, 123.4 mg, 123.5 mg, 123.6 mg, 123.7 mg, 123.8 mg, 123.9 mg, 124.0 mg, 124.1 mg, 124.2 mg, 124.3 mg, 124.4 mg, 124.5 mg, 124.6 mg, 124.7 mg, 124.8 mg, 124.9 mg, and 125.0 mg.

Embodiment 28. The method of any one of embodiments 1-14, wherein the therapeutically effective amount is any of about 115.0 mg, about 115.1 mg, about 115.2 mg, about 115.3 mg, about 115.4 mg, about 115.5 mg, about 115.6 mg, about 115.7 mg, about 115.8 mg, about 115.9 mg, about 116.0 mg, about 116.1 mg, about 116.2 mg, about 116.3 mg, about 116.4 mg, about 116.5 mg, about 116.6 mg, about 116.7 mg, about 116.8 mg, about 116.9 mg, about 117.0 mg, about 117.1 mg, about 117.2 mg, about 117.3 mg, about 117.4 mg, about 117.5 mg, about 117.6 mg, about 117.7 mg, about 117.8 mg, about 117.9 mg, about 118.0 mg, about 118.1 mg, about 118.2 mg, about 118.3 mg, 118.4 mg, about 118.5 mg, about 118.6 mg, about 118.7 mg, about 118.8 mg, about 118.9 mg, about 119.0 mg, about 119.1 mg, about 119.2 mg, about 119.3 mg, about 119.4 mg, about 119.5 mg, about 119.6 mg, about 119.7 mg, about 119.8 mg, about 119.9 mg, about 120.0 mg, about 120.1 mg, about 120.2 mg, about 120.3 mg, 120.4 mg, about 120.5 mg, about 120.6 mg, about 120.7 mg, about 120.8 mg, about 120.9 mg, about 121.0 mg, about 121.1 mg, about 121.2 mg, about 121.3 mg, about 121.4 mg, about 121.5 mg, about 121.6 mg, about 121.7 mg, about 121.8 mg, about 121.9 mg, about 122.0 mg, about 122.1 mg, about 122.2 mg, about 122.3 mg, 122.4 mg, about 122.5 mg, about 122.6 mg, about 122.7 mg, about 122.8 mg, about 122.9 mg, about 123.0 mg, about 123.1 mg, about 123.2 mg, about 123.3 mg, about 123.4 mg, about 123.5 mg, about 123.6 mg, about 123.7 mg, about 123.8 mg, about 123.9 mg, about 124.0 mg, about 124.1 mg, about 124.2 mg, about 124.3 mg, 124.4 mg, about 124.5 mg, about 124.6 mg, about 124.7 mg, about 124.8 mg, about 124.9 mg, and about 125.0 mg.

Embodiment 29. The method of any one of embodiments 1-14, wherein the therapeutically effective amount is within the range of any of 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

Embodiment 30. The method of any one of embodiments 1-14, wherein the therapeutically effective amount is any of less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

Embodiment 31. The method of any one of embodiments 1-14, wherein the therapeutically effective amount is any of less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

Embodiment 32. The method of any one of embodiments 1-14, wherein the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

Embodiment 33. The method of any one of embodiments 1-14, wherein the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

Embodiment 34. The method of any one of embodiments 1-33, comprising administering the modified oligonucleotide once every 4 weeks.

Embodiment 35. The method of any one of embodiments 1-33, comprising administering the modified oligonucleotide once every 8 weeks.

Embodiment 36. The method of any one of embodiments 1-33, comprising administering the modified oligonucleotide once every 12 weeks.

Embodiment 37. The method of any one of embodiments 1-33, comprising administering the modified oligonucleotide once every 16 weeks.

Embodiment 38. The method of any one of embodiments 1-33, comprising administering the modified oligonucleotide once every 20 weeks.

Embodiment 39. The method of any one of embodiments 1-33, comprising administering the modified oligonucleotide about once every 4 weeks.

Embodiment 40. The method of any one of embodiments 1-33, comprising administering the modified oligonucleotide about once every 8 weeks.

Embodiment 41. The method of any one of embodiments 1-33, comprising administering the modified oligonucleotide about once every 12 weeks.

Embodiment 42. The method of any one of embodiments 1-33, comprising administering the modified oligonucleotide about once every 16 weeks.

Embodiment 43. The method of any one of embodiments 1-33, comprising administering the modified oligonucleotide about once every 20 weeks.

Embodiment 44. The method of any one of embodiments 1-33, comprising administering the modified oligonucleotide any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, and once every 20 weeks.

Embodiment 45. The method of any one of embodiments 1-33, comprising administering the modified oligonucleotide any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, and once about every 20 weeks.

Embodiment 46. The method of any of embodiments 1-33, comprising administering to the human subject an initial loading dose of 120 mg of the modified oligonucleotide.

Embodiment 47. The method of embodiment 46, comprising administering to the human subject a second loading dose of 120 mg of the modified oligonucleotide 4 weeks after the initial loading dose.

Embodiment 48. The method of embodiment 47, comprising administering to the human subject a maintenance dose of 120 mg of the modified oligonucleotide 4 weeks after the second loading dose.

Embodiment 49. The method of embodiment 47, comprising administering to the human subject a maintenance dose of 120 mg of the modified oligonucleotide 8 weeks after the second loading dose.

Embodiment 50. The method of embodiment 47, comprising administering to the human subject a maintenance dose of 120 mg of the modified oligonucleotide 12 weeks after the second loading dose.

Embodiment 51. The method of embodiment 47, comprising administering to the human subject a maintenance dose of 120 mg of the modified oligonucleotide 16 weeks after the second loading dose.

Embodiment 52. The method of any of embodiments 7-10 and 15-51, wherein the HTT RNA is mHTT RNA.

Embodiment 53. The method of any of embodiment 11-51, wherein the HTT protein is mHTT protein.

Embodiment 54. A method of ameliorating HD, reducing HTT RNA, reducing HTT protein, reducing mHTT RNA, or reducing mHTT protein in a human subject in need thereof, the method comprising intrathecally administering to the human subject a therapeutically effective amount of 120 mg or about 120 mg of a modified oligonucleotide according to the following chemical structure:

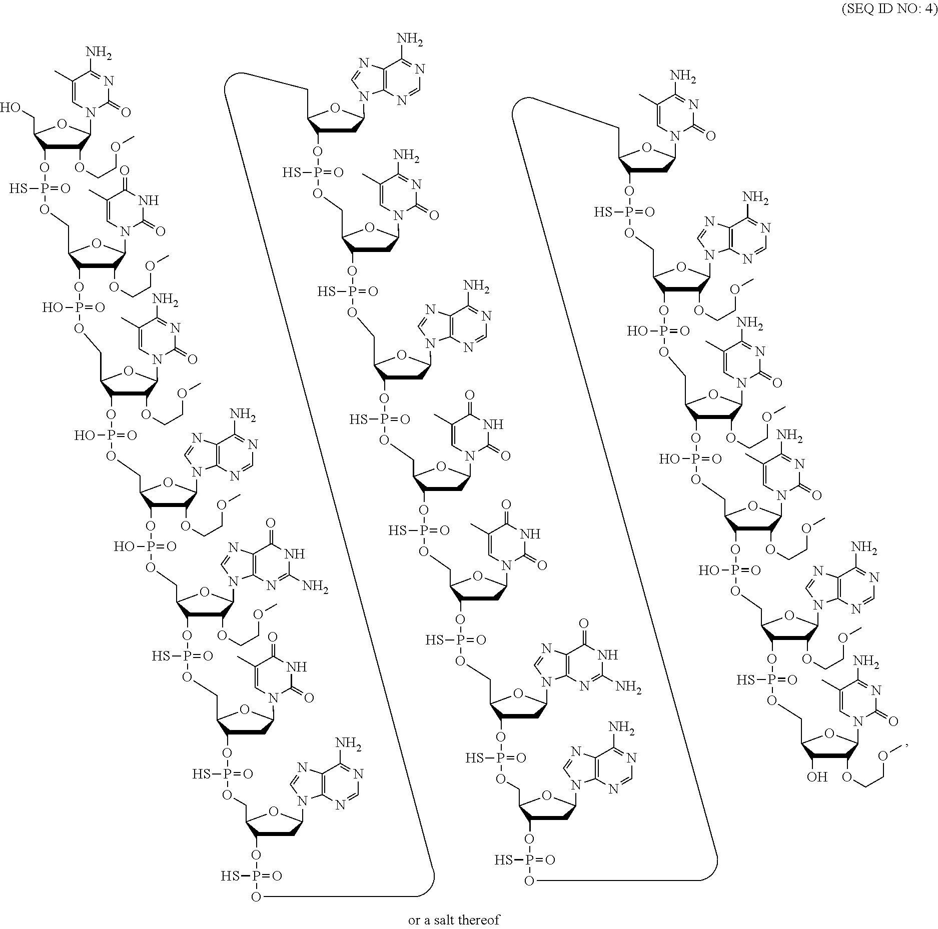

Embodiment 55. The method of embodiment 54, wherein the modified oligonucleotide is the sodium salt or the potassium salt.

Embodiment 56. A method of ameliorating HD, reducing HTT RNA, reducing HTT protein, reducing mHTT RNA, or reducing mHTT protein in a human subject in need thereof, the method comprising intrathecally administering to the human subject a therapeutically effective amount of 120 mg or about 120 mg of a modified oligonucleotide according to the following chemical structure:

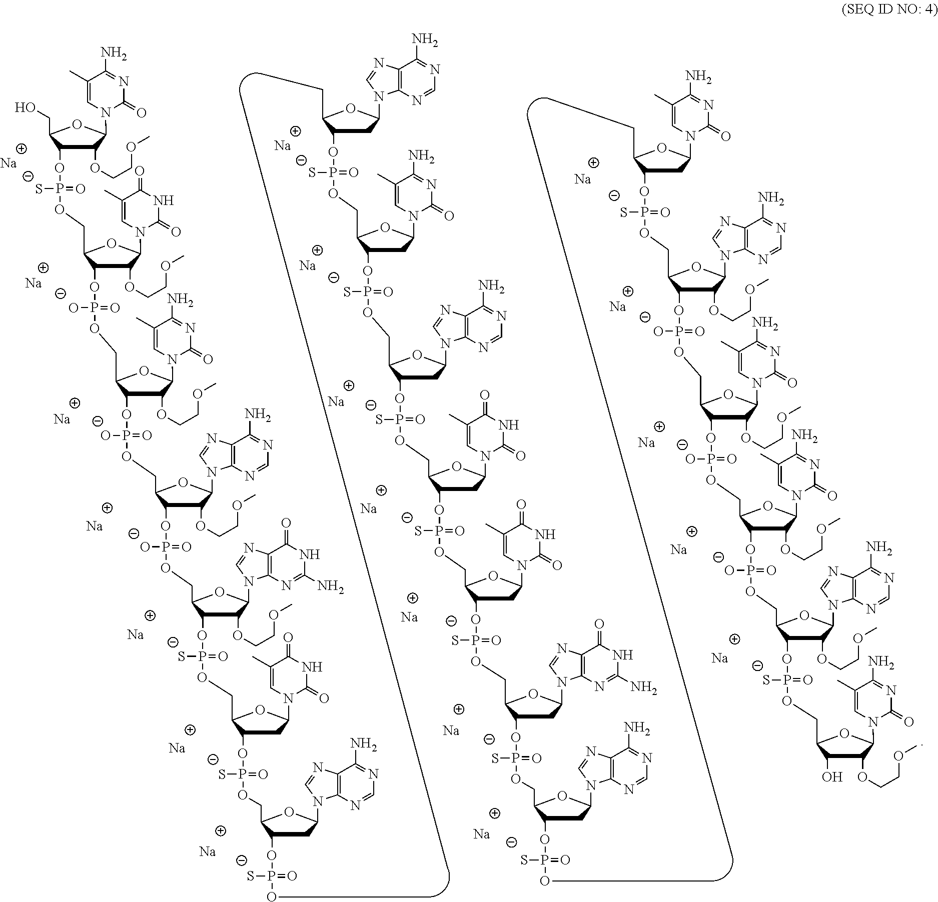

Embodiment 57. A method of ameliorating HD, reducing HTT RNA, reducing HTT protein, reducing HTT mRNA, or reducing mHTT protein in a human subject in need thereof, the method comprising intrathecally administering to the human subject a therapeutically effective amount of 120 mg or about 120 mg of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

Embodiment 58. The method of any one of embodiments 54-57, comprising administering the modified oligonucleotide about once every 4 weeks.

Embodiment 59. The method of any one of embodiments 54-57, comprising administering the modified oligonucleotide about once every 8 weeks.

Embodiment 60. The method of any one of embodiments 54-57, comprising administering the modified oligonucleotide about once every 16 weeks.

Embodiment 61. The method of any of embodiments 54-57, comprising administering to the human subject:
a) an initial loading dose of about 120 mg of the modified oligonucleotide,
b) a second loading dose of about 120 mg of the modified oligonucleotide about 4 weeks after administering the initial loading dose;
c) a first maintenance dose of about 120 mg of the modified oligonucleotide about 8 weeks after administering the second loading dose;
d) a second maintenance dose of about 120 mg of the modified oligonucleotide about 8 weeks after administering the first maintenance dose.

Embodiment 62. The method of any of embodiments 54-57, comprising administering to the human subject:
a) an initial loading dose of about 120 mg of the modified oligonucleotide,
b) a second loading dose of about 120 mg of the modified oligonucleotide about 4 weeks after administering the initial loading dose;
c) a first maintenance dose of about 120 mg of the modified oligonucleotide about 16 weeks after administering the second loading dose;
d) a second maintenance dose of about 120 mg of the modified oligonucleotide about 16 weeks after administering the first maintenance dose.

Embodiment 63. The method of any one of embodiments 54-62, wherein at least one symptom of HD is ameliorated.

Embodiment 64. The method of embodiment 63, wherein the at least one symptom comprises brain atrophy, reduced brain activity, reduced brain connectivity, muscle atrophy, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

Embodiment 65. The method of any of embodiments 1-64, wherein the human subject has a mutation in at least one IT15 gene.

Embodiment 66. The method of any of embodiments 1-65, comprising identifying a mutation in at least one IT15 gene of the human subject.

Embodiment 67. The method of embodiment 65 or embodiment 66, wherein the at least one IT15 gene has any of at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 contiguous CAG repeats.

Embodiment 68. The method of embodiment 65 or embodiment 66, wherein the at least one IT15 gene has 27 to 35 contiguous CAG repeats.

Embodiment 69. The method of embodiment 65 or embodiment 66, wherein the at least one IT15 gene has 35 to 60 contiguous CAG repeats.

Embodiment 70. The method of embodiment 65 or embodiment 66, wherein the at least one IT15 gene has greater than 60 contiguous CAG repeats.

Embodiment 71. The method of any of embodiments 1-70, wherein the modified oligonucleotide is administered to the CNS of the human subject.

Embodiment 72. The method of any of embodiments 1-71, wherein the modified oligonucleotide is administered by intrathecal administration.

Embodiment 73. The method of any of embodiments 1-72, wherein the modified oligonucleotide is administered by bolus intrathecal administration.

Embodiment 74. The method of any of embodiments 1-73, wherein HTT RNA is reduced.

Embodiment 75. The method of any of embodiments 1-74, wherein HTT protein is reduced.

Embodiment 76. The method of any of embodiments 1-75, wherein mHTT RNA is reduced.

Embodiment 77. The method of any of embodiments 1-76, wherein mHTT protein is reduced.

Embodiment 78. The method of any one of embodiments 1-77, comprising detecting an amount of mHTT RNA in a biological sample from the human subject.

Embodiment 79. The method of any one of embodiments 1-78, comprising detecting an amount of mHTT protein in a biological sample from the human subject.

Embodiment 80. The method of any one of embodiments 1-79, wherein the biological sample comprises cerebrospinal fluid.

Embodiment 81. The method of any of embodiments 78-80, wherein the detecting occurs before the administering.

Embodiment 82. The method of any of embodiments 78-80, wherein the detecting occurs after the administering.

Embodiment 83. The method of any of embodiments 78-80, wherein the detecting occurs before and after the administering.

Embodiment 84. The method of any one of embodiments 78-83, comprising adjusting the initial loading dose, the loading dose, maintenance dose, or therapeutically effective amount administered after detecting the amount of HTT RNA, HTT protein, mHTT RNA, mHTT protein, or combination thereof.

Embodiment 85. The method of any one of embodiments 1-84, comprising analyzing brain activity, brain size, or a combination thereof of the subject by performing an electroencephalogram (EEG) or magnetic resonance imaging (MRI) on the subject.

Embodiment 86. The method of embodiment 85, wherein performing the EEG or MRI occurs before administering, after administering, or a combination thereof.

Embodiment 87. The method of embodiment 86, comprising determining or adjusting the therapeutically effective amount after performing the EEG or MRI.

Embodiment 88. The method of embodiment 87, comprising performing the EEG or MRI after administering, and adjusting the frequency of administering after performing the EEG or MRI.

Embodiment 89. The method of any one of embodiments 85-88, wherein performing the EEG or MRI is performed within 1, 2, 4, 6, 8, 12 or 24 hours of administering.

Embodiment 90. The method of any one of embodiments 85-89, comprising performing the EEG before administering, and analyzing after administering, detecting less than a 4 Hz increase in EEG signal power from a first EEG to a second EEG, and subsequently increasing the frequency of administering the therapeutically effective amount of the modified oligonucleotide.

Embodiment 91. The method of embodiment 90, comprising administering a loading dose once about every 4 weeks and administering a maintenance dose once about every 8 or 16 weeks before recording the first EEG, and administering the maintenance dose less than about every 8 weeks or less than about every 16 weeks.

Embodiment 92. The method of any one of embodiments, 85-91, comprising recording a first EEG before administering, and recording a second EEG after administering, detecting less than a 4 Hz increase in EEG signal power from the first EEG to the second EEG, and subsequently administering a dose of the modified oligonucleotide that is greater than the therapeutically effective amount.

Embodiment 93. The method of embodiment 92, wherein the dose is at least about 10%, at least about 20%, at least about 30%, at least about 40%, at least about 50%, at least about 60%, at least about 70%, at least about 90%, or at least about 100% greater than the therapeutically effective amount.

Embodiment 94. The method of any one of embodiments 85-93, wherein the therapeutically effective amount is about 120 mg or 120 mg.

### I. 202HTT

In certain embodiments, described herein are methods of reducing HTT RNA and/or HTT protein in a cell or a biological fluid of a subject. In certain embodiments, the HTT RNA is mHTT RNA. In certain embodiments, the HTT protein is mHTT protein. HTT RNA is encoded by the human IT15 gene, located on the short (p) arm of human chromosome 4. HTT protein is the protein expression product of HTT RNA. HTT protein is highly expressed in neurons relative to other cell types. A representative nucleobase sequence for a human IT15 gene is provided at GENBANK Accession No. NC_000004.12 truncated from nucleotides 3072001 to 3247000, incorporated herein as SEQ ID NO: 1. A representative nucleobase sequence for a human HTT RNA is provided at GENBANK Accession No. NM_002111.6, incorporated herein as SEQ ID NO: 2. A representative protein sequence for a human HTT protein is provided at GENBANK Accession No. NP_002102.4, incorporated here in as SEQ ID NO: 3.

### II. ISIS 443139

In certain embodiments, described herein are methods of administering modified oligonucleotide, ISIS 443139, to a subject in need thereof. In certain embodiments, ISIS 443139 is characterized as a 5-10-5 MOE gapmer, having a sequence of (from 5′ to 3′) CTCAGTAACATTGACACCAC (incorporated herein as SEQ ID NO: 4), wherein each of nucleosides 1-5 and 16-20 (from 5′ to 3′) are 2′-MOE nucleosides and each of nucleosides 6-15 are 2′-β-D deoxyribonucleosides, wherein the internucleoside linkages between nucleosides 2 to 3, 3 to 4, 4 to 5, 16 to 17, 17 to 18, and 18 to 19, are phosphodiester internucleoside linkages and the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, and 19 to 20 are phosphorothioate internucleoside linkages, and wherein each cytosine is a 5-methyl cytosine.

In certain embodiments, ISIS 443139 is represented by the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

In certain embodiments, ISIS 443139 is represented by the following chemical structure:

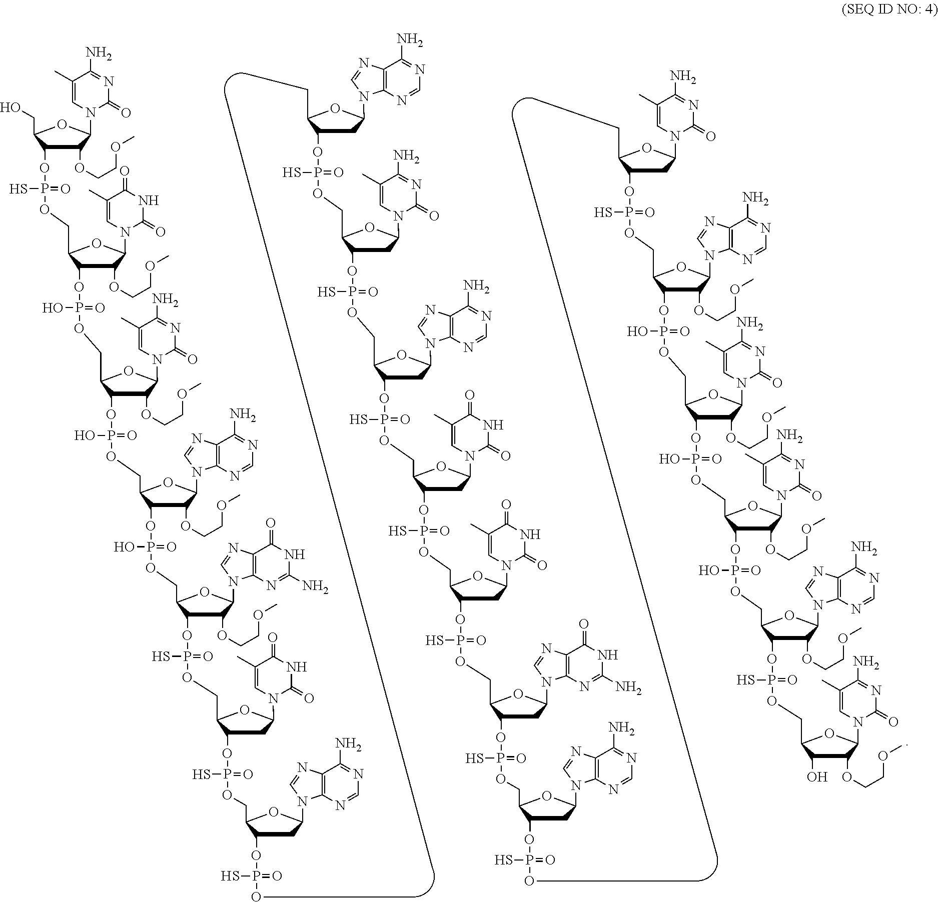

### Structure 1. ISIS 443139

In certain embodiments, the sodium salt of ISIS 443139 is represented by the following chemical structure:

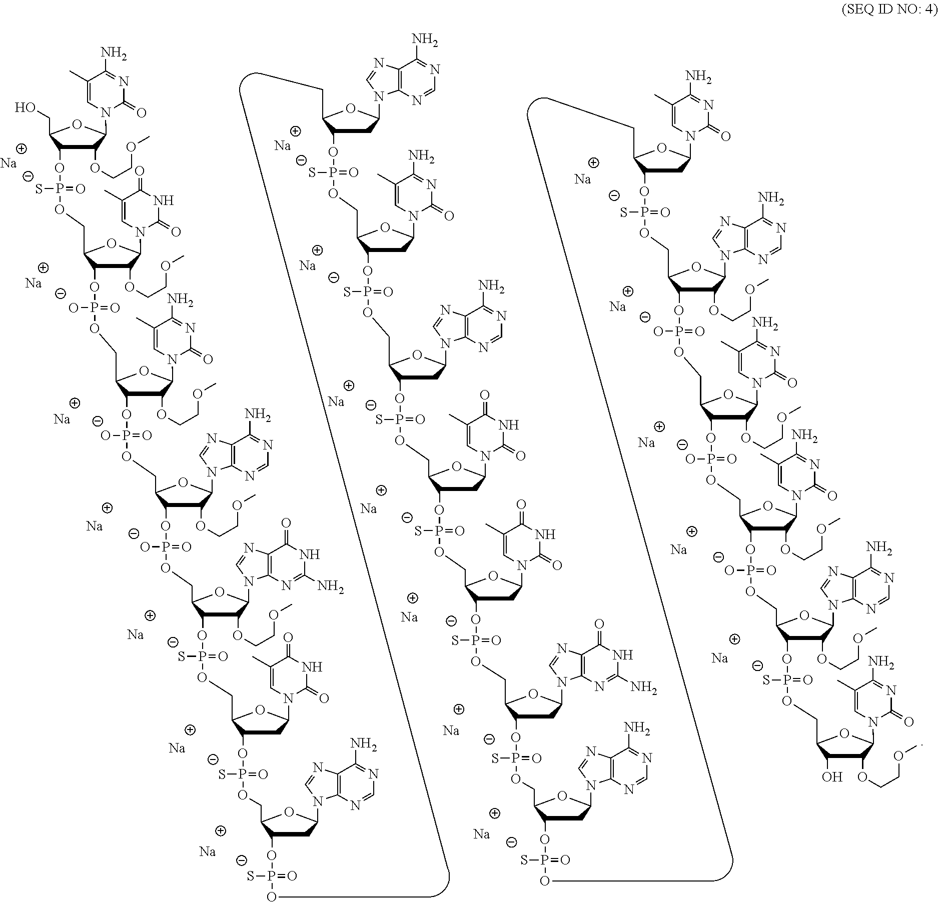

Structure 2. Sodium salt of ISIS 443139

### III. Certain Pharmaceutical Compositions

In certain embodiments, described herein are methods of administering to a subject a pharmaceutical composition comprising the modified oligonucleotide ISIS 443139. In certain embodiments, the pharmaceutical composition comprises a pharmaceutically acceptable diluent or carrier. In certain embodiments, the pharmaceutical composition comprises or consists essentially of a sterile saline solution and the modified oligonucleotide ISIS 443139. In certain embodiments, the sterile saline is pharmaceutical grade saline. In certain embodiments, the pharmaceutical composition comprises or consists essentially of sterile water and the modified oligonucleotide ISIS 443139. In certain embodiments, the sterile water is pharmaceutical grade water. In certain embodiments, the pharmaceutical composition comprises or consists essentially of artificial cerebrospinal fluid (aCSF) and the modified oligonucleotide ISIS 443139. In certain embodiments, the artificial cerebrospinal fluid is pharmaceutical grade.

In certain embodiments, pharmaceutical compositions comprise one or more excipients and the modified oligonucleotide ISIS 443139. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

In certain embodiments, pharmaceutical compositions comprising the modified oligonucleotide ISIS 443139 encompass any pharmaceutically acceptable salt of the modified oligonucleotide ISIS 443139, esters of the modified oligonucleotide ISIS 443139, or salts of such esters. In certain embodiments, pharmaceutical compositions comprising the modified oligonucleotide ISIS 443139 are capable of providing (directly or indirectly) the biologically active metabolite or residue thereof upon administration to a human subject. Accordingly, for example, the disclosure is also drawn to pharmaceutically acceptable salts of the modified oligonucleotide ISIS 443139, prodrugs of the modified oligonucleotide ISIS 443139, pharmaceutically acceptable salts of such prodrugs, and other bioequivalents. Suitable pharmaceutically acceptable salts include, but are not limited to, sodium and potassium salts.

In certain embodiments, pharmaceutical compositions comprise one or more lipid moieties and the modified oligonucleotide ISIS 443139. In certain embodiments, lipid moieties are used to increase distribution of ISIS 443139 to a particular cell or tissue. In certain such methods, the modified oligonucleotide ISIS 443139 is introduced into preformed liposomes or lipoplexes made of mixtures of cationic lipids and neutral lipids. In certain methods, DNA complexes with mono- or poly-cationic lipids are formed without the presence of a neutral lipid.

In certain embodiments, pharmaceutical compositions disclosed herein comprise a delivery system. Examples of delivery systems include, but are not limited to, liposomes and emulsions. Certain delivery systems are useful for preparing pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver modified oligonucleotides described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV)). In certain of such embodiments, a pharmaceutical composition comprises a carrier and is formulated in aqueous solution, such as aCSF, water, or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, the modified oligonucleotide ISIS 443139 acts as an acid. Although ISIS 443139 may be drawn or described in protonated (free acid) form, or ionized and in association with a cation (salt) form, aqueous solutions of ISIS 443139 exist in equilibrium among such forms. For example, a phosphate linkage of ISIS 443139 in aqueous solution exists in equilibrium among free acid, anion, and salt forms. Unless otherwise indicated, the term, “ISIS 443139,” is intended to include all such forms. Moreover, ISIS 443139 has several such linkages, each of which is in equilibrium. Thus, ISIS 443139 exists in solution in an ensemble of forms at multiple positions all at equilibrium. The term “ISIS 443139” is intended to include all such forms. Drawn structures necessarily depict a single form. Nevertheless, unless otherwise indicated, such drawings are likewise intended to include corresponding forms. Herein, a structure depicting the free acid of ISIS 443139 followed by the term “or a salt thereof” expressly includes all such forms that may be fully or partially protonated/de-protonated/in association with a cation. In certain instances, one or more specific cation is identified.

In certain embodiments, ISIS 443139 is in aqueous solution with sodium. In certain embodiments, ISIS 443139 is in aqueous solution with potassium. In certain embodiments, ISIS 443139 is in PBS. In certain embodiments, ISIS 443139 is in water. In certain such embodiments, the pH of the solution is adjusted with NaOH and/or HCl to achieve a desired pH.

Herein, certain specific doses are described. For clarity, a dose of ISIS 443139 in milligrams indicates the mass of the free acid form of ISIS 443139. As described above, in aqueous solution, the free acid is in equilibrium with anionic and salt forms. However, for the purpose of calculating dose, it is assumed that ISIS 443139 exists as a solvent-free, sodium-acetate free, anhydrous, free acid. For example, where ISIS 443139 is in solution comprising sodium (e.g., saline), ISIS 443139 may be partially or fully de-protonated and in association with Na+ ions. However, the mass of the protons is nevertheless counted toward the weight of the dose, and the mass of the Na+ ions are not counted toward the weight of the dose. Thus, for example, a dose of 120 mg of ISIS 443139 equals the number of fully protonated molecules that weighs 120 mg. This would be equivalent to 127 mg of solvent-free, sodium-acetate free, anhydrous sodiated ISIS 443139.

### IV. Certain Dosage Amounts

In certain embodiments, described herein are methods of administering to a subject a therapeutically effective amount of the modified oligonucleotide ISIS 443139. In certain embodiments, the therapeutically effective amount is 10 mg. In certain embodiments, the therapeutically effective amount is 30 mg. In certain embodiments, the therapeutically effective amount is 60 mg. In certain embodiments, the therapeutically effective amount is 90 mg. In certain embodiments, the therapeutically effective amount is 120 mg.

In certain embodiments, the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

In certain embodiments, the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, and about 300 mg.

In certain embodiments, the therapeutically effective amount is any of 115.0 mg, 115.1 mg, 115.2 mg, 115.3 mg, 115.4 mg, 115.5 mg, 115.6 mg, 115.7 mg, 115.8 mg, 115.9 mg, 116.0 mg, 116.1 mg, 116.2 mg, 116.3 mg, 116.4 mg, 116.5 mg, 116.6 mg, 116.7 mg, 116.8 mg, 116.9 mg, 117.0 mg, 117.1 mg, 117.2 mg, 117.3 mg, 117.4 mg, 117.5 mg, 117.6 mg, 117.7 mg, 117.8 mg, 117.9 mg, 118.0 mg, 118.1 mg, 118.2 mg, 118.3 mg, 118.4 mg, 118.5 mg, 118.6 mg, 118.7 mg, 118.8 mg, 118.9 mg, 119.0 mg, 119.1 mg, 119.2 mg, 119.3 mg, 119.4 mg, 119.5 mg, 119.6 mg, 119.7 mg, 119.8 mg, 119.9 mg, 120.0 mg, 120.1 mg, 120.2 mg, 120.3 mg, 120.4 mg, 120.5 mg, 120.6 mg, 120.7 mg, 120.8 mg, 120.9 mg, 121.0 mg, 121.1 mg, 121.2 mg, 121.3 mg, 121.4 mg, 121.5 mg, 121.6 mg, 121.7 mg, 121.8 mg, 121.9 mg, 122.0 mg, 122.1 mg, 122.2 mg, 122.3 mg, 122.4 mg, 122.5 mg, 122.6 mg, 122.7 mg, 122.8 mg, 122.9 mg, 123.0 mg, 123.1 mg, 123.2 mg, 123.3 mg, 123.4 mg, 123.5 mg, 123.6 mg, 123.7 mg, 123.8 mg, 123.9 mg, 124.0 mg, 124.1 mg, 124.2 mg, 124.3 mg, 124.4 mg, 124.5 mg, 124.6 mg, 124.7 mg, 124.8 mg, 124.9 mg, and 125.0 mg.

In certain embodiments, the therapeutically effective amount is any of about 115.0 mg, about 115.1 mg, about 115.2 mg, about 115.3 mg, about 115.4 mg, about 115.5 mg, about 115.6 mg, about 115.7 mg, about 115.8 mg, about 115.9 mg, about 116.0 mg, about 116.1 mg, about 116.2 mg, about 116.3 mg, about 116.4 mg, about 116.5 mg, about 116.6 mg, about 116.7 mg, about 116.8 mg, about 116.9 mg, about 117.0 mg, about 117.1 mg, about 117.2 mg, about 117.3 mg, about 117.4 mg, about 117.5 mg, about 117.6 mg, about 117.7 mg, about 117.8 mg, about 117.9 mg, about 118.0 mg, about 118.1 mg, about 118.2 mg, about 118.3 mg, 118.4 mg, about 118.5 mg, about 118.6 mg, about 118.7 mg, about 118.8 mg, about 118.9 mg, about 119.0 mg, about 119.1 mg, about 119.2 mg, about 119.3 mg, about 119.4 mg, about 119.5 mg, about 119.6 mg, about 119.7 mg, about 119.8 mg, about 119.9 mg, about 120.0 mg, about 120.1 mg, about 120.2 mg, about 120.3 mg, 120.4 mg, about 120.5 mg, about 120.6 mg, about 120.7 mg, about 120.8 mg, about 120.9 mg, about 121.0 mg, about 121.1 mg, about 121.2 mg, about 121.3 mg, about 121.4 mg, about 121.5 mg, about 121.6 mg, about 121.7 mg, about 121.8 mg, about 121.9 mg, about 122.0 mg, about 122.1 mg, about 122.2 mg, about 122.3 mg, 122.4 mg, about 122.5 mg, about 122.6 mg, about 122.7 mg, about 122.8 mg, about 122.9 mg, about 123.0 mg, about 123.1 mg, about 123.2 mg, about 123.3 mg, about 123.4 mg, about 123.5 mg, about 123.6 mg, about 123.7 mg, about 123.8 mg, about 123.9 mg, about 124.0 mg, about 124.1 mg, about 124.2 mg, about 124.3 mg, 124.4 mg, about 124.5 mg, about 124.6 mg, about 124.7 mg, about 124.8 mg, about 124.9 mg, and about 125.0 mg.

In certain embodiments, the therapeutically effective amount is any of 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

In certain embodiments, the therapeutically effective amount is any of less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

In certain embodiments, the therapeutically effective amount is any of less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

In certain embodiments, the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

In certain embodiments, the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

### V. Certain Dosing Regimens

In certain embodiments, described herein are methods of administering to a subject a therapeutically effective amount of the modified oligonucleotide ISIS 443139 one or more times. In certain embodiments, methods comprise administering the therapeutically effective amount at least 1, 2, 3, 4, 5, 6, 7, 8, 9 or 10 times. In certain embodiments, methods comprise administering the therapeutically effective amount once every 4 weeks. In certain embodiments, methods comprise administering the therapeutically effective amount once every 8 weeks. In certain embodiments, methods comprise administering the therapeutically effective amount once every 16 weeks.

In certain embodiments, methods comprise administering the therapeutically effective amount about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, or about every 20 weeks.

In certain embodiments, methods comprise administering the therapeutically effective amount for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

### Loading and Maintenance Doses

In certain embodiment, the therapeutically effective amount is administered as a loading dose and/or a maintenance dose. In certain embodiments, methods comprise administering a loading dose or doses and subsequently administering a maintenance dose or doses. In certain embodiments, methods comprise administering a loading dose once about every 4 weeks, and subsequently administering a maintenance dose once about every 8 weeks. In certain embodiments, methods comprise administering a loading dose once about every 4 weeks, and subsequently administering a maintenance dose once about every 16 weeks.

In certain embodiments, methods comprise administering at least 2 loading doses, at least 3 loading doses, at least 4 loading doses, at least 5 loading doses, or at least 6 loading doses. In certain embodiments, methods comprise administering 2, 3, 4, 5, 6, 7, 8, 9, 10, 11, 12, 13, 14, 15, or 16 loading doses. In certain embodiments, methods comprise administering a loading dose or doses about every 1 week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, or about every 12 weeks. In certain embodiments, methods comprise administering an initial loading dose and administering a second loading dose about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial loading dose.

In certain embodiments, methods comprise administering at least 2 maintenance doses, at least 3 maintenance doses, at least 4 maintenance doses, at least 5 maintenance doses, or at least 6 maintenance doses. In certain embodiments, methods comprise administering 2, 3, 4, 5, 6, 7, 8, 9, 10, 11, 12, 13, 14, 15, or 16 maintenance doses. In some instances, methods comprise administering a maintenance dose or doses about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, about every 16 weeks, about every 17 weeks, about every 18 weeks, about every 19 weeks, or about every 20 weeks. In certain embodiments, methods comprise administering a first maintenance dose and administering a second maintenance dose about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks after administering the first maintenance dose.

In certain embodiments, methods comprise administering a first maintenance dose or doses about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks after administering the last loading dose.

### VI. Potency and Efficacy

In certain embodiments, described herein are methods of reducing HTT RNA and/or HTT protein in a cell or biological fluid of a human subject, wherein the methods comprise administering a therapeutically effective amount of ISIS 443139 to the subject. In certain embodiments, methods reduce HTT RNA and/or HTT protein in the cerebrospinal fluid of the human subject. One may determine whether or not methods reduce HTT RNA and/or HTT protein, e.g., by detecting/quantifying a first amount of HTT RNA or HTT protein in a first biological sample obtained before administering and detecting/quantifying a second amount of HTT RNA or HTT protein in a second biological sample obtained after administering, and detecting or quantifying a reduction in HTT RNA or HTT protein by comparing the first amount to the second amount. In certain embodiments, the HTT RNA is mHTT RNA. In certain embodiments, the HTT protein is mHTT protein.

In certain embodiments, methods comprise reducing HTT RNA and/or HTT protein by 1-100%, or a range defined by any two of these values. In certain embodiments, methods comprise reducing HTT RNA and/or HTT protein by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. In certain embodiments, the HTT RNA is mHTT RNA. In certain embodiments, the HTT protein is mHTT protein.

In certain embodiments, methods comprise reducing HTT RNA or HTT protein by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%. In certain embodiments, the HTT RNA is mHTT RNA. In certain embodiments, the HTT protein is mHTT protein.

In certain embodiments, methods comprise reducing HTT RNA or HTT protein by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to 100%. In certain embodiments, the HTT RNA is mHTT RNA. In certain embodiments, the HTT protein is mHTT protein.

In certain embodiments, methods comprise administering ISIS 443139 to a subject and detecting or quantifying an amount of HTT RNA or HTT protein in a cell or a biological fluid of the subject. In certain embodiments, methods comprise detecting/quantifying a first amount of HTT RNA or HTT protein in a first biological sample obtained before administering and detecting/quantifying a second amount of HTT RNA or HTT protein in a second biological sample obtained after administering, and detecting or quantifying a reduction in HTT RNA or HTT protein by comparing the first amount to the second amount. In certain embodiments, the second biological sample is obtained less than about 24 hours after administering. In certain embodiments, the second biological sample is obtained less than about 1 week after administering. In certain embodiments, the second biological sample is obtained about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, or about 18 weeks after administering. In certain embodiments, methods comprise increasing or decreasing the dose after comparing the first amount to the second amount. In certain embodiments, methods comprise administering more frequently or less frequently after comparing the first amount to the second amount. In certain embodiments, the HTT RNA is mHTT RNA. In certain embodiments, the HTT protein is mHTT protein.

### Assessing Efficacy of ISIS 443139

In certain embodiments, methods described herein are sufficiently effective to ameliorate at least one symptom of HD in a human subject. In certain embodiments, the at least one symptom is impaired motor function. In certain embodiments, impaired motor function comprises restlessness, lack of coordination, unintentionally initiated motions, unintentionally uncompleted motions, unsteady gait, chorea, rigidity, writhing motions, abnormal posturing, instability, abnormal facial expressions, difficulty chewing, difficulty swallowing, difficulty speaking, seizure, sleep disturbances, or a combination thereof. In certain embodiments, the at least one symptom is impaired cognitive function. In certain embodiments, impaired cognitive function comprises impaired planning, impaired flexibility, impaired abstract thinking, impaired rule acquisition, impaired initiation of appropriate actions, impaired inhibition of inappropriate actions, impaired short-term memory, impaired long-term memory, or a combination thereof. In certain embodiments, the at least one symptom is a psychiatric symptom. In certain embodiments, the psychiatric symptom is selected from paranoia, disorientation, confusion, hallucination, dementia, anxiety, depression, blunted affect, egocentrisms, aggression, compulsive behavior, irritability, and suicidal ideation. In certain embodiments, the at least one symptom is reduced brain mass (brain atrophy), muscle atrophy, nerve degeneration, cardiac failure, impaired glucose tolerance, weight loss, osteoporosis, testicular atrophy, or a combination thereof.

In certain embodiments, methods described herein are sufficiently effective to ameliorate brain atrophy, reduced brain activity, or reduced brain activity in a human subject having HD relative to a healthy control subject. In certain embodiments, the healthy control subject is a subject that does not have HD. In certain embodiments, methods are sufficiently effective to ameliorate brain atrophy, reduced brain activity, or reduced brain activity as determined by performing an electroencephalography (EEG) or magnetic resonance imaging (MRI) on the human subject.

In certain embodiments, methods described herein are sufficiently effective to ameliorate at least one symptom of HD in a human subject as assessed by a clinically relevant test, score or scale. In certain embodiments, the at least one symptom is impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, and suicidal behavior, or a combination thereof. Non-limiting examples of such clinically relevant tests, scores and scales include the following.

### Total Functional Capacity Scale

In certain embodiments, methods described herein are sufficiently effective to ameliorate impaired global function of a subject having HD, as assessed by the Total Functional Capacity Scale (TFC). TFC is a validated measure of global function in HD described in greater detail by the Huntington Study Group, Mov. Disord. 1996; 11:136-42. The TFC represents the investigator's assessment of the subject's capacity to perform a range of activities of basic daily living, including working, chores, managing finances, eating, dressing, and bathing. The TFC score ranges from 0 to 13, with a higher score representing better functioning. A 1-point change in TFC score is a clinically meaningful change in subject function (e.g., a 1-point decline may indicate the loss of ability to work in a normal capacity). In certain embodiments, methods improve the TFC score by at least 1, 2, 3, 4, 5, 6, 7, 8, 9, or 10 points.

### Total Motor Score

In certain embodiments, methods described herein are sufficiently effective to ameliorate impaired motor function of a subject having HD, as assessed by the Total Motor Score (TMS). TMS is a holistic measure of motor function in HD that is linked to both functional capacity based on the TFC score, independence, and driving status (Beglinger et al., Mov. Disord. 2012; 27:1146-52; Schobel et al., Neurology 2017; 89:2495-2502). The TMS score is the sum of the individual motor ratings obtained from administration of the 31-item motor assessment portion of the UHDRS by an investigator. The score ranges from 0 to 124, with a higher score representing more severe impairment. In certain embodiments, methods reduce the TMS score by at least 1, 2, 3, 4, 5, 6, 7, 8, 9, or 10 points.

### Symbol Digit Modalities Test

In certain embodiments, methods described herein are sufficiently effective to ameliorate impaired attention, impaired visuoperceptual processing, impaired working memory, impaired psychomotor speed, or a combination thereof, in a subject having HD, as assessed by the Symbol Digit Modalities Test (SDMT). In SMDT, the subject pairs abstract symbols with specific numbers according to a translation key. The test measures the number of items correctly paired (maximum of 110 correct pairs) in 90 seconds. SDMT has been shown to have strong reliability and validity. SDMT is described in greater detail by Smith, A. Symbol Digit Modalities Test (SDMT). Manual (rev.) Los Angeles: Western Psychological Services, 1982. In certain embodiments, methods improve the number of items correctly paired by at least 1, 2, 3, 4, 5, 6, 7, 8, 9, or 10 items.

### Stroop Word Reading Test

In certain embodiments, methods described herein are sufficiently effective to ameliorate impaired attention, impaired processing, impaired psychomotor speed, impaired verbal motor output, or a combination thereof, in a subject having HD, as assessed by the Stroop Word Reading Test (SWR) Test. During a SWR Test, the subject is presented with a page of color names (i.e., “BLUE,” “RED,” or “GREEN”) printed in black ink and is asked to read aloud as many words as possible within a given amount of time (in 45 seconds). The number of words read correctly is counted, with a higher score indicating better cognitive performance. See Stroop, J. R., J. Exp. Psychol. 1935, 18, 643-662 for additional description of the SWR Test. In certain embodiments, methods improve the number of words the subject can read aloud in the given amount of time by at least 1, 2, 3, 4, 5, 6, 7, 8, 9, or 10 words.

Composite Unified Huntington's Disease Rating Scale (cUHDRS)

In certain embodiments, methods described herein are sufficiently effective to improve a subject's rating on the Composite Unified Huntington's Disease Rating Scale (cUHDRS). The cUHDRS assesses motor function, cognitive function, and global function. The outcome measure is comprised of an equally weighted sum of Z scores of the TFC, the TMS, the SDMT, and the SWR scores from the UHDRS. It is a multidomain measure of clinical decline that tracks underlying progressive brain changes and is related to changes in daily functional ability. The cUHDRS is described in greater detail by Schobel et al., Neurology 2017; 89:2495-2502. In certain embodiments, methods improve the subject's cUHDRS rating by at least 1, 2, 3, 4, 5, 6, 7, 8, 9, or 10 points.

### Unified Huntington's Disease Rating Scale (UHDRS) Functional Assessment

In certain embodiments, methods described herein are sufficiently effective to ameliorate impaired global function in a subject having HD, as assessed by the UHDRS Functional Assessment. The UHDRS Functional Assessment is a checklist of 25 common daily tasks. This checklist is described by Huntington Study Group, Mov. Disord. 1996; 11:136-42. An investigator indicates if the subject can perform a task by giving a score of 1 to all “yes” replies. The checklist is then summed, and scores can range from 0 (inability to do any task) to 25 (ability to do all tasks on the checklist). In certain embodiments, methods improve the subject's UHDRS Functional Assessment score by at least 1, 2, 3, 4, 5, 6, 7, 8, 9, 10, 11, 12, 13, 14, or 15.

### Independence Scale

In certain embodiments, methods described herein are sufficiently effective to ameliorate impaired global function in a subject having HD, as assessed by the Independence Scale (IS). The IS is a measure of disease progression in functional disability and degree of independence. It is a subscale of the UHDRS. The scale consists of 19 discrete levels ranging from 10 to 100 (by 5), in which a score of 100 indicates no special care is needed and a score of 10 indicates the subject is fed by tube and requires total bed care. The IS is described in greater detail by Huntington Study Group, Mov. Disord. 1996; 11:136-42. In certain embodiments, methods improve the subject's IS score by at least 1, 2, 3, 4, 5, 6, 7, 8, 9, 10, 15, 20, 25, 30, 35, 40, 45, or 50 points.

### Huntington's Disease Daily Activities Scale

In certain embodiments, methods described herein are sufficiently effective to improve a subject's score on the Huntington's Disease Daily Activities Scale (HD-DAS). HD-DAS assesses a subject's daily function. Following a semi-structured interview with the subject and/or subject companion, the subject's ability level to perform daily tasks such as eating or using a telephone will be recorded. Each item is scored on a 4-point Likert-type scale, where 0 indicates no impact and 3 indicates severe impact. The HD-DAS is described in greater detail by Bylsma et al., Mov. Disord. 1993; 8:183-90. In certain embodiments, methods improve the subject's HD-DAS score by 1, 2, or 3 points.

### Global Impression, Severity and Change Scales

In certain embodiments, methods described herein are sufficiently effective to improve a subject's score on a Global Impression, Severity and Change Scale. This assessment can be conducted by a clinician (CGI-S), a companion (CrGI-S), or the subject (PGI-S). The subject is assessed using an 11-point numeric rating scale (NRS), where higher scores indicate greater severity. The CGI-S is described in greater detail by Guy W: ECDEU Assessment Manual for Psychopharmacology Rockville, MD: U.S. Department of Health, Education, and Welfare; 1976. In certain embodiments, methods reduce the subject's NRS score by 1, 2, 3, 4, 5, 6, 7, 8, 9 or 10 points.

### Montreal Cognitive Assessment

In certain embodiments, methods described herein are sufficiently effective to ameliorate impaired cognitive function in a subject having HD, as assessed by the Montreal Cognitive Assessment (MoCA). The MoCA is a subject-completed assessment used to detect cognitive impairment. It contains a series of basic assessments, including attention and visuospatial tasks. The total score ranges from 0 to 30, where lower scores indicate greater impairment. The MoCA is described in greater detail by Nasreddine et al., J. Am. Geriatr. Soc. 2005 53:695-9. In certain embodiments, methods increase the subject's MoCA score by at least 1, 2, 3, 4, 5, 6, 7, 8, 9 or 10 points.

### Work Productivity and Activity Impairment Test

In certain embodiments, methods described herein are sufficiently effective to ameliorate impaired global function in a subject having HD, as assessed by the Work Productivity and Activity Impairment Test (WPAI). The WPAI contains 6 items assessing the impact of disease on employment status (yes/no), hours missed due to disease, hours missed due to other reasons, hours worked, and impact on productivity and on daily activities (both using an 11-point NRS, where higher scores indicate greater impact). The WPAI is described in greater detail by Reilly et al., Pharmacoeconomics 1993 4:353-65. In certain embodiments, methods reduce the subject's WPAI score by at least 1, 2, 3, 4, 5, 6, 7, 8, 9 or 10 points.

### Apathy Evaluation Scale

In certain embodiments, methods described herein are sufficiently effective to ameliorate impaired apathy in a subject having HD, as assessed by the Apathy Evaluation Scale (AES). The AES is an 18-item assessment of apathy, including overt behavior, cognitive aspects of motivation, and emotional responsivity. Each item is scored on a 4-point Likert scale, from 1 (“Not at all”) to 4 (“A lot”). A total score is created by summing the 18 items (scores range from 18 to 72; 3 items are reverse scored), with higher scores indicating greater apathy. The AES is described in greater detail by Marin et al., Psychiatry Res. 1991 38:143-62. In certain embodiments, methods increase the subject's AES score by at least 1, 2, 3, 4, 5, 6, 7, 8, 9 or 10 points.

### Neuro-Qol Cognition Function Short Form, Version 2

In certain embodiments, methods described herein are sufficiently effective to ameliorate impaired mental concentration and/or impaired learning ability in a subject having HD, as assessed by the Neuro-Qol Cognition Function Short Form. The Neuro-Qol Cognition Function Short Form contains 8 items (including “trouble concentrating” and difficulty “learning new tasks or instructions”), each assessed using a 5-point Likert scale, where lower scores indicate greater difficulty (4 items) or greater frequency (4 items). The raw sum score is converted to a T-score distribution (mean of 50, standard deviation of 10). See National Institute of Neurological Disorders and Stroke (NINDS). User Manual for the Quality of Life in Neurological Disorders (Neuro-QoL) Measures, Version 2.0, March 2015.

### Huntington's Disease Speaking Difficulty Item

In certain embodiments, methods described herein are sufficiently effective to ameliorate impaired speech in a subject having HD, as assessed by the Huntington's Disease Speaking Difficulty Item (HD-SDI) assessment. The HD-SDI includes a single question assessing difficulty speaking over 7 days. It is assessed using a 5-point Likert scale, where higher scores indicate a greater frequency of difficulty. In certain embodiments, methods reduce the subject's score by at least 1, 2, 3, 4, or 5 points.

### Symptoms of Major Depressive Disorder Scale

In certain embodiments, methods described herein are sufficiently effective to ameliorate depression in a subject having HD, as assessed by the Symptoms of Major Depressive Disorder Scale (SMDDS). SMDDS is a self-report assessment of depression (McCarrier et al., Patient 2016, 9:117-134). It contains 16 items, measuring concepts such as sadness, irritability, worry, and sleep disturbance. Each item is assessed on a 5-point Likert scale, from “Not at all” to “Extremely” (9 items) and from “Never” to “Always” (7 items). Item scores from 15 of the items (the least severe of the two eating items is not included) are summed to create a 0 to 60 score, where higher scores indicate more severe depressive symptomatology. The SMDDS is described in greater detail by Bushnell et al., Value in Health 2019 22:906-915. In certain embodiments, methods reduce the subject's score by at least 1, 2, 3, 4, 5, 6, 7, 8, 9 or 10 points.

### Huntington's Disease Companion-Reported Irritability and Angry Outbursts Scale

In certain embodiments, methods described herein are sufficiently effective to ameliorate irritability and anger in a subject having HD, as assessed by the Huntington's Disease Companion-Reported Irritability and Angry Outbursts Scale (HD-CIAOS). HD-CIAOS is a study companion-reported assessment of the subject's irritability and angry outbursts over 7 days. It consists of three items: frequency of irritable behavior (6-point Likert scale: “Not at all” to “Always”), frequency of angry outbursts (number of occurrences), and severity of the worst outburst (4-point Likert scale “Mild” to “Very severe”). In certain embodiments, methods reduce the subject's score by 1, 2, 3, or 4 points.

### EuroQol 5-Dimension, 5-Level Questionnaire

In certain embodiments, methods described herein are sufficiently effective to ameliorate impaired mobility, impaired self-care, impaired global function, pain/discomfort, anxiety, depression, or a combination thereof, in a subject having HD, as assessed by the EuroQol 5-Dimension, 5-Level Questionnaire (EQ-5D-5L). EQ-5D-5L is a validated self-report health status questionnaire used to calculate a health status utility score for use in health economic analyses (see Brooks Health Policy 1996 37:53-72 and Herdman et al. Qual Life Res. 2011, 20:1727-36). There are two components to the EQ-5D-5L: a 5-item health state profile that assesses mobility, self-care, usual activities, pain/discomfort, and anxiety/depression, as well as a visual analog scale (VAS) that measures health state. Published weighting systems allow for creation of a single composite score of the subject's health status (Index score) from the 5-item scores (i.e., does not include the VAS). In certain embodiments, methods improve the subject's EQ-5D-5L score.

### Health Utilities Index

In certain embodiments, methods described herein are sufficiently effective to improve the health status of a subject having HD, as assessed by the Health Utilities Index (HUI). The HUI is a multi-attribute system of health status (see Feeny et al., Pharmacoeconomics 1995, 7:490-502). The HUI2 and HUI3 questionnaire (commonly referred to as HUI2/3) contains 15 items with Likert-type response options. From these items, two scores can be produced: HUI2 (7 items) and HUI3 (8 items). Both scores are health utility indexes, where 0=death, and 1=perfect health. In certain embodiments, methods improve the subject's HUI score.

### Columbia-Suicide Severity Rating Scale

In certain embodiments, methods described herein are sufficiently effective to ameliorate suicidal ideation or suicidal behavior of a subject having HD, as assessed by the Columbia-Suicide Severity Rating Scale (C-SSRS). The C-SSRS is a structured tool to assess suicidal ideation and behavior. Four constructs are measured: severity of ideation, intensity of ideation, behavior, and lethality of actual suicide attempts. Binary (yes/no) data are collected for 10 categories, and composite endpoints based on the categories are followed over time to monitor subject safety (Posner et al. Am. J. Psychiatry 2011 168:1266-77). It maps to the Columbia-Classification Algorithm for Suicide Assessment and meets the criteria listed in the U.S. FDA draft guidance for assessment of suicidality in clinical trials (FDA 2012). In certain embodiments, methods improve the subject's C-SSRS score.

### VII. Certain Combination Therapies

In certain embodiments, methods comprise co-administering ISIS 443139 with at least one other pharmaceutical agent. In certain embodiments, the at least one other pharmaceutical agent ameliorates HD or a symptom thereof. In certain embodiments, ISIS 443139 is co-administered with the at least one other pharmaceutical agent to produce a combinational effect. In certain embodiments, ISIS 443139 is co-administered with the at least one other pharmaceutical agent to produce a synergistic effect.

In certain embodiments, ISIS 443139 and the at least one other pharmaceutical agent are administered at the same time. In certain embodiments, ISIS 443139 and the at least one other pharmaceutical agent are administered at different times. In certain embodiments, ISIS 443139 and the at least one other pharmaceutical agent are prepared together in a single formulation. In certain embodiments, ISIS 443139 and the at least one other pharmaceutical agent are administered are prepared separately.

In certain embodiments, pharmaceutical agents that may be co-administered with ISIS 443139 include antipsychotic agents, such as, e.g., haloperidol, chlorpromazine, clozapine, quetapine, and olanzapine; antidepressant agents, such as, e.g., fluoxetine, sertraline hydrochloride, venlafaxine and nortriptyline; tranquilizing agents such as, e.g., benzodiazepines, clonazepam, paroxetine, venlafaxin, and beta-blockers; mood-stabilizing agents such as, e.g., lithium, valproate, lamotrigine, and carbamazepine; paralytic agents such as, e.g., Botulinum toxin; and/or other experimental agents including, but not limited to, tetrabenazine (Xenazine), creatine, conezyme Q10, trehalose, docosahexanoic acids, ACR16, ethyl-EPA, atomoxetine, citalopram, dimebon, memantine, sodium phenylbutyrate, ramelteon, ursodiol, zyprexa, xenasine, tiapride, riluzole, amantadine, [123I]MNI-420, atomoxetine, tetrabenazine, digoxin, detromethorphan, warfarin, alprozam, ketoconazole, omeprazole, and minocycline.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Phase 1-2a Human Clinical Trial with ISIS 443139

A randomized, double-blind, multiple-ascending-dose, Phase 1-2a trial involving adult human subjects with HD was conducted. Table 1 shows the characteristics of human subjects at baseline. Human subjects had a total functional capacity score of 11-13 on the Unified Huntington's Disease Rating Scale, indicating little to no functional impairment.

TABLE 1
Characteristics of Human Subjects at Baseline*

| | | ISIS 443139 | | | | | |
|---|---|---|---|---|---|---|---|
| | Placebo | All | 10 mg | 30 mg | 60 mg | 90 mg | 120 mg |
| Characteristic | (n = 12) | (n = 34) | (n = 3) | (n = 6) | (n = 6) | (n = 9) | (n = 10) |
| Age (yr) | 49 ± 10 | 46 ± 10 | 44 ± 17 | 53 ± 7 | 43 ± 11 | 46 ± 10 | 45 ± 10 |
| Female (%) | 33 | 41 | 33 | 17 | 50 | 33 | 60 |
| No. of CAG | 44 ± 2 | 44 ± 3 | 46 ± 6 | 43 ± 2 | 45 ± 2 | 44 ± 3 | 45 ± 4 |
| repeats | | | | | | | |
| TFC score of 11 | 50 | 26 | 0 | 33 | 33 | 22 | 30 |
| (%) § | | | | | | | |
| TFC score of 12 | 33 | 44 | 33 | 67 | 50 | 44 | 30 |
| (%) § | | | | | | | |
| TFC score of 13 | 17 | 29 | 67 | 0 | 17 | 33 | 40 |
| (%) § | | | | | | | |
| Cognitive Score ‡ | 25 ± 2 | 26 ± 3 | 26 ± 4 | 27 ± 2 | 26 ± 3 | 26 ± 3 | 26 ± 3 |
| Total Motor Score ¶ | 24 ± 7 | 22 ± 10 | 21 ± 7 | 20 ± 13 | 25 ± 13 | 22 ± 10 | 21 ± 9 |
| Independence | 89 ± 8 | 90 ± 8 | 93 ± 6 | 88 ± 11 | 86 ± 8 | 93 ± 8 | 90 ± 6 |
| Scale Score † | | | | | | | |
| Disease Burden | 398.4 ± 50.1 | 383.7 ± 66.0 | 385.2 ± 109.1 | 366.7 ± 50.8 | 383.8 ± 34.3 | 364.5 ± 68.7 | 410.8 ± 75.1 |
| Score ** | | | | | | | |
| Concentration of | 109 ± 43 | 110 ± 46 | 144 ± 50 | 120 ± 45 | 117 ± 30 | 105 ± 65 | 96 ± 35 |
| mHTT in CSF | | | | | | | |
| (fmol/L) | | | | | | | |

*Plus-minus values are means ± SD. Patients were assigned to receive either placebo or ascending doses of ISIS 443139. Percentages may not total 100 because of rounding.
§ Total functional capacity score (TFC) on the Unified Huntington's Disease Rating Scale range from 0 to 13, with higher scores indicating less functional impairment. A score of 11 to 13 indicates little to no functional impairment across the items assessed (occupation, finances, domestic chores, activities of daily living, and care level).
‡ Scores on the Montreal Cognitive Assessment range from 0 to 30, with higher scores indicating better cognitive function.
¶ Total motor scores range from 0 to 124, with lower scores indicating less impairment.
† Independence scale scores range from 0 to 100, with higher scores indicating higher levels of independence.
** The disease-burden score is calculated as follows: (CAG repeat length − 35.5) × age in years. Larger numbers represent a higher burden of disease.

Human subjects were randomly assigned in a 3:1 ratio to receive ISIS 443139 or placebo as a bolus intrathecal administration every 4 weeks for four doses (Days 1, 29, 57 and 85). The primary endpoint was safety. The secondary endpoint was pharmacokinetics of ISIS 443139 in CSF. Prespecified exploratory endpoints included the concentrations of mHTT protein in CSF.

Human subjects received placebo or ISIS 443139 at ascending dose levels of 10 mg, 30 mg, 60 mg, 90 mg, or 120 mg. Each human subject received all four doses and completed the trial. All adverse events in human subjects receiving ISIS 443139 were mild (83%) or moderate (17%) in severity (e.g., procedural pain and post-dural-puncture headache). No serious adverse events were observed in human subjects receiving ISIS 443139. There were no clinically relevant adverse changes in laboratory variables.

CSF (20 mL) was obtained from patients using a standard lumbar puncture collection kit immediately prior to dosing on days 1, 29, 57, and 85 to obtain trough concentrations of CSF mHTT protein. CSF was similarly collected during the post treatment period on either Study Day 113 or Study Day 141. Human CSF mHTT protein was measured in triplicate using a human mHTT single molecule detection assay described by Wild et al., J. Clin. Invest. 2015, 125:1979-1986, using the MW1 anti-HTT polyQ antibody (catalog 03-0076-02; Singulex), described by Weiss et al., Anal Biochem. 2009, 395:8-15. In patients who received ISIS 443139, there were dose-dependent decreases in the CSF trough concentrations of mHTT protein at the last available 28-day post-dose sampling point, see Table 2, with a maximum reduction of 63% in an individual patient (in the 120-mg cohort). The 90 mg dose and 120 mg dose achieved approximately 40% mean reduction in CSF mHTT protein. Percent change was calculated from pre-dose Day 1 to the last available 28-day post-dose timepoint (latter of Day 85 and Day 113).

TABLE 2
Summary of percent change in CSF mHTT protein by dose group

| | | ISIS 443139 | | | | |
|---|---|---|---|---|---|---|
| | Placebo | 10 mg | 30 mg | 60 mg | 90 mg | 120 mg |
| | (n = 12) | (n = 3) | (n = 6) | (n = 6) | (n = 9) | (n = 10) |
| Mean (SD) | 9.8 (31.4) | −19.9 (12.7) | −25.0(13.1) | −27.5 (15.1) | −42.4 (13.0) | −37.7 (21.2) |
| Median | −1.7 | −25.9 | −20.3 | −30.6 | −43.9 | −41.8 |
| Interquartile | −10.3,29.2 | −28.5, −5.3 | −25.6, −18.3 | −32.8, −22.3 | −51.0, −36.7 | −43.6, −37.9 |
| range (IQR) | | | | | | |
| Min, Max | −27.0, 67.9 | −28.5, −5.3 | −50.7, −14.8 | −47.5, −1.6 | −58.4, −14.3 | −63.2, 18.5 |
| Difference | | −19.9 | −22.7 | −27.3 | −44.1 | −40.1 |
| (95% CI)* | | (−83.9, 3.6) | (−73.3, −8.6) | (−75.4, −10.5) | (−72.3, −30.0) | (−65.5, −30.0) |

*Hodges-Lehmann estimates of the difference (95 % Confidence Interval (CI)) between ISIS 443139 dose groups and the placebo group

In parallel with this trial, the composite Unified Huntington Disease Rating Scale (cUHDRS) was developed to serve as a measure of clinical progression in early HD. The relationships between the degree of lowering of the CSF concentration of mHTT protein and changes in the cUHDRS score and its four components were examined. Correlations between reduction in the CSF concentration of mHTT protein and improvements in the cUHDRS score and two of its components were observed.

### Example 2: Phase 2 Trial Human Clinical Trial with ISIS 443139 for HD (4 Week and 8 Week Results)

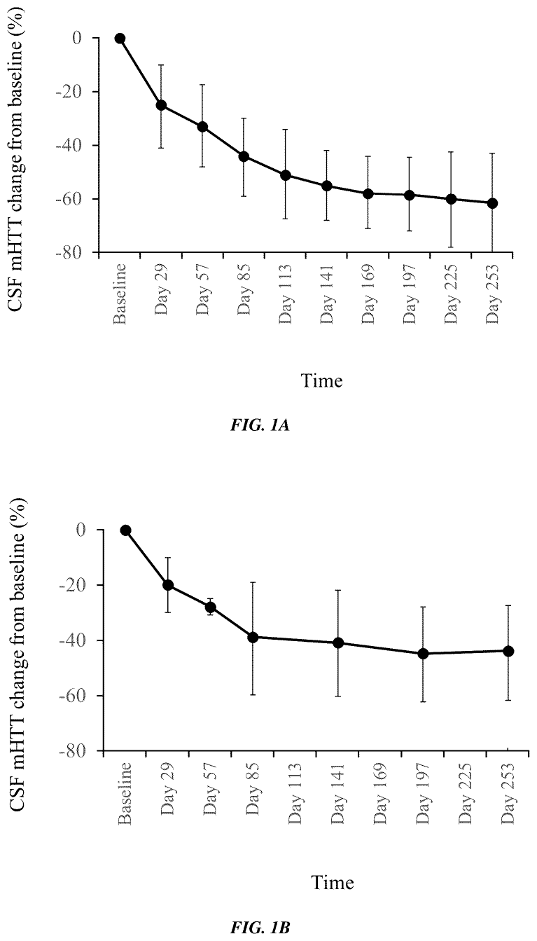

In an open label extension study following the Phase 1-2a trial described in Example 1, adult human subjects with HD received an initial loading dose of 120 mg on day 1, followed by a second dose loading dose of 120 mg on day 28. Thereafter, human subjects received maintenance doses of 120 mg of ISIS 443139 every 4 weeks (cohort Q4W), or every 8 weeks (cohort Q8W). All doses of ISIS 443139 were delivered via intrathecal administration. CSF samples were obtained and trough concentrations of CSF mHTT protein were analyzed as described in Example 1. CSF mHTT protein trough concentrations were used to calculate the mean reduction in CSF mHTT protein as a percentage of baseline, presented in FIG. 1A for cohort Q4W and FIG. 1B for cohort Q8W. As shown in FIG. 1A and FIG. 1B, ~30-50% CSF mHTT protein reduction was achieved with both regimens by day 85. In addition, CSF mHTT protein continued to decrease after day 85 in cohort Q4W. ISIS 443139 achieved 66% reduction in median trough concentration of CSF mHTT protein in cohort Q4W, and a 47% reduction in median trough concentration of CSF mHTT protein in cohort Q8W. Overall, ISIS 443139 was well tolerated in both cohorts.

### Example 3. EEG Activity as a Biomarker for ISIS 443139 Efficacy

Resting state EEGs of human subjects that participated in the Phase 1-2a trial described in Example 1 were obtained at screening, baseline, and Days 113, 141, and 197 post-treatment following intrathecal injection of four monthly doses of ISIS 443139. The 20 electrode B-Alert® X24 wireless EEG system by Advanced Brain Monitoring (Carlsbad, CA) placed according to the 10-20 system, was used for EEG data acquisitions. Resting-state EEG data were acquired during the study screening period for individuals with HD in alternating blocks of eyes open or closed, with four blocks per session (total recording time=20 minutes). For healthy controls (HC), two blocks were recorded (total recording time=10 minutes). Only eyes closed data were reported. Signals were referenced to linked mastoids and digitized at 256 Hz with a bandpass filter of 0.1-100 Hz. Offline, signals were filtered to 1-30 Hz and cleaned using visual inspection as well as unmixing noise signals from the recording, using independent component analysis (FastICA). Morlet wavelets (0.33 octaves) were used for frequency transforms of the time-resolved EEG signals. For between-group statistics, rank-sum tests and cluster-based permutation tests (electrode x frequency space), based on rank-sum test as a first level statistic, were used. All error bars represent bootstrap estimates of the 95% confidence interval.

Results showed that HD reduces EEG brain activity when compared with matched healthy controls. Results also showed that there was a significant increase in EEG signal power in patients treated with ISIS 443139 as compared with patients treated with placebo. Compared with baseline, ISIS 443139 treatment resulted in an increase in EEG signal power within the 4-8 Hz frequency range. The increase in brain activity with ISIS 443139 treatment was detectable across all dosing levels and was present throughout the duration of the post-treatment EEG monitoring. It was also observed that there was a positive association between the change from baseline in EEG activity and change in CSF mHTT concentration.

### Example 4: Phase 3 Human Clinical Trial with ISIS 443139 for HD

A randomized, double-blind, placebo-controlled Phase 3 clinical trial is conducted to evaluate the efficacy and safety of intrathecally administered ISIS 443139 in adult human subjects (25-65 years old) with manifest HD. Human subjects have a CAG-age product score (CAP score)>400. The CAP score is calculated by multiplying the human subject's age by (CAG repeat length-33.66). There are two arms of this study: a first cohort (Q8W) receives a loading dose of 120 mg ISIS 443139 on Day 1 and Day 28, followed by maintenance doses of 120 mg of ISIS 443139 every eight weeks; and a second cohort (Q16W) receives a loading dose of 120 mg ISIS 443139 on Day 1 and Day 28, followed by maintenance doses of 120 mg of ISIS 443139 every sixteen weeks. The primary efficacy endpoint will be change from baseline in the Total Functional Capacity (TFC) score at week 101. The secondary efficacy objective is change from baseline in the cUHDRS score at week 101. Change from baseline in Total Functional Capacity (TFC) score, Total Motor Score (TMS), Symbol Digit Modalities Test (SDMT) score, and Stroop Word Reading Test (SWR) score will also be measured.

1. A method of ameliorating Huntington's disease (HD) in a human subject in need thereof, the method comprising administering to the human subject a therapeutically effective amount of a modified oligonucleotide according to the following chemical structure:

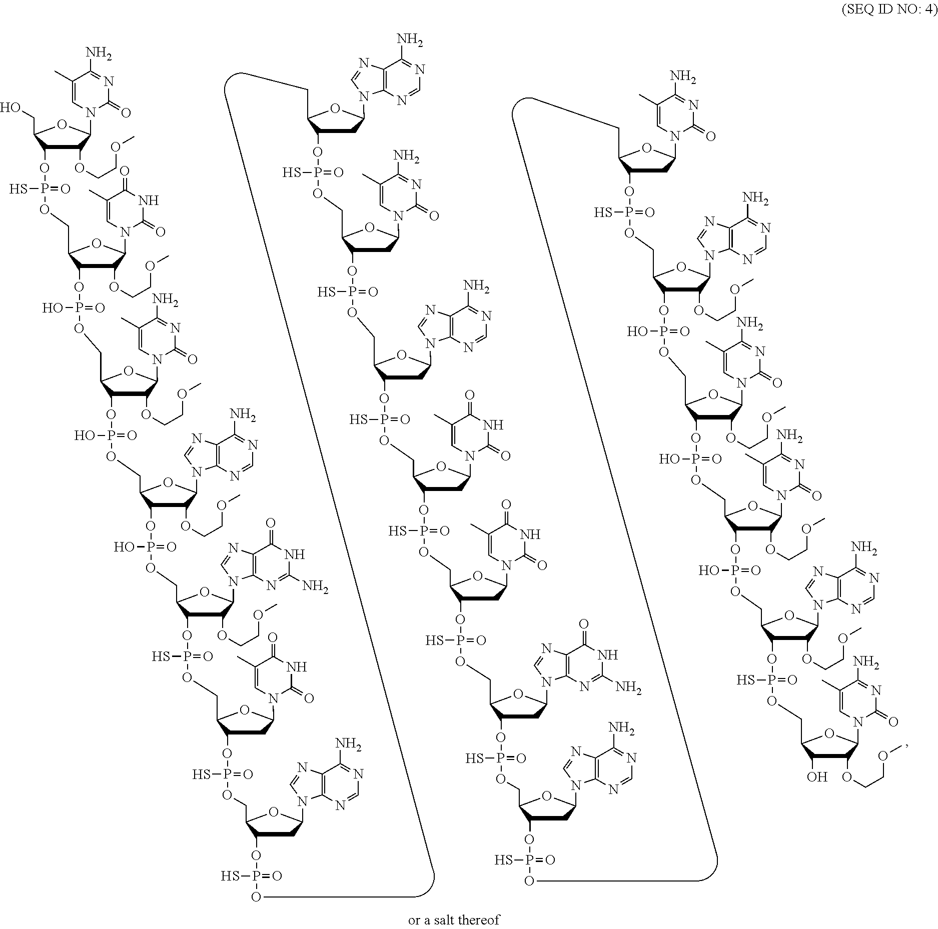

2. The method of claim 1, wherein the modified oligonucleotide is the sodium salt or the potassium salt.

3. A method of ameliorating HD in a human subject in need thereof, the method comprising administering to the human subject a therapeutically effective amount of a modified oligonucleotide according to the following chemical structure:

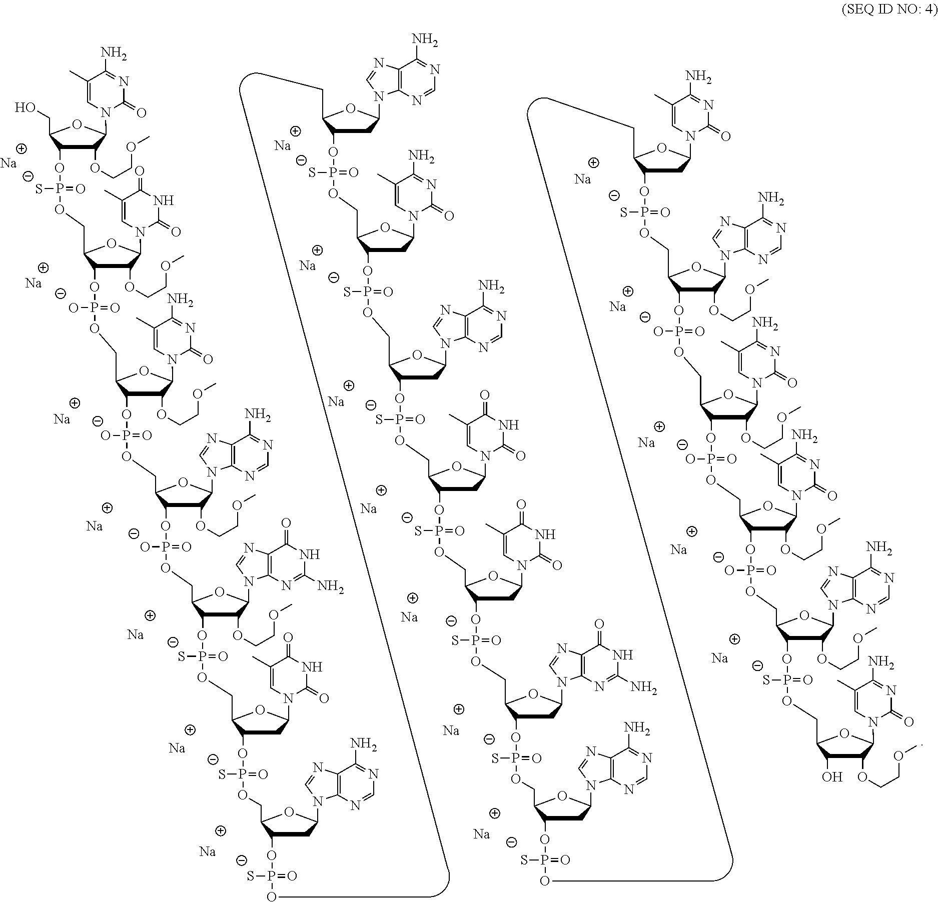

4. A method of ameliorating HD in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

5. The method of any one of claims 1-4, wherein at least one symptom of HD is ameliorated.

6. The method of claim 5, wherein the at least one symptom comprises brain atrophy, reduced brain activity, reduced brain connectivity, muscle atrophy, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

7. A method of reducing HTT RNA in a human subject in need thereof, the method comprising administering to the human subject a therapeutically effective amount of a modified oligonucleotide according to the following chemical structure:

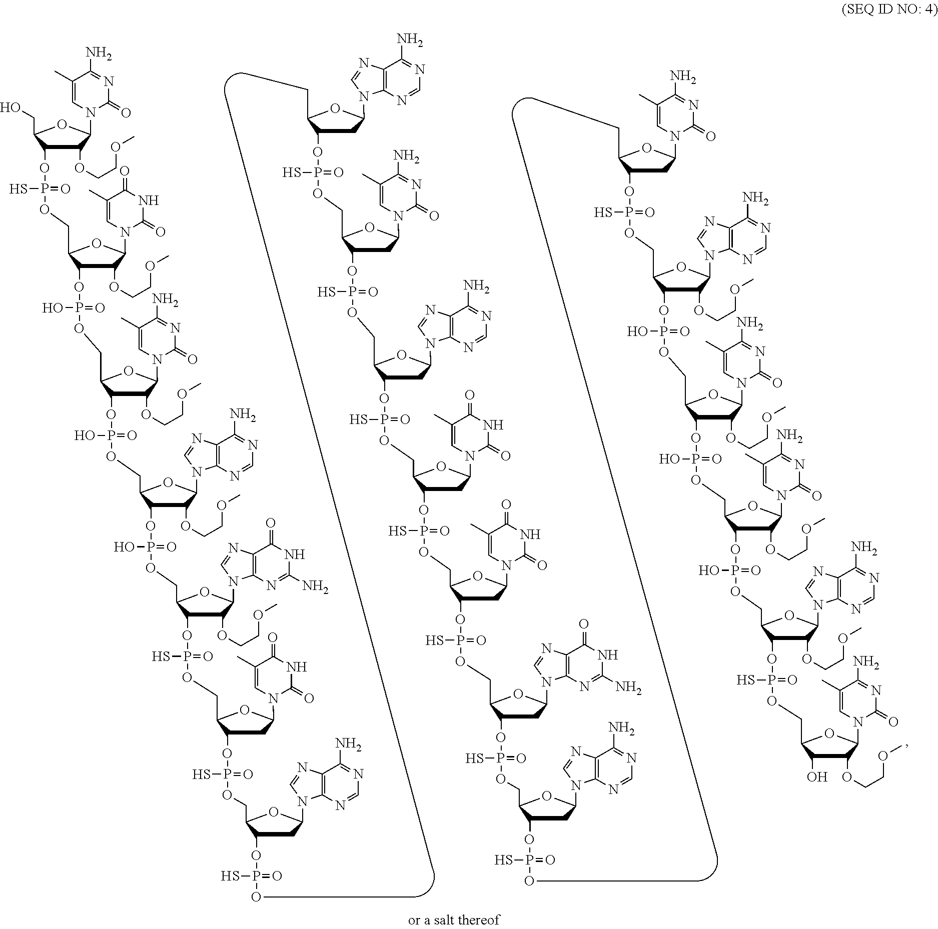

8. The method of claim 7, wherein the modified oligonucleotide is the sodium salt or the potassium salt.

9. A method of reducing HTT RNA in a human subject in need thereof, the method comprising administering to the human subject a therapeutically effective amount of a modified oligonucleotide according to the following chemical structure:

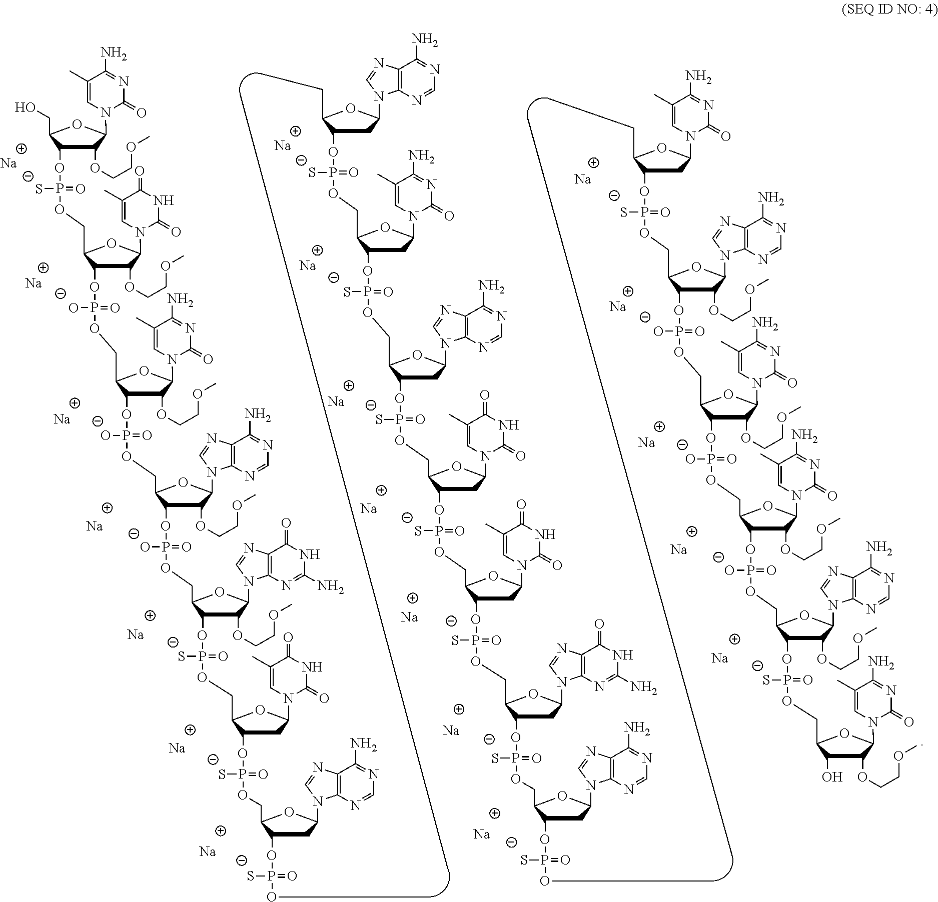

10. A method of reducing HTT RNA in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

11. A method of reducing HTT protein in a human subject in need thereof, the method comprising administering to the human subject a therapeutically effective amount of a modified oligonucleotide according to the following chemical structure:

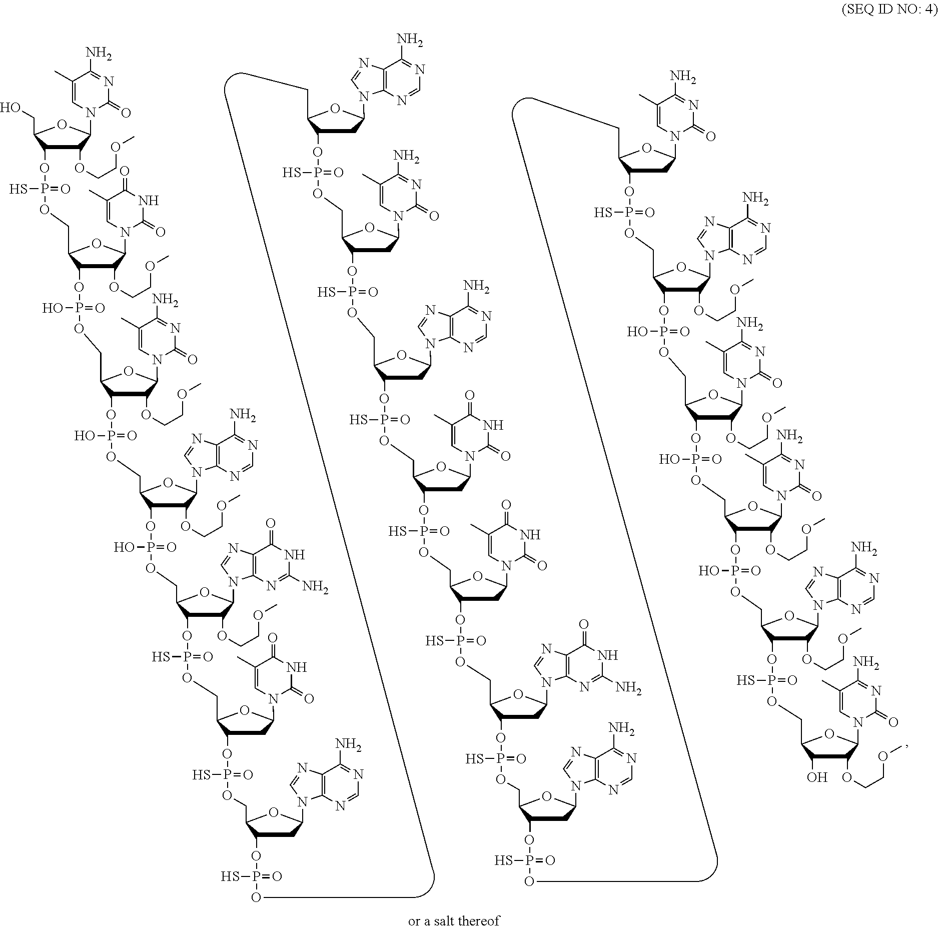

12. The method of claim 11, wherein the modified oligonucleotide is the sodium salt or the potassium salt.

13. A method of reducing HTT protein in a human subject in need thereof, the method comprising administering to the human subject a therapeutically effective amount of a modified oligonucleotide according to the following chemical structure:

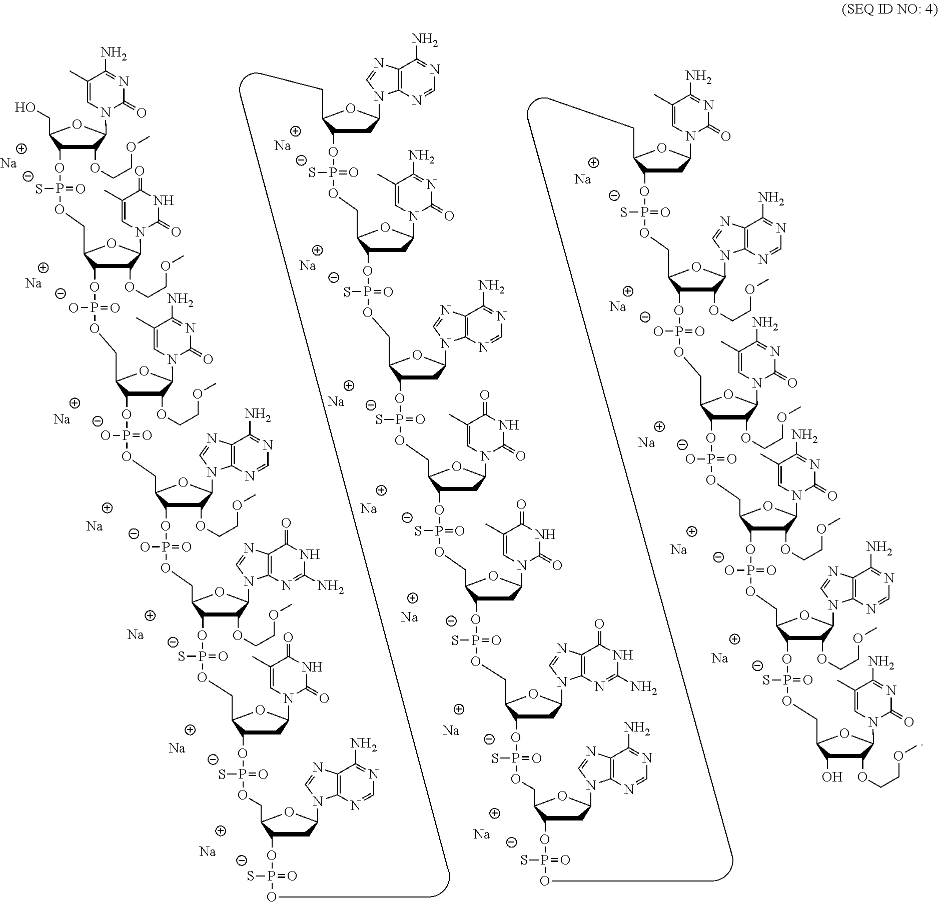

14. A method of reducing HTT protein in a human subject in need thereof, the method comprising administering to the human subject a therapeutically effective amount of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

15. The method of any one of claims 1-14, wherein the therapeutically effective amount is 10 mg.

16. The method of any one of claims 1-14, wherein the therapeutically effective amount is 30 mg.

17. The method of any one of claims 1-14, wherein the therapeutically effective amount is 60 mg.

18. The method of any one of claims 1-14, wherein the therapeutically effective amount is 90 mg.

19. The method of any one of claims 1-14, wherein the therapeutically effective amount is 120 mg.

20. The method of any one of claims 1-14, wherein the therapeutically effective amount is about 10 mg.

21. The method of any one of claims 1-14, wherein the therapeutically effective amount is about 30 mg.

22. The method of any one of claims 1-14, wherein the therapeutically effective amount is about 60 mg.

23. The method of any one of claims 1-14, wherein the therapeutically effective amount is about 90 mg.

24. The method of any one of claims 1-14, wherein the therapeutically effective amount is about 120 mg.

25. The method of any one of claims 1-14, wherein the therapeutically effective amount is any of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, and 300 mg.

26. The method of any one of claims 1-14, wherein the therapeutically effective amount is any of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, and about 300 mg.

27. The method of any one of claims 1-14, wherein the therapeutically effective amount is any of 115.0 mg, 115.1 mg, 115.2 mg, 115.3 mg, 115.4 mg, 115.5 mg, 115.6 mg, 115.7 mg, 115.8 mg, 115.9 mg, 116.0 mg, 116.1 mg, 116.2 mg, 116.3 mg, 116.4 mg, 116.5 mg, 116.6 mg, 116.7 mg, 116.8 mg, 116.9 mg, 117.0 mg, 117.1 mg, 117.2 mg, 117.3 mg, 117.4 mg, 117.5 mg, 117.6 mg, 117.7 mg, 117.8 mg, 117.9 mg, 118.0 mg, 118.1 mg, 118.2 mg, 118.3 mg, 118.4 mg, 118.5 mg, 118.6 mg, 118.7 mg, 118.8 mg, 118.9 mg, 119.0 mg, 119.1 mg, 119.2 mg, 119.3 mg, 119.4 mg, 119.5 mg, 119.6 mg, 119.7 mg, 119.8 mg, 119.9 mg, 120.0 mg, 120.1 mg, 120.2 mg, 120.3 mg, 120.4 mg, 120.5 mg, 120.6 mg, 120.7 mg, 120.8 mg, 120.9 mg, 121.0 mg, 121.1 mg, 121.2 mg, 121.3 mg, 121.4 mg, 121.5 mg, 121.6 mg, 121.7 mg, 121.8 mg, 121.9 mg, 122.0 mg, 122.1 mg, 122.2 mg, 122.3 mg, 122.4 mg, 122.5 mg, 122.6 mg, 122.7 mg, 122.8 mg, 122.9 mg, 123.0 mg, 123.1 mg, 123.2 mg, 123.3 mg, 123.4 mg, 123.5 mg, 123.6 mg, 123.7 mg, 123.8 mg, 123.9 mg, 124.0 mg, 124.1 mg, 124.2 mg, 124.3 mg, 124.4 mg, 124.5 mg, 124.6 mg, 124.7 mg, 124.8 mg, 124.9 mg, and 125.0 mg.

28. The method of any one of claims 1-14, wherein the therapeutically effective amount is any of about 115.0 mg, about 115.1 mg, about 115.2 mg, about 115.3 mg, about 115.4 mg, about 115.5 mg, about 115.6 mg, about 115.7 mg, about 115.8 mg, about 115.9 mg, about 116.0 mg, about 116.1 mg, about 116.2 mg, about 116.3 mg, about 116.4 mg, about 116.5 mg, about 116.6 mg, about 116.7 mg, about 116.8 mg, about 116.9 mg, about 117.0 mg, about 117.1 mg, about 117.2 mg, about 117.3 mg, about 117.4 mg, about 117.5 mg, about 117.6 mg, about 117.7 mg, about 117.8 mg, about 117.9 mg, about 118.0 mg, about 118.1 mg, about 118.2 mg, about 118.3 mg, 118.4 mg, about 118.5 mg, about 118.6 mg, about 118.7 mg, about 118.8 mg, about 118.9 mg, about 119.0 mg, about 119.1 mg, about 119.2 mg, about 119.3 mg, about 119.4 mg, about 119.5 mg, about 119.6 mg, about 119.7 mg, about 119.8 mg, about 119.9 mg, about 120.0 mg, about 120.1 mg, about 120.2 mg, about 120.3 mg, 120.4 mg, about 120.5 mg, about 120.6 mg, about 120.7 mg, about 120.8 mg, about 120.9 mg, about 121.0 mg, about 121.1 mg, about 121.2 mg, about 121.3 mg, about 121.4 mg, about 121.5 mg, about 121.6 mg, about 121.7 mg, about 121.8 mg, about 121.9 mg, about 122.0 mg, about 122.1 mg, about 122.2 mg, about 122.3 mg, 122.4 mg, about 122.5 mg, about 122.6 mg, about 122.7 mg, about 122.8 mg, about 122.9 mg, about 123.0 mg, about 123.1 mg, about 123.2 mg, about 123.3 mg, about 123.4 mg, about 123.5 mg, about 123.6 mg, about 123.7 mg, about 123.8 mg, about 123.9 mg, about 124.0 mg, about 124.1 mg, about 124.2 mg, about 124.3 mg, 124.4 mg, about 124.5 mg, about 124.6 mg, about 124.7 mg, about 124.8 mg, about 124.9 mg, and about 125.0 mg.

29. The method of any one of claims 1-14, wherein the therapeutically effective amount is within the range of any of 40 mg to 200 mg, 40 mg to 190 mg, 40 mg to 180 mg, 40 mg to 170 mg, from 40 mg to 160 mg, 40 mg to 150 mg, 40 mg to 140 mg, 40 mg to 120 mg, 40 mg to 110 mg, 40 mg to 100 mg, 40 mg to 80 mg, 40 mg to 70 mg, 40 mg to 60 mg, 40 mg to 50 mg, 50 mg to 200 mg, 50 mg to 190 mg, 50 mg to 180 mg, 50 mg to 170 mg, 50 mg to 160 mg, 50 mg to 150 mg, 50 mg to 140 mg, 50 mg to 120 mg, 50 mg to 110 mg, 50 mg to 100 mg, 50 mg to 80 mg, 50 mg to 70 mg, 50 mg to 60 mg, 60 mg to 200 mg, 60 mg to 190 mg, 60 mg to 180 mg, 60 mg to 170 mg, 60 mg to 160 mg, 60 mg to 150 mg, 60 mg to 140 mg, 60 mg to 120 mg, 60 mg to 110 mg, 60 mg to 100 mg, 60 mg to 80 mg, 60 mg to 70 mg, 70 mg to 200 mg, 70 mg to 190 mg, 70 mg to 180 mg, 70 mg to 170 mg, 70 mg to 160 mg, 70 mg to 150 mg, 70 mg to 140 mg, 70 mg to 120 mg, 70 mg to 110 mg, 70 mg to 100 mg, 70 mg to 80 mg, 80 mg to 200 mg, 80 mg to 190 mg, 80 mg to 180 mg, 80 mg to 170 mg, 80 mg to 160 mg, 80 mg to 150 mg, 80 mg to 140 mg, 80 mg to 120 mg, 80 mg to 110 mg, 80 mg to 100 mg, 80 mg to 90 mg, 90 mg to 200 mg, 90 mg to 190 mg, 90 mg to 180 mg, 90 mg to 170 mg, 90 mg to 160 mg, 90 mg to 150 mg, 90 mg to 140 mg, 90 mg to 120 mg, 90 mg to 110 mg, 90 mg to 100 mg, 100 mg to 200 mg, 100 mg to 190 mg, 100 mg to 180 mg, 100 mg to 170 mg, 100 mg to 160 mg, 100 mg to 150 mg, 100 mg to 140 mg, 100 mg to 120 mg, 100 mg to 110 mg, 110 mg to 200 mg, 110 mg to 190 mg, 110 mg to 180 mg, 110 mg to 170 mg, 110 mg to 160 mg, 110 mg to 150 mg, 110 mg to 140 mg, 110 mg to 130 mg, 110 mg to 120 mg, 120 mg to 200 mg, 120 mg to 190 mg, 120 mg to 180 mg, 120 mg to 170 mg, 120 mg to 160 mg, 120 mg to 150 mg, 120 mg to 140 mg, 120 mg to 130 mg, 130 mg to 200 mg, 130 mg to 190 mg, 130 mg to 180 mg, 130 mg to 170 mg, 130 mg to 160 mg, 130 mg to 150 mg, 130 mg to 140 mg, 140 mg to 200 mg, 140 mg to 190 mg, 140 mg to 180 mg, 140 mg to 170 mg, 140 mg to 160 mg, 140 mg to 150 mg, 150 mg to 200 mg, 150 mg to 190 mg, 150 mg to 180 mg, 150 mg to 170 mg, 150 mg to 160 mg, 160 mg to 200 mg, 160 mg to 190 mg, 160 mg to 180 mg, 160 mg to 170 mg, 180 mg to 200 mg, 180 mg to 190 mg, 190 mg to 200 mg, 105 mg to 135 mg, 105 mg to 130 mg, 105 mg to 125 mg 105 mg to 120 mg, 110 mg to 135 mg, 110 mg to 130 mg, 110 mg to 125 mg, 110 mg to 120 mg, 115 mg to 135 mg, 115 mg to 130 mg, 115 mg to 125 mg, 115 mg to 120 mg, 115 mg to 125 mg, 115 mg to 120 mg, 120 mg to 135 mg, 120 mg to 125 mg, 125 mg to 140 mg, 125 mg to 130 mg, 130 mg to 135 mg, 135 mg to 140 mg, 120 mg to 129 mg, 120 mg to 128 mg, 120 mg to 127 mg, 120 mg to 86 mg, 120 mg to 124 mg, 120 mg to 123 mg, 120 mg to 122 mg, 120 mg to 121 mg, 121 mg to 130 mg, 122 mg to 129 mg, 122 mg to 128 mg, 122 mg to 127 mg, 122 mg to 126 mg, 122 mg to 125 mg, 122 mg to 124 mg, 122 mg to 123 mg, 123 mg to 130 mg, 123 mg to 129 mg, 123 mg to 128 mg, 123 mg to 127 mg, 123 mg to 126 mg, 123 mg to 125 mg, 123 mg to 124 mg, 124 mg to 130 mg, 124 mg to 129 mg, 124 mg to 128 mg, 124 mg to 127 mg, 124 mg to 126 mg, 124 mg to 125 mg, 125 mg to 129 mg, 125 mg to 128 mg, 125 mg to 127 mg, 125 mg to 126 mg, 126 mg to 130 mg, 126 mg to 129 mg, 126 mg to 128 mg, 126 mg to 127 mg, 127 mg to 130 mg, 127 mg to 129 mg, 127 mg to 128 mg, 128 mg to 130 mg, 128 mg to 129 mg, and 129 mg to 130 mg.

30. The method of any one of claims 1-14, wherein the therapeutically effective amount is any of less than 300 mg, less than 295 mg, less than 290 mg, less than 285 mg, less than 280 mg, less than 275 mg, less than 270 mg, less than 265 mg, less than 260 mg, less than 255 mg, less than 250 mg, less than 245 mg, less than 240 mg, less than 235 mg, less than 230 mg, less than 225 mg, less than 220 mg, less than 215 mg, less than 210 mg, less than 205 mg, less than 200 mg, less than 195 mg, less than 190 mg, less than 185 mg, less than 180 mg, less than 175 mg, less than 170 mg, less than 165 mg, less than 160 mg, less than 150 mg, less than 145 mg, less than 140 mg, less than 135 mg, less than 130 mg, less than 125 mg, less than 120 mg, less than 115 mg, less than 110 mg, less than 105 mg, less than 100 mg, less than 95 mg, less than 90 mg, less than 85 mg, less than 80 mg, less than 75 mg, less than 70 mg, less than 65 mg, less than 60 mg, less than 55 mg, less than 50 mg, less than 45 mg, less than 40 mg, less than 35 mg, less than 30 mg, less than 25 mg, less than 20 mg, less than 15 mg, less than 10 mg, and less than 5 mg.

31. The method of any one of claims 1-14, wherein the therapeutically effective amount is any of less than about 300 mg, less than about 295 mg, less than about 290 mg, less than about 285 mg, less than about 280 mg, less than about 275 mg, less than about 270 mg, less than about 265 mg, less than about 260 mg, less than about 255 mg, less than about 250 mg, less than about 245 mg, less than about 240 mg, less than about 235 mg, less than about 230 mg, less than about 225 mg, less than about 220 mg, less than about 215 mg, less than about 210 mg, less than about 205 mg, less than about 200 mg, less than about 195 mg, less than about 190 mg, less than about 185 mg, less than about 180 mg, less than about 175 mg, less than about 170 mg, less than about 165 mg, less than about 160 mg, less than about 150 mg, less than about 145 mg, less than about 140 mg, less than about 135 mg, less than about 130 mg, less than about 125 mg, less than about 120 mg, less than about 115 mg, less than about 110 mg, less than about 105 mg, less than about 100 mg, less than about 95 mg, less than about 90 mg, less than about 85 mg, less than about 80 mg, less than about 75 mg, less than about 70 mg, less than about 65 mg, less than about 60 mg, less than about 55 mg, less than about 50 mg, less than about 45 mg, less than about 40 mg, less than about 35 mg, less than about 30 mg, less than about 25 mg, less than about 20 mg, less than about 15 mg, less than about 10 mg, and less than about 5 mg.

32. The method of any one of claims 1-14, wherein the therapeutically effective amount is any of at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least about 100 mg, at least 105 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185, at least 190 mg, at least 195 mg, and at least 200 mg.

33. The method of any one of claims 1-14, wherein the therapeutically effective amount is any of at least about 5 mg, at least about 10 mg, at least about 15 mg, at least about 20 mg, at least about 25 mg, at least about 30 mg, at least about 35 mg, at least about 40 mg, at least about 45 mg, at least about 50 mg, at least about 55 mg, at least about 60 mg, at least about 65 mg, at least about 70 mg, at least about 75 mg, at least about 80 mg, at least about 85 mg, at least about 90 mg, at least about 95 mg, at least about 100 mg, at least about 105 mg, at least about 115 mg, at least about 120 mg, at least about 125 mg, at least about 130 mg, at least about 135 mg, at least about 140 mg, at least about 145 mg, or at least about 150 mg, at least about 155 mg, at least about 160 mg, at least about 165 mg, at least about 170 mg, at least about 175 mg, at least about 180 mg, at least about 185, at least about 190 mg, at least about 195 mg, and at least about 200 mg.

34. The method of any one of claims 1-33, comprising administering the modified oligonucleotide once every 4 weeks.

35. The method of any one of claims 1-33, comprising administering the modified oligonucleotide once every 8 weeks.

36. The method of any one of claims 1-33, comprising administering the modified oligonucleotide once every 12 weeks.

37. The method of any one of claims 1-33, comprising administering the modified oligonucleotide once every 16 weeks.

38. The method of any one of claims 1-33, comprising administering the modified oligonucleotide once every 20 weeks.

39. The method of any one of claims 1-33, comprising administering the modified oligonucleotide about once every 4 weeks.

40. The method of any one of claims 1-33, comprising administering the modified oligonucleotide about once every 8 weeks.

41. The method of any one of claims 1-33, comprising administering the modified oligonucleotide about once every 12 weeks.

42. The method of any one of claims 1-33, comprising administering the modified oligonucleotide about once every 16 weeks.

43. The method of any one of claims 1-33, comprising administering the modified oligonucleotide about once every 20 weeks.

44. The method of any one of claims 1-33, comprising administering the modified oligonucleotide any of once every 1 week, once every 2 weeks, once every 3 weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, and once every 20 weeks.

45. The method of any one of claims 1-33, comprising administering the modified oligonucleotide any of once about every 1 week, once about every 2 weeks, once about every 3 weeks, once about every 4 weeks, once about every 5 weeks, once about every 6 weeks, once about every 7 weeks, once about every 8 weeks, once about every 9 weeks, once about every 10 weeks, once about every 11 weeks, once about every 12 weeks, once about every 13 weeks, once about every 14 weeks, once about every 15 weeks, once about every 16 weeks, once about every 17 weeks, once about every 18 weeks, once about every 19 weeks, and once about every 20 weeks.

46. The method of any of claims 1-33, comprising administering to the human subject an initial loading dose of 120 mg of the modified oligonucleotide.

47. The method of claim 46, comprising administering to the human subject a second loading dose of 120 mg of the modified oligonucleotide 4 weeks after the initial loading dose.

48. The method of claim 47, comprising administering to the human subject a maintenance dose of 120 mg of the modified oligonucleotide 4 weeks after the second loading dose.

49. The method of claim 47, comprising administering to the human subject a maintenance dose of 120 mg of the modified oligonucleotide 8 weeks after the second loading dose.

50. The method of claim 47, comprising administering to the human subject a maintenance dose of 120 mg of the modified oligonucleotide 12 weeks after the second loading dose.

51. The method of claim 47, comprising administering to the human subject a maintenance dose of 120 mg of the modified oligonucleotide 16 weeks after the second loading dose.

52. The method of any of claims 7-10 and 15-51, wherein the HTT RNA is mHTT RNA.

53. The method of any of claim 11-51, wherein the HTT protein is mHTT protein.

54. A method of ameliorating HD, reducing HTT RNA, reducing HTT protein, reducing mHTT RNA, or reducing mHTT protein in a human subject in need thereof, the method comprising intrathecally administering to the human subject a therapeutically effective amount of 120 mg or about 120 mg of a modified oligonucleotide according to the following chemical structure:

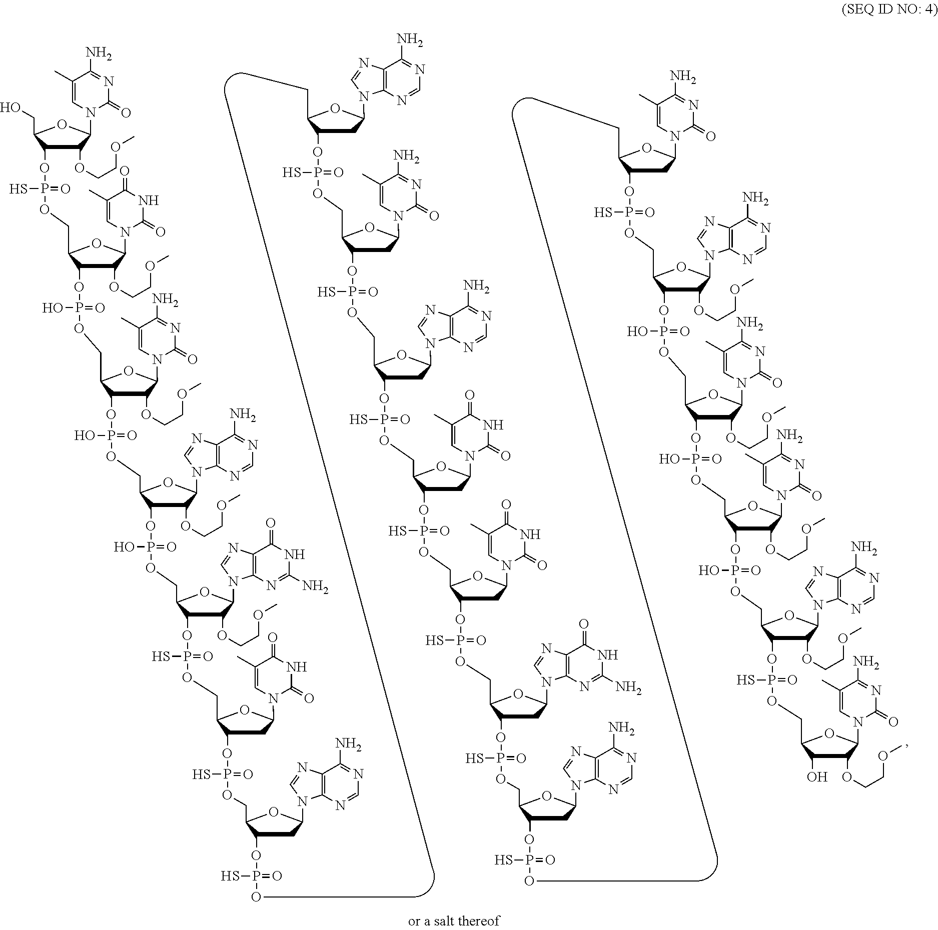

55. The method of claim 54, wherein the modified oligonucleotide is the sodium salt or the potassium salt.

56. A method of ameliorating HD, reducing HTT RNA, reducing HTT protein, reducing mHTT RNA, or reducing mHTT protein in a human subject in need thereof, the method comprising intrathecally administering to the human subject a therapeutically effective amount of 120 mg or about 120 mg of a modified oligonucleotide according to the following chemical structure:

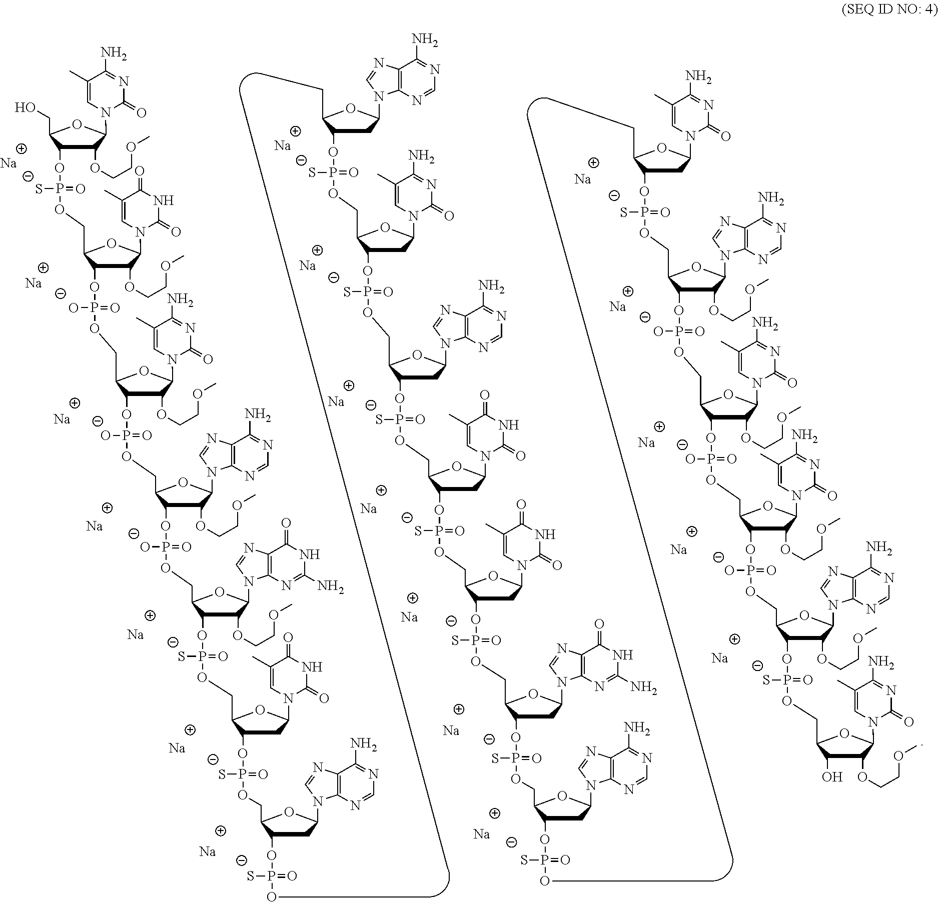

57. A method of ameliorating HD, reducing HTT RNA, reducing HTT protein, reducing HTT mRNA, or reducing mHTT protein in a human subject in need thereof, the method comprising intrathecally administering to the human subject a therapeutically effective amount of 120 mg or about 120 mg of a modified oligonucleotide, wherein the modified oligonucleotide has the following chemical notation (5′ to 3′): mCes Teo mCeo Aeo Ges Tds Ads Ads mCds Ads Tds Tds Gds Ads mCds Aeo mCeo mCeo Aes mCe (SEQ ID NO: 4); wherein,
A=an adenine nucleobase,
mC=a 5-methyl cytosine nucleobase,
G=a guanine nucleobase,
T=a thymine nucleobase,
e=a 2′-MOE sugar moiety,
d=a 2′-β-D-deoxyribosyl sugar moiety,
s=a phosphorothioate internucleoside linkage, and
o=a phosphodiester internucleoside linkage.

58. The method of any one of claims 54-57, comprising administering the modified oligonucleotide about once every 4 weeks.

59. The method of any one of claims 54-57, comprising administering the modified oligonucleotide about once every 8 weeks.

60. The method of any one of claims 54-57, comprising administering the modified oligonucleotide about once every 16 weeks.

61. The method of any of claims 54-57, comprising administering to the human subject:
a) an initial loading dose of about 120 mg of the modified oligonucleotide,
b) a second loading dose of about 120 mg of the modified oligonucleotide about 4 weeks after administering the initial loading dose;
c) a first maintenance dose of about 120 mg of the modified oligonucleotide about 8 weeks after administering the second loading dose;
d) a second maintenance dose of about 120 mg of the modified oligonucleotide about 8 weeks after administering the first maintenance dose.

62. The method of any of claims 54-57, comprising administering to the human subject:
a) an initial loading dose of about 120 mg of the modified oligonucleotide,
b) a second loading dose of about 120 mg of the modified oligonucleotide about 4 weeks after administering the initial loading dose;
c) a first maintenance dose of about 120 mg of the modified oligonucleotide about 16 weeks after administering the second loading dose;
d) a second maintenance dose of about 120 mg of the modified oligonucleotide about 16 weeks after administering the first maintenance dose.

63. The method of any one of claims 54-62, wherein at least one symptom of HD is ameliorated.

64. The method of claim 63, wherein the at least one symptom comprises brain atrophy, reduced brain activity, reduced brain connectivity, muscle atrophy, nerve degeneration, cardiac failure, impaired glucose tolerance, weight loss, osteoporosis, testicular atrophy, impaired global function, impaired motor function, impaired cognitive function, impaired daily function, impaired attention, impaired visuoperceptual processing, impaired working memory, impaired psychomotor speed, impaired verbal motor output, impaired degree of independence, impaired apathy, impaired learning ability, impaired mental concentration, impaired speech, depression, irritability, anger, impaired mobility, impaired self-care, pain, discomfort, anxiety, suicidal ideation, suicidal behavior, or a combination thereof.

65. The method of any of claims 1-64, wherein the human subject has a mutation in at least one IT15 gene.

66. The method of any of claims 1-65, comprising identifying a mutation in at least one IT15 gene of the human subject.

67. The method of claim 65 or claim 66, wherein the at least one IT15 gene has any of at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, or at least 60 contiguous CAG repeats.

68. The method of claim 65 or claim 66, wherein the at least one IT15 gene has 27 to 35 contiguous CAG repeats.

69. The method of claim 65 or claim 66, wherein the at least one IT15 gene has 35 to 60 contiguous CAG repeats.

70. The method of claim 65 or claim 66, wherein the at least one IT15 gene has greater than 60 contiguous CAG repeats.

71. The method of any of claims 1-70, wherein the modified oligonucleotide is administered to the CNS of the human subject.

72. The method of any of claims 1-71, wherein the modified oligonucleotide is administered by intrathecal administration.

73. The method of any of claims 1-72, wherein the modified oligonucleotide is administered by bolus intrathecal administration.

74. The method of any of claims 1-73, wherein HTT RNA is reduced.

75. The method of any of claims 1-74, wherein HTT protein is reduced.

76. The method of any of claims 1-75, wherein mHTT RNA is reduced.

77. The method of any of claims 1-76, wherein mHTT protein is reduced.

78. The method of any one of claims 1-77, comprising detecting an amount of mHTT RNA in a biological sample from the human subject.

79. The method of any one of claims 1-78, comprising detecting an amount of mHTT protein in a biological sample from the human subject.

80. The method of any one of claims 1-79, wherein the biological sample comprises cerebrospinal fluid.

81. The method of any of claims 78-80, wherein the detecting occurs before the administering.

82. The method of any of claims 78-80, wherein the detecting occurs after the administering.

83. The method of any of claims 78-80, wherein the detecting occurs before and after the administering.

84. The method of any one of claims 78-83, comprising adjusting the initial loading dose, the loading dose, maintenance dose, or therapeutically effective amount administered after detecting the amount of HTT RNA, HTT protein, mHTT RNA, mHTT protein, or combination thereof.

85. The method of any one of claims 1-84, comprising analyzing brain activity, brain size, or a combination thereof of the subject by performing an electroencephalogram (EEG) or magnetic resonance imaging (MRI) on the subject.

86. The method of claim 85, wherein performing the EEG or MRI occurs before administering, after administering, or a combination thereof.

87. The method of claim 86, comprising determining or adjusting the therapeutically effective amount after performing the EEG or MRI.

88. The method of claim 87, comprising performing the EEG or MRI after administering, and adjusting the frequency of administering after performing the EEG or MRI.

89. The method of any one of claims 85-88, wherein performing the EEG or MRI is performed within 1, 2, 4, 6, 8, 12 or 24 hours of administering.

90. The method of any one of claims 85-89, comprising performing the EEG before administering, and analyzing after administering, detecting less than a 4 Hz increase in EEG signal power from a first EEG to a second EEG, and subsequently increasing the frequency of administering the therapeutically effective amount of the modified oligonucleotide.

91. The method of claim 90, comprising administering a loading dose once about every 4 weeks and administering a maintenance dose once about every 8 or 16 weeks before recording the first EEG, and administering the maintenance dose less than about every 8 weeks or less than about every 16 weeks.

92. The method of any one of claims, 85-91, comprising recording a first EEG before administering, and recording a second EEG after administering, detecting less than a 4 Hz increase in EEG signal power from the first EEG to the second EEG, and subsequently administering a dose of the modified oligonucleotide that is greater than the therapeutically effective amount.

93. The method of claim 92, wherein the dose is at least about 10%, at least about 20%, at least about 30%, at least about 40%, at least about 50%, at least about 60%, at least about 70%, at least about 90%, or at least about 100% greater than the therapeutically effective amount.

94. The method of any one of claims 85-93, wherein the therapeutically effective amount is about 120 mg or 120 mg.


SEQUENCE LISTING (4 sequences)

SEQ ID NO: 1 - DNA, 175000 bp, Homo sapiens
  source 1..175000: mol_type = genomic DNA
aacaaatcaccccagcactgtggcttggcagcacacatgtctagtcatagagttatatgtagttacgtgtagagccatatgtatcgtcacacgttctgtgggtcaggaatttggacccagcttaaccagctccacttctcgccagggttcagtcaaataccagctgcctcccacctgagagctcagccggggaagggtccctttccaatctcacgtggtgttggcaggatccagttcctcatggcctgctggactgagaacctcagttctcactgcctgttggccagaggccgcctttatgtcctcgccatgtgggcctctccaacatggcagctgacttcatcagagcatccatgccaagaaggcaacagagagggccagggagactgaagtcatacccttttgcgacctagtcatggggtgacattccatcacctttgcccattggttagaagcaggccaccaggtacagcccaagctcacggggaggggtcatacaagggtgtcaataccaggaggtgaggggtgctggggccatcttatgagtctgcccactgaggtaactaacaaccttgaggcctgacacagtggacaaaggcccttattaacagcagagaactgggaactttatttatttatttatttttgagacagagtctcactcttgtcacccaggctggagtgcaatggcatgatcttggctcactgcaacctccacctcccaggttcaagcaattctgcctcagcctccggaatagctgggactacaggcatgcaccactacacccggctaatttttgtatttttagtagagacagggtttcgccatgttggccaggctggtctcgaactcctgacctctggtgatctgcctgccttggcctcccaaagtgctgggattacaggcgtgagccaccgcacctcgctggaacttaatttttttagagacagtgtcgctctatcacccaagctggagtgcagtggtgcaatcctagctcacttgcagcctcaaattcctgggttcaggtgatcctcccacatcagcctcccaagaactgggaactaacagctgtttctctgctgtccttctcaagaaaagggaggctactgctaccccactggggacaatgctgggtttccctttaggacaggctctgagacaaggcggaggtgctgtttgtggccacagagcaggggactctgggttgcaggtgtggcctggctaaagtaggctttactgggctcctctctgcctgcatcaccccccggctgggcggttgtctctgaggccaaccttactccctgctgggcaggctggacagctgccctctccgtttgcccctctaccacccaaaaggcaggaggctctggagaccaggaccctgcccgccacggcctgtgtcccaggcgtgagggggtgccccacagacctctgctgagctgctgctgaatgacgccccttgggggtcctgccggaaggtcagagcaggggtgcactcccataaagaaacgcccccaggtcgggactcattcctgtgggcggcatcttgtggccatagctgcttctcgctgcactaatcacagtgcctctgtgggcagcaggcgctgaccacccaggcctgccccagaccctctcctcccttccggggcgctgcgctgggaccgatggggggcgccaggcctgtggacaccgccctgcaggggcctctccagctcactgggggtggggtgggggtcacacttggggtcctcaggtcgtgccgaccacgcgcattctctgcgctctgcgcaggagctcgcccaccctctccccgtgcagagagccccgcagctggctccccgcagggctgtccgggtgagtatggctctggccacgggccagtgtggcgggagggcaaaccccaaggccacctcggctcagagtccacggccggctgtcgccccgctccaggcgtcggcgggggatcctttccgcatgggcctgcgcccgcgctcggcgccccctccacggccccgccccgtccatggccccgtccttcatgggcgagcccctccatggccctgcccctccgcgccccacccctccctcgccccacctctcaccttcctgccccgcccccagcctccccacccctcaccggccagtcccctcccctatcccgctccgcccctcagccgccccgcccctcagccggcctgcctaatgtccccgtccccagcatcgccccgccccgcccccgtctcgccccgcccctcaggcggcctccctgctgtgccccgccccggcctcgccacgcccctacctcaccacgccccccgcatcgccacgccccccgcatcgccacgcctcccttaccatgcagtcccgccccgtcccttcctcgtcccgcctcgccgcgacacttcacacacagcttcgcctcaccccattacagtctcaccacgccccgtcccctctccgttgagccccgcgccttcgcccgggtggggcgctgcgctgtcagcggccttgctgtgtgaggcagaacctgcgggggcaggggcgggctggttccctggccagccattggcagagtccgcaggctagggctgtcaatcatgctggccggcgtggccccgcctccgccggcgcggccccgcctccgccggcgcagcgtctgggacgcaaggcgccgtgggggctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccgtgagtttgggcccgctgcagctccctgtcccggcgggtcccaggctacggcggggatggcggtaaccctgcagcctgcgggccggcgacacgaacccccggccccgcagagacagagtgacccagcaacccagagcccatgagggacacccgccccctcctggggcgaggccttcccccacttcagccccgctccctcacttgggtcttcccttgtcctctcgcgaggggaggcagagccttgttggggcctgtcctgaattcaccgaggggagtcacggcctcagccctctcgcccttcgcaggatgcgaagagttggggcgagaacttgtttctttttatttgcgagaaaccagggcgggggttcttttaactgcgttgtgaagagaacttggaggagccgagatttgctcagtgccacttccctcttctagtctgagagggaagagggctgggggcgcgggacacttcgagaggaggcggggtttggagctggagagatgtgggggcagtggatgacataatgcttttaggacgcctcggcgggagtggcggggcagggggggggcggggagtgagggcgcgtccaatgggagatttcttttcctagtggcacttaaaacagcctgagatttgaggctcttcctacattgtcaggacatttcatttagttcatgatcacggtggtagtaacacgattttaagcaccacctaagagatctgctcatctaagcctaagttggtctgcaggcgtttgaatgagttgtggttgccaagtaaagtggtgaacttacgtggtgattaatgaaattatcttaaatattaggaagagttgattgaagttttttgcctatgtgtgttgggaataaaaccaacacgttgctgatggggaggttaattgccgagggatgaatgaggtgtacattttaccagtattccagtcaggcttgccagaatacggggggtccgcagactccgtgggcatctcagatgtgccagtgaaagggtttctgtttgcttcattgctgacagcttgttactttttggaagctaggggtttctgttgcttgttcttggggagaatttttgaaacaggaaaagagagaccattaaaacatctagcggaaccccaggactttccctggaagtctgtgtgtcgagtgtacagtaggagttaggaagtactctggtgcagttcaggcctttctcttacctctcagtattctatttccgatctggatgtgtcccagatggcatttggtaagaatatctctgttaagactgattaatttttagtaatatttcttgttctttgtttctgttatgatccttgtctcgtcttcaaagtttaattagaaaatgattcggagagcagtgttagcttatttgttggaataaaatttaggaataaattattctaaaggatggaaaaactttttggatatttggagaaattttaaaacaatttggcttatctcttcagtaagtaatttctcatccagaaatttactgtagtgcttttctaggaggtaggtgtcataaaagttcacacattgcatgtatcttgtgtaaacactaaacagggctcctgatgggaaggaagacctttctgctgggctgcttcagacacttgatcattctaaaaatatgccttctctttcttatgctgatttgacagaacctgcatttgcttatcttcaaaatatgggtatcaagaaatttcctttgctgccttgacaaaggagatagattttgtttcattactttaaggtaatatatgattaccttatttaaaaaatttaatcaggactggcaaggtggcttacacctttaatccgagcactttgggaggcctaggtggacgaatcacctgaggtcaggagtttgagaccagcctggctaacatggtgaaaccctgtctctactaaaaatacaaaaattagctggtcatggtggcacgtgcctgtaatccaagctacctgggaggctgaggcaggaaaatcgcttgaacccgggaggcagagtctgcagtgagttgagatcacgccactgcactccagcctgggtgacagagcgagactctatctcaaaaaaaattttttttaatgtattatttttgcataagtaatacattgacatgatacaaattctgtaattacaaaagggcaataattaaaatatcttccttccacccctttcctctgagtacctaactttgtccccaagaacaagcactatttcagttcctcatgtatcctgccagatataacctgttcatattgtaagatagatttaaaatgctctaaaaacaaaagtagtttagaataatatatatctatatattttttgagatgtagtctcacattgtcacccaggctggagtgcagtgatacaatctcggctcactgcagtctctgcctcccaggttcaaatgcttctcctgcctcagccttctgagtagctgggattacaggcgcccaccaccatgtccagctaatttttgtatttttagtagagatggggtttcaccatgttggccaggctggtcttgaactcctgaccttgtgatctgtccacctcggcctcccaaagtgctgggattacaggtgtgagccaccatgcctggctagaataataacttttaaaggttcttagcatgctctgaaatcaactgcattaggtttatttatagttttatagttattttaaataaaatgcatatttgtcatatttctctgtattttgctgttgagaaaggaggtattcactaattttgagtaacaaacactgctcacaaagtttggattttggcagttctgttcacgtgcttcagccaaaaaatcctcttctcaaagtaagattgatgaaagcaatttagaaagtatctgttctgtttttatggctcttgctctttggtgtggaactgtggtgtcacgccatgcatgggcctcagtttatgagtgtttgtgctctgctcagcatacaggatgcaggagttccttatggggctggctgcaggctcagcaaatctagcatgcttgggagggtcctcacagtaattaggaggcaattaatacttgcttctggcagtttcttattctccttcagattcctatctggtgtttccctgactttattcattcatcagtaaatatttactaaacatgtactatgtgcctggcactgttataggtgcagggctcagcagtgagcagacaaagctctgccctcgtgaagctttcattctaatgaaggacatagacagtaagcaagatagataagtaaaatatacagtacgttaatacgtggaggaacttcaaagcagggaaggggatagggaaatgtcagggttaatcgagtgttaacttatttttatttttaaaaaaattgttaagggctttccagcaaaacccagaaagcctgctagacaaattccaaaagagctgtagcactaagtgttgacatttttattttattttgttttgttttgttttttttgagacagttcttgctctatcagccaggctggagtgcactagtgtgatcttggctcactgcaacctctgcctcttgggttcaagtgattctcatgcctcagcctcctgtttagctgggattatagacatgcactgccatgcctgggtaattttttttttttcccccgagacggagtcttgctctgtcgcccaggctggagtgcagtggcgcgatctcagctcactgcaagctccgcttcccgagttcacgccattctcctgcctcagtctcccaagtagctgggactacaggcgcctgccaccacgtccagctaatttttttgtatttttaatagagacggggtttcaccgtgttagccaggatgatcttgatctcctgacctcgtcatccgccgaccttgtgatccgcccacctcggcctcccaaagtgctgggattacaggcatgagccactgtgcccggccacgcctgggtaatttttgtatttttagtagagatggggttttgccatgatgagcaggctggtctcgaactcccggcctcatgtgatctgcctgccttggcctcccaaagtgctaggattacaggcatgagccaccatacctggccagtgttgatattttaaatacggtgttcagggaaggtccactgagaagacagctttttttttttttttttttggggttggggggcaaggtcttgctctttaacccaggctggaatgcagtatcactatcgtagctcacttcagccttgaactcctgggctcaagtgatcctcccacctcaacctcacaatgtgttgggactataggtgtgagccatcacacctggccagatgatggcttttgagtaaagacctcaagcgagttaagagtctagtgtaagggtgtatgaagtagtggtattccagatggggggaacaggtccaaaatcttcctgtttcaggaatagcaaggatgtcattttagttgggtgaattgagtgagggggacatttgtagtaagaagtaaggtccaagaggtcaagggagtgccatatcagaccaatactacttgccttgtagatggaataaagatattggcatttatgtgagtgagatgggatgtcactggaggattagagcagaggagtagcatgatctgaatttcaatcttaagtgaactctggctgacaacagagtgaaggggaacaccggcaaaagcagaaaccagttaggaagccactgcagtgctcagataagcatggtgggttctgtcagggtaccggctgtcggctgtgggcagtgtgaggaatgactgactggattttgaatgcggaaccaactgcacttgttgaactctgctaagtataacaatttagcagtagcttgcgttatcaggtttgtattcagctgcaagtaacagaaaatcctgctgcaatagcttaaactggtaacaagcaagagcttatcagaagacaaaaataagtctggggaaattcaacaataagttaaggaacccaggctctttcttttttttttttttgaaacggagtttcgctcttgtcacccgggctggagtgcaatgatgtgatctcagctcactaaaacctctacctcctgggttcaagtgattcttctgcctcagcctcccaagtaactgggattacaggcgtataccaccatgcccagctaatttttgtgtttttagtagagatggggtttcaccatgttggccaggctggtctcgaacttctgacctcaggtgatccactcgcctcagcctgccaaagtgctgggattacaggtttgggccactgcacccggtcagaacccaggctctttcttatacttaccttgcaaacccttgttctcattttttccctttgtatttttattgttgaattgtaatagttctttatatattctggatactggattcttatcagatagatgatttgtaaaaactctcccttcctttggattgtctttttactttcttgatagtgtcttttgaagtgtaaaagtttttaattttgatgaagtcgagtttatctattttgtctttggttgctgtgcttcaagtgtcatatctaagaaatcattgtctaatccaaagtcaaaaaggtttactcctatgttttcttctaagaattttagagttttacatttaagtctgatccattttgagttaatttttatatatggttcaggtagaagtccaactttattcttttccatgtggttattcagttgtcccagcactgtttgttgaagagactattctttccccatggaattatcttagtacccttgttgaaaattaatcgtccttaattgtataaatttatttctagactgtcagttctacctgttggtctttatgtcgatcctgtgccagtaccatacagtcttgattactgaagtttgtgtcacagtttaaattcatgaaatgtgagttctccaactttgttccttttcaagattgatttggccatgctgggtcccttgcatttccgtacgaattgtaggatcagcttgtcagtttcaacaaagaagccaagtaggattctgagagggattgtgttgaatctgtagatcaacttggggagtattcgcatcttaacaatattgtcttccacctatgaacatgggcaaactttgtgtaaatggtcagattgtaagtatttcgggctgtgtgggcacagtgtctctgtcacagctacgcggctctgccattgtagcatgaaagtagccataagcaatatgtatgagtgtctgtgttccaatagaattttattaatgacaaggaagtttgaatttcatataattttcacctgtcatgagatagtatttgattattttggtcaaccatttaaaaatgtaaaaacatttcttagcttgtgaactagccaaaaatatgcaggttatagttttcccactcctaggttaaaatatgataggaccacatttggaaagcatttctttttttttttttttttttttttttgagacggagtttcactcttgttgcccaggctggagtgcagtggcgcgatctcggctcactgcaacctctgcctcccaggttcaagacattctcctgcacggcctccctagtagctgggattacaggcatgcgccaccacacccagctaattttgtatttttagtagagacggggtttctccatgttggtcaggctggtcttgaactcctgacctcaggtgatccacccgcctcagcctcccaaagtgctgggattacagggtgtgagccaccacaccctgctggaaagcatttcttttttggctgtttttgttttttttttaaactagttttgaaaattataaaagttacacatatacattataaaaatatcttcaagcagcacagatgaaaaacaaagcccttcttgcaagtctgtcatctttgtctaacttcctaagaacaaaagtgtttcttgtgtcttcttcccagattttaatatgcatatacaagcatttaaatgtgtcattttttgtttgcttgactgagatcacattacatatgtatttttttacttaacaatgtgtcatagatattgttccatagcagtacctgtaattcttattaattgctatgtaatattttagaatttctttttaaaagaggacttttggagatgtaaaggcaaaggtctcacatttttgtggctgtagaatgtgctggtgacatattctctctaccttgagaagtccccatccccatcacctccatttcctgtaaataagtcaaccacttgataaactacctttgaatggatccacactcaaaacatttagtcttattcagacaacaaggaggaaaaataaaataccttataaagcactgtttaatattgtattaaattggatcaatttgggggctagaatgtatgttagagacatgatatgtccataggtccttgctatcacagtgaggtctcagggacagtcgtttggtatcatttgggatctcataagcagactctctctgcttgacctgacaaatcagagtctgtgttttaacaggttcagtgagtgacttacatgcacattggagtttgggaagctccactgtaggtgcttagaccttacctttgttgttgctaataacaatgcaagcatttgggaggaagacctgtgttgctcatatgtgtccaggtgtagctgaggtggccttgcttatctgctgtagggccgttgagcatttctgtagctgtgatgagtgagctgaggtgagcctgcggagagctcccagccattggtagtgggactcgcttagatgaactggaaggaccctttcatctgagcagccactatggagaaaaacaaccgaatgaggggagagacaatgtgcaattttatttagggcacaaaggagagctgtggttagaaggtgacatttgagtggaaagggggcaagccatgtgtatagcgggagaagagaggtccaggcagagttaacagaaggcagaaatgctttccatgtttgagaaccagtaaggaggccagtggctgaagtaaggtgaagggcagaaataaggatgaggctgcgagagatgagaggttagagacgagcgtcttgtgcaccaagataagcttgtgtggtcaaaacaagtagtttaatttatgtttttaaaagatcattttggctgggcacaatggttcatgcctgtaataccagtagtttgagacggtgtggtgggaggattgcctgaggccagacgaccagcatagccaacatagcagcacctataaggtctctacaaaaaactttaaaaaattagctgggcatagtggtgtgtgcctgtagtcccagctactcaggaggctgaggaggctggaggattgcttgagtccaggagtttgaggctgcagtgagctatgattatgccactacactacaacctgggcaagagagtgagaccctgtctctaaatatacacacacacacacacacacacacacacacacacacacacacacacacacacacacatatatatgtatatatatgcatttagatgaaaagatcactttgacaataccacatgctggtgaggatttagaaaaactaggtcacttattgctggtgggaatataatatagtacggccactctggaaaacagtttggcagtttgtcataaaactgaacataccgttagtatacagcccagcagcaactacaatcctgggcattaatcctagagaaatgaaaccttaatgttcacataaaaacctatactcaagtatgcatagcagctttacccataatatctaagaactggaatcagctcagatgtccttcaacaggtgaatggttaaactactcagtaataaaaaggaatgagctactgatagcatgcaacagtttaggtgaagttatgctaatgaaaaaagccaatcccaaaaggttatacatactgtatgattctatgtttttttgcaatggcacagttttagggatggagaatagattagtggttgcctggggttagagatggggtagtagagtaggttagtggtggcagaggagagaaaagagagggaggtgaatgtggttataaaaggacaacacaggggaatacttgtaatggaaatgctttgtctttttttttttttttttttttttggcgacagagtcttgctctgttgcccaggctggagtgcagtggcatgatcttttctcactgcaacctctgcctcctgggttcaagtgatacttgtgtctcagtctcccatgttcagagtgaaacaaaccagaggtaatgttcatccaaataatccaacacacatgacattaaaacatcaagatcaggtcggacgtggtggctcatgcctgtaatcccagcacttttgggaggccaaggtgggcagatcacttgaggtcaggagttcgagaccagccgggccaacatgatgaaaccccatcttgactaaaaatacaaaaattagccgggcatggtggtgtgcacctgtagtcccagctacttgggaggctgaggcaagagaactgcttgaacccgaggggcagaggttgcagtgagctgagagtgcgccattgcacttcagcctgtgtgacagagtaagactccatctccaaaaaaaaaaaaccaagatcaattaaaatacagcattactgggccgggtgtggtggctcacacctgtaatcccagcactttgggaggccgagatgggcagatcacgaggtcaggagatccagaccatcccggctaacacggtgaaaccccgtctctactaaaaaatacaaaaaattagccgggtatagtggtgggtgcctgtagtcccagctacttgggaggctgaagcaggagaatggtgtgaacccgggaggcagagctggcagtgagctgagatcgcgccactgcactccagcctgggcgacagagcaagactccgtctcgggggaaaaaaaaaaataaataaatagaatgctgtagtgtccttgagtttacatgcccctccttacgcttgtgtgcccgtgcagattgcttgattacacaattagaggaggctggcggaggattgttttaatttttttttttttgagacagtctggctctgttccccaggctagagtgcaatggcgcaatcttggtgcactgcaacctctgcctcctgggttcaagcagttcttctgccgcagcctcccgagtagctgggattataggcgcccgccaccacgcccaactattttttgtatttttagtagagcagcgtttcaccatgctggccaggctggtctcgaactcctgacctcagatgatctgctgccccagcctcccaaagtgctgggattacaggcgtgagccacacctggccgtttgttttaattttgaaggtgaagtgaaagtgactacatttaccaaaagtgattgaaaagccaggactgttcttaccctgtttttccagttcttgctcagagcaaggtggtttctttttcacttaatcaccatacttacttttcatgtagaacaagtcagtttgagttatcagttcatcatcttaactaaattccatgggggaaggaattagttttagtttcttaaacttccaggtttgcttattggacaaaatgagatagcaaggcagtgtttttaagttagattttttatttctttggtaatacaattttctcagaaacttagtagtcttttagtttagttgtttttagttggtcctatgttttggatcacccctctctactttattttgatagtgccaactgtgaagacatctgaagccataggtttggatgggaaggaggcatctttagcctgatcatcttcgccaggctgtttatctccttttgcttggctgagaagtcttaataggaggcttattcccagctatttggggacatagaagcagttagccattgcttatattttactgaggtctgtgtggtatgttgattgtagtcagttaacgattttgagaactgaaggcagcctggtatatatagagtaggtattagactgtgtttcttctaattgaatttcccatctcttgtaatctatgccatcatcttctgtactgctgagaaagaaagaaagtttctaatcaaactataccactggttgtaagatgcagtttggctttagtgatgttaacacatgattcaaacgtgaaattgattgagtattggtgaaatacagaggagatttaaagccagaagacctgggtttaaatgctggctgtatgacttcatatctgtgtgatcttgggcatgtcatggttggcacttcaatttcttctctctataatgggggaagtgaggccagtcatggtggctcatacctataatcccagtgctttgggaggccaagatgggaagatcgcttgaggccaggagtttgagcaattgggcaacatcgtgaggccccgtctctacaaaatattttgaaaaaattagccaggcccagtggtgcgtgcctgtggtccgcgccactcaggaggctgagacgggaggatcctttcagcctaggagtttaaggctaaagtgagccatgattgtgctatcgtactccagcctgggcagcagagcaagatcctgactctaaaaaaaagtaaaataaagtaaaatgggggaaatgaactgctttagtaacatcatctgttttttctgtgagcagcgtagcttgacagccattggtgaactcgtgccctgtgcttccctgtccagatccccattctgcccgcaacatggagtataacggtttattcatagtagtcgagaaacactcactgaatgaatgaatgaggtgtagaactaagtggagtgggtaattcaacacatattaatttccttcttttttttatttttagaaagaaagaactttcagctaccaagaaagaccgtgtgaatcattgtctgacaatatgtgaaaacatagtggcacagtctgtcaggtaattgcactttgaactgtctagagaaaataagaactttgtatattttcagtcttaatgggctagaatattctttgtgtcccagctattttaaatggattcagaaatccatttaagatgaagaaggacccttttcccatatttctggctatatacaaggatatccagacactgaaatgaataatgttccctttttgtaatcttttatgcaaaaattaaaaccattatggtaattgaacaacatgtttatgtttagttaacacccttagcaactatagttattttaaaaccatctatggtttgatatttttgcatttgttgcaatagtaggaacagcacaagacagttcagtttgtctctcttatttgctttttcttggcagtttgctgtcctattgtacctctgctcctagcagtggctggagcccactcctctgtgcttcgggattagtggggatcgtggggcattgactgtaggtcagctttccttgcttgatctttctcactgggatgaactagcagcaccttcttttgtagctgctttgcttttgactatctttctgaccgttgttcctagtagctgtagatggtaaatatatttaggcctgtttccaatggctcagtaggagacatattcacctatgatatctgaattctgttacccacatgggcatgcgtgaaatagttgccttgccttactttcccttggaataaataattcatgttattctcctggtagaagctagaaaaagcctttatagtcagtcagaaaaaaatttttagacaaataatcttgattttagtactgacaaaaacgtgtggtgattctttttttaatttttttttgagacggagtttcactcttgttgcccaggctggagtgcaatggcgtgatctcggctcactgcaacctctgcctcctgggttcaagtgattctcctgcctcagcctcccaagtagctggagttacaggcatgtgctactgtgcccagctaattttgtatttttagtagagatgttggtcaggctgatctcgaactcccaaccttaggtgatctgcccgcctcagcctcccaaagtgctgggattacaggcgtgagccagggcgcccggtgattcatttgttttttcaaaaaatttcctcttggccattgcttttcacttttgttttttttttttttttgagacggagtcacgatctgtcacccaggctggagtgcagtggcatgatcttggcttactgcaagctctgcctcccaggttcacgccattctcctgcttcagcctggcgagtagctgggactacaggtgctcgccaccacacccggctaattttttgtatttttagtagagatggggtttcaccgtggtcttgatctcctgacctcatgacccgctcaactcagcctcccaaagtgctgggattacaggcgtgagccaccgcgcccggccctctcttgtctttttattgtggtaaaatgcacataaaattgactgtcttaaccatttttaggggtacagttcagtatatatattcgtaatgttgtacagccatcactgccatctacttcataagtttttcttctgtcaaaactgaacatctgtcttcattaaactccctatcatccattctttcctgtagtccctttctactttctgtctgtatgagtgtaactgctctggagacctcatgtaagtggattcctacaggatttgtgttttttttttggtgatctgcttatttttaatgcctctgtgcatttgtattatatactttcaaagtgatttcacaaaaccgtttcattttaggttaactcatttctgttgtttgtgaaatactgtgtatgattctgttctgtttctgtctaatttgtggaaatgttgtgggaagaaaatgaaataacaaatgagcatatgtcctgaaaataaaaatataaaaattctaagttagcatgctattgtagaatacaacgctatgataaaagtaggaaaaaaaaaggtttgaattctatctctgctacctgtgtaagctgggtgactttagataagctgtaacgtgtttgagccttactggctcatttttgaaatgtaatccctagttacacagttcttgtgggatcagatggtacatgtgaaacactgtgaaaaagcaactgcatagatatgttcattagccacctgagcgggaagcgtatcccattgcgatgcccatcatccaaagctatatgttatctttacttttttttttttgagacagagtcttgctctgttgcccaggctagagtgcagtggtgcaatctcagctcactgcaagctccacctcccgggttcacgctattctcctgccccagcctcccaagtagctgggactacaggcacccgccaccatgcctggctaaatttttgtatttttagtagagatggggtttcaccgtgttagccaggatggtcttgatctcctgacctcgtgatccgcccgcctcggcctcccaaagtgctgggattacaggcgtgagccactgcccctggccatctttactttttttgtgaaatgactttaaatacttggcaaacatttggtcattgttcatctgatctccaccatccaggtctcagagaacataatttctctctgaaagcttattgacccaggaaataagatctctttcaatctgagtgcgtcaggctttattcttgtcattttgtcttttgataattttcaaatggaattcatggaatgttggcttatattcatatattagtaaagtatgttgagacatcttaagattgatttgtggttctatatgccatattaaatcaaaataatagctgttaatggttttcacattagtctgtctcttgtttttatggagtaatgctgagagttcattatgcttgttctacagaagagcatgttaaaaggagtttttggagtcagagaggttattcttggtttcataggatacactctatactttttagggatttcagagtatatagctgaaggtgatattttatgtaaatatgttttatggaaacttattgctcatcgctgtttcctgttaactctcctaaaatataattaaacttttggaacttttttatagcttttgtgctagactaatttttgtctctaatgaggttatataaatggcagcttctgacgttttcaatgtaggaagtcatttaaaacttcatgtatattgtgaaaatgtagtctgctttaagctctctaaagtggtctaagttactggttcctaagtatggatgagcatcaaaatcatctggaaaatttgttaaaaatacagtaatgaaggcacctcactgtcctttttcccaaacatacttctgcattctgtttgagtaggtagggactacacatttttcacaagtatcctcttgggaatacccaggaatgcttacttgagcaacctcttactaatatgtaccttgataaggtggctaggtaaacataaatatacaaaaatccatagatctcccatatattagcataaatcagctagaaaatataacgtttaaagatctagttcacagtagcaccaatatatcgaactctaaggaatcgataaatatgcaaaaactttataaaaacttctgttaatgtttctgaaagatataggtgaccactttctagataggaagattttatattactaagttgaattttctctaaattaacacagaaatttaaaataatcttgatcaaaattctagtagaggtatttttgaacttgttcactgcaagaataaatacataattgcaaagaatatctcaaaatcatcaccaggcctggtgtggtggcccatgcctgtaatcccagcactttgggaggctgaggcaggcagatcacctgaggtcaagagtttgagaccagctggaccagtgcggtgaaacactgcctctactaaaaatacaaaaattagctgggtgtggtggtgcatgcctgtagtcccagctacttgggaggctgaggcaggagaattgcttgaacccaggaggtacaggttgcggtgagcctagatcgcaccactgcattccagcctgggcgacaagagcaaaattctgtctcaagaaaaaagagaaaaaagaaaaagaaatcaacactaatatggtgagacttaatgtatgtgacattaaaatagtgattggatgttaaaacaggtatagaacagaaagaagagtgtatgtgtgtatctgtatgaatttatgatgggtgtaacatatatgtattagggaaatgagggaaatgatacatttctctgactttgggagaacattatatctctacctcatattgcaaacaaacataaagttcagattaattacctaaatgtgaaaaaatgaaataatttctttaaaaaatgtaatcttagtttgaggaaggttaacattataaaggaaaaaactgttttgagtggaatatagttcaatatgtcaaaatccaccttcaacaaaattgaaagtaaattgaacttggggaaagtattgacagcatatagatcaaaggttactagcctgtgtaaagagcagttataaatatcgttaagaaaaacactgtcgacctgtcggcaccttgttctccgactcccagcctccagaactgtgacgagtaagtgcttattgtttaaaccacccagtctgtatgtggtattttgttatagaaactcaagctgattaggacactagtaatcagtagactgaaactgaaacaaaaataagaaccttttttacctgtcaaattggcaaacattaagaatattcagatttttgtcagaggtgatacaaccttctaagaaggcaatttgggaaaatataaagctttagattattatatgtctgacctagcagttttacctctagggtgcttacccctaggaaagtgtgtaatgatattggtgcagtgcccttcatcccattagaaaattaaaaataaccttaatggcctaccactaaaaggggattgaaaatttaagatatatttatttatgtgtttattgagatggagtcttgcactgtccgcctgggccagagtgcaatggtgcgatctcggctcactgcaacctctgcttcccgggttcatgtgattctcctgcctcagcctcctgagtagctgggattacaggctcacaccaccgcacccggctaattttttgtatttttagtagagatggggtttcactgtgttggccagactggtctcgaactcctgacctcatgatccgcgcccctcggcctcccagtgttgggattacaggtgtgagccactgcgcctggccagatacatttatacaagagaatgttagttaacattcatagatatttatattttgtttactttttattaaaaaaattttttttagagacaggatcttactctgtcacccaggcaggatgcagttgcacaatcatagcccactgcagcctgaactcctgggcttaagtgatccttctgcctcagccttttgagtacctgggggactttaggcagtgctactatacctggctaatttttaaatgttttatagatgagatcttgctgtattgcccaggctggtctagaattcctgggcccaagtgatcctcccaccttggcctcccaaagcgctgagattacaggcatgagccaccacttctgaccaatagatatttatatttgtgactggaaaatatattaacaatgtgttaaaaaattcagttaaaaaataatgaaagatttttgcttctggctaagatagaataacaaggacagcatttatcttcttgccttgaaatagttgaaaacggaagaaatatatgtaacagtggttttcaagttattgggcatcaggcaaagaagaatagttatcccaggaaaatgaatgtggagagccctacaatttccttacattactgcctggtcatggcaagaggaaaaactgagaggagactgaggctgagccagtggtttgctgggttgaggaggcagagctgggagtgcagagatgcaaggtggtgagagcccatatggaagaataccagggaagagagctgcagagggagctccggagacctgcaccctgccctctcagtaccctgtcatgtgtgtagctgagtactgacgagcacttgcttgtgcggaaatgacccagggctggaggtagagccacctgaaaggattagaaggaacagttgctgaaagtcacacagggccaggaagaatttctaatcacaccagttggagtggaaaacctcagctctcatagagcaggtagggtactcagaagggtttgcccacctagccccagactaagtttcgttactctgaccctacctaatattaaaaagagattaattaaattgttcgcaacaaaaataatatatttcagtgtttgtaacacgtagaagtgaattgtatgacaatagcataaaggctggaagagcagaaattgacatgtatttgcgctgggcagaataatgctcccctctttccccaaaagatatcaagtcctaatccctggagcctgtaaatattactttatatggaaaattgttttatgatgtgattaaattcaggatcttgagatgagggggctatcttggatgatctgggtaggcactaaatgcaatcacatatatataaaaaggaggcagagggagattttacacacagagagaaggccctgtgaagatggaacagaaagatttgaaggtgctggccttgaaaattggagtgatgaagctataagccaaggaatgcagcagccaccaaagctggaagaggcacggagcagttctcatttagagcctactccagagggaatgtggtgctgccaattccttttttttttttttttttaagatatcatttacccctttaagttggttttttttttttttttttttttttagtatttattgatcattcttgggtgtttcttggagagggggatttggcagggtcataggacaatagtggagggaaggtcagcagataaacatgtaaacaaaggtctctggttttcctaggcagagggccctgccacgttctgcagtgtttgtgtccctgggtacttgagattagggagtggtgatgactcttaacgagtatgctgccttcaagcatctgtttaacaaagcacatcttgcaccgcccttaatccatttaacccttagtggacacagcacatgtttcagagagcacggggttgggggtaaggttatagattaacagcatcccaaggcagaagaatttttcttagtacagaacaaaatggagtgtcctatgtctacttctttctacgcagacacagtaacaatctgatctctctttcttttcccacatttcctccttttctattcgacaaaactgccaccgtcatcatggactgttctcaatgagctattgggtacacctcccagatggggtggcggccgggcagaggggctcctcacttcccagatggggcggccgggcagaggcgccccccaacctcccagacggggcggcggctgggcgggggctgccccccacctcccggacggggcgggtggccgggcgggggctgcccaccacctcccggacggggcggctggccgggcgggggctgccccccacctcccggacggggcgggtggccgggcgggggctgccccccacctcccggacggggcggctggccgggcgggggctgccccccacctcccggacggagcggctgccgggcggaggggctcctcacttcccggacggggcggctgctgggcggaggggctcctcacttctcagacggggcggctggtcagagacgctcctcacctcccagacggggtggcagtggggcagagacattcttaagttcccagacggagtcacggccgggcagaggtgctcttcacatctcagacggggcggcggggcagaggtgctccccacttcccagacgatgggcggccgggcagagatgctcctcacttcctagatgggatgacagccgggaagaggcgctcctcacttcccagactgggcagccaggcagaggggctcctcacatcccagacgatgggcggccaggcagaaacgctcctcacttcctagacggggtggcggctgggcagaggccgcaatcttggcactttgggaggccaaggcaggcggctgggaggtgaaggttgtagtgacccgagatcacgccactgcactccagcctgggcaacactgagcactgagtgagcgagactccgtctgcaatcccggcacctcgggaggccgaggctggcagatcacttgcagtcaggagctggagaccagcccggccaacacggcgaaaccccgtctccaccaaaaaacacgaaaaccagtcagacatggcggtgcgtgcctgcaatcccaggcacttggcaggctgaggcaggagaatcaggtagggaggttgcagtgagtagagatggtggcagtacagtccagccttggctcggcatcagagggagactgtgcgagggcgagggcgagggcgagggaattccttaatttcagtttagtgatactaattttggactctggcctctaaaactgtgaaagaaaaaattttttgtttgtttgtttcttttaagccacatagtttgtggtaatttgttacagcagctgcaggaaactaatttatgctgcatgtgaaatggtgtaataaggtagattgtgatgaagatacatagtataaacaattaagcaacaactaaaagcacaacaaggaattatagctaatgaaccaaaaaaggagattagaataataaaaatggtgaatcccaaagaagccagaaataggggaagaggcaaataaaggaaagaaagagcttgatggtagatttcaacctaactatgtcaaaaaggacattacatgtaaaaggcagcgatttttcagattgaatggaaaagtaagactcggtatatgctgctgcctgcaagaaacacattctaaatataaaggcaaaaataacctacaggtaacagaacggaaagaagttcactgtgcttacaagaattagatgcaagctagactggttctgttaatatcagacaaagtggatttcaaagcaaaggctcttgcccaggatgagatggtcatttcataatgatgaaggggattcgttcatcagcctggcatagcaagctgaaatgtttatgcaccggactacagagctaaaatacatgaagcaaagcctgacagaactacaagtagaaacagacaaatccacagtgatagagatttcagtagccgctctcaatgatttgtagaacacgtagccataatatctggatctagaacacttgaccaacactgtcccctgtgcaacctcattggcatttacaggacactccacccagcaccagcagaagagacactctctcaagtgctcacagaatgtttgccaagatagagcagatgctgggccataaaacaagtctctaaattaaaagcattcaaattattcagagtatgttttctgacctcagtatcattaagttggaatatattataggaagataacctggaaaagcctcagatatgtggaaaaacccatttccacatggcccatgggtcagaagtgaagtcaaaagggaaatttgaaagtcttttggattgactgatataaaaacaatagatttctaaacttgtggggtgctgttacagcatagtaaatggaaatttctagcattaaatgcctgttttaggaaagaaagatttcaaatcaatgacctcagcttctacctttggaaacttgaaaatgacaagcaaatggaatccagagttaccagaagggccaggtacggtggcttatgcctgcagttctgccactttgggaggccgaggcaggtggattgtttgagactggcagttgaagaccagcctgggcagcctagggagaccccatatctacaaaaaacaaaaaaattagccaggtgtggtggcatgtgcctgtagtcccagctaaccaggagtctaaggtgggaggattgcttgagtctgggaggttgaggctgcagtgaactgtgattgtgccactgtgttccatcctgggcaacagaatgagaccctgtctcaaaaacaaaaacagttactagaagaatggacatcataaagataggagcagaagtcagtaaaatagaaaacaaaaatacataggaaatcaataaaaccaaaagctggttcatcaagaacatcaataaattggtaaagctgataggaaaaacagtgaagtcacaaattagcaatatcaggaatgagggagatgacagtagtatagattatatagatattaaaaggactgtatgaggcaggtgtggtggttcacgcctgtaatcccagcaccttgggaggccgaggtggacagatcacctgaggtcaggagtttgggaccagcctggccaacatggtgaaactctgtctctactaaaaatacaaaaattagttggtcgtggtgctgtgtgcctgtaatcccagctacttgggaggctgaggcaggagaattgcttgaacctgggaggcggaggttgcagtgagctgagattgtgccgttgcactccagcctgggtgacagagcaagactccatctcaaaacaaataaataaataaaaaggactatatggtaatattatgaacaactttatgccaataaatttgacaacttatagatgaaatggatgagttccttgaaagacacagaaactattaaagctctctcaagaagatatagataagctgattagccctatatctattttattgaatttaaatgtaaaaatcaatatttagttactggaaaacttttaagtgtggttggaaatggtatacgaactttttcaactgaattttatgaagtctaatcacaggtaaaggttttctgatgaaaatttagtgtctgaattgagatatactgtaaaaaatgttatatatcttaattatttcttcacattaattacatgttgaaataatactttgggtgtattgggttaaattaaatattatgaaaatcttgcctgttttctttttacttttgatgcgtcagctaggaaatataaaagtgtagctcacattctgtttctgttgacagtactgctttggagcacagtgtttgaatgatctatcatttcaaagacctttcctcagttcgttattcatggctgtctgtattccacatagataaggtctgaaatactgctaagtggcatgttttgttttatgcttttataagtttgttgatcattactgatgtggacttttggtgcctcttaggctcattgctatcttccaaccattgtttgcaatttttacctagagataaagagaaagagacatttggtttcagagtagttagattgggatcatgaaagagcaacctcattttgatgcttcaaaaatagcacatcccccgtattactgggatttgctattcttgggattacttcaagaacatccttgtgttactggtttggatgcttctgaatgctgtgaagtcagtttcatgtacatggctcatcagtttagctctctcttggctttgtttagacagttggagcatgatggcctaaacagcttctttcaattaaacattttaaaatagtttacaaatagtaaacaaactccagtttttgtgactctttgtctcgcacaacaaaaacacaatctgaccatgatcatctggcatcttagggtgaaatatggttatactttggcccataccgaaagcaagattaaaaaggggcaggagagatagactgctgaactgattttcaaggttccaagaatattgtaggttaagagtaaaagtaaacttttggtagaaagcagtgggttgtctaggattgaagtatctgaagtttttaaacgaaaatttaaaaagaaaaatgagaattgccttacaagtacaatctcttcttttttaaaaaataaactttattttgaaatagttttagatttatagaaaaaaattagatagggtaggaagttttcatataccctacatccagttaccccagttattatcatcctaatttagtgtgagacattttcatgtttaatgaatcaatattgatatgctattaacttaagtccagactttattcagattttcttaatttctatgtaatgtcctttttctgttccagaattccatgcaggacaccggatacctcattacatttcattgtcatgtcaccttaggctcctcttgacagtttctcttctttttttgcttagaaattctccagaatttcagaaacttctgggcatcgctatggaactttttctgctgtgcagtgatgacgcagagtcagatgtcaggatggtggctgacgaatgcctcaacaaagttatcaaagtaagaaccgtgtggatgatgttctcctcagagctatcattgttgtaggctgagagaagaagcgatcattgagtgttcttctgttttgagtccctgaggatgtctgcacttttttcctttctgatgtatggtttggaggtgctctgttgtatggtttggaggtgctctgttgtatggtttggaggtgctctattgtatggtttggaggtgctctgttgtatggtttggaggtgctcttgtatggtttggaggtgctcttgtatggtttggaggtgctctgttgtatggtttggaggtggtcttgtatggtttgcaggtgctctattgcatggtttgcaggtgctctattgtatggtttggaagtgctcttgtatggtttggaggtgctcttgtatggtttggagatgctctattgtatggtttgcaggtgctctattgtatggtttggaagtgctcttgtatggtttggaggtgctcttgtatggtttggaggtgctctgttgtatggtttggaggtgctctgttgtatggtttggaggtgctcttgtatggtttggaggtgctctattgtatggtttggagatgctctggtatctgcctgcattgcttgccacacctgcccggtcagaaggcgctatgttgacaattgtgcctgcacggtgcctaggtcaatgaagggaaccgatggtagccactggatgctcctgggaaaatgtcactacaggcaccagagaagccagagctatgcccaaatttctatgagtctcagttttcttaaccataaaatgggatcaatgtttttgtggcatgtgtatgagtgtgtgtctgtgtatgtgtgaggattaaattgtgtatgtgtgaggactaattgccactactggatcctcaaagtggtaagaagtgttcttattaataatgacatccttacactcttacccagcaagattgatgggtgtggcactgcttctctttttccatcacatggtttccatggtatccttttgcccagggaatctttgctttgtggctagcactttgttgtttggctaatcacgctttctgtggtcaggacgctggcttctctggagccatgggattctagctccctgtcttgtccctagagtggtcactgtcttctctctccgcttgcaattcctgctttgctcgcatctcacttatgcagtgacgtatatcagtttcaccttgttctccgtgcctgctgatcattggcaccacttgcatggtgccatttagggcctgcttccagttaagcttgcttctccacaggcctaaatatccttgcttgcttcttttattctcactggcaggaccagggcggtctgtctttgcatgagacagggtctcgctcagtcacccaggctggagtgcagtggctgatcacggctcattgcagccttgagctaccgggctcaagctatcctcctggcttggccccttgagtagctgggactacaggcgtgcaccaccatgcccagctaatttttaaaattatttgtagagatgggatctcgccaggttgcccaggctggtcttgaacgcctgggctcaagtgatcctccctccttggtttcccaaagtgctgggatcacaggtgtgagccactgtgcctggcccttgatgtttcagttcttgatatttgatcctcagagtcagaaaatctaaaaagagggctatcccaggttgccttggttcatggcaaatgggacgttaagagggcagagagaatatgaacagaaactgttctaatattggtcatttaatgtgtaagtattgttcttttttaaacctccttcattttttttccaggaattgctggacacagtggcttggtgtgtgtctgaggactgtaggccatggccctaggttgtggttttaggtctcaggtgctcttcctggctgtctccttgcttctttcccatgtcctcttctttgtttccagccatttctcccttatgcttaagtttggtgcagcagggtttggctgctctcagattcctgcttcctcagatgctgtagttgtcaggcccagcgggctggcagcgggatcaggatctggctaggtttgctctcactgtggcagagtagggggaggcgtgggagagcacgtgtgaccccaggccagctgtagggagcataggcatggtcacgtagccttcaggtcctagactttgtcttctcatgagtatggctgtgtgtgtatggtgaaaactaggttctacttagcccaagaaaatgggcacattttgcatgtggtttctgtagagaaatgcactgggtatctgacatagcctggcagcatgcctccctcaggtaggttagtctcaggcggtgaagcacgtgtgtccagcaagaacttcatatgtggcataaagtctccgttctgtgaggtgctggcaaatcaccaccaccgtcaagaggctgaagtgatttttgtctagggaggcaggaaaggcttcctggagtcagcagccagtaggtgaaagagtagattggagaccttcttaatcatcaccgcctcttgtctcaaggggtgccaggaagctgtggaggctgaacccatcttatgctgccagagagtgggacaccatgagggtcaggtcaaggggttgtaccttgtttggtagagaattaggggctcttgaagactttggatgtggtcaggggagtgtatcatttaggaagagtgacccggtgaggacgtggggtagaggaggacaggtgggagggagtccaggtgggagtgagtagacccagcaggagtgcagggcctcgagccaggatggtggcagggctgtgaggagaggcagccacctgtgtgtctgcggaagcaggggcaagagggaagaggccagcagcgtgctgccatcacccagcgactggcgtagattgtgagagaccattccctgctcttaggaggggctgagttttagttttctcttgttatacaataagcttggtatttgtttacaaaacatttgtaaagctaaatcaaggtttgataaggcttctagttttatttaagaagtaatgttgaaataaatgtttgtccaattcgctttgctcatttaaggactttcagtacaaactgcaacaacaggattaggatttaaacgtttctgagatgtttttactcctcagaatttcccagaatgtgatctggttttgattttcaagcttgctgacccaataggttaacccacaagttttacgaagaccatctcagtccacttacatcaactgcccatgccacggttaaagagatcatcgactgatgtttggcacagcttcctccctcttgggtgggcaagcatttggaagagaaggctcctatgggtgagagtggggcaccaaagtcttccctgtcccatcccctagcttgagaagcccttctctaatgtggactttgtgccgttagcatcgttactagcttgaagttgaccatctggacgtactttctggtttagcctcacaagtgagcaaggagggttgagagatgtgctgtgaggaatgtggggccccagctggcagcaggctctgggtcaggggggcagggaccacgggcatacctgacagtgaggaggggccacacctgcagaaaaggatgcaggactccgccttgggaagtgttctaggccagagcgagggtctgtggtttataagtacacccacagtgctcgggaccctgcagatgtccagggtgccgtctgagcccgtatcatccaacagaatgttctgctagtgaagattaaagatttactccaggggctttaggatttattatatatatataaatcctatatatataattttttttttttttttttttgagatggagtttcgctcttgttgcccaggctggagtgcaatggcgtgatcttggctcactgcaacctccgcctcccgggttcaaactattctcctgcctcagcctctcgagtagctgggattacaggcgcccaccaccacacccggctaatttttgtattttttagtagagacggagtttctccatgttggtcaggctggtcttgaactcctgacctcaggtgatctgcccgccttggcctcccaaagtgctgggattacaggcatgagccaccccacctggccaggatttattgtatttgaaccatctaccattttaattttgatgttatgtagtatttgatgataatgaaagttaaattgtttttctttccatttttctgtttaagtgaatgacctgtatctagtttattcagtaacttcctgcatatatttgtttctttcattcttaatgaatatattcttaatttagttgctattatgttttgctttgccccaaaattgaaatcttagtttccttttagctcgttttagaactagtgatgggatgtgtcttccataaatctcttgtgatttgttgtaggctttgatggattctaatcttccaaggttacagctcgagctctataaggaaattaaaaaggtgggccttgcttttcttttttaaaaatgttttaaattttaaatttttataggtacacgtattttgtaggtacatgtaaatgtatatatttatggggtacatgagatattttgatacaggtatacaatacataataatcacaccatggaaagttggatatccatgccctcaagcatttatcctttgtgttacaaacaatccagttacatgctttacttattttattttatttttgagacagagtcttgctttcacccatgctagagtacagtggcatgaccttggctcactgcaacctccgcctcccgggttcaaccgaactttgggctggtctcaaactcctgacctcaggtgatccgcccgcctcggcctcccaaagtgttgggattacaggcgtgagccactgtgccgggcctgattgtacattttaaaataactaaaacagtcagggcacagtggctcatgcctgtaatcccagcattttgggaggctgaggcaggtgatcacctgagatcaggagttcgagaccagcctggccaacatggagaaaccctgtctctactaaaaatacaaaaattagccaagtgtggtggcgggcgcctgtaatcctggctactcgggaggctgaggtaggggaatcgcttgaacctgggggtggaggttgcagtgagccgagatcacgccactgcattccagcctgagcgacagagtgagactttgtctcaaaaaataaaaatgaaataaaattgggccgggtgtggtggctcacaccttagtcccagcactttgggaacctgaggcaggtggatgcttgagaccaggagtttgagaccagcatgggcaacatggcaaaacgctgtctgtacagaaattagctgggtgtggtggtgcacaactatagtctcagctacttgggagattgaggtgggaggattaattgagcctggaaggttgaatctataggtagctgagattgtgccactgcccttcagcctgggcgaccaagtgagaccctgtctcaaaagaaaaacaaaaaaacaaaaaacaaaccactattatcgactatatattattgtctatgatccctctgctgtgctgtcgaataccaggtcttgggcccttatttccatcactgagcaaacttcactctgttaagcagcaggtgtgggatttcatcgttattcagtaattcacaatgttagaaggaaatgctgtttggtagacgattgctttacttttcttcaaaaggttactctttattagatgagatgagaattaaaaatggtaacttactttatatctttataattgaagcccactagaccttaaagtagttaccagatgttttatgcatttaaatggccttttctctaaaattagaaagtaacaaggaaagaaaatgcttcgtttctatgcaaccctcttggtgactagtatgtgactcttaatgcaaccctcattgcaccccctcagaatggtgcccctcggagtttgcgtgctgccctgtggaggtttgctgagctggctcacctggttcggcctcagaaatgcaggtaagttgtacactctggatgttggtttttgtcgggggccagctgctactgatcctttatgtctcagctcagatgtcatttcaaaagtctgctctgccctctccaaattgcagtcgaccttgccctgtttatgtttccctcatagcactaatccatgtcagaaattgtcacgtacagtctatctgtgtgcttgtttattttctatcccacccttccgcaagagacttatgggatgtgtgccccaggacagcaggggtcttactgtcttatgctctgttgcagcccagcagcgataacagtgtctgcacatagtacttgcttaaaagatacttgccaaattgttgaaggttgaggtaccaatttcattattgctgactataggagttatagcaaaatatccatttgtctgttacatgagttaaaaatatggttgttgcactgtgaatagtttggtttagtcaaaacagttgtatcttaacggattgagaaacaaaagcaggaccacttttcatcagctccctccttctccttaaccagcaatacatgctgatgctgatatcccatagaccctcagctccatcctgagtcactgggaatgtggtctaaaccctcactattaatatgaactgagtttcaataagaatcttatatgggtcgggcatagtggctcatacctttgatcccagcacttcaggaggccaaggcaggtggattgcttgacccagactaggcaacatggtgaaacgccgcctctacaaaaaatacaaaacttagccaggcatggtggtgcgtgcctgtggtcacagccactcgagaggctgaggtgggaggatcacttgagcctgggaggtggaggtcgtgttgagccaagatcgcaccactgcactccagcctgggcaacagagtgagacctgtctcaaaaaaaccaaaatccagaaaagaacttatatggctgcagaggtataatcactaaggaaatttccttttgtataatcttttttcttttactatcatttaaaaaaatgtgttatatttctgaagcaacacatccaggttctgcacatagcagccaaagtgaccttaaagaatataactgggtcttgtcattcccttatttaaactcttgtacccatttcccagtgccgtttagatagagattccagactcgtcaatggctctgtcacctcagacaccctgcattgactcattagtctgattagagtcaggtttttcttcctcctgatggtttttttttcccccttagttctcagcggaacagtcacttccttagggaggtttccccagccaccctctgaggccgtgcttgttgccagactctgccactagagggcagggctgcaccactcctggcacctcgcacccggcctgccctgtcactctgtgtgttgggtgaattcctgtgatctgtgactcactgctctgtgtcctacacattcggcttttcttctctccccacaaccccattttataattctcctttttcaggaaagctttattcccatttaaaaatttttgtttttaaaatggtattttcttacacttattttctaattaaaaatgagtgttttaagaagtattatgatttactgcaaataatttttaaacccagccttttagatcctctgtgatcataagagaaatgaaggatgtctcccaacacttgagcttcatccacatttcatcctcctgttctttcagctgagttttccccatcccattagggactgttggaatataaaactggcttttccctaacagggaatgaattgcttctgtttctcctgaaggagagctggaagaatgacttgcgttcttttgcatacacaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggtatgattgttgcctcaggtcacaaacatgcgagtgatgctgtgagtgagtctgtggagggtgagggcttctgaacagggagtcctgtgggagtgcttcttggggtatgttgtatgtcgtaatttagactaccatcatttgtgttatttttgaggcacctaaggacttctttccacttctcatttcttactgtggggtgaagagttgaattgggagatggtttctagatgcaaattgaaaaggcatttttccagagcagatttgttttcggcgtactagagtgactctttaacctagctgcgggaagatgactgtgccaagactgcaggtaggagaaagctcactgacgaggccttgtgggtctgaacgtcctgcagctatcagagcctgttggcttcctgttgtgcattccaacaaatcatcttcaaacccactttagtgttttgtttataatgtccagaaatagtgaccctgtcacatgctctacagattacaggattcttagcctcttcctttttggtaggtcagtcctgggtttgagcccaagtgaccctcctgggaggtgatgatacacactgggtagagtggaatcagatggacttggattagaattctgtcctctttactagttattttcctctaggcaaactgcccaacagctctaagctatttccttcgtattctgaaaaataagccttaatgggacccatatagggcaactctgagagtaaaataaaggaatatgtgttagagtgtagcatagtcacccacgggaagggcttagatgttagctgctactgctcttattagctgaatgatttggaataaactgttagcctctctcatgttttttctcttgagcttcgaagttttcttgttaatactaaggagatattcaaactagtcatggggttttggaatgacgaagggagatgatgaatctaaagaatttagtgtaatatttcttcatgctcagtaaatggtagtttctgctgctgttatttttattaccatctctttggaatgggagtaggtgctcctttgtggtcagaggctgtgagagctccacagcgccagtttgcccatctgtacactggggtctgttgaaggcagtcccctctgtgatatctctggctgtcagagctcagatgatagatggtatttttgtactcttagttctcatcattttcatgatttcgatcaccatttgagtatgatgatgctaacactttgttgaacgtagaatccgttaattacttccttcctgaacctttggcattaaaaaaaatctattctgctacctctctgctcatttatggttattcaaatttattatcaagagcctggtacagtggcttgtgcctataattgtagctacttgggaggctgaggtaggaggattgcttgaggccaggagtttgagaccagcctgggcaagatagtgagaccctatctctaaaaaaactgaaaaaaaattagctggacatgatggcatgtgcctgtggtcctagctactcaggaggctgagacaggaggctcggttgagcccaggagttggagttcgaggctacactgagctgtgattgtgccaccacactccagcatgggtggtaaaacaagatgccatttcttaaaaaaaaaaaatatatatatatatattatcaatgaaattcagtagtaccaacaggattataaacaaagatagtagttcccttcctactttttctcttaatccttgtgtctcacaggcaaacataactcttagtatttcttccaatatttactttcatgtttctttctttctttctttttttttctttgagatggagttttgctcttgttgccaaggctggagtgcaatgacgcaatcttggctcaccacaacctctgtctcccgggttcaagcgattctcctgcctcagcctcctagtagctgggattacaggcatgcatcaccacgctcggctaattttgtacttttagtagagatggggtttctccgggttggtcaggctggtctcgaactcctgacctcaggtgatcctcccacctcagcctcccaaagtgctgggattacaggcgtgagccactgcgcccagcaacttccacatttctaaataacatgcttctactgctattttttttttcaattttagacatttttttactttcactatagttctatcagaattcagtgtgtacgttattatgcctaagtaaatagtcatggttgcttacgtattatatttctttgattgtgtttcttatttgatgagaaagctgtgttttttgctctgggttgaaactggagagaggacctggggaggaggaggaggacagatgaagttggtgactgtaccttcatggccatagctgggttctcagcacccggggatctgctgatcacctactcataggccaggcccctatcgaagttctaggtgacccagtgctggggacgggggggccacctgcaaggtctaatcatggaggtgggggctacagtgttggcttgtgctggggccagcatccttaggaaggcatcttggaggtggaggagacagccgcccacttcttgattggggccttcagcagcaccagcttcttgggcaggctggtgctggctttcatcaccatgtcgtgttcaatcttcttccagatcctgacttctaggttcagctttcctcagaccctggttcctttcagaggccattgctgctgccttgctctttgctggcttgtgccttgattatatgtctttgtacaactttttgttttcctggagttaatcttcacatctgttttcttggagttaatcgttacctctatatcgcttgcttattattctttggcctttttgtcttctcacaccttccaacttctttgtaatatgtgtttagtacaatttttcatgacaggtagtttactgaatcagtttttccccagtgtggtcatccaacttgagttatccagctctctgccccagtctgggcaggttgatcttcaggtctgtagtacacttgtatcctaggacttctctttgccattagcctggaatttcctttgcagttctcccgttggatgcccagttcctagatgccatatgtttttctatcgtctagtagcttcctgagagaagatgaatgggagggaaattgtatgaggttttgcattcataaaaatgccattttttttcctgtacacttggctgggtatggtgttctggggtagaaatcattttccctcagaaatgcaaagtctttgccctgttgtcttaaaatctccaacgtgacccgattccttaacctatgaatgtacttttctttggaagctttccatttttggggaggtgaagtgctaggtacttagtaggccttttaatttggaaacttacatcccttcagttctgggaaaattttcttaacatttctctgagaagttcttgccttttattttctgtgttctctcctgaaattggttagttggatgttggtcctcctagattgactcacatcttacctttttcttttctttttctggtactttttagatatccatctcaaactcttctattcattgttatgtttttaacttctttcttttctttgtctcttgatggggtcttgccctgttgcccaggttgtggtgcagtggtgcgatcatagctcactgcagcctcaaattcctgggctcaagcagctgttctgcctcaccctcccaagtagttgggactacaggtatgcaccaccacgtccagctattttctttacttttttttttttttttttgagatggagtcctactctgtcgcccaggctagagtgcggtggtgggattttggctcacttaagcctctgcctcccaggttcaagcagttctcctgcctcagcctctcaagtagctgggattacaggtgtgcaccaccatgcccggctaatttttgtatttttagtagagccagagtttcaccatgttggccaggctggtctcgaacgcctgacctcaggtgatccgcctgccttggcctccgaaagtgccgggattacaggcgtgagcccatcattagatctttaaataccagtatctataagtcttttcctcttgagtcagctagtatccctggaaggaaattactcattttcctgcttggaggctataagcttggctatgtttatcctgcaaccggggactggaagggaggggactgacagtgttgctggtcagggtgccctcttactttttgttttctgtgtgcatctcacgtctgtcctcagcctatgtaaacacctcttgagattatccctctcaatctttgccggaggtgggggaggggctgcttcctgggctgccttggattggagggaagacctcaggtgagtgggtgggaatttgcccaaggagccatgagaccagccactatttcaccctctccatccctccactttcagatgtatgtggcgcctccaaagcccgagctcttcttggcgtctgtggcttcaataagcttgctttttgctggtatccctcctaccctcccctgtccccagcaaagcttgcatttgaacttcttcctacgggctaacaaatcagtcagttatgtagctcttgttactttttagcttccgaagttttgttgacacccgtagtctgctaatgtccctgttctgttctttctgttcgtgtaaatatatgctttatacaacttctttacatgatttttgtggggtttctgggtagcagagcttcacaagttcaatccagcgtgttggattagaaatctcccaccctctggtttattcttattctcaaaattacctgccaaacactgatactcccttgtttttccttttcctgacaggaaatgtacataccatacaggacagaaatcattagtgtatcccttggtgaataaccacaaagtgaacttaacccttgtaaccgccacccaggtcaagacagaatattaccaagcactcagaagcctctcccctattcccccgtcactgctcctgccttcctccccaaggtcatgactgctggcttctaattccagagtctgtttttaaattctgtgtacatagaccatggattaagtgttctttttgtctggtttattttggtcgacattaagttcatgagagtcttctatattatcgtgtgtattagtattcctgtagttttaggagcttcatagcattccattgtagggatataccacagtttattcattgtattatcactgggttgtttctagttcttggctattgcgagcagtgctactgtgaccactcttaggtgtgtcttttggagtacatgtgcaggtttccatcttgcacagctagaggtggagttgttgggtgatagggtgtgtgcatctcagctgcagtagaaactgccaaatagctttccttgagtgcttgtaccagctcacccttttgccactgtgtatggggattccaggagctctggtcctcgctagcacttggaattgctgatgcttttactcttagccttcctgatgggtgttttctggaatcacattatgattttaatttccattccttaaagtacccttggctctgaagtttaatgattcatgcatctcttcccttttgaagtactcttacaggtatgttgtgcatgtgttgaaaagtggcactatctattctaaaatacagtatgcctcctctgtgtttgaacagttgtagcgtggccttggggcctcctgttagctggcttggagaagggattcttgggattgtagagattagacctgaggaggccccttggagctctctgactaaattttattctttattattccaaactatttaagctcaccgtgtgctgactcatcataataatgagtagctctcattgtgcttgtctatttggactcatacaatgattttttttttttctttgagacagagtcttgctctgttgcctaggctggagtgcagtggcacaatctcggctcactgcagcctccacctcccaggttcaagtgattcttgtgcctcagcttctcaagtagctgagactgcaggtgcgtaccaccatgcctggctaatgtttgtatttttagtagagacggggtttcaccatgttggccaggttggtctcaaactcctgacctcaagtgatctgccttcttcagcctcccaaagtgctgggattacaggtgtgagccactgagcttggccaaagtagttttttaagatgttagtatcttttcttgcagctaaaaaagtttgtcagagatgattctactttgttctccaggtgttttctcagggagaaattggaggcagtaagccactgggggagtcctgtggctggggggtggggtagtcctgtggctccttgtcagggagtcctgtggctggcaaggagagaagtcctgtggctgggttgggagggagtcctgtggctggggtctcatcctgtgcctaacagtgtccagaggtgccgagaccagctcagtcggggagaccctaacccagcagcgctagaggaattaaagacacacacacagaaatatagaggtgtgaagtgggaaatcaggggtctcacagcctttagagctgagagccctgaacagagatttacccacatatttattaatagcaaaccagtcattagcattgtttctatagatgttaaattaactaaaagtatcccttatgggaaacgaggggatgggccgaattaaaagaagaggttgggctagttaaccgcagcaggagcatgtccttaaggcacagatcgctcatgctattgtttgtggcttaagaatgcctttaagcggttttccaccctgggtgggccaggtgttccttgccctcattcctgtcaacccacaaccttccagtgtgggcattagggccattatgaacatgttacagtgcttcagagattttgtttatggccagttttggggccagtttatggccagattttggggggcctgctcccaatacagaggtctcgtgtaaattccctgggaggcgataagcctctgagaaacagactatgctaaccacgccatgaaagagaaacttatttataaatcagatgccagttactagtttactgcttatttgcccaggcgtagctctgacagagtccccgactcatagtgcttgctcagtgcatgctgaacaatgattggaatcaagtcatggctcagagcatagttttgaataatgggaaatggatgttcttaagtaacatagtcaccaagataatgcgactagctgggtcaccccttttcaattttaggatatttttatcaagatttaaatggccatcattagagttatagcactttctcctttggattgtcctagaggcccatgagaaagtattccctaatttcttaggagaacagtttgtgggtagtatgcggtcatgtccagttaaattgcagatatttccgatcgaagatgttccagtcctgagaacttcgtgacattagcaggacttctacaagccatctcttagggtggggcatttactgcagttggctagtactcttttctccttaactttgtcatttgttgatttttttttaactgtccccaaatactgtgggcagagtgtatctagaattgaggcctccaccattgcggagaggacatggatgctgagcagtcccctgagtgaaggttataaagaagcaaatagactacacatgtctgtaaactgctcttgagtgtcccaaatttggggtacttcagttcagctgtaggaaaagcctcaaactgtttatactttgcaagaattggaaacttctaattcacgttaagttttatgtaatacatgataagcttcataggagcttcatcttttatctacttggacttttgcttccgtaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggtaaggtggaggcatatgagtggaagagtctccagcatgtactcaagatagacctttgaaataaataaaaccagatgatccctcagcttctagaccaggctatttggcactggttgattgaatgtgaactgcactggggctgctgtgagcccgcatgggtctctgtgaccctgcagatgcagccgtgcccagggactgggcagtgggtgtgggctggtgtgagccctgtctgccacccagggcctggccctctgtctgtgtcggccatgactatggtgagtcttgtaggcttgagactgtgcctcgggttcctgcgggttctctgtaggtcagttgacagtttctcctgttgtttgggtaactgtggaaacgaacactggcaagtgctgaagcgagcatgtggacgtgcgatatgaaataacgacctggctttcaaaggcagtgaggctctctggaaaggaccttgctgagctagggatgtgggtgtgtagccattcccagtgggcctcatggcgtactcgttcatgatcatgtttgtgccatcttgatctctcaggatctcttcttttttaacagattaagccgggaatctccaaacagtgagtcagatgttaagatgtcttgcttccacccccacaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtaggagcacagggtttactctaggccctgcatgtgaatgactgacattcaaagaaccgattaatttggaagagaagcggcagaaccgagagttagaggtgtggactctggagctgcgctgctcgtttccaaccctaggtgctgacctctagctgtcttccctctgtatgtccctgtcaccgtgagtcaaatgcgggtgatgcctcctcaggtgccgtgttacctaagcctctcagagaccactgctaccctgtttctaaaaccagaggtcacgatatgtgttcatccacccagtaaatactgattgagcacccactgtgtgctaggctctgggataggggctgggtatacaatggtgagtatttcagctgcagcttctgccccgtggaggctgtggcctagcacactggtctaggcacggtggtatatgctcactcaaggagatagggacgtggtcgtttggggtgtcggaacaaaatgtcggaacttctctttccaatgcagagaaaccttgcagtaattctaatgtactgtgattggcagttgacttcagttctttgtagcacgcttactcaggttatttcactaactatgtaaccatgcagcctcattttaagcaattggattttttgaactttacttaaaatgttatgtcagggtttttattgtgcttaatgtgtgccatttagctaagttttgtaggatacgaaattgtaagtggcttaaaatgattcttaatagaatcatgaattgaagataatgctaataatttaagcactgagttaggtagtgtttgtaaaatgcttagaatgcttcctggcacatgttaaggccatgtaagtgctgcgtgttgataaacagctgagcaaaagtggactcttaagaaagtattggggctgagagttctgttccaaccagctgccctttggttatttttcagaataaaagcagagtctcatgggatatgacatttatatttccttcacaaaaaacactgctgagtgttttgttgagtaaaaagggtgtagccatggtaataatacatttaaaatatagtttatttcatctttaccttgccttgttttttttttaagctagctttttattgagaattccacacatacaaaagtatcaactcatgaccagttatatttcatttataatcctacttctccctttttttattatttgaaagcaaaccccaattatcctcttatttcatctataagtatttcagtatctctatagatgaggactcttctttatttttaaaactttatttttaaaatgatggtcagatgcagtgttcatgcctgtaatcccagaactttgggaggccaagctgggcggatcacttgaacctgggagtttgagaccagcccgggaaacatggcgaaaccccatgtcttaaagaaaaaaatcagccaagtgtggtgatgcatgcctgtagtcccagctacttgggaggctgagatgggagggtcacatgagcctggaagatcaaggctgcagtgatccatgattgtaccactgcactccatcctgggtgatggagcaagattctgtctcaaaaaaacaaaactgcaaaacaacgtcacaaaacagtgccattgttagacctgaaaatattaaacatttcctacatcaaatacccaccaactcattatcaatttttctctctactcttttggaatcagcatctaaataaaattggtcgataaggattgtaaatctctttgatgaactggttcccctccatcccagtttttttcccttagagttcatttattgagaaaccagattgtttgtcttctaagttttcctgtggtctgatatactgcttccatctccactgtgtaaattaacacctttttctcttctctgtatttcctgtaaatcaataattggaggaaaagccttgtcagatttagtgtatattttatatctgagtccagtatttcttatataatattttaagataagtgtactcttttaaaaagtattgaaactatatgctcaattttttttaactgatgcttttaagaaggctgcttgatcataaaagtttagagatcattggtctgatgggaaaagcaaataattactaaaccgtttagcaaggttgaggtgcacatggtggggcctggagaagttcagtcatgagccgtcacttatgggcacgtggaatctgacccggcacagagttgggagaagacaggagctttatagacagaaaatgtggtctttgctaagtcccaggagtgaaagggtgagacagtgctcacagcacacgagtgtgggtgcgtagacagagcaagggtgggtcctgaaaaggcctgcaggctttctcatagattagcaagagtgctggttacggaggtttctaacatttgtgaacagatcgaaactgtgttaaattgggattgcagtaatcctggaaggacagggatagagggtgaaggggaaaaaagggtatggatgtgagacttaattgctgattttcttaagacctttctccaaagtaaataaatgatgtggcacatttttgaactggcaaattctaaactctagatatgattatctctataacatatcttactccatcttcttttgactaaaaactgttcttaattaaattaccatgagacgttcaattcagcaaatgtagtttggctaaccatatttaattagaatttaatataatcctaggcctggccaaactattaagcaagtgtgggcaaaatattgataattttagatatgcaggaacttagtttgctttccatgtgtgcttttcgaaaaaggaataaattgaaaaatagaggaagccctgaaatccaagaagcaaactctctcacctaggcatgcagtaaaagcaattctaggatgattgctgtttggcgcgtagttcgtattagaaaccattcttcttgaataaatagtatgtttaagaagctgggcagagggaaggcatatgcatatattatcaacaaggagggagaaaaaggcaattagtaaccatccataggagggtcagcaagatttataaaggaaatttgtgatccaagtatgaagcaaaataaggtgcagaataaattttaagcaagtaatagattagagtaagagaacccatttgaccattaaccttgggacattctctttcaaatgacatggagtagtactgaaatctttctttctttctgagtctaggttattgtgactggactcagaaagaaatatttcattattgcagtgaataacatttgtgaacattattgttcataaattatgcagtgaataacatttatgaacacgtgatgtgtaagatacatactgtttatttttagttaagttttttggctcaacttctaggcagagaacattaaatgtaaatagtgttacctaggagcatgtaaatggaaatctccatagtatgaaagcagtgctgttgctaacagaatttaggagggggcagatgaggtgaaggaaatgtgggtgctgatttccttattacattgagaggagccaggagattctttgttcaaaatggatggcttaagaagtcaaagtataagctgattacgtagagcaggtacccaaaaatgttttgtgtaaggggccagatagtaaatattttcagtcttgcaggccatcccaagtctgtggcagctactcaacactacctttgtagcatgaaagcagccacaggcagcccataaatgtggctctgttccggtgaaactttaggtacaaaagcaggtgcaggccagacctgacctgtgcactgtggtttgctgacctgggattcaggggtatagaagttaccatcagaagagctaaaagtgagactttttactttatactcttctacactgtctgattttgaaaaaaagaaacatgtattttataatattaaagatagggttggcaaatagcaaataaaaatacagaataccagtgaaatttgaacttcagatacattatgagtaattttatggtgtaagtatattccaaatcatgtgggacatacttacactacaaaattatttgttgtttgtttacagtttaaatttgagtgccttgtattttatctggcaactgtaattaaagggaaaaagaataaattcattatgttcatataatgtgatatagcaggggtccccaacccccaggctgcagagtggtactggtccatgggtccccaacccccaggctgcagagcggtattggtccatggcctgttaggaaccaggctgcccagcaggaagtgagcagcaggtgagctggcattcccacctgagcaccgcctcctgtcagatcagtggcagcattagattcccataggagtgcaaaccctattgtgaactgcacatgtgaggggtctaggttgtgcgctccttatgagaatctaatgcctgatgatctgaggtggaacagtctcgtcttgaaaccatcccctggccctgtggaaaaattgtctcccatgaaaccagtctctggtgccagaaaggttgggtagcactgtgatatagtattaaaagtgctaataaatatggcatactgcctttaaaatgtctggtagctctttctcagtggcactcataatagtgttttttgatttttaaatgtgtgtcaagctgactctcccctccgtgtatgctgggctttattttccctttcctagtcaccagttttgggaaatagagatcttcattctcatgctgctcctctagtgcaagtgctccatttatttttaaggaattaatataacaaaaaatcatgggaatttagaaaacaacatggaagctaatgatcacattggtggaagtgatagggaaatatttagggggagaagttaaggtataaactttgtcaatgaagtcctattaaaaacaacaaaaaagtgaagcttaggatgcattttataaactctgaccagaacacctgtgtttctctgtttctaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttataggcaagttattagcaaggtctactcttacaattaactttgcagtaatactagttacactctattgattatgggcctgccctgtgctaagcagtctgcattccatcttccttgccaaaacttataatacaaatttcatctttattttataaataggggagttgggctgggtgtggtggctcacgcctgtaatttcagcactttggaaggatcgcttcagcccaggagtttgagacaacctggccaagtgagaccctgtctctacaaaaaaaaaaaaaaaaaaaaaattagctgggcatggtggcacatgcctgtagtcccagctgctttggaggctgaggtggtaggattgcttaagcccaagaggttgaggctgcagtgaatcttgatggcagctgcactgagcctggtgacagagcaagatgctgtctcaaaataaatttaaaaataaaataagagaattaaagtttagcaggttgggtggcaaaatgaggccacacatttaaagcccctcctcctgattcttttctctgccttggctgcctcctgtggcattttaggtgctgagaaatgaaaacagtagggaaaatagttccaggatcctcatgttaatttgccagaaatggcatcttcaagtcgtcagagggatctgagagttccttcctggcctgacttgagaaaatccgtctgtccccagctctgcgtctgcctccactgcccagtcacctcctctccatgctcttggggctgggccctaccccaccatgcagtgctgccctggagcagtgagcttggtgggtcctgtctggcatgagagctgcctttgggagctggatcccagcctctaccactgggtctggtgcctagcaggctatggataaacttctgctgactccggcctctcctaagccactgcaacgtggtcggtgtagtgcacagtgtgtgtgcagcgtggccttactcacagcctccacattagagagaatctgactgaagtcttactgctgcctcgtgtgaacataaatgtttgccagaaccatgagcaggaaatgttaatctgccttgtttcctgtcctttacacggaagaatttttttctgtatggaatgcgtgccttacaaataatgagtggaaatacccatcgctaatgaaaagttatacttgactgttagtcagctaaataatctgagatttctaatacttttaatttggcttttacaatgcaatttatcttagcttttttgatttcttaggtcatatctttagaactatatatttgaatgttaatgtaattttcatattgaaattaaaatgttgaactgcgatgttaagtgtttcctgtggaaaaacgttcacattttctctagttttaaagttgaatcaagctgtttgaagattttcacatttcttctagattttatcagcttgttactttatctgtcactttctgtgatttgcagctggagggggttcctcatgcagccctgtcctttcaagaaaacaaaaaggtgattatttcagaaatcagagtcttgtgttgaatcttactgattttcttgtatttctgtaatgtaatgtatcttgtatttcttgtaatactgtattggactctgtgtatatctcttctcagatgagtgattatatgtgtgaatgttgctggaatctgataaccaggcctgaatagttttgtagggtggcttttaaaaattactttcatatcagaattgctttgtcataaattttgaacgcatcataaatttctaatgttcggggtcagcagactttttttgtaaagggacagagtgtaaacatcttagctttatgggccatatggtctcttttgcaacattcagctctgccctgtgacaggaatgcagttgtaaagacatgagctactggccagctatgttccagtagaactttacttacagaaacagacaggctgtagtttgccaatacctgccttagggaatgtgttgttatattttgtgagttaccttctcagtaaattttatttagtattagtcaggaatattattaagtagcttcttttccagcctggtcaacatagtgagacccggtctctaccaaaacaaaacaaaacaaaaaaacagccacgcatgtggcatgtgcctgtagcctcagctgctgctcagggggctgaggcaagaggattgtttgagcccaggagtttgaggtcacagtgagctgtagtcatgccactgcactccagcctaggcaacagaatgagaccttgtgtcttaaaaaaaaaaagtttcctttgttgggttattttaatttggacctggttatcatttttcagccatatttaactttgtacatatcagaatgttctgataaaacttaacttttattaaagtgtttgtgatataatctgctagttttggtacacattatcttttgcaatgccagttattttcttttccagtgtgggtttgcataggaaaagaattgctgtcactttctattttgaaatcttaaaagactgatccttttttgtgtcatgatttgagtatttaattgagagcctaatgcctaatattatttgcagtattaaatgggatcttaacaggaatagcattctagccttcattgaattaagtaaacatttcttaagagaacttggaatctataatatttgcgtcatcatagtatgagatacttaatcaagtttgagattttagtgaaacattgtttagaagccaaaaggattctaggaaaaattaatgtctatattcttgaattaggagagattttgggacgtgtgactaagttacgctgacacttgtttgtttcttagtcgctttttccagtggcggtgagaacgaagatgactgattcacattgctcagatgagtttatcctcttctggctgggacatgggatatatcctgtctcttttaagcctttttggtatttttcccccattgagagctgtgtcttcaaactcttctgttatagctggaaaatcctttttaagtgaaatctgcccaaattataagacagatgaaggtagagttgtgttggatataggattagggtgaaagtagtgggggtgtcctggagcctctcttctggtggcagcctagctcttgtgcctttgaggaaattaccctggggacggctctgtggaacatatttgcaaaccactgatttggaagatagagatggcttttgttaagatctgaattcacctttttggcattttatttgatttctcaaggtaaagaacttattttgtaataaagtttcctattatttagtagataggccaagttgctgtgttaattccatgtagattttgggtttcctttgctcattttttcactcttaatctcacatcattgtaagtttatggaagttatcatacttctgactttttctttgaagagcagaaattagaaattcccaataattattttgatagtgtcatttaatgacactcacatgtgatgtagccacaaagatttaatgagttcagttttaaatcatattaagactgttggtttcatttgttctcattaatgtaattctgaagatgaacaataaaatgtatttttagaactttcaaatgaaatattatttcatccttccagatcatataatgcttaagttctgattgttaatcataaagtctagaaaattaaaagataataaaatgaaagtgacttttaggtattagagttttattataaattctggtgtgtcattggagctatgacatgaatatttcaaaggccaatagcattggatctttacagttataacttaccatttttaagtttaagtagtaatatagattatttaataatcaaaatcaataaatattaattattaaaatgttttgtggtatagtttgagaatcattgcttttaactttttccatataggtttattgactttaatagcattctaaacataacatctctacattctttgtgtttaatactgtggaggtataaaaatacttatatatgatgataaactatattagagtaaattaaatattcttatgagtttcattttagagtgcatttacttaattttgaagtccttatttttagcaaactaaaaggaatgttggtacattatttactaggcaaagtgctcttaggagaagaagaagccttggaggatgactctgaatcgagatcggatgtcagcagctctgccttaacaggtagttctcactagttagccgctggtgtggaccttcactgtctgccttccaccccttgcccttcctgctcgtccccctgcacctggtggacagcacgactgggggcagcagtggagccaggttgcttaaatggggcatattcgggcttcttttataatacttactctgaagcttgtgtgtctgtggtgtttgcatcatatatttgttgttttccatggtttaggctgttttaaaattaggtttatggcttgagcatagggctttgtgagtaggggatggcaggtcgaaacatctcatgagttggatgggttatgctgggggttgggaaatgggatgaaaaattatgggatgaaaaattgcctatggatagtttaacttgaaagaatctgcctttgtttacagatagttatcttttttcttttttgagatagagtctcacactgtcacccagtgcagatacccagtgtcactggagtgcagtggtgtgctcttggtgcactgcagcctccgccttctgggttccagcgattctcctgcctcagcctcccaagtagctgggactacaggtgcccgccaccacgcttggctaatttttgtatttttttgtggagacgggtttttgccatgttggtcaggctggtcttgaactcctgacctcaagtgatctgcctgcctcagcctcccacagtgccgggattacaggagtgagccactgtgcccggccagttacagatacttatctaatgaaattctctgtgtactttataaaagatgaggattaactgaaggtactaataactggattatatgagggtggttttggttgtataatcctatctaaaagaatattttagctataactgaaagtaagacttaaatatttagagaggaaaatctgaataattctagtagtaattatttatttacaaaataaaaatagatttttttttgattacacaaattaaacaacaataaaacatcacagcaatccggatactataaagctcacatgcttaccgacccaactgccccaggagtgaccactgccaacagcttcatgtcgacctttttgccataatttttatatagccttttttgtttttaaatggtaatttagaaagtcaactaggaaaatgtgttacaggtttatcttccaggagaataggactggagtcgagatcttgaatgtggcttggaagaaggcaagcccaccccagagagatgagttgacagttgtttctgaccactgcttgcttagagggcctgcgtgtctgtgaccgcctagctttgcgcccctgactaggctgccccttaattacaaatgtctttatatattgctccagctaaggcttggagtagtcggttaagaacttgaacttcggtttttgcagtgaaacagcatttgagaatatcaccttctgataagccttattttataaggtgggtactgtagtgggaggcagtgtgagagatgcttgaaggatgcactgctgtcctgcatttcagcatcttcaggatgctgtgcagctgaaacatttgataacggtggaactgttcgttattttgcaagcctgtgattccctattgaatgttttctctcgccatttgacaaatgagtgtttctctgtcttcagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtaagtgggcagaggggcctgacatctttttttttattttttatttgagacagagtctcactccatagtgcagtggaggccgggcacaggggctcatgcctgtaatcccagcactttgggagactgaggcaggcggatcacttgaggtcaggagttcgagaccagcctggccaacatggtgaaaccctgtctctactaaaaatacaaaaattagttgggcgtggtggcacatgtctgtagtcccagctgttagggaggctgaggcaggagaattgcttgagcctgggaggcagaggttgcaatgagccgagatcgtgacactgcactccagcccgggcaacagagcaagactccatttcaaaaaaaataaaaaaataaagtgcagtggctcgttctcagcccactgcaacttctgcctcccaggctcgagcgattctcccgcctcagcctcctgagtaggtgggattacaggtgggcaccaccacactcagctaatgtttgtattttcagtagagacagggtttcaccatgttggccaggctggtctcaaactcctgaccttagatgatccacccaccttggcctcctaaagtattgggattatagttgtgagccaccatgcccggccctgccacctgccatcttttgagttcttccctggagacctagacctgaaccctcctgcttgttctcttgttatctaatacccctattgacagcgcagcttagatcattaatggagagcttgacctcatctgataccttcactgaaggaaacaacttagtgtcttttgtgttgaacactgaggtaaaaaattggaatagttgattatatgaactctgctaaaattgagtgcattttacattttttaaggccttgttgggccctggttaaataattatttttaaaaatccttaaggagcctattataaacagatctgtggtcttaatgaaatgtgattaatactgtgcattattttaagaacttttgacttttcaaaaaacttttacaacatttcccatttgatagcggcataggtttaagcacttctcatctctaagttagtggacaaaaaaccctcatggatagtctaataatgtttgctacaagtccatgttgagttttatactccattttattttcagttttaaaaactgtggttaaatatgtgtaacataaaatttatgttcttaaccattttttgcgtatacagttcgctggtattaaatacatttaaataatgtcatggaatcattgctaccacccatctctgtaaccttttgatcatgtaacactgaagctctgttcccattgaactctattcctcctttcccgccaagtccctggcaaccacgattcttctttctgtcttctgaatttgactactttgggttctcatatactttaggagtcacacagtatttgttttacttagcataatgtccccaaagctcatgcatgttgtagcctatgttagaacttcctaatgtttcaggccaaatactattccattgtatggataggccacattttgcttttccattcctctgtccatggacacttgtattgcttcatgttttagccattgtgaatcatgctgttatgaacgtgggtgtacagatagctcctggagactctgctttccatttttttggctaaatacccagaaatggagttgcttttacattccaattttaatttaaaacattcatatcattgagtgttttacttaatagtatagtagttaacaaacttaataaaatagtattttggtaataatttgctggtagtccattgttcagtttttttaggtaaattacacaggacatttcaagtggacatgaaacatcttgtgatgtggaatcatgccccaagctgatggctaaacatatgaaataccataccctaaatttagtagatttagtctttgcaatttaggagataacctgttatattgttaggtttttgtcgaaaagctttgtcctcatatttccaacttgctgtaaaatttgtttgtgaagacaaatatttttgtatgggttttttctttttcatattaaaaagaaatgtccacattggaatttttttggagtttttagagctaatagagcttttcataatgtagtgggaatgagtgatcagtaagctcttagcagtttccatgcgtgcatttctgtgccttgaaataaatgacagatgagtacatttgtgttctgtgtgtaaaatgtgctctttcctcattgcacttccatgttggagggcttgtctcttggtgatcacacttcaaaattctcacagccccccttgaaccgtttaggtgttagacggtaccgacaaccagtatttgggcctgcagattggacagccccaggatgaagatgaggaagccacaggtattcttcctgatgaagcctcggaggccttcaggaactcttccatgggtatgtggactacaggtgatgcgctacaaagtggtttgtattcagacctggacatcttaattatatctttgcttccaagaagaagtcctttgatactgttttctgagttctgaatagctgatgaaaatgaccaattgaggaataatcatactttttcttgatctaaatcttatacttttgagttatcttagcataaatgtataattgtattttaagtggaaatttgtcacttaatcttgatttctctgtttttaaagcccttcaacaggcacatttattgaaaaacatgagtcactgcaggcagccttctgacagcagtgttgataaatttgtgttgagagatgaagctactgaaccgggtgatcaagaaaacaaggtgagggacataggcttgagacgacttggtgtttctgagcttgtgtgaggatttaaaatcgccctggctactgtctactttattgctttcccatccctgggcctttaaatttcccctttaaataccagctcttcccaggcctgttgttttctgcctttccaggtactacccacagccttgagaattgcctgagttctgcctcctttgagagtgtgccccagacaaatctattctgtactgaatgtttccttgtctgatttcttggatcattcatttgatggttgcgtatggcctgcaacgtttcttgttttggttctactgaactgttctaaaagtctctcttcatattatctttttacatgtaaatgtaactgtcttcacttttaattcctcaaggacaaggaatagcgtttcacagttcgtcccatcaatcagaattatagcctttggcatctccctatctaccaggcccacttcctcttagatttgggcttccccaggctgttgcctttccccaagtagcttctgcttgtcctgtagaagacctttcatgctttgcttctgcagcagccgttcctgaatgcctagtgtcaactgccttcttaccacgcccaccctccctgcatgctgcatttatcccctgccacagccctgtgaccctgtgtcctgctgcctctgacttgtctgtttctgcttggccatggtctctgtgaggtcaggtgtgcatatgggcacaaaccagggcatctctttatccccagcacctggcttaagtgctgctctggaactatctgttgaatgaactaatgcatgaatgtattgttgagtatgagacaaacaagtgtcattgtctcctttctagccttgccgcatcaaaggtgacattggacagtccactgatgatgactctgcacctcttgtccattgtgtccgccttttatctgcttcgtttttgctaacagggggaaaaaatggtgagtacaaaaggggatgtgcacagttgaaggaaataactaggtttcagaggtcagcttggtggcctgtttttgccttgcgtgcagcagaggaagtagaatctgaggatgagtttggttttcactagccgaggggagggaggaaatgatgggagcaggtaggttattgggtctggttttgttcatttgaaaacaatctgttgtttgaggctgaaggtggcttgggtgatttcttggcagtgctggttccggacagggatgtgagggtcagcgtgaaggccctggccctcagctgtgtgggagcagctgtggccctccacccggaatctttcttcagcaaactctataaagttcctcttgacaccacggaataccctggtatgttaaaagttcacatcttattttctcagatttaatcattattgtaaaaactatttcagtattgactattttagttttagagcagtaagtgttttgagttcatttgggatatttgacctgcgttgtagctcttcagaaaacacatgaatagtgaagttctttgtttcatgggttccctttagatgaaacccatagaggagaaaagtagaaacctcagcacgtaagagccaacatatatacacatcggatttaaacctaaagcacaaattgtgcctggtcgcagtggcgctgagtcgcactcagccaggccaggcattcacactcagggtgagtgggaaccaggactggctgaggcagcagtggacccaagtctccatcgcgcccatgcttactatggagccttctcgttctctctttttctttgggtgagagggtacacttgtgtttttgaatttatatgaggtaagtgtgtaatagggttttttctaatcttttttaagtggaatctggaattttaatcagatttattatctgacaacctagaattataatccagaaagtctgtggtattgaggacatattggcaatatgatgaatctctaattcttaaatcctgaaacttttttttttttaatcacttagggttattatagtgaagtcatttctgaatttggatcttctcttcacacctctttttctctttcctgagaattaagcttttgtttcgagttagaaagttgatagtagggaattgttccatggctgagcaatttatctccacagaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggtaacggccagtttttcagctgtgttttttctagttatgcttactaaggtttaagtttagatgatgatgtttgttgcttgttcttctggttaggaaatacattttctttggcggattgcattcctttgctgcggaaaacactgaaggatgagtcttctgttacttgcaagttagcttgtacagctgtgagggtgagcataatcttctgtggaaccatttcttcacttagtggacattttatcattgctacaattaaaattggagcttaataggaaatatttccatgcactctaaagctgtaaccagtaatacccaccatgtatccatctctcagctttagaaagaaaacgttgccagtaaagttaatgcttcataaacttcagtttaagttctaattctcagaatatttgtttgaaatagacctcttcctaaaggatatatttagaaataacctatcattaagtgtaaagtctgttgaatatgctgggcacggtgactcacacctgtaatctgaccactttgggaggccaaggtggaaggattgcttgagcccaggagttcaagactatgggcaacatagttgaccctgtccctacagaaaattaaaaaaaaaaaaaaaaaaagtagctgggtatggtggtgcatacctgtagtctcagctactcgggaagctgaggtggaggggggattgcttgagccccagagatcaaggctgcagtaaggcgtggttacaccactgccctctagcctgggcaacagagtgagactgtctcaaaaataatagtaataataatcagttgaattaaaaaaaaaaaaaaaaaaaccactgtgctaggcccatagtatggtaagagttaaagtgagccttagggattatttactcaacctctgtttctgtataaagtggaataggctcaattctttaagtgatagcatgttgaacctttccataccaactggctcataagtcacaactggccagtcaacaagagtaaaaattaactggtaaaaatcaaagcaaaaaacctacaattgtcaaatttgtgggataactcccccttttaaaatgtcatgcctgacagtaatttctctctagtttccaggttttcagtcagttgtgtcttttttgagcagaaggaagcatgctaagagctcaatcttgtggctagctgggggtctttgtgtcagccatgcatgtgatggtgcccctgggtgcttggggctgcaggggaggggtacagcagtaggggcctgttctgttctctcgtgctgtggagtacatagtgacatagtggggtggtccttggtgtaggtcccttgttcctacccctgggtctgagatttatttagaagtggtgttggggctgtgcggcaggcccctctgtaactgatcaatgtttgtgaagttgctgtttgagagttgaaaccatgacataagcagaaatggaaggaagaaagaaccagttatgtgaaagggacacatttacttttaagcttgtatttactgagataaagtattcttaatcaatgttcttgagaggtgtgggaaaaatgcaacatcctggttgcagttaaacccagaacattgtgtgttgaagagtgacggttctcaaaccgtcaagacgcgggtactgagtgggactaacctgctgtcctcttgccttggaccttgtgttccagaactgtgtcatgagtctctgcagcagcagctacagtgagttaggactgcagctgatcatcgatgtgctgactctgaggaacagttcctattggctggtgaggacagagcttctggaaacccttgcagagattgacttcaggtaagtgagtcacatccattagatttcatgaactaagctcaattgaaagttctgggatcacttgatgcaaggaatgatgttatcaagtaccctgtccatcagaaatccgagtggtttaggtagatgacagtgattttctcctcccagtggctttttgctgaactttgccctatgcttggaattttattttattttattatttatttagagacaagatcttgctctgtcgcccaggcttgaatgcagtagcacaatcatagctcactgaagctttgaactctaggactcaagtggtcctcctgcctcagcctcccgattagctaggagaataggtgtgtgccgtcacactggctaatattttttgtagaaatggggtcttgctatgttgcccaggctggtctcaaactcctgggcttgattgatcctccatcttggcctcccaaagtgctgggattacaggcatgagccactgtgcctggcctagaattttaaaatataagtagaagagtagatttttttttttggtagtcctcgtcatttaagtattctggatagtgggaataaaagagcttagaatttttcatctttgtcttaaacttttaaaaaaatgtagcttatattaattctgcttgtttaaaaagaatatactcttcattatactgaacctaggtaagacagctggtttatattttgttgcaattaaaaaacgtgagctgtggttgcagtgagccaagattgtggccattgcacttcagcctggcaacagagtgagacttggcctcaaaaaaaaaaaaataacatgagctgtgttggcactttcattttctaagagtagttttggctggagaagttttctttcagtactttcttttagaagggaaattttcctttataatttagggtttgttttttttttttccaagccaccttttatagagcccttgtgggttatttcatttaatccttagaatgtttataaatctgggcttgttctcggctccacccacagatagggacgctgagcgtgcatgagtgggcagcaagatagcaggttatggagggcccagctcaccccttctgtggcttgagccaattttatagggcacttacagagtcttttgaaatagtatttattttgaagaaaaagaaaaacagtttactgagtactgtcttattgagtctggaattgtgagaggaatgccacctctatttatttaaagccattggccttttttgttgttttgagtaagtgctgcccaaggtccttccagggcacctggatgagcctgctctggagcaagctggcggtaagtgtttactgagtaactaaatgatttcattgttaaatgtgctcttttgttaggctggtgagctttttggaggcaaaagcagaaaacttacacagaggggctcatcattatacaggggtaagcggtttatttttgtgagatgctgttttaccttcaagaaggtgaaagtgaggctttccttgtggaatttctctaaatgcattcgtcatgttttagatgtttatttcacagtttatatcatgaaagttataatcttgtcatatggatttaagtctagtaatgttgagttctttctcactagctttccaaaatatcttacctaaaatttagtcaaatacaagattatgtttatttttattatccttctctctaaagcttttaaaactgcaagaacgagtgctcaataatgttgtcatccatttgcttggagatgaagaccccagggtgcgacatgttgccgcagcatcactaattaggtatttaccaatattttatctcttttccttttttggttgaagtactaaaagatacgagaatggaaagagagggaagaattcaaaggatgtagagcagtattcctgaatctgagctcatttcagccattctattcttaaactataatgaaaaaaaaatccaaaaaagtctaaaattataattaaaaaaacaacaaaatactaactgtccattgtaaaaagtaatgcactttcattgtaaaaattttggactatagagaatagtactaagaagaaaaaaaaaatcaccttcaattctgctgccacctggaggtaatcactgttaatattttgctatatactctatgagtttcttgttcaaaatcaggtcaaaattacatgcaattttgtaatctgacaatttccacttaatattttattagcattttcctgttatgaaacagtaattttagttatgggtcgttgttttgctatgcggttgggataaaattttatatactttttttggcaattacttattatacataaatgtttgtgtatagttttctttttctgagaattcctggaagttgagttaccaggcccggctttgaatttttttttttattttttttttgagacagagtcctgctctattgtccaggtgctatctcggctcactgcaacctctgtctccctggttcaagcgattctcctgcctcagcctcccgagtagctgggattacaggggcacaccaccacgcccaattaatttttgtatttttagtagagacagggtttcacgatattggccaggctggtctcgaacttctgaccccgtgatccacctgcattggcctcccaaagtgctgggattacaggcgtgagccatggcgcctggccaggctttaaatttaaaacaaatcttctaatagctttatggaggttataatttacatttcttgaaatgtactcactttgagtgtatagtaaactccaattttatcacatttctgtcaccccaaatgtatccttgtgcccatttgctgtaacctccggttcctgccccaactcctaggcagccactcatctattttctgtcccttaagatttgtgttttcgccaggcgctcatgcctgtaatcccagcactttgggaggccgaggttggtggatcacttgaggtcaggagttcgagaccagcctggccaacatggtgaaaccttgtctctactaaaaatacaaaaattagtcggatgtggtggcacacgcctgtaatcccagctactcgggaggctgaggcaggagaatcacttgaacctgggaggcggaggttgcagtgagcagagatcgcgccactgccttccaacctgggcaacagagagagactgtctcaaaacaaacaaagatttgtattttctggacattttatagtactggggtcatagtatagatggacttttgcatttggcttcttttacttaattgtgagattggttcttgttgtagcatgtatcagtagtttgttcatttttattggcgaaagtattctattatatgaataataccatattttatctatccatcagatggatattatagagttcatgttttggctaatttatgaattatggtactgtgaacatttgcctgcaagattttgtgtagacatgtcttcatttctcttgagtagatcacctagaagtggatttttaaataattttggtacttactgtgaaactgctcttcaaaaacataccattgttccttccttccttccttccttccttccttccttctttccttcctcccttcctccctcccttccctacttccctctccctttccctttcccttccccttttcccttccccttcccgcctgcctgcctgcctgccttccttccttccttccttcgtttctttctacatatacacatttttttaaatttcaatggtttttggggtacaagtggtttttggttacatggctgaattttggttacatggtgaagtctgagattttagtacacctgtcacccgagtagtgtaccttgtacccaatatgtagttttttgtccctcaccttccagccttccgccttgtgagtctccaatgtccattataccacactgtatgcccttgcgtacccacagctcagctcccacttctgagaacatatagcagaaacatgccaaagtatactcccactaccagaatgtgattgtgcctgattcttctcaccagtacaaatatttcaaaaaaagttaaatatgtatcagttttttgggcagaagttgatacttctctttatttatttattttttttgagatagggtctcattctatgatgcccaggctggagtgtggtggtgcgatctcggctcactgcagtctctgcctcccaggttcaagtgattcccacgtcagcctcccaggaagctggaattacaggcgagggccaccactgccagctaatttttgtattttttggtagagatggggtttcaccatgttggccagactggtctcaagctcctgacctcaagtgatccacctgccttggccttccaaagtgctgggattacaggcgtgagctaccacacccggctgatatttctttttaaaataacttaccttcttttgaaagtaatacatgtttaatgaacagaatttaaggaaaatataaaaaaacgaaataatctttgtaatcaaactactgaaaagaaaaccaaagttacattttggtgcatattctttttcattttcatcattgtaatttgcatttctttgattacttgtgagacactcctttcatttacttaataggtttatatgacttgcctattcagagattttgcagctttaccattttctgcaaatgatagcaacttctttttgtttgtttgtttgtggagacagagtctcgctctgtcactcaggcaggaatgcagtggtggaatcttggctcattgcaactattgcctcctgggttcaagcgattttcctgcctcagcctcccaagtagctgggattacaggagtgtgccaccatgcccggctaatttttgtatctttagtagagatggggttttgccatgttggccgggctgatcttgaactcctggcctcaagcggtccccctgtctcggcctcccaaagtgctgggattacaggcgtgagccaccgtacccagccagtagttacttcttatattctagaaaaaattctactcatgatcaagtctccatgaggaaagagactttaattgaagatcatggggcttgcagaccaatatgataaaatagttcattgtttctaaaagtattactgagtgttgatggcagatatgaacccttttgtttttgtaggaaaatgttacccgtattctccatttgaattcagtttagatttgttaggaatcgcagcttaagctttgccatctgggagtgtttgggacagttttgcagacaaaattgcaaaagtgcctaaggaatgcagctggcattcagacctgctctgtgctcagtactctgtggacagacactgttcagcacttgttgatcagaaggtttagaaagagaactttcaaagttggtttttaattaaagcatttaatagtgtaaatagaaagggattaaattttatgacagacaaaagaaagtacagcacccagctgggcgtgggggctcacgcctgtaatccagcactatggggggctgaggtgggtggatcacgaggtcaggagttcaagagttcaagaacagcctggccaaggtgatgaaaccctgtctctactaaaactacaaaaattagccgggcgcggtggcaggcgcctgtaatcccagctactcaggaggctgaggcaggagaatcacttgaacctggacggcagaggttgcagtgagccaagattgcaccattgtactccggcctgggccacagagtgacattctgtctcaaaaaaaaaaaaaaaagaaaaaaagaaagtacagcacccagttatgtccgagtgggtgcatgagagtgaccctgagattggagacaacgctgtcacgtgcttgaagaacgccacctgagaaagggggcgagaagtggtgtccgctggtaaccagaggtgttggcttagccatctgcagggaggagggtggtctatcacaggtgagtttcatctactttcttaagcaaattaaccttacttttgtgttaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccaggtatgctgacccagtggcatcttcacattgtcgggaaaatgccctttcctgatgcctttctttaggctttaattgaaaacattttattttctagaaaaaagcttcagctcaggatgtttgagtgtaggtcagtcctttgataggatattatcattttgaggattgaccacaccacctctgtatttaagctctgccacaatcactcagctgtgacactgtaaatctcttaatagtttattacattccatgtgctgacagttgtatttttgtttgtgacacttacgtattatctgttaaaacattttcactttagttgtgttacctttaaagaggattgtattctatcatgcctgttgattttttggtgagcgggctattaaagtcagtgttatttagggttatccactagttcagtgatttgcgagattatcattcacatttattgtggagcttttgaatatcgtgtcaaatggccacatatatcccattcttatctgcttcttaggtgagtgggacacagtgctttaatgaagctataatcttcagaattctagcttgcagagaagattgcagaagtgataagacttgtgctttttaattttgtcttttaaatgttattttaaaaattggctttatatgatactctttttttctgctgagtaacagtgttttacaaaacttggactaaatgacttctaagcttaaatgatcacttgatgctttttttctgaattaggaactcagcttatcaaatatcaaagtcataattcctgaataaataacgtcttttttcatgtaaagactgctttaaaaaacacatggaaggctgggtgcggtggctcacgcctgtaatcctaacactttgggaggcccaggtgggcaggtcgcttgagctcaggggttcaagaccacccagggcaacatggcaaaacccacctctactcaaatacaaaaaattagccaggcgtggtggcgggcccctgtaatcccagctactcgggaggctgagggatgagaatcacttgagccccggaggcagaggttgcagtgagccaagattgtgccattgcactcccagcttgggctacagagtgagactctgtctcaaaaaaagacacacacacaaacaaaaaaaacatggagacatttttttggccaccttaatatttcccctcagataatttcctttgtttaaactcagaactggcattttctctcttggagaagattcaggacaaatactcctttaagataagtagaagcagtgaaagaggatttgattatcaggaatttgataagcttagaataaattgttgcttcttaatgtcatttcagaagatgaatatttattaatagatgccaactgagatatcattaaaattgattactaactactacttggaaaagtctcccagttccaaacttcagcaggcctcttgacaattcagctgtggtcaattgggtcttgcgtgatagatacaatgaccaattgtgcagcagagtgtgctgcttagctgcctattctgttagcattcatgtgttaacttaaaatcataatctccttagttttgttgagtgtctccgtggacaagacactgtgagggatacaaaatcagattggctttattcaaaccactggggtattataattcatttataatttattttattttttgccttttttccatgtgttctaaaggaattagagtttgtatataactataatgggggatagaaattgacatgtgccatgaagggaatgcaaaaaagtgccgtgggagatgagaagtggagaaaggaatttcttttttcttggaagcaggaataacttcatgaagcatgtatttcaacttaaacagatagtaggcaacgctgtaaggggagtatggctgcagcaaaagtgttcggggcagactgggaggaagggagggaataaattcagccattgttatggaataatgatcaaaatttattttcagcccgtttcacttaaaagttgagactgcttaactttttttaatctttaatcttaaacttttaaatgccatttgatctttaaaaatatatgttttaatagtgtattttaagtctctatatttttgttattagaatatatagaggctataacctactaccaagcataacagacgtcactatggaaaataacctttcaagagttattgcagcagtttctcatgaactaatcacatcaaccaccagagcactcacagtaagtctctttcttgatcggtcttactgacattgtaatagtttttggtagcttgtatggccagttagttgtatggtcatcttacggtgaggtgcttgtcttacagctcttacttatccatgaggcttgctaagaaattgtgcttctgtgaaaagaatctcagcttactccaggaatgtaaatgactatgttttttctgattattaaagtaatacacgcccaaaataaaaaaattcagccaatttaggaagacacaacaattaaaataagccaggcatggtggctcatgcctgtaatcccagcactttgggaggccaaggttgggggctcacttgaggtcaggagtcggataccagcctggccaacgtggtgaaaccccatctctactaaaaatacaaaaattagctgggcgtggtggcgggcgcctgtaatcccagctactcaggaggctgaggcaggagaatcgcttgaacctgggaggtagaggttgcagtgagctgaggtcaagccactgcactccagcctgtgcaatagagcgagactctgtctcaaaaaaaaaaaaaaaaaaagaaaagaaaaaagtaaactactgtcacctgcattggtaatgtatcagaagtttaaaatgtctagattataattaactcagtgacctggtaatatatactaagggaaaaatatttataatttacatttttacatttttatttttttaattttattattttttttttgagacagagttttgctcttgttgcccaggctggagtgcaatggcatgatctcagctcaccacaacctccacctcccgggttcaagcaattctcctgcctcagcctcctgagtagctgggattacaggcatgcaccaccatgcccggctaattttgtatttttagtagagacagggtttctccatgttggtcaggctggtctcaaactcccaacctcaggtgatccgccctcctcgaccccccaaagtgctgggattacaggtgtgagccaccatgcctggccttacatttttataataagaatttatgttgctgacattagaaaagaaccataatatccaagaatccaagaataattaaattatgtacatatgctagtatatagtgtgatgctttggagaatttttaacaatatggagatgtataatctggattgtaatattgagtgaaaaaaggcagaatacaaacctggtgggggtatagtcggatttcagttaagaaaaataatatttacatatatacatttctcacactggcagataatcaccaagataaattttgggattgtggatgatttttttcttctttatatttttcagatattctcaaattttctaaaatgagcaagtataacttttgttatcagaaaaaaataatatacaaaagtaatgttaatttgctggtgaccaggttaaacctttttatttttattttttgagatggaatctcactctgttgcccaggctagagcacagtggcatgatcttggctcactgcagcctccgcttcctgggttcaaatgattctctggccccagcctcctgagtggctggaattacaggcgtgtggcaccacacctggctaatttttgtatttttagtagaggtagggtttcaccaggttggtcaggctggtctcgaactcctgacctcgtgatccacccacctcggcctcccaaagtgctgggattacaggcgtgagctactgcgcccagccagacctttttattttatttgacaaaagaaatacttccatgttatagaagactaaatattgtttgggctgtctgcagtatggtcttcccttgatttgttcaaaatatcgtaaactttgcttatttatttttattgtggccgactgtgtcgggcactgttgtaggcttgggatggaaaaacaggattcctgcccttagggtttctgcaggctggtcagggagacgatgtggtaagctggagctcagctcctaaggatgtgcaggggcagttgagaggcggaagggtgggagatcattccagggtgtgggcagcacaggaacctctcttcattgggatataattgccattctgataacacgtgtttgaggtgtctaaagtaggaagttgtaccatggtgggacagatatcctgtggttatcatacacagatctcagttttcttctcattgtttgtactttttataaagggtaacaggagatataattcaataaacctttgtggtgtttgggtgtgattttattgtttctttcttctcagtttggatgctgtgaagctttgtgtcttctttccactgccttcccagtttgcatttggagtttaggttggcactgtgggtatgtattttcctcagtatatattaatagttgtctacaacagtatgacataaacatagttattaggatgccctttttctttctttttaagtcttttatcaatttggctttttggaaaaatatctgatggaatacttgtttctgctatattagctgtgtgagactagtgacaggagctgtgggaaatgaatgccaaatgttcttaggcattgatgggaatttcagggtgtggtcttcaagttcatttaagggaattttcatatgctggcaaaaggcttttctcattagcttgactctttccaaaattatttgctgtgaattagaagtttaggaaccttttttcacttaattgtgacctagcatacgaaatggtgatgatttaggaactactgttcttgtattaacagcttttatttaaaaatgattttcctccagtagatggccctactagcatctgggaaataatttcaagtcttctccagcattcaggaataggctttcattttgtgtatcaattactgagaatgattttggtgactcacatcacatttgagaagtaaacctgcagatttcttgtgtgtgtcagcaaatgaccaactgatatttgcttgaagtggattacattatctgctctagaatgattgctttcccaccttcctcacatacagactgagcagctacggtttctaatcataggtctggcactagacttcacttctgggcaactttggcattggagtaaaatgtattaatttaaagaaagttaaaaatccgttcaagtaaacatacagttctaatactttttacaatttaaaatatagatttaaatgataaaataaaaaagaaaatatgggtagacaccataatcctcgtttctgcatctgttcacaaggggttgatatttatgagttctattctccatatccattctatgttctcttaatgctcagtcagcacctcaggtggttggagttcaatgcttggtagtttgacttacactgtcttttctaggggattgagccctgggtagtcctgcttatttgaggttgcaatttgtctttcaataacttttactacaagatatggcgtgttaaaggataccattggggaaccaacataataatatcaggaaaactaaccacgtcagacctgccccattgtgtatcaagtacactatttttccatagtaataaagagttcaccccagccaattctcttttattttgtgcctgtttactcaatggcattaacatgcccaaatgtctgggtagctgtctcatctccagttcagcagaaccattgtcatatgccctagtaaaagcattccttcattggacacttaggccccaatactttcattcagatctactacctgatttcatttctcaaatgatttttatggagctctgatttataggaaagatgttagttgattaaaaataaaacaatttctgagctggtataaaatgtattgtgacatgccttcctcttggaattgcaagagaaaggaagactgttgtttgcttaaaaattgtctataatttgactttgcaaatgtctgcttccagagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcaggtactggtactgagttgaaacagggactccaggacttggattttgatttccttagggggaatgggggtggtgagcatatgaggggaaaatactataaggtcattgccagtgatggcttgtccctttagtcaaatttcagatgttacctatatgcataaacacatgcagttggcagctgttctgtgctgagtattttaaagtagcctcttcccaatatagcccctcagttaactacaagtaaactcattttgaatttcattttaatgggcaccatatgccagtactccctcgggcactgggatgttaagaaagtataatgtatggacttcattctcaagttagttttagattagagggggatacacgtaaacaaaagtgcagtggtcacacagagtggccctaatcactctccttgggcagatttatgggctggtaggaaagagcacaacacggagagggtgtagcaccttggcgatgataatggaggatgtggccagcaaggaagacggagtccattgaaattgattttgggagaagttgccaatctccatgaaagaattggggcctgtgctatttgcttcagggggctataggagagtttcgtgaaagggactaaaagatgagtattttaataagatcattcatccaacttgaacatgggctggaggagaaggtagggagactcaggagattaatgttgatgctaaggcaagataatggctttgggactgtagggaagacactgattgtaagagaatgaaggaggcagaattgccaggcctggttcaccaactgaacttcggttgtgaagacaaagaaacctgggatgacttcacatcctgggcaggtgtgtggtggtgacagtcatggaaattgggaacacagatttgtgcgggaaacatcagtttcagtttgagtttggcttatcagttgaatatcaggcacagatgtctggccaactctcaacatagggtcttaaatgacttcagttccccaagcaatttgtccttcccatgctattggggtggagaggtaatgtctgtgcccatatcacagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggtaatgtcccacttgggtgctggattcatacagccttaatgactatgggtttccagactacctttgtttagtaatctgtcccttctttattctctttttgctttaaatgaacaaaattgctcagattgtgacactaaatttaacatcaaaatgtgaccatgtggatgggtgcagtggctcgtgcctgttattccagcactttgggagactgaggcaagtggatcacttgaggccaagagttcgagaccagcctgggcaacatcacgaaaccccctctctactaaaaatacaaaaaattagatgggttgggccgggcgtggtggctcaagcctgtaatcccagcactttgggaggccgaggtgggcggatcacgaggtcaagagatcaagaccatcctggctaacacagtgaaaccccgtctctactaaaaatacaaaaaaattatctgagcatggtggcgggcgcctgtagtcccagctgctcgggaggctgaggcaggagaatggcgtgaatccgggaggcggagcttgcagtgagccgagatcgtgccactgcactccagcctgggtgacagagcgagactccgtctcaaaaaaaaaattagatgggcatggtggtgcgtgcctgtaatcccagctacttgggaggctgaggcaagagagttgcttgaacctgggaggcggagtttgcagtaagccttgattgtgccgctgcactccagcctgggtgacagagtcagactctttccaaaagaagaaaaaaatgtgaccatgtgttttatagctcttttagtatcatcagtcactgttatccctaagagggaaatacctagctttagttttaggtttccagcattagccaagaaagctcagaattgatgttcctggccaagtacctcattgctgtctccttaaatcttggttaatggctactgtcctggctagcatagttatggagcatttccatggttgtagaatgttctgccaatctcagggacagttttgcttttctgtgaagcaataaaatcaacttcaaaacaaatgttaactatttgtacaatggatttaagatagaccagttcacatacttttttttttttttttttttgagatggagtttcattcttgttgcctgggctggagtgcaatggtgtgatctcagctcactgcaacttctgcctcctgggttcaaacgattcttctgcctcagcctctcgaggcagattacagctgggattacaggcatgcaccaccacacccagctaatttttttgtagttttagtagagacggggtttcaccatgttggtcaggttggtctcaaactcctgacctgaagtgatctatccgcttcggcctcccaaagtgttgggattacgggcatgagccaccacgcccagcctaagatagaccagttcacttactgtttatatctgattactctctctttgccttgtcttctacctttaaaaatctccctactaacttcccattctcctttagctgccatcagtcttctcccttctctgcaaacatctctggagagtcccagcctcagcccacagagcttcccactgctctgaggtggaccttgtttgcaaggcttctttggctctcttggcctggaccctgtctactacttcagccatccttccttaacccctgctggtggtttctgttgccacactccatagcagcgtttcccgcccagatcatgtctttacatctctgggcactgctctggtcctgcctgcctttccctctttgtatcctgcaggctgctacccccatcttgagtgtcctcttcagttggctttcagagggcctcctgggtgttcccttacccacttgccactccccagtcactgggttcagtccttcctgcccaccagcacatgctttctaggctctgtcctaggccgtcttctctctttgtagtctctgggccagtgctgttctagagagtggcagaattttctataaccatggcagtgctccatagctatgccaggcaagacagtagccactaaacacatatagctgttgagcccttgaaatgcagctagtgtgactgaagaactgaaccccgattcggtttaattttcattaaatttaaatttaaataaccttatgtgggtagtggctccagtattgggcagggcagcctgagagtcggggctgttctcctgtcttcagtgtctagatgagggacctcagaggacctgtctctggagctgcagttcaatgtagccagctgccccgtgacacttacatatagctgatttgtggatatgtcagacacggtgtgatgagctcagctttctgtcctcctccccacatctgcccctgccccatttaccccactttgtgtcttatcaagctagaaacaggtcaccacaagtcttcatttccactcaccaagtcttttgtttcccctactaaatattttgcgagaagaaagtgtgtacctttgtattcacatacatgtacatgcacatatacatgcacatatgcaggggtccccaacctctgttaaaaaccggactgcaggccgtgcgtggtggctcacgcctgtaattccagaactttgggaggccgagaccagtgcatcacaaggtcaggagatcgagaccattccggctcacacggtgaaaccccgtctctactaaaaatacaaaaaaaaattagccgggtgtggtggcgggcgcccatagtcccagctacctgggaggctgatgcaggagaacggcgtgaacctgggaggcggagcttgcagtgagccgagattgtgccattgcactccagcctgggcgacagagcgagactctgtctcaaaaacaaaacaaaacaaaaaaaaaaaaaaccaggctgcacaggaagaagtgagcaagcattaccatctgagctctatctcctctcaggccagtggtggcattagattctcataggagcgtgtatgagttcgttctcacacttctgtaaagacatacctgagacatataaagaaaagaggtttaattggctcacagttctgcaggctgtacaggcttctgtttctgggaaggcctcaggaaacttgcagtcatggcagaaggtgaaggggaagtaggcacatcttcacatggcccacaggaaaaagagagaaggagagagagagagagacagagagagagagagaaaaagaaagattgagagggagagaggagggagaaaggagagtgcctgtagggggagttgctacacaaaggagcaccagggggatggtgctcaaccattagaaactacccccatgatccaatcacctcccaccaggccccacctccgacactggagattacaattcagcatgagatttgggtggggacacagagccaaaccatatcagagcatgaaccctattgtgaactgcacatttgagggatctaggttgcatgctccttatgagaatctaatgcctgatgatgatttgaggtggaacagtttcatcccgaaaccatcccccgccaaccctggtttgtggaaaaattgtcttccacagaaccggtccctggtgccaaaaagtttggggacctctgcacatatgcatgcacctgtacatggacacataatacatgtacatatgcatactttatattctctgccacttctggtccagactgatatactatctcatttggattactgcactagccttttgttttggaaacagcattttttaaaaaatttaatttaatttttttgagatagggtgtcattctgttgcccagcttggagtgcagtgtcatgatcatagctcactgcggcctcgatctcccaggctcaagtgatccttctgcctcagccttctcagtagttgggactacaggcatacccaccatgcccagctaattttttgatttttttttttttttgagacagagtctcagcctgtcgcccaggctggagtgggttggcgcgatctcagctcactgcaacttctgcctcccaggttcaagtgattctcctgcctcagcctcccgagtagttgggattacaggcgcctgccaccacacccagctaactttttgtatttttagtagagacggggtttcaccatgttggccaggctggtctcgaacttgtgacctcgtgattagcccgcctcggcctcccaaagtgctgggattacaggcgtgagctaccgctcccagccaggaaacagcattcttgagataattcatataattcacccatttaaagtatataattcattctctttagtatgcccacagagttgtacagccatcaccagaatcagttttagaacccataaaggaactctgtactctttacccaaaacctccatgcctccagctgcaggcagccactaacctgccttctgtctctgtgactctacgtcttctggacattactgtggatgggctcatacagtcagtgagcttgtgactggtgccttctaccaagcagggttttcagtgtagcagcctctctgtttttcttttttttttaaattgtgacggaacttctgcctcccgggttcaagcgattctcctgcctcagcctcccgagtggctgggactacaggcccatgtcaccatgcctggctaattttttttttttttttttttagtagagatgggtttcaacatgttagccagggtggtctcgatctcctgacttcatgatccgcctgcctcggcctcccaaagtgctgggattacaggcgtgagccaccatgcccggctaacctttcatttactgtctgcatttcttccctgatgccttccagtccatgcacccgattgtagccattcatcctattatggtttaaggtgactgtcttagtcagcatgggttgccataacaaaataccatagcctgggtggcttcaacaacagaatttacttctcacacttctggaggttgggaagtccaagatccaggactttcgccttgccctcatgtggtgagggggtgaggaagctctgtggggcctcttatatatggatgctaatctcattcatgaggggtctgccctcatgacccagtcacctcccaaaggccccacctcctaataccatcaccctggtaattaagtttcagtgtataaatttgggggactatagacattgaaaccataacaagcacttttctaagatcagggagtgagtaagtagcagagctaggacctcaattccacatgtcagtcatcttgccttcactctgctccatgatggctgcctcctagagcattgggagtctcgatgttctatatgctctcatgtgttgtgtattggagatagttgaggctttatgaatacatctggatttgttgacttctagctttgctggtaaccagctgtgaccttgaataagttacttcatctctgagcctgtttcctcttttagaaacaggagtttaaaatgctgctttgggttgggcacggtggctcatgcctgtaattccagcactttgggaggctgagatgggaggatcactggagcttggagttcgagaccagcctgggcatcatagtgtgagatcctgtctcctcaagaaattaaaaaattagctgggtgatgtggcgtgtgcctgtggtcccatctactctggaggctgaggtgggaggattgcttgagcccaggaggttgaggctacaatgaaatatgattgcaccccatcctgggtgacgagtgagaccctgtctcaaaaaagaaaaaaaaaatgctgctttgtacccctttcatgtcatggcgtcatggccaacatagaatgccctggttgtttgctgttggagggcatgggcctgggggctccctgagggctccttccatcttcaactcattctctgtgcacctgttaggaagttgtgggccagtccctaccatgtatcattgtgtgggtaaaagtaaataaaatgtgtacagtgtctgaactgtacatatcagggtccaagaacaaaatgagtgacatgggttagctctttttaataaatggtaaaaccaaatattctaattttcagttttgttatacttccatcacatgtttttgtttttttgttttttgtttttgtttttctattttaggcagccttgccttctctaacaaaccccccttctctaagtcccatccgacgaaaggggaaggagaaagaaccaggagaacaagcatctgtaccgttgagtcccaagaaaggcagtgaggccagtgcaggtaggaaacagcgtggggaagggagggacatgagtgcagcatctgtcatgtagaaacataggatttaagtaacttggtgttttagagaaataaatataatacacatcagtaaagtgagagaaagtttctccaggtgcggttcaagatattagaaactaatgactgatgtacacagaccaccttttggtctgaagcatttctaagtgccactggctgacatgcagcccctacagcctccaggcttccagccctagcatggagcatcactctcctatgcttccctggttgcaggtgatggctggagaggcctcctgattttcagtaagggaagtggtgtagatgcttaggaatagatgtagtgagtgaaaaaactgattctgatatgtcaaaaattctgattggaaatggaatatttacatttggaagagctaaaggcgagagaaagtggggataaagtcatctgagttggaggagcttaaaccattcacaagtttggaggacctttttttacccatgaaaaggtcagaacagaaggggctaggatttaggtgtgactgcagtttattgaattcccatccatactgctctcggtgggcagtggcaggggcaggagaggagcctggcaaagcatgaagtgactgctgctgcctctgctatctgggacgcctggccacctgtctgtacagtctccctccagacccattctcacgctgtctcttggcacccaggggccagtgatggttctcccatttgttttgtgtatatagcatttatatcaaggctatttatttatttatttattttatttatttatttttttgagacagagtctcactctgtcacccaggctggagtgcagtggtgcaatctcggctcagtgcaagctctgcctcctgggttcaagcaattctcctgcctcagcctcctgagtagctgggactacaggtgtgcaccaccacacctggctaattttttgtattttttattagtggagacggggtttcaccttgttggccaggatggtcttgatctcctgacctcgtgatccgtccacctcagcctctcaaagtgctgggattacaggcatgagtcactgtacccggcctatttatttatttttaattgacaaaattgtatatatctgtaatatacaacatgatgtttgaaatatgtgtacattggccaggcgtggtggctcacacctgtaatcccagcactttgggaggctgaggtgggcggatcacgaggtcgggagttcaagaccaaactggccagcatggtgaaatcctgtctctactaaaaataccacaaaaaaaaaaaaaaaaaaaaaaagccgggcatggtggctcgcgccagtcgtcccagctacttgggaggctgaggcaggagaattgcttgaatctggcaggtggaggttgcagtgagctgagttcatgccactgcactctagcctgggcgatagagcgagactccgtctcaaaaaaaaaaaaaaaagaagaaatacatatgcattgtggaatggctaattaacctgtgcatcacctcacgtatcattgttttgtggtgagaacacttaaaatctactctttcagtgattttcttgcatatggtacattgctattaactgcagtcaccatgctatacagtagatctcttgaactcattcctcctgtctataaatgaaattttgtatccttgaccaacacattcaaggttttttttgagatggagtcttcttcacccaggctggagtaccatggcacgatctcatctcactgcaacctccgcctcccaggttcaagcaattctcctgcctcagcctcctgagtagctgggattacaggcacatgctactgcacctggctaatttttgtatttttagtagaagtggagtttcaccatgttggccaggctggtctcgaactcctgacctcaagtgatccgcctgccttggcctgccaaagtgctgggattacaggtgtgagccactgcacccggcctcaagcgttttaaaagatgctcttttctaaggattgactgtagtacaggaggaagattgacctgttgaaaagcctcagcctttacaagtgtaaaattatcagtatattactatcatctttctgatgaattaaataaactaaggactccaagtcaaaagtcttcaaactgaagtagaatagttgtatatagtgcttggcactttaatatttagtatcggtttaatgataatgtttgtgcctttgccgtctttaaaacatttttacatcatccctgtttgattacttggtgtgctcatgaagttgttggccactaaggaatcttaggctcagagaggttctggaattggccagtggtccttgaatcagctgctcctatgattctctaactgatttctcacaaagcaaacaagcaatcataacaaaacaactgtgcacactgctcttcttattttgttatttaaaaagtacttaggctctacttatgtttgttagtcaatttctcattacttctagttaatcaaaaggtcagaggaaatacttgaatattttcatactagaatactttaaaaaatcatgatttccagtaatctctttaaaacttggcaagttattttgatctaaaagtttatcttttgtgtgcatatttttaaagcttctagacaatctgatacctcaggtcctgttacaacaagtaaatcctcatcactggggagtttctatcatcttccttcatacctcaaactgcatgatgtcctgaaagctacacacgctaactacaaggtatgggcctctgcatcttttaaaaatatatatgcacacatacttacgtctaatggatagttgatgtttttcttatgatttgtaggatgtataagccctttgagatatgagttacatttagttttttcaagtttgtttgtctttcagctttgtttatgatagcttctatcatacaggtgttttggattttcatattgtttgtactcacagctaagattgattacagtgacagagctaggatgtgcagccaggttatagggggaagtggccctggtggagtctggagggatccgtgtacaggcttccttccctcccgtgaggctcacacaaaaatacagcaacatgctggtcctgcaggtaccctctgcctaacatgagccacaattccagactcacagaagaaaagcaggtgttcggcataaaccatgtgtttcaaatagtctgggcatggtgagccacttgttatcagctagggaaagtttatgtcagcgtaagaaactgttcaccagatacccccaagagccagcctttctgtctagggatgttttagttttttagttcatttttttttttaactttaaaattttctgttcatctgcaatttgttagatatgaagtatgtgtctaatttaatttttgtttttggttgtccccaataatgtttacagaagaatttttctgcactaattggcttgagttacttacattctcatagttctctagtttcagtagtttcatttattattttgttatatcaatctatctgtctgctcatctattagaagcatccttgttttttttttttcttttttagacagagtcttgctctgtccccaggttggagtgcagtggtgcaaccatgcctccctgcagtctcagggctcaagtgatcctcccacctcagctcctgagtacctgggactaccggcatgtgccaccacacccagctaatttttacattttttgtagagacagggtctccctaagttgcctgggctggtctcaagctcctggcttaagtaatcctccctccttggcctcccaaagtgctgggattacaggtgtgagcaactgcacccggctacaagtatacttcttaattattgtagcttaatggtatttatgaggggatcagttcccctgttgttctttagaattttctggatattcttctttattgattttgggatgtgaacaatagaatcaacttctacttgtagattgatttagggagaacttatacctcagatgttaagtcaccctgtccagaatgtgggatgctttcctatttgttcagaactttttaaattacctcagaagcacatgaaatttaaaggattttaaaaaaaacttaaagattatttcacatagctcttgcacatttcttgataaatgaatcctcaggtattcctctgtttttgttactaatagttacttcttatgggttttttttcccctgaaaatcatttatcaaacgtatgtggcttattttctgaaggatgtttgataattttggaagatatgaaagtcttcatattttacaaggtttgaggtctctttaagctgcatggttctcatgtcagctcccaaagcagaagacggcatgttgaaaaatgccgtagagaagatacttcttttccacctgttttcaactcatatcatcttgaatttcagggcacctttccatgctcctagtgcttgctatctgtttattattttccttcctgaataccctgaactccagcatgttctgctgtaattctggcctccctggcatcttggactcctgtttcctttgctctgtcatccccgcggtcagctcctgctgcgcagcttctcagctgaagtgcgtttggagtgcctggcgtgtcttgctggatctttgagtattgcctctggtttccttggttccttctgctgagttgctcagcgtctccactccccatttcttgtgtggcccttcctgcactcctctgattccttttgtcttccctggtttcttgctttggtttcgagtctccacagaacttttgcagctcttctgaagacctggaagctttttcatcttaattctcatctcatgacctcttttcccttctttgagagctagaacttcccatggtgaacttctctttccagaattccatgccttcttttccctcccacttacctgttgtccaggagaggtcagattgctgtgcatattggaggagaaccctttcttccctgggctcttcatctcacatgacatcaccacatcacctcgttccttggaccctcagtggtgtcactgctggatttttctttcctttggctggccttagggcacacccaggttgactagcgtagtcatggtatttagatccactcacattttcagtttctgtgtctgtctcttgcctgcttctgacttcgcccagagaaagcttctctttcacaagggttcttagatttatgttcactgagcaccttcttttctgaggcagtgttttaccaatatttattttcctagtcagtctcgccttacctttcttgttatgcatgtctttggtcctgacccattctctgagtctgtaaaatagaattgctgtataatttaattacatgaaatcctttagaatcttaacacatcttacacctgatttaatattttattgtatccaaattgaaccaaccctatgtgaatttgacagtgatttctcccagggatcctagtgtataaggaataggacttagtattttctattttttgatataccacataccagatactgattatgatggacatttaacccttttttctcattatgaaagaaagttaggaattatttcttccagtagcgccagtgtaacctgaaagcctttgaaagagtagtttttgtatagctatctgaaaggaatttctttccaaaatatttttccagtgctgacaacaaacacgcagacacaccctgcaaggtgagtgtacggcgccgcacagtggaggcatctgctgcagccgtcgatgtttgtgtctttggttgtacattatgagatcgtgacagggccagtaaccgtgtgttctctccttcaccttcccaaggtcacgctggatcttcagaacagcacggaaaagtttggagggtttctccgctcagccttggatgttctttctcagatactagagctggccacactgcaggacattgggaaggtttgtgtcttgttttttctccttgggttgtggctggcacacttgatgtgcgtcttctgggctgagttcatctaggatggagcctggttctccagggtgcctccgggagactcctccctgccccacgtgcttgcgtcacaggacccaagtctgactctgccttagccatgaagtttagggggaagtttctatttgtattctatttttgtctgttatcatgtattagcttagacccagtttagtttggaaaatcagtgggtttcaaaatgtgtttgtagagtcctttatttcttaacttgaccttttcaagtggaaaggggcaaaacagacgggtaagggggcggggcgggaggtgtgacttgctcttttgtgcctgaggaagtaacagagctggggttgacagtcatattctctgacacagatagtctctgacttatctcacagaaagtcagcggcagagcctgagttaaaagtctcgtagattttctttttcttttttttggtggctaatttcagttttatttatatttgtttatttatttattatactttaagttctgggttacatgtgcagaatgtgcagttttgttacataggtatacacgtgccatgatggtttgctgcacccatcaacccatcacctacattaggtatttctcctaatgttatccctcccccagtcccctcactccccatgggccccggtgtgtgatgttctcctccctgtgcccatgtgttctcattgttcaatttccacttgtgagtgagaacatgcggtgtttggttttctgatcttgtgatagtttgctgagaatgatggtttccagcatcatccatgtgcctgcaaaggacatgaactcatccttttttatggctgtatagtattccatggtgtatatgtgccacattttcttaatccagtctatcattgatggacattcgggttggttccaagtctttgctattgtgactagtgccacaataaacatacatgtgcatgtgtctttatcgtagaatgatttataatcctttgggtatatgcccagtaatgggattgctgggtcaaatggtatttctagttctagacctttgaggaatcgccagactgtcttccacaatagttgaactaatttacactcccaccaacagtgtaaaagtgttcctatttttccacaacctctccagcatctgttgtttcgtgactttttaacgatcgccatcctaactggcgtgagatggtatctcattgtgattttgatctgcatttctctaatgaccagtggtgatgagcattttttcgtatgtctgttggctgcataaatgtcttcttttgcgaagtgtctgttcatatcctttgtccattttttgatggggttgtttgcttttttttcgtaaatttgtttaagttctttgtagattctggatgttaatcttttgtcagatgggtagattgcaaaaattttatcccattctgtaggttgcctgttcactctgatgatagtttcttttgctatgcagaagctctttagtttaattagatcccgtttgtcaattttggcttttgttgccattgcttttggtgttttagacatgaagtctttgcctatgcctatgtcctgaatgttatggcccaggttttcttctaggatttttatggtcctaggtcttatgtttaagtctttgatccatcttgagttgatttttgtgtaaggtataaggaaggggtccagtttcagttttctgcatgtggctagccagttttcccaacaccatttattaaatagggaatcttttccccattgcttatgtgtgtcaggtttgtcaaagatcagatgattgtagatgtgtggtggtatttctgaggcctctgttctgttccattggtctatatatctgttttggtaccagtaccatgcagttttggttactgtagtgttgtagtatagtttgaagtcaggtagtgtgatgcctccagctttgttcttctagcccaggattgtcttggctatgcaggctcttttttggttccatatgaagtttaaaatagttttttccaattctgtgaagaaagtcagtgatagcttgatggggggatagcattgaatctataaattactttgggcagcaaggccattttcacgatattgattcgtcctatccatgaacatggaatgtttttctatttgtttgtgtcctctcttatttccttgagcagtggtttgtagttctccttgaagaggtccttcacatcccttgtaagttgtcttcctaggtgtttcattcccttagtagcatttgtgaatgggagttcactcatgatttggctctctgtttgtctgttattggtgtataggaatgcttgtgatttttgcacattgattttgtatcctgagactttgctgaagttgctaatcagcttaaggagattttgagctgaaccaatagggttttctaaatatacaatcatgtcatctgcaaacagggacagttttacttcctctcttcctatttgaataccctttattgctttctcttgcctgattgcgctggccagaacttccaatactatgttgaataggagtggtgagagagggcatccttgtcttgtgccggttttcgaagggaatgcttccagtttttgcccattcagtatgatattagctgtgggtttgtcataaatagctcttactatgttgagatacgttccatcgatacctagtttattgagagtttttagcatgaaaggctgttgaattttgtcaaaggccttttctgcatctgttgagataatcatatggtttttgttgttggttctgtttatgtgatggattacgtttattgatttgcgtatgttgaaccagccttgcattccagggatgaagctgacttgattgtggtggataagctttttgatgtgctgctggattcagtttgccagtattttattgaggattttcacatcgatgttcatcagggatattggcctaaaattctctttttttgttgtgtctctgccaggctttggtatcaggatgatgctggcctcataaaatgagttagggaggattctctctttttctattgattggaatagtttcagaaggaatggtaccatctcctctttgtacctctggtagaattcggctgtgaatccatcctggactttttttggttagtaggctattaactattgcctcaagtttagaacctgttatcagtctattcagagattcagcttttttctggtttagtcttgggagggtgtatgtgtccaggaatttatccatttcttctagattttctagtttatttgggtagagatgtttatagtattctctgatggtagtttgtatttctgtgggatcggtggtgatatcccctttatcgtttttattgagtctatttgattcttctctcttttcttctttattagtcttgctagcggtctacctattttattgatcttttcaaaaaaccagcacctggattcattgattttttttggagggttttttttcgtgtctctatctccttcagttctgctctgatcttagttattttttgtcttctgctagcttttgaatttgtttgctcttgcttttctagttcttttaattgtgatgttagggtgttaattttagatcttttctgctttctcttgtgggcatttagtgctataaatttccctctacacactgctttaaatgtgtcccagagattctggtatgttgtgtcttcgttctcattggtttccaagaaaatttttatttctgccttcatttcgttatttacccagtagtcattcaagagcaggttgttcagtttccatgtagttgtgtggttttgagtgagattctcaatcctgagttctaatttgattgcactgtggtctgacagacagtttgttgtgatttctgttcttttacatttgctgaggagtgttttacttccaactatgtggtcagttttagaataagtgcaatgtggtgctgagaagaatgtatgttctgttgatttggggtgcagagttctgtagatgtctattaggtccgcttggtccagtgctgagttcaagtcctggatatccttgttaattttctggctcattgatctgcctaatattgacagtggggtgttaaagtctcccactattaccgggtgggagtctctttgtaggtctctaagaacttgcttcatgaatctgggtgctcctgtattgggggcgtgtatatttaggatagttagctcttcttgttgaattgatccctttaccattatgtaatggccttctttgtctcctttgaactttgttgatttaaagtctgttttatcagagactaggattgcaatccctgctttttttttgctttccatttgcttgttagatcttcctccatccctttattttgagccaatgagtgtctttgcatgtgagatgggtctcctgaatacagcacaccaatgggtcttgactctttatccaatttgccagtctgtgtcttttaattggggcatttagcccatttacatttaaggttaatattgctatgtgtgaatttgatcctgtcattatgatcctagttggttattttgcccgttaactgatgcagtttcttcatagcgtcagtagtctttacaatttggcatgtttttgcagtggctggtactggttgttcctttccatgtttagtgcttccttcaggagctcttgtaaggcaggcctggtggtgacaaaatctctgcatttgcttgtctgtaaaggattttatttctcgttcacttatgaagcttagtttggctggatatgaaattctgggttgaaaatactttttttaaagaatgttgaatattggctcccactcttttctggcttgtaggatttctgcagagagatctgctgttagtctgatgggcttccctttgtgggtaacccgacctttctctctggctgccctttccttcatttcaatcttggtggatctgatgattatgtgtcttggggttgctcttctcgaggagtatctttgtggtgttctctgtatttcctgaatttgaatgttggtctgccttgctaggttggggaagttctcctggataatatcctgaagagtgttttctaacttggttctattctccccatcactttcaggtacaccaatcaaacgtagatttggtcttttcacatagtcccatatttcttggaggcttggttcatttcttttcactcttttttctctaatcttgtcttctcgctttatttcattaatttgatcttcaatcactgatatcctttcttctgcttgattgaatcggctgtcgaagcttgtgtatacttcacaaaattctcgttctgtggtttttagctccatcaggtcatttaagctcttctctacactggttattctagccattagtctaacatttttttcaaggtttttagcttccttgtgatgggttagaacatgctcctttagctcggagaagtttgttattaccgaccttctgaagcctacttctgtcaattcatcaaactcattctccatccagttttgttcccttgctggtgaggagttgtgatcctttggaggagaagaggtgttctggtttttggaattttcagcctttctgctatggtttctccccatcattgtggttttatctacctttggtctttgatgttggtgacctacggatggggttttggtgtgggtgtcctttttgttgatgttgatgctattcctttctgtttgttagttttccttctaacagacaggcccctcagctgcaggtctgttggagtttgctggaggtccactccaggccctgtttgcctgggcatcaccagcagaggctgcagaacagcaaatattgctgcctgatccttcctctggaaacatcgtcccagagcacgaaggtgtctgcctgtatgaggtgtttgttggcccctactgggaggtgtctcccagtcaggctacatgggggtcagggacccacttgaggcagtctgttcattatcggagcttgaatgccgtaccgggagaaccactgctctcttcagagctgtcaggcacgtatgtttaaatctggagaagctgtctgctgccttttgttcagatgtgcccttcccccagaggtggaatctagagaggcagtaggccttgctgagctgcagtgggctctgcccagttcgagcttccctgctgctttgtttacactgtgagcatagaaccacctactctagcctcagcagtggtggacacccctcccccagccaagctcctgcatcccaggtcgatttcagagtgctgcgctagcagtgagcaaggccccatgggcgtgggacccgctgagccaggcacaggagagaatctcctggtctgctggttgtgaagactgtgggaaaagtgcagtatttgggcaggagtgtactgctccttcaggtacagtcactcatggcttcctttggcttggaaagggaagtcccccgaccccttgtgcttcccaggtgaggcaacaccccgccctgcttcggcttgccctccgtgggctgcacccactgtccagcaagtcccagtgagatgaactaggtacctcagttggaaatgcagaaatcacctgtcttctgtgtcgatctcactgggagctgtagactggagctgttcctattcggccattttggaagcatcccttgttttttgaggtggagtcttgctctgtcgcccaggctgacgtgcatcggcacaatctcggcccactgcaacctttgcctcctggtttcaagcgattctcctacctcagcctccggagtagctgggattacaggcacctgccaccatgcctggctaattttttgtatttttagtggagatggggtttcaccacattggccaggctagtctcgaactcctgaccttgtgatccacccacctcagcctcctagagtgctgggatcacaggtgtcagccaccacgcccagccatattttcagatctccctctctttgccctaaaccactgtgcttaataagtagtttttagtggccagcagtctccatgtataacacattttagcaaaatggaaaatactatatgttttaaatttgaacgtgagattatactgaaataaaaatcatctaactgggattctttaaatagtaagattttcttttttgtatgtgggtttttttttaaccttattattatgactgtcatatatagaaatggctgtttttcagttacagtcagtgaatgtatcaaatgctgccttatccaaataataaaagtaaattattaataagtcacaatttaatgaagattgatgttagttgatctttatattcttgaaatcagccatatggttgtgtgtgtatgtatatatttttaaaggtacataaagataataagctcatctctgaaaatttttacatttggcataagaataactggataattaagcatcttattctctggcctgtgtctttacagttaaaggtagatttactcacctctccttttttgtttttctaagttcatcttttttgctgtttcaagacagaggcccattttagctttctcgcatatccttttgtttgtactttggaagcctcacctgcttaattgttgagtttttatccgtggtcttttagagggggatatgtagggtagaagctttcacaggttcttgtttgcacttggcccctgactgttttgaggaatctccctcactgactcacagcatggcaaggtttcagatctctttctgccacacagcagttctgaggcagctggaaagatatccagatgcttagattgtcaggccaggcttgagatatacaaactattgagccttatctgtgaccttgcttaggtgaaggcatcagagcccctgcaccaacatgcataggcctctgcatgtgtgcggggctgggtgttgaggtctgagcacaagtgtagctggagaggtgagcttgatgtggcgacgggtatgagcaggttttcttcagacttctgtgagtttacctagttccaggatttaaaggcacagagactttagaattaaaatagaatcattttctttttctaaatagcaacactaggaataaaaaataataattccacattcttgacaggtaatgttttttcttgtcttctaatccttatttattccatactcatttttatacataattgaaatgtattatgcattggatttttcttttgcattatattatagacgatttttcatgtaactccttactgttccattttatatgttttgtctggtttaagactttatctgcaaaccgggaaactgtctctacaaaaagaaaaacaaaaatagttggccgcagtggcatgcgtctgtggtcccagctactcggggctgaggtgggaggattgcttgagccttgggaggttgaggctgcaaagagccatgatcatgccattgcactccagcatgggtgacagactttatactgtctgttttgggtgatttgataatgatatgccctgatgtagtttttttatatcttgtgtttcttgtgcctgggtttattgaggttgggtctgtggcttcatagtatttttaaagtttggaaaattttaggccattctttctttctttctttcttttttttttttttgagacagtgtctcgctctgtcgcctgcgttggagtgcagtgacactatcttggctcactgcaagctctgcctcctgggttcacgccattctcctgcctcagcctcctgagtagctgggactacaggcgcctgccaccacgcctggctaattttttgtatttttagtagagacgaggtttcactgtgttagccaggatggtctcaatctcctgacctcgtgatctgcccgcctgggcctcccaaagtgctgggattacaggcgtgagccactgcacccagctaggccattatttcttcaaagattttttttctgccctgcctccctccttttttccctctcttaaaggggctgtgatttcctgaatgattgcttagtgttgtcccatagcttactgatgctcttttcagtgtttgattgttttatgtgttttctgttttgtatagtttctattattgtgttttcaagttctctgatcttttcttctacagtgtctactctgttgttaatctgttaatctgttgttaatcctgtccagcgtatttttttttttgtttttgaaacagtctcactctgttgcccaggctggagtttagtggtgcgatatcagctcactgcaacctccacctcccaggctcaagcaattcttctgcctcagcctcccgagtagctgggactataggcacgtgccaccacacctggctaatttgtgtatttttattagagatggggtttcaccatgttggccaaactggccttgaactcctgacctcaggtgattcatccgcctcggtctcccaaagtgttgggattataggcatgagccaccgtgtctggcccctgttcagtgtatatcactaattttgtttttatctctagaagtttgatttaggtcttttaaaaatgtctccctgtgtttctgtttagctttgtgaacacaattgtaataactgttttaatatccttctctgctagttctaagatcttctaataacttcccagttcttggtgtttctcattggttgattgatactcctcgttttgggttgtattttcctgcctctttgtatggctgccaattttttattggatgcccaaccttgtgaattttactttgttggatgctatatatttttgtgttcccatagatcttcttgagctttgttctgaggttagttgagttacatatagatggtttactcttttgggtcttgctttataatttgtcagatgggttggagcagtgcttagtttaggactaattttttttttggactaattattcctctttaggaataattaggtaccatgcttaggaggcaagaccatcctgagtactctacctaatgaaccagaaagtttgggttttccagtccgcctgctgagaacagtgactttctagccctgtgtgagcgctgagctctgctccttctaatcctttccaatgcttctttccctggcctcagggagttttctcacacacatatctctgctgagtactcgagagggaccttccccagatctccagagctctctctgtcttgttttctcttctctggtgctctgtcttatgaactgtggctgtcttggtctccttagattctcagcacctcttcaattcagagggttgcctgtccctcctccttgtgccacagcctaggaactctctcaaagcagcgagttggggcagccatagggctgacttagtctctcgtctcccagggatcactgtccttcattgctcatgtccagtgtcttgaggactctgggttttgtctgttttgttttttggtttgctttggttgtctcaggcaggagggtaaacccagtccctcaccctcattgtgctcagtagtggaagtctcactctattacattagatattagtatttgtagcagagccctggttccctggtacttggggagctcttgaaaggccagaaacagcatgctttctcaccttttccagggcttcagtttctggtgcacatcaagcattccatacacatttgttaaagtcctttgttagacaagtagtgattcacaggttctatttgtaattttttcagttaacatgtattgggtatctgctgggagctagtaaaaacaaaaagtggtgtgtgacaaattcaattctgacaagaacaaccttaaacacttagaatatactttgagcatatcagaattttaaaaatgtgtggcccttgagtatttgaaaccaacaagaatctattgcttattagtagaggatattttgttaaacaagtggagagagaggcattttcagtctaattggtgttggcttttagcagctgatggaaaccagttcgtgattagccaggcagtggtgaaacaggctgtgcattctgaatgcctaggtatctaggcattcagaatggtggcgctctttgagttagcatcttcttctttcttgattcttttttttttttttttgagatggactttcgctcttgttgcccaggtaacaactccagtgcaatggcgccatctcggctcactgtaacctctgcctccctggttcaagcgattctcctgcctcagcctctcaagtagctgggattacaggtgtgcgccaccacgcctggctaattttgtatttttggtagagatggggtttcactatattggtcaggctggtcttgaactcctgacctcaagtgatgcacctgcctcgatctcccaaaatgctgggattacaggcgtgagccaccactcccagccccttcttgattcttgaaaaggacattgggtgctgtacatctcgttatagatgttgataaaaatgcttgtgagaagagtaacattaaggtagttatttggtcatttttgcagattattttaagacaattctaggactgatttgtggtaaatcacacattgctgtatcatagttgtgttcactgaacatattcaggggctctacagatgcagggctcttagctgctttgcacacttctgaattcctgccctgcgaacaggactggatacctaatagacaacaggtacttgataacagtttattgaattaatgagtgaatgaacagatacataaatgcatgaaagaatggttgtaatgtatataacttggatttcaagactttttactgactgttcaaaataagaaattgaaaactttcctctgattttcctctactatttacacaatttaaatggaagttatcttgtaccttcaatttctgtctaggattcgtacaataacgggtcatctctgagtcgcttaatgtctcacttgtctttctacagtgtgttgaagagatcctaggatacctgaaatcctgctttagtcgagaaccaatgatggcaactgtttgtgttcaacaagtaagagcttcattcttttcctcttctgttaagacgttcgggtatgacagcaaaacgctgctactccttaagaggcaggcgctgttggcataatcagctgggaggattgtggggtccagcgcagcactttttggctcagtccatgattgagccaagaggccatccttcccttcactccccaggaggacgaggtctgtcactgtggagggcagaggacaccagaagctcctctgcaacctcgctagttaacttccagtccctcggagtttctgtttagaatgctcaatctcatttagaattgcaaggaaacccaaaacgcctatttaaggtacaaacagcacttcatacaatatctcatgaggtattaatagtgattcacaggaagaatttcacgctgtgagtctttgctaacatatccagttatttacagatggatttgatatttgtgtgggagattcttaaaagtgttgttcacgccacattgttgatgcctcatttttttcactgtagttgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcggggtaacagttgtggcaagaatgctgtcgttggtggaagcacgaaagagcaagcaggaaatactttgtaaaagaataaaaacgaaaaatgttagcgaacatcttctaatagtctgctgtattcagagaactctaggagatatatatggttgatgcaaagatgatttaaggcatagcccggccttccaagaagtgtgtggccagtgagtgagatgggcttgggacttacacatctcagaggtgggggtagaggaggaggaacactgagtgggctgagaagcagccagctctcattgccaaagtgtgtcagcaaaccagaatgcagttcataatgtccccacccattcaaagcacaggacctgtagagtggtgtggcatgtgttggtggcacttttcaggcctgtaacaaggatgaaagaacagcttcatagcagcacagtagtgctggtgttcagaggtgtgtgaaggccatagaagcatcttggatatattaccttgtgttttgtcagctttatgactagaagtctcttttcacttaaatttgttttttttttttttgagacggagtcttgctctgtcgcccaggctggagtgcagtggtgcaatctcagctcactgcaagctctgcatcctgggttcatgccattctcctgcctcagcctcccgagtagctgggactacaggcgcctgccatcacgcctggctaacttttttttgtatttttagtagagacggggtttcaccatgttagccaggatggtctcgatctcctgacctcgtgatctgcccgtcccggcctcccaaagtgctgggattacaggcgtgagccaccgcgcccggcctcttttcacttaaatttatgtttgtgtttttaatgcctagtatacaggacttcttaaattgccttaagtatgaacaggtatttgagttgctaatctgtatagtagcaataatagaatcccttgtttttccttttataaatttagcgattaaatagctacaattaaaacactagagtcaggagtcaaggaaaatacccatgttccaggctgtatgttagtgatgtacttactatatattggagtttcaggagtaagtctgtttcaatgctttctgtaaccatttggggtattaataagcatgtgagtgtgtgcatgtttgggttaatttcatatatgtttcttagaagggatatcattgatgtaaatattttaaaggcttgtcctccaaaaaaatcatgtaatttcttctaaattactgatcttttaaatgaccttcacctttctctcaaatctcacttaagactgggctgagtagtcagtttcctgtagcagaaaaaagctcagacttgagtagccttctgcgagtgaggagacttgatggctgtcaggcagctgtaaactctaaatagagtgtcattatctgaagagggcgatgctgccacactgagtggcctttcaagttgtttctcaatctgacacgttctgatcgtgtgaatgtgaaattggtttgagcaggagtatatctgagtgcagaggagattatttaaagatattctcattctctgcttcccttttattcccatttggcagatggtttgatgtcctccagaaagtgtctacccagttgaagacaaacctcacgagtgtcacaaagaaccgtgcagataaggtaaatggtgccgtttgtggcatgtgaactcaggcgtgtcagtgctagagaggaaactggagctgagactttccaggtattttgcttgaagcttttagttgaaggcttacttatggattctttctttctttttttcttttttatagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtttgtcacttttatctttcatccatcatacctgttcctaatttagtacaaattaccctaaaagacactgaaatctactttaaagaaatgtggtctgcatgtttccctcatcagttgctgctgcttatctttttcatgcacctagctggtgcagaaggcctggggcatagccagcctcagcaagtcagcatccttgccccagctccctggactcaaggctaacctggggttggctgttagggatttccaaaggtttgtcccatccacttgcctcccctccaaaataagtttgaatttaaattgtgagatacaattaagatttattgtttggggaacatttttgcaaaatctagagttagtttaaacagattatcaattattaccataattgatcatctgcagtttcaagctatctaacaggttcacttacctctttaaaaaggaatggaatttagcaggacagtaactgagacccgtgctcctggagtccatgtgggagctgtgtggctctgcacaagcatttgcacgcttcccctcttgactgcattaccttcctcctatagttgctgtgggcaccagattctggctagtcctgtcccttcatgatgcacattttcctcaagattcgtcccagttaaatcactgcagatgaaactgccttttcatcgtcaaaatttaactgtcatttttgagccgtgatcttgggctactttcttatgtggggtaggaatatttgtgagttagaaatattacacttctctatttccttctagacgtaaatctgttaatcctgtcagcactgttactcacctgaaagggtctgtttccctaggagaactgagggcactcggtcaacactgattttccacagtgggtattggggtggtatctgcttgttttttttgttgttgttgtttgtttttttttgttttttttttgagatggagtctcgctctgtcacccaggctggagtgcaggggtgcgatctcggctcactgccagctccgcctcagaggttcacgccattctcctgcctcagcctcccgagtagctgggactacaggcacccaccactacgccaggctaattttttgtatttttagtagagacgaggtttcactgtgttagccaggatggtctccatctcctgacctcgtgatctgcccgcctcggcctcccaaagtgctgggatgacaggcgtgagccaccgcgcccggcctggggtctgcttttaatgaaggaggcatcaaggggtgggctttgcgttggcctgatgctttcatctttctttcacaaaacctgtccgaagaaaatccgtctaaatgggccattgctctcctcaggaaatagtcattgggaacttcttttcctttcctttgacactaggaggctgactggggagaagccctggtctatggctgtgggcagcaggggctgagaggagcaggctctcaggggggcacgggtaccccaagggaagccagagccctgatttgttccattctagtaagaacaaagactgctctggtttcatgtttgttctgattgcctttcatcaaccggtcccctttctcccagttcttaagattcagtacagtgacagttttatgaacaagaatagaacactagaacagacaaaccattgaactctatgctgataaagatttattgagctcctgctgtatgtttgcattctgcccagaggctctgagaaaaccaggccatatgctccatgctttatccatggaagctccccgtcaggttgggaaagctgacagctgcagggaatacagtgtgacacaaaactggctcccatgcagcccttacgtgtcgcctctcagatggttgggggacgaaggtcgactcctttgggtatcttattactaaaccagtttcagggaatctgtgccaccctatctgccattaacgtgaacagatgagtccccaaggtgtaattttgggtattgtctgatgtctcttggaatttattatttgtttttccaatgagatttcacctcagggtatagtaaagttgttgaggggattcctggatgtgttctgcaattatctaggctgatttcagaatagagttatgcttatagtcaaatttatcagctgtcaagaattttatttaaaatttatgcagataagcaggaggaaaagaagcctggtttttacattttaatcctattattgatgtgaaattttattttccttcctgtaggtgtttattggctttgtattgaaacagtttgaatacattgaagtgggccagttcaggtaatagcattttattattttagatttttttcttcttcttgtgtacttacatgtaatttaggttattaagtgaatgtttaaactactgttaggcatttttgctgttttctttaaatggaaatctgactaacatactgtgcatttttgcttctcttaaaaattaatgtatatctcaagacttgtttggaagtagttatgtatctgaaaattccatatgttgtcagtattcattgcacatttcaaagcatttaattgtgttgacagatggtggaatgaaatcttgtggtggagcactagtttttaaatcttcttagagaaagcagttttatataatgttgtctttagtaattattatgcatttgtattctctgcagctttttcttgctagatgttgaggttttaatacttcttgctagtccattacaggtttataattattaaaagttaaaattcttttagtacctaaaatgcttaataaacattgtaattaggaaaatttagtgcagaaggaaagtgttcccagattccctggggtctggaaacatagtgtttattctaattacatgacacctccactgtgttttggggcaagttactgtttctcttttgagtttcaatttcttcaagagcaaagaggcagaggagagctaggaagatcgtagctgctgtgcccctgtgccgtcgggtgccttctacctgctgcctccgaacctttacacatgtccctgctctgcgcgagggcacagatgggatgcactgtggcaggggtggggttagagtagatcacggacacctgttagcttgatgtgtgcttgctgtcaaggttgaatcatgaattattttatgttgcttatattgatatgtatcttaattttaaaagaaaggtctaaatggatgtttttgtttttagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatggtaacgggacacacctttcactgtcgtcttcggtgtcgtgatgtgcttggcagtgttcgttttcatatacccactttgaacgttgtcagtggcagccatgtgcttctcaggctctgcatgtgtgtctgtgtatgtgaaggtactggttagagacgtttcaaaagagaagagagcatattctttactctcagcaatttgtaatcttctcagggaaaaaaattcaagaaacagtaagataacctaaggtacagatagattctgaatataaagttcctgttcattcacatgaaacgctaaaagttcttcacttgatcttagccaaaaggccaagaagcgatgcaacactaaaaattcttaaatcgaacttgccgtgaattaaattttgatctctcatccagtggtattggagatatagtttgacttgggttcagggctttctgttttgcctgatgattttgctggagcttaaataaggaacccaggagatggccagctgtgcaagcccccagcctgtggaaggagctagtgtggttttatgaatgagttgcaaatctttctttgagctttttgaactgatcttccagcattgccctattgacccctccctgactcctttgctggaatctgtaggcttttgaactttgacagggacacatcctaagacccttgcaaactcccagatgtgagaatggcactactacttagagtcttttcgactcagcgtgtgtgcagaagagcatcaaccgggctgtgttgcgaggcagggccttggctgacctctcagtgtttacatagctaagccagttagtgtttgccacggcctcacaagggcttcagattcacacagccaaagtatagattattaaaggcataggtgtttggtttcctggacttggagggtctttggacagaaaatcagtaggcaaccacacccagtactttgtgctgggaagcttggtcatctgtgagagggtcagagagtatacccatgcgtgcatgccaccgaagggtcagtgagtattcctgtgtgtgcatgtctcagggccggagagagtatgtgtcactgagaggtcagagtgtttgtgtgtgtgtcaaagagggttgcattgtgcccttcactgaggggtcagagggtgcctcgcgtgtgtgtgtgtgtacgtgtgtgtgtgtcactgaggggtcagagtgtgcctgtgtgtgtgcttgtgtgtgcgtacatgtcactgaggggtcagagtgtgcctctgtgtgtgtgctcatgtgtgtgcatacgtgtcactgaggggtcagagtgtgcctctgtgtgtgctcatttgtgagcgtatgtgtcactgagggggtcagagtgtgcctctgtgtgtgtgctcatgtgtgagcgtatgtgtcactgagggggtcagagtgtgcctctgtgtgtgtgctcatgtgtgagcgtatgtgtcactgaggggtcagtgttcctatgtgctcatgacattgagggtcagagtgtgcctgtgtgccaatgaaaggcatttcttatatttttttatatgtggtcatagtagaccagttaatttattttgactcctgtgttagaccaaaataagacttgggggaaagtcccttatctatctaatgacagagtgagtttacttaaaaaagcataataatccagtggctttgactaaatgtattatgtggaagtctttattgtcttttcagatgaatcaagtagattattcttgagaccaggaatgttgctgttttggttatttggaaagttttatcattttcaaattgacttttgaatttgagtcaccttttttcagaagtggtgttaaattataggagccctaggttttttttctttttttagaagtcatcacaaaatgatcagtgttcagaggaagagctttgaccttccacatggtataatgattgataaccttaattcatctcttaccataaaccaagtatgtgtaagggttttctttatttcttgaaagcattttgtagatgttgagagcagttttccaaatgtaatttccatgaaatgcctgataagggtacccttttgtccccacagccataccggctctgcagcccatagtccacgacctctttgtattaagaggaacaaataaagctgatgcaggaaaagagcttgaaacccaaaaagaggtggtggtgtcaatgttactgagactcatccagtaccatcaggtaagaggaatgtatgttggaactgtcgtggatactttattgacccgtgcagatggaaggaagtgccatgtggtaacgctcactgttaactgtgttactttgaaccaggtttgggctttctggggcctgggtagatgccggtgcagggggatggggagggaggcggggggtgggggggtgtggtggagttggggaggtgcagtggcaggaggtgttgttggtgtgtatcctttttttttttttgagatggagtctctctccgtcgcccaggctggagtgtggtggcacgatcttggctcattgcaagctccacctcccgggtttaagcaattctcctgcctccacctcccgagtagctgggattacaggcatgcaccaccatgcccagcaaatttttttttttgtatttttagtagagatggggtttcaccatgatggccaagctgtttcgaactcctgacctcaagtgatcctcctgccttggcctcccaaagtgctaggattacaggcgtgagccaccatgcccagcctggtgtttatctttaaagtgggcacagccacaggagttcacctgactcctggtctgagagtcacgagatcgttcaagatagtgaggccctcttttccaaaacgaggaccaaaaatcaattgacagtgttggtcaagatggtagaaaccttaaaatgatagaaatctcaactctgaaataaaaactttatttgtatatttatttaccactattttgacatagggctaaggtctttttctttgagctgatttctggttttgttttcttaaagtggcataagaattcaaagacattttgaggaaggctgagtgcagaaatctctctttttaaatgacttctcctttcttttaacttgcactgttgtctagccctcacttattttgtcaattctttttagctgtttgtctttgaatcttcataaagccatagcttttctcataagaagcagcactttctttgttcattcatattttaatgaacccctgtagtatttaattaaatacttaatgcctaattaaatcacataattgcaatgcaaaagtacatgtatcataaagaggtctgaaaatgagcaactggcaagcaggtggtggcaggcagagctgcttgggtgggtgggtgtcatggagaggagttcatcagccacatgttcagtgagctctggatatgtctgtttagaaatgatcactaataaacttgtgctcaaccatgtatacctctgggaagcaggtgctcttcagtagattgcctctgcagagaacacagaattgaagtgaatgtccacaaaggcaatgagccacctgcagaatagtttagtcaaggctgtgtttgaagtttgccaaagattaatatacatttgattttcatgttgtgccttttctctgattgtgaaatattacaaattctatacaaataacaatgatggcaaatcctcctgagcaaagtgtgcaccttgtatgtgccctagaggaacttgtgtttcgttctgattcccctacatttctcatgtcatagagtgggggttgcattagtgtccccctgtcctcgctgggatcacatctgtttggatcctagagtcttccagctgaactgggacaagtataacagacggacacgtaggggtggaaaggcgtctcttggcagcagactttctaattgtgcacgctcttataggtgttggagatgttcattcttgtcctgcagcagtgccacaaggagaatgaagacaagtggaagcgactgtctcgacagatagctgacatcatcctcccaatgttagccaaacagcaggtttgtccccgcagccttggcttgttgttgcatagtgatggtagcttaaggtccttgtgaaaggtgggtggctggaatcagctcttccttcagtcctaatctgtgccttgatagcagttctccgtgctagtcatgggacagctgacttcatttcttctcacaatgccatctcaggttggtattgcccacctactttacaggggggatcccacagctccgagaggttatggaggtgatcaggcagcacacagctttagagtgctggggtgagggcgggccaaggctaactctaaagcccgaacccttacctcctacactgcctcctgcattctggtcaacccagtgttttatttggtggttagatttttgtttttgttaccttactgcttgtaatttagcagttttcctttcctttcccttcctttcctttccgacagggtctcactctgtcacccaggctagagtgcagtcgtgtaatctcactgcaacaacctctgcctcccaggttcaaccaattctcccacctcagcctcctgagtagcaaggaccacaggtgtgcaccactacgcctggctagttttttgtatttttagtagagatgaggtctcgctgtgttgcccaggctggttttaaactcctgggcgcaagtgatccaccaaccttggcctgccaaagtgctggcattacaggtgtgagccacctcgcctggcctattcatcactaatcagaatttctatgatcaaatgacatgaatcattgtttccacaactgcagtggaaggaaatggcctggcagtgccagtttcagaagcagcctgcccccagtcaggcacaggccactgtgcccccagtgtagcagcacctctgtagctcacagagaagggtggtggggacctccttgaggcagctctgccagaaaatctcatgagctgcctggcacagcttgaggttgccttttaagtggactcagcaaatacatgtttgttcatcttgattatacacaataaacaactactctgtatagtacgagtagtccgtggtttttggcatttgatttaaacttagaggcatgtgatattgatgttactgccttcatgactgcacccccattctgatttcataatggaatgttatcttgagaccagttagacaacaggacagggatcttggcttctggtgagattgacagcagttttagtgtggtcagggtctccctgcctacagatggttttagaatggtgccctggaagctttatcccattcttttctgtgcgtaatctgagtagagtggagatcgaaggcctgaatacatagtaaatacctgacttaatatctgccgcaatggaaattgtgtgatacaacatttatgaaacgcttagtgcagcacctgccaggtagctcaccacaggtgcatgttgcattcagaagtagtgctagatactatcctgttactggcagtgcatacatcagtgatcaaagcagattaaagaaagaccccctgccttcttggagtgaagattttgttgggatgcgggtaaggggacagacaatagaaaagcaagtgagtgaagtctataccatggcggctgatcaggaacaccgtacagaagaatccaggagggaagagagttaggtggtgtctgcggtgggagtggcattgttcagctggtgatgagaagaagctttggtgatctggtgacatttgagtgaatttgcagaaaggaaagatacaagcctaggagatacctggggaaggaacattccaggcagagcaaatagcagtgcaaaggccctggcggggggcggacatgctgttagggtacaagcaatgagggtggaggagtggggcagccatggggagggaagggagtgaggcctggtggggtgaggccagtgtggaggagccttgagagggtttgcgctgatgtggtgtaggttttagcaggatcattcttattcctgagttgagaatagccttgagggggaggtgagggcagagcagggccacccatgtgagacccggcactggagtggaatggcccaagtcagcatcccttggcagcatgaaagcaaaaccagcaaggtttgctggtggcttagatgtggcatgtgagagagagcagggctttgggggtgatttcagggtgaggacagggtggctgtggacaaggtagggcagacattgggggcagcaggaggtcagagcctgtctggatgtagcagttgagaccccataggtgcctaatgaggtgaggccagcatcaggtgtatgagcctggagttgtcgagagactgtggggcagggggtcagcatctgagatgtccactcacagtggacccagactggctggagaggaggaggagcttgaataccgagcctgctgagtcccagctccaaggtcaggtaggtgaggggagccagtgctggggcagggggagtaggcaggtgtggggttcctaaagccaagattttttttaaggcattttgtgcaggagggcgacatctgctgtcagcaccttgggaacttggcccaggtttggcagcaccgagggcactgatgagtgcttttggaggagcaaagggagccaaaccctaatgggaatgtgttcctgaaaggacaggagagagacttgggaaaaggttttacttgaagagggaacggagaaatagggcagtagccagaggaggagaggagtcggcaatgggttaagttggcagaaatgaaggcctgtttacgcactgagggcagaagcaacagggaggatcagttcatgacacaggagacacaaatcgccgttgtggtgttcacagacatgggttaggattggctgcatggatgacagagcactgtgggttctcccagagttgctggggaggaggcagagttggtgagcacaggcgagggtccaggatgcaggaatcctggagctcaagtcagttgttcccttgttgtaagatgtggccagtgttgtgagcttcacatctgtgccttgaaaaacaccacatctgtttgcagagttgtttactatgtatacacactcagtagaaacaaaaattggaaacagtcagtgcccaccatcaataagtaatggttgaacacactgtggtataagcttagactattttagcttgggctattttgcatgattaaaaatgttctggccaggtgtggtggctcatgcctgtaatcccagcactttgggaggccaaggcaggcagattgcttgagctcaggagtttgagaccagcctgggcaacatggtgaaaccctgtctctactagaaatacaaaaagtagctgggtgtggtggtgtgcgcctgtagtcctggctaactcaggaggctgaggtgggaggatcacttgagcccattcgtgcgccactgcactcctggggcacagagtgagactctgttagaaagagagagagagaaagaagagagagggagggaggaaggaaggaaggaaataaatggaagaaatggaagggaggaaggggagggaggaaggaagaaaggaagttcagccagttgccttgggagttctccattgcactgggttaagtgagaagagcagagacgtttatgatttttcaaaacaactaaaacaaaacctctgtgggtgagggggcaaggatatggctataggaacatggggcagattaagaaagggatatacacacaccacttagcatttgttacaactgttgtgggagggatggagtgcagaaaaagaaaaaaaaaagtgcacaccatcccatgtatgtgtatacaaagggacgcttggaagactggtccccaaaatgttggtaatgattgtgtcagggtgctgcagtgctagttgattttttttcacacttttgtatatttgagtcttttacagaaagcatttattatttatgtaataaaaatctaaatgacaagatttctgttatgggaaaaatgtagctatacagtgttgttgtaaaaatgtttgcttggttcaccactgaacttaaaatgcttttaaatgagggaaggtgacgatgagatgattatgatgatttgcccttgagttacatagctggtgtacaggaagctgtcgtttcttttggcttacgtagaaatgtttgtggtgtctaattccacagatgcacattgactctcatgaagcccttggagtgttaaatacattatttgagattttggccccttcctccctccgtccggtagacatgcttttacggagtatgttcgtcactccaaacacaatggtgagtctctcgcctggctcagcagatgaatctggacggcttgttcaggctctgattactgggaccacccccagaatgtctgagtcagtcagtttgggtagggcttcttgagagtttgcttttttttttttttttttttttggtgtgggggtggtgcggaacagagtctcactctgtcgcccaggctggagtacagtgtcatgatctcggctcactgcaagctctgccttccagcttcacaccattctcctgcctcagcctcccgagttgctgggactacaagcgcccaccaccacgcccggctaatttttttgtatttttagtagagatggggtttcaccgtgttagccaggatggtcttgatctcctgacctcgtgacccgcccatctcagcctcccaaagtgctgggattacaggcgtgagccaccgcacccggcctttttattttttttggagatggagccttgctctgtcacccaggctggagtacagtggcgctacctcgactcactgcaacctccgcctcccgggttcaagcaattttcctgcctcagcctcccgagtagctgggactacaggtgcgtgccactgtgcccggctaattttttgtatttttagtagagacggggtttcactgtgttagccaggatggtcgcgatctcctgaccttgtgatccgcccgcctcggcctcccaaagtgttgggattacaggtggctctcgcaccaagccaagagtttgcatttttagcaaattcccaggtgaaactaatgcctgcttttctgggagcacactttgggactcagtgatagagaggtttattggtaggatagtaaaataggagttattttctttcacaaaattggcaattgggggaaatttaatcttccttttttcttcagctgtgacttatgtattatgtttattttaggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtatgctttctatctgagcctataactaacccatgccttttgggaagtcacgtgatgtttcacagtcagtaagtctggaataatacctggtcttgcttcacttctgagttgggtaaagaagtctgtatcagtgtaattttctaatccgtcctgcattatctatggctcttggttcatacctgtcttgaagttctgtcatgttctgtctcttgtcctcagtagagatgctacagcagtggctcgcctcaggcagggcagggcagtggggtggctgtcctgggggcaggcagtaggggcacgctgacgtcagggaagttgaaacccaagagaagccagtaaaagtgagtctcagattgtcaccatgtgctggcagttttacacgctgtcagtaataaaagtcttctccctgcagggcagcctgcctccaataaatacgtgtagtatcaaatcctgtcttccctcataaattgtttggaagctccccaaggacagtgatgaggcactcgtaagtgcttgctgcctagatgggtccctctccacctttgctagattctgagcattcactgagttagagctgcttctgcaaatgtgctgcttctgctaagtggctgtgacttcatgcagccttcacttggtttgtcatcagtggagatgccctgtgttgtcgaaggagataagcccagtaagcctgctgggcaccttttggtttgcaggttcagcaggcagcccatggctttccctgtgtcgcattgaagcagctggctaaaattgatgatacattaaattcctgtgacagatgatcagcttgtatttgtgtaatggtgtacagttcacaaagcttaaaaaaatgctacctgccatttcatcctcagtgaggaaggtgatacacagagagaccaagtgactgtgtccacggcgacggcgctctgcatttcactttagcggttaatgtactctacctatatttttactttatatttaccatatatcttttcatgtatacttggcgtaagtgctttatagtagtcacctaattcactgtcatcttttttgtttcttggaaggtttctattacaactggttggtattcttttagaagacattgttacaaaacagctgaaggtggaaatgagtgagcagcaacatactttctattgccaggaactaggcacactgctaatgtgtctgatccacatcttcaagtctggtaggtgaatcacattagtcttcctggagtgtctcgttccccattctgcactatacactctcagagtgtaggagctgtgctgcccggtagaaactctgccttgcccagtgtgccagttgaaaatatttgttgctgtaagagtacacctgataccatgtgacccagcagttccactcttgggtatatacccaaaagaatggaaagcagggtggtgaaaagatatttgcatgccagcattcatagcagcattattcacgatagctaaaatgtggaaccaactgaagtgtccctcgatggatgaatggataagcaaaatctggtgtatatttacagtggaatattattcagccttaaaaaaaggacattctgacacatgctacaacatgggtgacccttaaggacattatgctaaatgaaataagccagtcacaaaaggacaaatactatgtgattccacttacatgagggacctggagtagttaattcatagatatagaaagtagaatggtggttgccaggggctgcaggggaggggagttatttttacaagatgaagagagttattctagaaatgaatggtggtgatggttgtataacattatgaatgtacttaatgctactgaactgtacagttaaaaatagttaagaggaccaggtgtcatggctcatgcctgaaatccaagcactttgagaggccaaggcaggaggattgcttgagccaaggagtttgagaccagcctcagcaacatggtaggaccccatctgtacaaacaaactagccggggatagtggtgtgcatgtggtcccagctactcaggagactgaggctggaggatcgcttgagcccaggaggttaagtctctagtgagatgtgttcatgccactgcactccagcctcggctatagagtaagaccctgcctcaaaaaaacaaaacaaaacaagacaagagccaaaaatggttaagatgggccaatcacagtggcttatgcctgtaatcccaacactttgggaggtcaaggtaaaaggatcacttgaagccaggagcttgggaccagcctgagcaacatatcgagacccctatctctacaaagaaaatcaaaaactagctagatatggtgggcacatgcctgtagtcccagctacttgggaggctgaggtgggaggatctcttgagctcaggagttcgaggctgcagggagctattattgcactccagcctgggctacagaatgataccctgcctcttattaaaaaaaaatccaaaaaaaaaaaaaagtaaacctgagagcttcctcctcctgtgttaaatttggaggccaagatgtttttgttacttttacaaatgatcaaggacggtgaaggttgggcatggtagctcacacctgaaatcccagcactttgggaggctgaggcggggtgatcgcttgagcttgagaccagcctggacaacatagcaagagaccccatctccacaaaaataaaaaaataaaaaaaaatagccaggagtagtggcatgagcctgagcccaggaggtcaagctgtagtgagccatgatcatgccactgcactccagcctgggcgagatcgagaccatgtctctagagaaagaaaatgacaaggacagtgaacccaagaaagtcataagatgccagctgtgcagcaagcatggaaagcagccagtccaaattaggacagtgtgttttccaagaagaacgatcgtttgtaatgagaatgctttgctttaaataaatgactaaatagctagaagcctagttctaggggataggcacgtctttcttctctcaagaaaatagaaaggcaattctaatttctagtaacagcaaacagcattaagtcatggtccaaatatgaggcaaaccaaaatgtggcttgattgttcagcagttgatctgttggaagcccttgatattaaaaaggttctcctttaagcggcttaggagtcacgatcaaagacctatagaaagagatgccatccttctaggatccttggctctcttgggaactagattcagatagtcataatgtaaatactgcttgagctttctttctttctttctttctttctttttttttttgagacagagtttcactcttgttgcccatcctggagtgcaatggtgccatctcggctcaccgcaacctctgcctcccaggttcaagcaattctcctgcctcagcctcccgagtagctgggattacgggcatgcaccaccacgcctggctaattttttgtatttttagtagagacagggtttctccatgttgaggctggtctcgaactcctgacctcaggtgatccacccgcctcggcctcccaaagtgctgggattacaggtgtgagccaccgcacccggcccgagctttcatttttgaaatcaatgtatgactgaaacactgaagacttactgacttaattatggtttcagaacagaatgaaaatgtcttcggttctgatgaatataaaaggaaaactaaccaagttaatttggcaagtagatggtagagatagaggtggggagtggaaggggaactaaaatcttcacctagcattgttgggattatatggttacatcatctgaagttgacagaccaaaatatagaggcttcagaggtctccaaatagaactaaacatgtaattcagattgttaggaggtagtataaatgagctaaatctcatctttattacggtagagttaatgggtgatgtctaaagttgtctgaagtctataaatcatgacaaattatgatgtggtgattgtattcaacagtctttcagttgcagggataaaaccccagtttaaactagagtaagagaaagaatgtgttggtttaagctcctggaaagtgcaggcaagggtagttggtaggactgcatctagtgttgtaattctgtggtctgcattgtatatttatgcatctcagctctgctttcttcttttcatttatataatttttaaattttattttaaagatagggtctcactttgtcgcctaggctgaagtgcagtggcatgaagtgcagtgcgaggctcactctagcctcgaactcctgggctctagagttcttcctgcctcagccttctaagtagctgagacaataggcatgtaccaacatgcctggataggttttaaaatttttttgtagaaatggaagtcttgctgtgttgcccaggcgggtctttaactcttagcttcaggcgatcctcctgcctctgcctcccaaaatgctgaggttataggtgtcacccaccacgcccagtctcatctctgcttcctgtgttagttttgttctctggtgggctgttttcacatgaccgaagatgacctctagcaggctgtgttctcagcccctcaagtaggcctatgtgattggccttgcatgagtaatatgggtgaccataaacccctgaatgctctggtccacatgggccaaatgggagactggacagcattccattgatgaggaggtggggctggtctccgggagtaagggagaggagcacatgcagtaactgatggtctgctgcaagggatagcagcacagcagttagaattttggaggtaactaccagaactgaaaacagaaatgataacaagtagttgccttaaaaagggatgggagcagggtgcttttgtgatcaaagctcctttctcttactggatttttgtacacattttgcatacatatcttagagtaaaagatagcattttcagccttggtccatttgaggatactcttggcgtggcccgcctccatgctagcaggctctggttgtgccaagttcagttgagcatcctggctcttgcctgcacggaacttccagtcagtgcgtcagtatcacaagtcttgatatttcctatgaagaagaacagtagtgcagtgacagacgaaatgggtgggcaggcagaggcaggatttctgagggagagaagtagctagctttttgcagagaagagttccggcacccaagagagcagctgagagtacaggcaggcaggcaggatgccggtagggcccggccgcacggcgccacagaatcctggagaaaggggcctcttcatggcctctgcattcagctgctgtcaccctccgcacaggccatggccaaaatttaattttcatagtggactctagtttttgagccttacttgctattattgaaataattttcttgtttctttttaaagatcttcggattatgcttcactgaccactgtaataagtttaaagttgagaaaatatggcttgttaatgaatgataggtcaattttagtatgttggtcattttaatattttgccaccagttggtttggatttgatgccaggaggagacagcctcatttctaaggactagtcttgcctttgtgggataagggtggtgtgttctgtgtccttctacatgtccgagcgatctctgtgcagctcaaatgtggtcactgtcttattgcgctgatttcctctccttccatctcacaattgaggcaaaatattgttactgttgaagtgttgtccaataggacttccagcagagacaggatgtctgcactgtctaatttagttgcctttagccacatgtggtgttctgtacctgaaatgtggctggtctgattggatagcttaatttataattttatttaattttaattaacttaaatttaaacagctctgtgtggatagtggctcctgtatgagacagtgcaggtctgttgagaagcagctttactggtgggagtggagggcttggagagggcacgtgggtttcctgctggtatcttttgaccttatttaatctgcccaacatttgcaagtaagttgtgtgtgtgtgtatatataaatgtgtgtttctgtcttcttgtttcctttgactgcatttatttgaaagacactaggtggcagaattactgtatttgattggtttcaagataagagttgaaataattcatctcgtgtttttatataagtaaggtgtgtttagcatgtaaaattggtaatatgtattcacgtactgcttaaacaaaggctatgaattccacccataaaccgaaaatgaagacctttaaatttgtccatttcaggcgtgggtacttcttaaataatacctggttcaggaactagtcagaatggcacccttgactttttgtttcctgcttttcctcttgttgggagaggagggtattcatcccaaagtggtttgcctatttcacattccatctaggataagcagaatagccaagaaagatagctgtcctcctgtttacaacatttggggtaaccagcatccctctcttttggtccaagatagactggtttagaaacagatgatggcaccagaggcccaggaggtggaaacatcagctttgtttgttgtccatgtggctgaattagagctgtctggccttgtagcctcaacacggccttccagctttgctcaccgtgattttcaaggacacatcttgtgctcttccctgcctgccatccagactatacccagtcagggtggcaggagctgctgccccttcctccctgagtcctggtcgtgggtggtggagatgtgccatgacgctcacggaggcatgctcaccccttcctctgtggcagaggggatggctgcacgacagctcttccctgtcctttccaaagcgtctgtggttccactttttggggcaaagcaggaatactggaagagagagaaagtggtcctttctatagtaataaagttgacattgattcaagttcatgcttggggaaaggacagggctactaacaattataatgctgggagcaatggaattttctcatgggtatgtggtaggtttaattttaattatcccagttaattcttagaactgctctgtgaagtatttcccgctttgtgcttaagttctaaaagatcctgtgccaaaaccaagaatgaaaacccaagcattctttcttgcccatcgatctttctctcatcaggccacttcttgggttgatagtggtgagtgtagccgctgccactttcagaatacccaccatgggccccagtcactgtgtggcgtggagaagagatggttctctctgtgtcatagctgaacaagcccagcccagagaggtttctgccctaggagctctcgatggtggaattgggatgcgatcccacatcctgcctgttttgaaaacagcattctttatttccaattcctgcttccattgttccttttaatatttctttgtttagctcacaaaaacacggcttgcggagctgctgcgtgcagctgtagctgtttctctgggtgcagcctgcatccgccttcctgcccgcctcctttcctgcactgccatcgtggtctccgggcacttggtccctttctcttcccctgagtccctttggctcccctgtgccacccttgtgatccacaggctctgccttctttctgtctcagactgctgctcatcactactcgggaccctaggaagggaggttccaccgagaagcatcttctcatctcagccacgttctcagtgccactgttgtctttgttaggtaatggtagctactgtaacaaataaaccaacatttccatggcttcacaccagagaaggttgtttcttggttttatgacaatgtattgagggtgttcttggttcacggatggttttcctccatgtgggaattcggggacccaggctcctttccttcttttggttctgttctccaggccttcacatcctctgtgtctggttggggacaaggagagggaaggtaaagaaggctttgtggccttggataagtgacaggcatgcctttgctggtgttctctcgtggtgacaggtcacagccccaccctgtaaaaggggactgagagacgtcgtcctgctgcttcccagcagcagcactgtggtctctgatgtgttttctgtgaggataaaaacaggtgattccaggatgaggaaagtcagggaaacccttggaaggaggggaccaggcgggtgtcaccatgggattagtggtggcttcagaatgagctgcagcgagtgccatgccttctaaagcttttgctattctgatatgcccacaccatgcccagcaggtgtctgccttgctctccgcagagagagtgatgaatccttctcatgagcctctgtccagttgttcctccctccacctggaagggaccctgggttcctcataacatcccagcggaacaggggaccttctatcctgtccccaagttcatcctcatcctcctgccggcttcctggcccctcttatgtctgcttcctgacgccacatccttctggattctctggaattgaattttgcctttgatgcttatttaaaaatatccattgcaggccaggtgtggtggctcacacctgtaatcctgtgcactttgggaagccaaggtgggcagattgcttgagcccaggagtttgagattagcctgagcaacatgttgaaatcctgtttctatagaaaatacaaaaattagctgggcatggtggcgcacacctatactcccagctactcaggaacctgagacaggaggatcaattgagccccggaggccaaagctacagtgggctgtgatcgtgccactgtactccagtctggtcaaacagagtgagaccctgtctgaaaaaaaaaaaaaaatccattgcatacttcaccgtagcgaaacatgtatgtcttacctttcctttcctgcctgtagctgctcttttacacttaacagccacactaagccagccttaaatgaaaaacaaaccagcacttcctgtgccctcctgcttccttcatgaggggtccctccctctgtgtacactccattctcattgcccatggtggtttgtttccctcttgtttctcaagccatggcagcctgcctcttgccctctttactaaaaaggcctttgcagaggctgcctgtgttctttctttctaggtctctctcatcctaggccctccagcttgattctgtggagctgccctcttgtcactcagtagcttgtggggtcttctctgtctagccacttaattgattgtgttcctcgagttgctgtccatggtctctcgttactgttttctctgtgtttctgcctctctccttggccttggtaggtccatcccctttgtgaccttggctgttgctctcatggacaactttctcttgctggtccttgtagtcctggcatccagcttctcgacacgggacttgtcctgccagtacctcagacttgcacttaaaattgaactagcaccactgtcactctccagggcctcttcttgttaattagatcattagggatgttcagaatcccagcatcatagtatgttcctcctcccgctaccccaggaaccctaaccttacctcctcctctctatctactaggaggtggccctcagagtccgtctcatcttccacctgaacttccctaataggctccagcagctgccaccccgggggctgagtacttcctccatgccttgtgcagtgctgagccctttacctgggttctcctgtttgctccttattacagccctgcgaacagatactgctcttaattccatcttacacctaaggaagctgaggccccaggtaaggtgcatccaaggtcacccaggtagtagacagtagagccacgatctgaaccaggcagtctgattcagagcctgtgttgacactcagccacctagaacacagcttggattgtgggtttctattacctgttcaaaacccctacatcccgggtctgtccctgcacgtgctctgtggcctggctgcatcttccttgaaggcagtgcatgcctcttcactcagggggcccatgcaggaacagagggccccacagaaggatgaggccagtgcagaatgggctggaggggacaatgctgaccaggaagcaagtgtagagaaatcccaggaaacctggaggagccagagacaaggcattagaactcctcgtcgtgacctggtctgcattctctgagtgtgctgcttctgttagctcgcttccttggtctcaggttatagtttaaggcattgtggagccctaaaaagcctgtactctgtttttacctgttttaggaccctttcactttggggatgtgttgattttttttttttttttttttttttttttgagatagagtctcgctccattgcccaggctagagtgcagtggcacgatcttggccactgctgcccctgcctcctgggttcaagcaattcttgtgctcccgcctcccaaatacctgggattacaggcacccgccaccacactcggccaatttttgtatttttagtggagacagggttttaccatgttggtcaggctggtctcgaactcctgacctcaagtgatctgcccaccttggcctcccaaagtgctgtgattataggcgtgagccaccacacccggcctgaaatttaaatcagaaataaaattttgatcccaacagtgatgccaggcagcccagatctgggggagagggtggccttggccagctgggcctttctctgtttcccaagtcttgctgcctctccctgctgggctttgcagcctgtgcatgtctctgtgcctttgaccttgtttatccaaaggagaggatagaatgaagtcatgattcctggagccctgagaaggatgctgtggagaaatttgccggtagaatctagctgagtgtgttgctgaggtgccagcattgtgtgtggggaggctgaccgcttggcctgcctaggcccaggatgctccatggccgggcacagaggccacttggctgtcaggtgtcaggagcctgcagagggcacacagagcctggaccgcaggggggtcctgctttctcacctggcctccttcagcatttctgtccctcagtccttagcaagcccaggagctgttgagtttggcaggtgccgagtgctgttcctgcctgtgtagctgtggctcagtcctgtgggggccccgctgtggcccgagtgcagtgattcgaggcgctgagtgttccctgactccttctccaggagctgtgttcagactttcgcagctcttggcttggagctcctggagggcttggcattgccgaccaatgtggaggtcgacagtgagagaggaggaatgctagctttcttgaccagtccattaaataagtgggatattggccaggcacggcggctcacgccttaatcccagcactttgggaggctgaggcgggtggatcacgagctcaggagttcaagaccagcctggccaacatggtgaaaccccctctatactaaaaatacaaatattagctgggcgtggtggcaggcgcctgtaatcctagctacttgggaggctgaggcaggagaacagcttgaaaccggaaggtggagtttgcagtgagccaagattgcgccactgcactccaacctgggcaacaagagcaaaactctatctcaaaaaaaaaaaaaaaagtaggatatctgtttctgcttagaaaaatcagaattttctaaatgccaggtgttctgaatacgtaagtatgggagacgactcagcctgtttcatttttatgtaaaatcttcgcgtagccatgtggcactggaccgagatgaaagcaaagacatttctccttaactttgtttctaggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaagtaggttcataatgccccacagcccagggcgccagcccagcaccctgtcctgagactcccagtaacctgagctttggccaccgttaaagcattttcattttccattttttgtgagggcttgtgaaatttctgctgcatattaatattcctttcatggacagcatattattgggacaaacatgcggtccagctaaaggcattcaaaatagcagttgctttctaaatgcgattttctttggcaggttctttgacaccattgcatcttgtgggatatgcttgtcatgctctgtggctcctactaagttctagtccttaaattggttccatagccagacatgttgcaatgtcttaacctcattataaagtaaatgtggttctggttatccttagataatgaagtaacagtgtagcaaatttcaaaacctcttggaaatgttattttaccattcaaaaaggcttactaaggttctcgttatgggtggccctctttttgcaaaaggttttcaggcttaagctccatttctaggtgctccaacactccattatttgtatatgtatggaaataaaagctgtgaccacccccaaccctggcccccgcccagctgaatcctcagcacagtatttctggaaggctcaagatcccacgctggggaaaagaagttctggagacaaaagagggcaggtgctgccgtgcctctctgctcagtatggatactggaccttgtgctgccagggctcccagtagggccagttcatggcactcagctggaaagtccactgttgggaggcattcttaaccatccactctgtgccgtatgtagtggggtctggtcattctgttggaggagacagaccagtgacgacatttgaaatgcttggtggatgtcttaggcctgttacgatgactgagcactgtgggggcaggagacagaaagtcagtgtctcctagttctgtgctgctttaacgtgcatagaaatcagctgcggattcagcagatcactccttttctgacagatgggcctgcttactctgatgttatatcagaaagctctgaatctgggaattgtgtcccctgaattggagtaacagaaatgcttagatgatgagtgtttaaaagaaataaaccaaaggtaaatttagtttggaattcagcaagcgtcttcattcagccctctgagggcaaactacagctttttgtaaatgtaggtaaattctgtgactgtttcgtgaccccctctgatccagttttcctttataaccttctgtattgttccttctattatcctgaaataacattaatagattaggctgggcgtggtggctcatgcctataatcccagcaccttgggaagccaaggcgggcagatcacctgaggccaggacttcgagaccagcctggccaacatgatgaaatgctgtctctactgaaaataacaaaaattagccgagcatggtgacaggtgcctgtagtccctgctactcagaaggctgaggcgggagaatcgcttgaacctaggaggaaaaggttgcagtgagctgagatcgcgccactgcactctagcctgggtgacagagtgagactccatctcaaaaaaaaaaaaaaaaaaaaaaaattaatggatcaatggatttttaacctaataattaaatttcaaaaaatatcgttctttaatggtaatgtaaaggtaaaattaagataatatgtaacaagcatgtgagtgtctaaggtgtccccgtggtggaaggaaaaaataaatccccataagtgtccaagatgcccatagagagcagagctgttctggtttaaacccctgctcttagcactgtgtttttccagctgtgggtggtgggggatgagtatctttttatttccatgagatgagaaaaatgaattactagaagtgtgaaatacaaaacacagctgctctttttttagccatagactcagcagccataaaattgctgtatccagttgcagaaattcctgctgcttactcttgaccctctctcggtttgtgtgcatctcctctcaggctggctcccagatgggagctggctccaggcgacactgggtgctctgctccaggaggtccttatgtgggtcctgccctagcctagcccctctcttatggactctgtcactgtgggtttatgattcactctcaatctgtcttacctcttggtgaactgttagagtcctgcctatactttggcgcttgtgggtgtgttgtggtacacatgatgtgttggtcacttcccagctcatcttgttctgagtcaccctagatttgggacattcattcgccaccagtaccgggcggtgtatggcctgagatttgggggggcttgtgctgctacaaattggggctgaatttgagttgacagtggaccttctttatgtctactgctcatatttgaattgcaaatactgcctcttctctttcagaggctcattaccctatagctgtattattgcaaagtgcacaattacagcttgagtgtaagtcacactgcgctggcaggacggcccactgagaaagggcacgtttcctgttcgttagttttcacattgacacataatttacaatacagtaaaatgtacttttctatcaactgtagtcagtaacagcccccctcccccaaccacatcaagatatagaggagtgctgtcacttcaaacagttccctcttcctctgccacatcctgcccctccccaggtctaaccaccaatccgtgctctgtccctctgttcagcccattgcagaaggccatagaaatagaatctataggctaggtgtggtggctcatgcctgtaatcccagtattttgagaggctgaagtgggaggatgacttgaggctgggagttcaagactagcctgggctgcctagcaagaccccatctccagaaaaaaaaaatttaaaaattacaatcacgtccctgtagttcagctgcttgggaggctgaggcaggaggatcacttgagctcaggagttagaggttacagtgagctatgatcgtgccactgtgctccagcctaggtgacacagcaagacgttgtctctggggaaaaaagaaagaaacggaaccacgcggtgtgcagccttctgagtctggcccctttcggtgagcagtgtctaaagttctgtcgcgtgttgcccacgcgtcggtggctcgctccttgcaactgctgagcattgtatggctaggctgtagtttgttttcacttcaccagttgggaaacagagaaaaggcactttttaaaaagtttaaatctgtagaattttggtttttaccagttctcttctaaatcctgagggattacaggaaaagttgttgtatttcagaatattcttagcttgatgtgacctctgtccccgttaaggccctttgccgcaatgggaaggacgtcgctcggtcagaccctgaaggtcagaggggcagtttgggagtgtgtcaacattttaactgtatggactagagccaagagtctcaaggtttataattcccacgtattcaaaaagaaaaaaacaataaagtgagaagtcagtgtagagtgaaataacctgtgttagtggggaagaagtgtttttaaacaggatttccataacgtataacatcaacatgtttagagtggtgatgtttcattgggaaacgaacagtaaaacatgaaagcagggaggttttcattctggcagttggcaactttcacggcagatggagaatttcaaaagcaattgctcaattatcaaacatagccagtgtgagttctgaaataaaggtgctgattgaatgtgcagctttatggtggattttgctattcaggcaagcattttaattttctgcctgttaaattctgttttctttagtttttcatatgtggtttattgtagcttaggaatagataactgagagtatatattacacatacaacattctgatatggcaatatttaaaacaacttgtctgttttagaactagaattaaacataatcatcttcagtattttgcaaataagctcactgccatccagaaacattgtcaatgcatctgttgctccttctagaagacacagtctgtccagcacaaagttacttagtccccagatgtctggagaagaggaggattctgacttggcagccaaacttggaatgtgcaatagagaaatagtacgaagaggggctctcattctcttctgtgattatgtcgtaagtttgaaatgcctgtaaacggggttgagggaggtggggaccaggagaacatcctgtgtagatgacacttgcatggaccctctggaacccagaccgcccggtgtcctgccaagctccatcgaaactaaatctagaatgaatgtttacttctgctgtgacatataattggagaccaggcctggccttccagtcactggattctaagttggactgtgagagtttttgcagctgactcatttatcaaatgcccggctattggctcacgcctacatgatgctgggtatgtttgttaatttgagggaagcaatggaataataataactaatgatttaaaaaacaaagtaagtgcattgactgtagtggggttctgattttaaatttttttaaaaattaataccaggagcagtggcttatgcctaaattccagcaactcgagaggctgaggtaggaagatcacttgagcccaggagtttgagacaagcctgggctatggtgtgagacacccatctctaaaaaaataaaaaataaaaaattatccaagtgtggtggctcgtgcctgtaatcacagctctttgagaagctgagggcggaggatggcttgagcctgggagttcgagaccagcctggcaacacagagaaaccctgcctctaccaaaaaaagaaagagaggaagaaagaaaaattagcctggcgtggtggtgcatgcctgtggtcccagccacctgagagactgagaagggaggattgcttgagcccagaagtttgaggctgcagtgagctgtgactgtgtcactgcactccggcctgggtgacaaggcgagacccctgctctaaaataatttttttaagttaatttgtagaaaaggtgttagatgttctttgtcacattttatgatggattcctgtttaaatgccgttctctttaaagaaaaaaaaataacttgtgggagtttttaaccataaaactagcatcacatatttaccatggagaatttacaaaaaaacaaataaacggaggaaaataaaacctcctgtaatcatactactcagagataacttgctgttagattttggtctagatttaatactttttctatatttatattaaaaatatttaaaacatatgcatttctttgtcacaaacatggtatcttatagatactactgtcacatagcaaaacagtgttaaatattctgaatcagaaaaggaagccgactctccaactgaaagaggtgttatcctagagactttttctggtgatgacaatttattaatagtcactttttgctttactttctctattgaagtagtttttctattttgttctacttttaaggataatataatttataatgctgtttttcacagaaatataagaaaaaagatactaattttataagttaataaagtttgatcatcccaaatccaaaaatctgaaatccaaaatgctccaaattctgaagctttttgagtgctgacattatgttcaaaggaaatgttcattggaaggtttcagattttcggatttagggagctcaacaaataagtataatgcacatatttcaaaacctgaaaaaaatcctaaattcagaatacttctgatcccaaacatttcagataagggttattcaacctgtactgtcagatgatcccaaatgaaaaatattaatcgttaaccaaatatcaaggaattgatcacattttacagtttctgcctaggattatgaatcaagatgaaaaggctctgcatgtttaaaaatatatatttttattttcttataaatcttaaatatctacacttaagatttatttgatatgtgggatccattcatattttggattcaacagttctgtcaaaactgtggcagtgataggggattctttttttcccactgaactatcacaaaattggaaaaagagtaattggagaaccccactggcttagccggcccgaagcccgggagagggcaggcagtgctgtggatggggtcatcccagcgcaacgctgcccctgctacctgcggatctcgctgaggcctgcctttgtcctttgacccttggccatttgttagtgtctctgagagctggactgctgtaccctacttccccagggggcctaacttcacacagcctctgccgcagtgcgtggttggaggtgacggccttggtaaatcgagtttcctacctcctcaattatttgtgctcatacactgtatatttttagtgaggtttatatttgggatgtgttttctccttcttaccctttctggcctttctatggcattaatacctggtctcttcttgtgtacttgaaaatgaatctctcatcatatttttccttagtgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactgtacgtcttcatcctgccgactattgccagttgcagttttccctgccttaaaaatggagtattgaaatttttaactttaatttctgatttgcaaaatagtcatcttttgttcttttccttcttgctgttagccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacaggtattgggaagagaaaccctgatattgatttatattgaaaatttagcaggccaagcaaaacaggtggctggctttttcctccgtaagtatggtcttgacatggtcaccgatagaaacatggaaacatctgcaaacttgccgttactcgtgtgtccgatctgactgtttcttgtatttttttctagtctgcccttactaggatgaactgtacacatcagttcatcctttttaaatgagcatgaggttattttgggttgttaggtgttacaaacacactaatgtgtttttgtctattagagcagcatggcccagttgccaatggaagaactcaacagaatccaggaataccttcagagcagcgggctcgctcagaggtaatgctggaaacacaggtcgtccttgtgttaggacaacccaggatataaaggatatagatttgtacgggaataaattcacaggacaagaaatcgatgtgccttataggtgggtttactgcagaagtgccataatagaaccttcctacttttaaaacaaccagatctcactttctaaagagtaaaggatgaccggcaggatcacgtctgtgacgtgagtggaggcagtttgcactcctggtggctgtttgagaggtagcatttagaatgcctgtattcactgtcctgtgatgagtgggaaaataggttatcaggtttatcttagcaaaatcaaagcatgtcatctaattgctaaacaagagttggcaaatctgagagacattactcaatccttggcatgcaggacttacatctgcatcctgttgccattttatgtcttcaaagcatttaatcatttagttgtgtttgcaaagtctttgagaagcctttgtcagaaatccctacatctcctatgtgagtgtatttccatgactgcagaataagttaaacttttacctttttccttcccttgcggggcggggtggggggcagggattgtgtgtgtgagagggagagagagacagcagagaaggagaatataattatcatgctgtgtactttgagctgaaactgcaaaaaaggaaaaacacacaaaaattattatgcttttcagtctttagagtaccttgtctattatgcttttcagtctttagagtaccttgttgatggtgtttttaaatgggattgggcacaattaggtggacagtttgggatgatttttcagtctgtagggccaagctcttttgtaatttgcattatgaagttgtcactctcatagcagatggcgggagataaactattattactttttgaccctagacttagtcttcagtccagatgagggagattaaaagattataaatatcttgtgccagatgaggtgattttattttgaaatgaccatgaattcctatcagttgtcttactgggatatttgatagtggaatttgtgcatttgagtcttagatgatctgttttacatttattaagaaagcctttattagcttttatactgtgtattgcctgttgcagtgtttgagtataaatgaaatttctggaaaatattaatggagtacaaactgtgatacttaaaagtaaactagggcctgcatttgtatcatgacctgtttgagtattgatgagaagatagctgtgaagaaaaaggtttaaacaagtgtattttcctttaagaagccactaatagtgcatctccttagagtgtatatttctagaatcctagtgtgcagagtttagactaagactaaaaaaaaaaaaaaacaaattatactgtaatttcatttttatttgtattttagacaccaaaggctctattccctgctggacaggtttcgtctctccaccatgcaagactcacttagtccctctcctccagtctcttcccacccgctggacggggatgggcacgtgtcactggaaacagtgagtccggacaaagtaagtgtccagcgtgtctgcatgggaggcacagggcgctgagtgcctctgtcacctgtggcagatacagagagtgcagaggaggtgccgtggacccaaggagttctggcgctcggctcggctcagtgaagctgtggttagagacgtggggggccatcaaggtctgagggagccaagcagtgctgatgtgggacccttttggtaggagtgtggggtgagtagttagtgggtgaatcaaggaatagtcggccgtggcctgcaggcccctgactgcacaggccttcaagcacatgtcaatgccgttagcctccctccatctcctcataccttctggccacctgtgagttgcactgccactgccagccattctggtatgttgtcagcacctccactgctcatacctcatggttagggaccacctggagccttggtagagccttggtagagccttggtactctactttcctggacaaagttcagcttatgaatatgaatttagatttcaaaaaccagcagcccaagtataagaaagcgaaggttcagtcctgccttcttaggctctattcgctaagcacctgccctgccctggttgctggggagagatgagtaaagcagacaacccaggagaggatggcaaaggggccgctaacccttagtggtttagctatatttggaaggcctattggaagttcaccaggtgaagggggaggctgtgagggtgcccaggcaggtaacagaagtccaaaggggaaaacctgtggtgtggtgagccgtatagccacagcctgccggccggcagccctctcagcctagtgcggtgttcccaagcactggcctaggcctgtagctccagggatgtgaagtccccttgaacgccgcccatcatgttccccttatccatttttttcttcccaggactggtacgttcatcttgtcaaatcccagtgttggaccaggtcagattctgcactgctggaaggtgcagagctggtgaatcggattcctgctgaagatatgaatgccttcatgatgaactcggtacggggggagcagtggaggcaaggaatcctcagcttttcttgtgacttccaagtgggatttgtctcatcatcatgtgacccacttgttgacaacacatgttggggactccagtctgggcagggacgggatgtcggagagactccactctgaatggggccgggaagtggggaggactccatttcagatggggtcgggacatgggggttatgctgatcgagacagaaaagcacattgtttcagccacattagaatccacggaggtgttgttttgaaatccagctggccccaaggctgggtgtatggtttgggatgagaactatctggcctccactggaggaacaaacacaggatgttatcatctaagctccatggccaagacagaatggaagtcaaggttgcgtatttgccgtagacttcaacacagtgtcgtaatgcgtgacgtcaataacttgtttctagtgtcttggaagttgatctttagtcgtaaaagagacccttggatgcagcgagatttcctctactcacacctctgttagatgtagtgaggttcttcaccccccaaccccagatgtcagagggcaccctgcgcagagctaggaggccatgcaaagccttggtgtccctgtccctcacccgtgggcaggtcctgtgagcagtgggggggccacctcttgggtatggtgcagccatggcccaagcagggcttcttctcagacctactaggacgggagaaacctcctggtgctttagccctgcgttgatatgcagcaaatgggagggaagtgggcacctgggaggacaaatgcctgtagaggccgggagtgacggcaggtgttcatgaaaagagaccttgtggggagggcaacacaacagtgtgttctgatgtactgaagagctcaactgaaaacaacaggagaattagcccaaaatccatttactaaaattgtttatctttttttttttttttgagacaaagtctcgctgttgtcccccaggctggagtgcaatggcgctatcttggctcactgcaacctccgcctcctgggttcatacgattctcctgcctcagcctcccaaatagctggtattaacaggcatgcaccaccacgcccggctaatttttgtatttttagtagagacgggatttcaccatgttggccaggctggtctcaaactcctgacctcaggtgatccgcccacctcggcctcccaaagtgctgggattataggcctgagccaccacgcccggcctaaaattgtttatcttaagattcatgcagtgaaagctaacttactgagtgataaatttgcttagtgatctgtttattaggttttccaaatttgctaattgggctttgaacagctgtaaaagttctgactgtaaaagaaagcttcaacttttggcattcatgatgcttttctgagtattaaactaagatagatgttttacctgaaggatcggccaccaatctttaaatggctaaacaaaagggttgctaaaacataatccaaattgacataagaaataccatttttccaaccaaaattttggcattcatatggctacttttacgtatttcagctgcatttgaacatctttttcaaactttagggtggttggtgtatcactgaggtcttggatgacactttagctttgattttgtttttatgaattaaaattgtcataccaaaatttttatttcaagcaaatccaagagcataaaaaattaaaatattacttaaaatactaagagagaacagatatatattttactaagcatatgttgaatgaaattgttcaaatatttataacaggcatagagtagaattttcttaaaaatatttttgatggtataccaatttgtattttctcagaaacatttgccttattcttttttctgttgtgtttttcttacctgattgaaagctcataatctgttgttattgtttgttaacctttaatgctctgatttcaggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggtaattaaaattaaaatttatcttatttttaaaaagcattccagggccagtatagtactttgcaccaagtaaatgtacaataaaggcagtggatctaatacattgaaagcgtttacagaggtagctaaagagcagcacgggtgtcctcggctcagaatttcttcctgtgtgtttgccactttgccattcattgacatggtcatggacatagggctctaagcccttgaggaaggctgggccagacctcaggggagatgcagccccaaaccacgtgcagtcctgtggacggatgtgtagatgtgccactgaggaacaatgtcttgagctttcatcagattctcagagaattgcttgactgcctttcgaagttgatgcatctgtgctcacgtttgcacccacccacgaggtccttctgtttcaggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggtaagaggcagctcgggagctcagtgttgctgtggggagggggcatggggctgacactgaagagggtaaagcagttttatttgaaaagcaagatctctgaccagtccagtcacttttccatctcagcctggcagtaagtcttgtcaccgtcaagttattgtagccatccttcaccctcacctcgccactcctcatggtggcctgtgaggtcagccaggtccccttctcatctgcacctaccatgttaggtggatcctaattttagagacatgaaaaataatcatctggaagtactttatgtcttaagttggcctggacatgtcagccaaggaatacttacttggtttgtgttagtgcttgtaattcgcccccagaatgtgtacacgttctggatgcattaaagtctggcctgtatccttaaagggccatcgctgtgctgcctgccctcagcaaggacacactttgcagacccacagaggctccgcctccacctcacaccaaagaaagggaggagtccaaagggcatcagtgccattactcacaaaatgataaatacacccttattctgaaccacgtggagtcatatggtttgtgatccctgtccttcaggtttcagcttagtggggaagtgggaaagtcagcgtgtgatcacagcacagggtgattgctgctgattatattatgtgcctgctgtatgcaggatgaaatactttatatgcgtcatcttatttgactctcacaaccccctgtgagataggctctgttactcccatttgacaggtgaggaaagcaaggcttagagaatttcagtgacttgcccaggtcctctgagctaggaagtagccattctggcatttgaacccaaggcctgctatccctagaacccacgctctcaaattcaacctatgacagaggcaagccctggtgctgtgggagccccaaggaagagcctctggcctggtggccacgtagcccaggagagatttctacaggagcccacagcgctgaaggagagagaggcagcagagtaagggggctttgtggcagagaggggactggcactttggggaataggtgggtcaggactgaatgtaatggagccatgtcagagctgtccttctggaagggcaagggcacctggacgcgctgcccctcagtgctttggacggttccacaactgtgattcacacggcttccccaaacgaaggtacacgagtgggcattctgtgactcggtacttccctttaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccggtgagtccccgtccatgaacggtgggttcctatcatagttcctgtctgcttcaccatgtttttattttgtgctgcctgtttgccaggtactaagctaggaattggggatggagaggtagataaaatatgcatcaggaagggctgggccccatctcttactctccaatatattggagtctacactggaatttaactggaatttgcttttttagtcattttatttagattttgaagtttcagctttcatcaaaaatacctctaaactttatgtctctgtgatctttggtcttagctgttttatgtatttagtcttatatgatcataagattaataacattacattcagaagattatttgttttctgtcagagttaaaatgtttgtttttatactgcattgtaatattaacgtactgtaaaataaaagtggcttgttcttttcaaggaacagtatcctcaacaagggtcattagccacaatttttaaaaaattggacgtcatagtttacatgttagagggcgttttgaagctttgtatttttaaattaaatgttatagagtgatgttttcatgtttcataattgttttcatctgtgcatttgtagccaacttgaaaacaaagatccagggattactacttaaaagccagacttcttggaggttatagtgatgattttgatagtatcttgagccgtctcataataacctcagggtgagagatggccaacaggagacagtcgagggacttagaaatctgaatgaaatctgaagttcaaatcttcagacatataccactaaccaagagattggtacctcagtctagtattgtctgtttgtctaaaattggttctaaggaatctaggctagtctgtctatccctttcaacttttgtgaggctgcacaaatgtaaaatgttgaataaaaagcactgatggaagtgtgtagaaattcttctctttgttctgttgtaattttagttgcagtgcagcctggagagcagcttcttagtccagaaagaaggacaaataccccaaaagccatcagcgaggaggaggaggaagtagatccaaacacacagagtaagtctcaggacccatttttttcttacatgttgttcctccaggacttaaaaatcattcacagagacgtgcaccgcggtgagtgtggactcctggaagcgcaccgtagctccgctgtgtcctgctgctcctccctagctgtcagggaggctgtagtccattgctttgccagctcttttgtttccgagtgaacaccttatccgtacacatgcggctgtctctgaccctacagaccagctgggatgccactgggggagcgctcccttccccccgcacttcccacactctgcagttattctgagatccttgagggcagggaacaggtttgtcttctttgtgttctcagaaattaatgctcggcctctggtcagcaagcaacaaccttttgttgagtgataatgaataaataaatgtttcccacatgagtattcagtaacctcagtgtcaggttcagccatctgttttggtggatatttaaaagaaaattccgcttttcctacagaaaaaaaaaaaaatccaaatcccagtgatttaagccagttatagacttagacatatactacggcttttcatgcactttcctcccaattctagagtaggtattttactaggaaaatggtggcagtgcctgttgggaggaagattctttggccaagtgtcttttgttcttgccagggcccctaggctgctggggtgcttcagcttctttagcccagtgtctggtggggaatggcccctgttgcctgtcccacagaggtgggggtgcctcacctggagcctgtccacacattttacacagcacgcttacctggagcatcaggcatcttttccatgctctgtggctcaggaaacacgccttttcaatcatgagtgcaccagtgcttttgggctttttctccccgcttttgtgcaatcctggttgtggatggagttttcctgtctttagtcttctgcatagtacttttctcttctggttcccggttcaaggttttgtaattagagaatgacccagaagcaatggcattttaatgcacagccaaggacttctctgaatttgtatctcaaacctctgtgggtccttcaggcttcagtttgtgatttcatgatttcttgttgctacctaaggaatatgaaaacacccacctccctactctgcatcttccagccgagtggcacctcaggctgtggatcctgtgcttctgtggtgaggataagaatagtgccaaccgtgtggattgaaatcaatcagttaatccctccatgtaaagcacctggaacggatgacagtcttgttatgaatactcaacaaatgctatcatgatttttagttagatttccattgctttaaaacagttgagacatcttggcggtttgagttagagcaacgggccctgaagtgggttctgtttgggtgaagatgattatgcttattccccatggccctctttaggcaagagtgggaagctttctttgtttttttaatcacctcgataggacgttacttcttaaaggtcatccaataaatattaataggccgggcgcggtggctcacgcctgtaatcccagcactttgggaggccgaggcgggcggatcacgaggtcaggagatcgagaccatcccagctaaaacggtgaaaccccgtctctactaaaaatacaaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcaagactccgtctcaaaaaaaaaaaaaaatattaataaagccaactcgttagcgtggggcttaattgcttaagtccaatgagaagtccttctctatcctaggaagttgcccaaactgtagaatctcgtggcctgtgggtaatagccacgtaatacacactcactgcctcaacaaatcatattttagtaggtatgatattctagactcaagacaccattctgtggatcttcccaagggtgtgaagtgtccacagcgtctgccttgggagtttccatgcccaccagaaccatgccccaagcccctcaagcactctgacctaggaaagccagtgaagcaaggatgacaacatggccctttgatactagctgagggacagacacaggtcctgggagaccagagaaagacgaggggcagaggaggtgtcctaaaggaagtctgaggctgaggagccacaggatggcttccagctgtcacaggctgctgctggccttatcacagagagtgggccagagggctgggaaccaaggccagagctcaggttcaggaccattccagcaatcccagcagaaaatggggagaattgtatggtataggcggatatgaaggtagaatctgcaggccttcagtggccaactcagagtctaagtggattccacagttacagcttgagcagctggttgtaggtcatgctttctacactgggcatataggatgtgttttttaaaaagtcctctcttaaccgttgcttgtttagatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgagtctgctcgttccttgcagaagaccaagtacggtgaaaggcaccggtaggccctgggctgggcacacgtgagagggcgggacagaatccccgcagcccagaggctgcctgctgtggttctggtgcccactgtggttctggtgccaggctgctttcctcaggcaccacgtgtggaggtcgctagtagaaatactgggttttctaaaatgaactgaggccctacatccctaagagattagtgttagacctgattctagagcaactagaccactttgcttaatagcagaccagaaaccacaccccctcgagtgagtgagattttcctttggagataattcatgtttttctacacagttttgcagttgtcttcagaattggtttaaagtaggtgttattgccaggcgcagtagctcatgcctgtaatcccagcactttgggaagccaaggtgggcggatcacttgaggtcaggatttcgagaccagcctggccaacatggtgaaaccccatctctactaaaaatataaaaattagccaggtgtggtggtgtacgcctgtaatcccagctactcaggagactgagacaggagaatcgcttgaacccaggaggcgaaggttgcagtaagccgagatcgcgccactgcactctagcctgggcaacagagcaagactccgtctcaaaaaaaaaaaaggtaggtgttattgatcagaacccttgtttcagataacatgaggagcttagcttgaggagagtgagggttgatggagggggactgacttctgcccagtgaaatggcatcatctcccaccagcccgctgaaataagatgatggggcctgttccttagggcctgcagcatcctcaggcaggaaagaaaggccgacctggcagggtgtgagccagcaggtgtaggtcagggagaatggagccaggtcccagggaagaggcttgtggctgcctgagaagggtgcgtgcctgcctgtgtgtgtgtgtgcacgtgtgtgtatgtatgctggagagtctagggaggcttgctccaaggacgcagtattgtttgatcctgagagataaggattctgccgcagggaatgaaggtattccagatggcgggcttattccgaagaagaggccagtgcctggcggtgctggaagcagttgcagaacagggagttgtaggctttcctgggaagagagcagcaggggtgctggagaagcaggccacacttgctgcatggggttgctctcggccccactcttggtgcacagcgagtcactgtgggttcattagcatctggttatgagacagtaactgctcctttggaggggctcgtggagaccatgcaggagggcacggtcttgaggtcatgccgtccagagcacacctgaggataggccaggacgggctgcacgctgtaggtaaaattcctccagcaagctcttcactggcattgaggagttccctgagtgcggtcatctggaaggcagctgtaacaggcactgcagtctctccctgggtgggtaccagagaggagcataggggagcataaccgatttaaagagagggctttcctgtggtgaggtaagagattagctggtcattatcatagagccccctctgcctttgtgcagatgggctgtgggaatcctggggttccgttgggtcctttgtcacctcactgaaggcatgtaagctgagctggccagaccgtgagctgatcctgccacttgaacagcatcaagcctgcctctggattcttctgtgcatggcacttgtctgagcacctcacgcacagagaactggacttcagagtttacagaaataagctgtatggttcattttcatgcctgcttgccaataaacatatctgagctgaacctcattgaacgcctgcctttattctagcacagcacctgctgtttgtgggcgaggggtgctgtctctaactcctgcctgcttctcccagcactccctgagtggggtgtgccagcagcctcaggatgaggacaggaagtgggagggcagagcagatttgggagggccacttgatggggaaggaagtcccaggaagcagttggagctgttttctgggggagaaggtgccagctctgggacagtgttggggtagtgaggagggagcccagtggagagaagtcgggcttcctgcttcctcacagtatgtctgtcctgactcaactcggatgatgtcacttccttttcatcttctcaggtgtggaagcttggatggtcacccaaaccgggaggggattttggcacagcattccctgagatccccgtggagttcctccaggaaaaggaagtctttaaggagttcatctaccgcatcaacacactaggtactcttggggcctctccttcaggtcaccattgtcggacatctaccgggaggaaatccagagcccccagtactgggatcttctcatttgactccagaaaagatttaagcatgataataatacaaacctatgtgaatacattttgcagtgttggcaaaactccttttatactgagaaaatagatcccagttcctgtgttttgtggcttgaatcccagctttgtgtattccgggcttgtttgaagtcaggaaaggttcatgtgtagtggacaacgtgagaccaaattctgccttagattttgcatttaggctaaacagtggcagcacttgtctcagaatgttttcttgtgttcaccagtctgatcctgttgtgtctcagtggtccattttctcatatgggaacaagcagacgggagcagatggagtcaggtttcttggcactcgccttccccagagcctagaggcagcatggggagaaagcaggcttggggctcagacagtcctggtctgcttccagccctcctacctgagcagcgcagggcaagtccgtctaacctctagagaccctcagttttgtcatatgtaaaatgggggtcgtgtctatttcatagaattgttgcagatttagaaattacatttctaaacaaatgttaccccttatttctaaataagtgtctaaatgaataagtcaccacttttgcccctatttgatggcaagaggtgtgatcttgtggtgggactgtaatcagtcagttctcagtgactgtgccctgctgtggtgtttcctggaatgttcctgtcttgtcctagaaagtctggcaggggcaccctgactccactgtccagtcctctccccagtccctcgggcttctgcagatttgaggcttgtttggatcccagaaggttgtggcaggagacaccttgcctctactttcccctttataattcaatgtccaaagagagccctgagcaggtacctcacgccagctgcctcacggagctcctcctcttcctggctgtgaggatcggtatcagtggcctcctgctctctcccccttgcctaacacgagcacctttgcttacttgggtgcccttgctcttgaactgcccatcggacgtgcgtgacccaagactgtgccgcagtccttgccttgtctgtgctcattttctttgttcatttttttccctgtaacgtaaattgttatatttgtctgtatctgtgtctgaatcagtcctgcacgctctccttctctctgtctcttgttctttctttaccccgtttatcacggggaccccgatgtccattgctctagttctcctgtcctaagcaccccatcccgtctctctggccttaccacaagtggcgtggctgcctcagacatcatgatggggacatgaagcacagctgtcagaaacaactgttcgttagatacactcgaatgcagctcatcaatagggatggagggtctgtcggatgtattttcactgaatccccgttcctaccttgatacactctttttaatctattcttctagacaggtcagaggaaccattactttgacttttaaatttttagcagctttattgaggtagaattcacatactacagatttcacccactctaagcggacagcttggtggccattagttttatccacagagttgtgcagccagctgcacagtctcagggctggactccagggaagattttagcccatttagtgagtggggcagaagtggccctggccctgcacgaggttgcctgcatgggcgtccctgccctgtccctgtgtctgctccactgggggttgaccaggctgccagggccgacttgggcctgtgccacctgcctctcatgtgtctcggacagtgcagccgatgtctatacttcggtttcctcaatgatgaaatggaggggatagtgttccccgcatcatagaactgtgtgaggtttaagggactcactgcccttggcgtggagccttctccaggggccgtgctgtgtcggcgtagctgtcagctctccgttacaggcttgagaagggttgacactctctcatgtaacatttatatttctaggctggaccagtcgtactcagtttgaagaaacttgggccaccctccttggtgtcctggtgacgcagcccctcgtgatggagcaggaggagagcccaccagaagtaaggccacaccctgtgctggttggcacatgggcagttatggccgcttgcaggcctttggtggggaataaaataaggcagcaagctggtgttctttttttctcttaccttatttttgaaagagtagctgaatggtgtcttgactgatattccagagcagggacaaagcctgctgaggtctgggggctgcgattaccaatggctggaatgcattttattacggtgcattccatgttaaggatcaatacgattgtgccctttctggaaaatatcttttagtttatcaatattcagaggagtgtaggttgaattaaaatgaaaaggcactttataaaggccatgagtagtacctggtttcatttttctaatgtcttgcagagattttatcaggcttcttgaagtgttcacgtacattacgctaacacgatattaataataactgtgctctggtacagcggagccagcagaatgggaagttgtggaatgcaggcccttgattctgatagaaggtgtggtttgaactcacagaaatgacagtttggagggtagacatatgtcacaagtcatcaagattgtctttaaattcatgcatagaagctaacagggtgtcataagcaaggcctgtaaaatgtatgagggaattcaaagataatttattaaaaagtaattcatgtttggagttttgtgcccaaaggagtccttgatttgaaaaatgggcttttgcccatcagattgtttcagggcccgtgtgtgcggaggccctgccttgtgccccgtgagctcagcctgacagaaatcctttggtagcacttaaggctcctcttcctcccattgaggcagggaagactctgggttctgcaggcagaggtggttgtgggtgtcttgctgctcttgttgacatgtgggctctccttccaggaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgcttgagttcccacgtgtctctgggacatagcaggtgctggggacagtgggttccccgctgaagcgtccagcagcttcaaccaggccgttttccttcattgctagaattgaaaacaccgtccgtgtggcctgtgcaggagatgcagacccaaaggtggcctcctggtcagtgagaagctggaaacgtgacaggaactgacgtggggttattgagcatttaggggaagacgttagcagagcaggaatgagcaggcaactagtagaacacccacttaagggctcacggacaggtgctcacttaggaagtgagtttcatttggtattacaccaggttcctttaggcaaagcggagggaaagttctggtgtttttcacttgtaagattttgaaggaaacaaaacactctttaccttttttctaaaatgtaggtttgggaggaagctgagcattatcagagggattgtggagcaagagattcaagcaatggtttcaaagagagagaatattgccacccatcatttatatcaggcatgggatcctgtcccttctctgtctccggctactacaggtacctgagggaaagggtgcgggggagcggttgtacttgggctagaatgagagaagactggcatgctcaccacaccagtgatgcgggaagacctgagtgtggtctgagttggaggctgtggtgctaaatacgctgcccctttcataagcaggagtcttagtcaggcccagggaggaagtaaaatctggaaatgaatgagaagcattctctcctgccagtcaagaaatgagaagcgaaagaattctcacgggctgtaagaccagcaggatttaaaagttgaattagttgcttatgttaagaactcaaccaagttcatctacacaagctgaatctccagcttttcctaagaaaccatgtgtggcagtggctgcagggcagggcacagctgggcctgagcaccccgctccctgcacctctcccctccctgggccctgcctgtcactgcccactctcccaccaagccttccggttgtgtgcctgccctatcacaggcatcggagcttgtcacctggtttaaaagaagagagttgtgtggggatttgggatgcacgtttttcactcaaaagtattttagcgtagagctctgtgattccgtagctatttaggagtttaagcaccttgaaggctttaattgcagaaagttctatgtggacgtgcaatgtgttatacgcagtgtctatgagactcaaatgtttattagggcgttgaagtaaactgagcacttggagggccatggatccagccttcaaggagctcataagtcaggaggacccaggagcaatgacctgtcatagaaggcagaaaagaggggcacagaggtgggtgggaggcatacacaggcagctcctggagctccaaggggagcaagtgcttccagggaagggggcgtggaggcccctttggaggaggcaagttgatctggggtctggcagagggttagctggggacatttagcgggaggctggtgcccgggaattggggggatgcccagcagaaagacatgaggaggctggcctggggcgtgggggggtgtgaaaggttaagtgggggcattatcctgctcccgctcctgccggctgtatctggtcagcctgggcaccgaggtggggttctggaaggcactgttcaccaaaatgcttatctgggtcccccagagagcttgcctgcctggactgtcggctcgcctgcaactgctgactcctaagcttttgcagctcagcccacaaccagttcctattcacagaggtgggagctgaggggtgacaagtgactgctgcagtcttatttgtcatagagaaaaagtgacagagtccagcttgcccactggccctgccagcttaactggttataaagtgacaaatccccaagacccacagggctctgcacaacctgggccctcctgccagtggcggcgagggcaggtggctcacggctgggtgcctgtctgggcaggagctgggctggtatggggtgggcctgcggccctgcccccctgtgcagatcaagactcagggtgctggtgttcacaggtgccctcatcagccacgagaagctgctgctacagatcaaccccgagcgggagctggggagcatgagctacaaactcggccaggtcagtctcgcgcccccgccgcctggcctctgtccgtttctgtcctcagactttggcgcttgacacacccaggagaaaagctcagtgcactttttaaatgaaaggaagttttccttttttttaaaaaaaaatttaatgttcattgtttttatctgttttattcctaggtcccgcaagcagaggaagcattagttttgtttttatttatgttctgtattccagaaagtagttaagagacctcacatgtagcgatagagatgtgtgtaagagacagtgagagggcgtgacttggacttaagcaaggaccgtgagacacaaaaaggggggtgaggacagagtggagtcagctgaaatgctcaggaggaagtagacgccatgaagggccatggtatggggggccgcaggcgtggccgtgagtgtccctggggccagctcttggggggctccctgagtgtccctgtccctgtggccagttctgggtgggagccccgtgtgcaggcagacagctcggccacttcctagcaggtcacattggtctgtgcttctgtttcctcctcagataagtgaagggattcaagggtctgggtgtggtggctaacacctgtaatctataacattttaggaggctgaggcaggaggcttacctgagctcaggaggttgaggctgcagtgagccatgattgcaccactgcactccagcctgggcaacagaccagtactctgtcccttaaaaaaaaatgtaaacagaaacgtagggccatttgcatatgatggcacatggcgtggagccctacaggtgtatgctgggcggggcccggctgtgctggccgacttgcacctttccctccaccccggtgctgtgtctttcgctcaccgggttcctgatttagtgaaagcagttgtgcaggacagttctctttgtagcttttgtttctgtggaaatgggtcagaatatggtgtttagaaacacttatgagctctgagagtttcctcttctgagttcctggcctgcagccttcacagcagaaaccctgtgatgtcacaagcctgtttctgttccctgctctctgcctgtactgtcctgttttgtgcctgccggtttcagtgacaggaagcagggagctactggaccagcctgtatttttctagacatagttggaaaaagaagtcccactcttctgtcctttcacctttgacagatgtttccaccccaagataagtgaaaatgaccaataggatgcactgtatttttcatgaaagtgtttctgaagggcaggctgagagtgagaggcctggggctcactgggtgcctctggccttgtcctgggcccagggacactggtctgtgcccgaggtattccctatccccccaaccccgctgcatttggccacatccttcaatgtttgcgttgtgtccagcgtccgcaaaccaactgtcatgggatcatactggggctgaagtacggtcccacccctgccctgtctggggctgaagtacagtgccacccctgccctgtctggggctgaaggacagtgccacccctgccctgtctggggctgaagtacagtgccacccctgccctgtctggggctgaaggacagtgccaccccttccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctggggctgaaggacagtgccacccctgccctgtctgggatgtttagcccctagatgccactggactgagccgctacttgcttttgggaaagaggggtgggggttaggggtctgggcgaggggagtgcaggggctcctccttggcctgagagctgttcatacagactcctcgcccactccctgcagggtgctgggtcccaggggggaaatggcccttggtgccaagaacgtgagttggggctagtgccagtgatgatggagaacagctttttatgggcacacagcccacagcactgtgccaagtgctcgaggcttcccgagaaccaggcagaaaggaggacagtcgaggtgtgctgactgcgtggtggctgcgtgatctagagcgcgggtcacaaaggcgcgagggagctctggccttgggtttaccgcaatgactgccagtgcgggagactggaaaaggaatctcacgtattggttccgtgttttggggactccattcagatgtcacttaggagtgaaagcatcccttcgtagagcctctttctgtgtcaccctcctcagctgctcctggggttgactggcccctgattcatgcctttagcatgtgctggagcttcccagcagctgtccagcccctgccccaccctctctgtgggctcccttgcccgtaacctggggtgtctgaacgacccttgctaaggggcagactgttagacggtaggcatgtgctgagtcccagtggccacacccacccaccaggagcctggcactgtggccgcagcactgagcagtgccccgtttctgtggcaggtgtccatacactccgtgtggctggggaacagcatcacacccctgagggaggaggaatgggacgaggaagaggaggaggaggccgacgcccctgcaccttcgtcaccacccacgtctccagtcaactccaggttttccaatggcctttttctttttaacagaaatttgaaatttcttatcagtcatttgatttgtttgaggtgcttcttgaaatgagcctctcatctcatgtacttggaaaatacccatctcgcatattccacaggaaacaccgggctggagttgacatccactcctgttcgcagtttttgcttgagttgtacagccgctggatcctgccgtccagctcagccaggaggaccccggccatcctgatcagtgaggtggtcagatccgtaagtgagccttcccattcccctcacacctgcacgtgccacacgcaccacacacgccacacaccccacacacacacaccgcccacacacatgccacttgcacacacacccctcatgcatgcaacacacacacaggccacacgcaccatagacaccacacacacatgccacatgcacacacatacacggcatgcaccatacacacaacacacacagcacacatgccacacacacacgccacaccacatgcaccacacacatgccacatgcacacacactccacatgcatgcaccacacacacacacacacaccacacacaccacatgcaccacaccacacaggttacatgcacacaacacacacatgccacgtgcacacaccccacacaccacatgtatgtgccacacacagcacacaaccacacacatgcaccacacacatgccacatgtgcatgcaccagacacatggcacacactacacacacgccacgtgcacacaccccacacacatgtacgcaccacacacatgccacacacacatgcaccacacacatgccacatgtacacacatgtatatacacaccccacaccacacacacaccacttgcacaccacgcacacacaccacatgcgcacacacacaccacatacgccacatgtacacaccatacacacaccatacatgcaccacgtgtaccacgcacccacacagacacagcacacgcatacaccacacacacacgcacacatgcgtcccgcacagtaatgtctcttgggtgtaagaacacgacttgccagtagtagcgttctggatgcgttgcctggattctaacagcgcgattctccccttgccctcctggttttccacatctccagcttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggtaagtgacaggtggcacagaggtttctgtgctgaagccacgggggcccatctgccttgggacctggtgttggccagaggtgccgggtgcggctgcctccttccaagagttgacccgaaccggactccacggcccacgtgagctgcagtgcttctcagatggagggggttcagcgacggtcagtgccattcacaggtcactgtgatgtgggttgtggcggccaagccatggtttggggtcccgtatccctgggcttatgacatcattgtagtagcccatccccacagaaccacggtgtgtggtggcgctgaggcatcgtagatggtggaaatgctactggcttccccatgctctgccctgaggcctgactgcctcactccccttctcagttatgttccaggccccccgagcttcctggctggacagcttctctcctgggggccgttttgtcacagtgaccctgtgtttctagtcccaaatctgggtgctatagtctctttttagcgtggtggttgtcttagtcttttttggctgctaccacaagttaccttagactgggtaatttataaacagtggaaatttacttctcaccgttctgggggctggaagttttcatggtcaaggtgccagcagatttggtgtgtgatgagggctgctctctgcttcatagatggcatcttctggctgggtcctcacggtggaaggagtgaacaagctccctcaggccttttagaagggccccaatccacaagggctctcccatcatgacctcatcacctcccaaggccccaccttcttgtactgtggcactgcaaattaggtgtcagtgtaggagtttcaggagggatagaaacattcagaccatcccagcggtcaagtgttcatcctcttgagttcctccttattctgcttctggtttatcaggattcagccagtgcagcatggtacctgtattctgtggcacatcaccacatggtatttgccaagtatccatcacctgcacacgtgaaatcattgcccgtgggtcccgacatctggcgaagcatattcaaggatggcagaactgtcagagctggcacctctggttccttgtcatgtggcattacctagtaatccattttatgatagcaatggaaactcatttcttcaacaaacacctgagtggctgccgtgtgccagccgtctggggcccttggtgagaatggcatggtggtgcccatcagggcctgcctagcccgtgctctggacgggctcctgtgtgtcaggaacgacaatgctgtcatgacggtgaatgatttttttttttgccatcactccagccgctaacatttgcggagctcttcctcccgcacccccacctgacaaggccaagggtgaccttggccccaccctaggcggccaaggtcagaggttagctggcttgtctgggtcacacaaaatgcagcagaggttgaggtgagcacatgtccgtgacctggagcctgactccctctctgcgagtcttgactgctcttgcctagactctgtcctccccgagcccaaacgccagtcatcttcccttgtgggtgtccttcagcctggtgccatgctggtgactcagcagccgtccagggagtggaaacaattgagtgtgtgggttccctgtgtgggcatctctcttcacggcgaacaccctctgggtgttgcccacacgatgtcaaagcggctcttggaaggggtccttctcctttgtgggaagtttcagctgctgggctaacttgaattgtaactgtggttttgtgctcaggcccagatccccctaggcaagtgttgtgccatcagtaatcaaatgagaaataatcattttgaaaagcagatcctaaggcaggatggtcatggacactcactcccagctctttgtgcactcatgctttctggaagatggccatcctctgtgaaggttttcagcgcgtcatgcttggtacccacgtatccagagcatgtcgttttgaggtatttgcccaccgttgtgaaatccgtgccacccgagagcaggtcctgatgtggggctttcagaagtgggacctggggccgtacgcagtccttagggaggggccgtgtggcgttgtgcgtgtgaggggatagcacagggtgaggtgggggcccaagaaggaagtgacccacaaagaacagcctcctcttttggtccttgttcctgggatggctgggagtggcttctgtgtcgtccggccatttcccctgcggagaggctcctaccactgccgagaacctcatcattccacaaaaacaagaggccgcctggccatccagcgctccatgggaattctgtgtccccatagtcttgggctgaaggagggtgacattccttgctgacttctgcaggggtctcctcactgttaaagagcagattgaaagtgaagaacgtgggctaagtgtttaggtcgatatttaaccctgctaggttttggatactaagtgaaattgaggccattttggttgaagttgacagaaaccactatcagggatccccaagactaccccaggcttttctagaaagactctcagctaagatgtgttatggtaaaagcacacaaaacaaaatcagcaaagaaaattagcaagggcagaggcccatggggcgatgtcccgaggacaccaggcttgagcttccagaatcctctcccagcggggtcgtgcaggacgcacttaactccccgcacagtgagccgtgacagcgcgtgtgcagtgtcgtcgccaggaaagcacactagagactcggtgccagggtttttactgggggctgggcacatgggcaccctctgcctgcctcgtgcccagactctggactcccggagggaaggcaagttctcagcaccaaccctggtgcccacacaagcagctgagcacagggagcccctcctcagtgaggatggtgggcaccgtcccaacaccagccaggggccagccttgcacacaggcctctcaggatggtctccggcctgctgtgtagtctcttctgcacacaagcgtgagggcagcgcccccgcctcggctgtggggaggagccactgggacgtgagctctggtggcatgcagcagcttttgtctgtgtgtgcctaggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccagtgagtgggagcctggctggggctggggcgggggtctcagaatgagctgtgaaggaagcagcatcaccctctccaagtgcccaggctcctggccagatggcaggccaggtatcagtgggaacccaggtgggtgccatggctgaggtcagtgagacgcaagagcacaggtgcgtcctagaggcttcctcgggcacctccagcgagctggagctctcgcctctgctgctgtctcatgtggcgcttagcacactctcccacgtgcccattcctgactctgctctcgaggccatcggctctcattctctgctcccagaaccctgttattacccaggctagcctcctctctgcaccttccccgccctggcccagtacctccctcttgtttccactgtgattccgacctcaccttatcttaaagctgctggacggcaggttctgtacacacgtgtccttgacaaagcacggctggtgccgcaacccctcagcgagcaagtcaagctcttcacagcgatgtcttacaagcgcagagggctctgtgacaccctggtctcaccgccactcttccaaagtcgcagaggctttagcagagatgggcccagcctctctgagtcataggcttctgcacacgggagctgtctttagagggagggtggaatttcatcagccacccacatgggggagttgagggcaagaattaggagcaaagatgggaaggggtctgggaggaatggccagtgatcccctttgacaagtgggcaggaaacgggggctaggtcaaagttgagtggaagacctggagggagacgggaaggtctctgtaggcacagttcagacaggagggaggtgtgagccagggcacatgccggtggccgtctggcaggatttgggacatgctggagcagggacagcggctcatcaggggccattgccctcatccaggccagagtgtcacaagcccgtggggaggcccttctcgcctgtcatccttgctgggcagtgggtgctgtgctagcaggacaggcggacggctggcaactgtctctgcatccctggagcctggcatagggccaagtcacacggggcacaggcctgcaaatcaggcacatatgttggtgcagtgacgtgattttggggggcagccccagaacaggccccagacacaggccaaagccctgcctgtgctggtgtgttgggctgttctatggctcttgctgtgggcatggaggactcagggaaggagagttgaggtggtccaggagttgcgtttgggatgcagagagcttgtggcatccaggtagaaatggtgcgtggggctgacctcagcaccatgggcagaggggccgtgtcacgtgcctccgaggtggaggtgggaccacgtggtgacagatatacgcatcactgggcacgtttttgtgggtgttggggggcatcgtattggctcctctgttcacagtggccactcattcagtccctggctaccaggtcctcactgtgccatggggaaggccggcgctgtcgggggatcacagaaggcagcacgtcatgatggcatgtgccatgaaggaaaagcacagggcactcaggaagtagaggggactggcctggggtgtgggaatctagggcctcgttgagggacagagagaggaagtgtgtggtggccagcatggaggtggccacaggggaggctgagttaggccgagagggcagggcgttggggaggtagacgggctcagccactcagggagtggtcaagcagaggctgaagggtcaggccaggttgcaggggcctgggggagccactcagggtaggcgctcccgggagcccgcctggcccatagctctacactcccgcgtggggccggacatgctgtgaagccctctccacgttggatgggggtggctgagcctggatgctgtctcccgttttcagctgcgtgaacattcacagccagcagcacgtactggtcatgtgtgccactgcgttttacctcattgagaactatcctctggacgtagggccggaattttcagcatcaataatacaggtgagtgggccctggctgtcttcctctgcacacggggagtgggcttcccttctcttttccttgcaggatcataccagtgggccagttttgacttggtcgggaggaggcatgaacacctgagactgtgcagcgattctttgacacagaggcctttctccctgtgcagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggtgagcatgtacacggtgcccataaggccagcccaagtcctgttcaagggaggcaggagcatgctcactcaagggacctcgactaggtgccctctgatttcacacttctggtgttgccccaagccggccccatcaccttgcaagaaaggctctggagcccccagggctggagtacctggtcagggttgaccgtccctgtggtcactcatcccatgtggctgagctgggctgggtcctgggcaagcaaggggctgatatcacctgctttcagatctccagggactcactggacccctgtgtacaaagcactgtctacagagcctattgggttgtatagaggtaaccttcgtactgaacacttttgttacaggaaaggagaaagtcagtccgggtagaacttcagaccctaatcctgcagcccccgacagcgagtcagtgattgttgctatggagcgggtatctgttctttttgataggtaagaagcgaagccccatccctcagccgttagcttccctagaactttggcctgaagctgtgcttttgtgtgtgtctgctgatcccctggcgctgttgctggagtcctgccagtgattccccaccacagcctgaccatgggctgccttggctcagggttccactggcgagctggtggtccttggaccccagcactcaggtgtagcgttgaccagttccaaggttgtcccagtgcctgcccatctctcctgagggctcagggacagtacctggcagttgggggtgtggcagggggcaggaatgaccagcctctgggagggtggggcagaagcctgtacagtgaggaggagctggctcagcctggctgcctatcgtgagaggggagcccacggggctgtgggaggggggccgtggtgcctgtgagcagggtgaggagcagcggcaggaggatgaaggtggaacccacacatgcatctttgagacccgtgtggtcagtggcttctgccccccaccaccccccactgctgtgcgtgcatagaattggcttccctcacctgctctggaagtgggttaggagcttggtagggctttttctcaaggacaagggcccctgatttgctctcaggcctcagtcctggcgacatggtggatctggagccttgttgcactgccttgcctgtgctctccaatcagggtggccagtggggagccatttggcttttctcaagagcatactcaggtggaccttgctccactgtttgaccagatgaggcattctgaacagccaagcctgtgctggtctgttttcatgttgatttttttttttcttttctttttgagatggagtttttcccttgtcacccaggctggagtgcaatggtgtgatctcggctcactgcaacctccgcctcccgggttcaagtgattctcctgcctcagcctccctagtagctgggattacaggcacacaccaccatgcccagctaatttttgtgtttttagtagagacggggtttcaccgtgttggctgggctggtctcgaactcctgaactcaagtgatccaccctccttggcctcccaaagtgctgggattgcaggcgtgagccactgcgcccggcccccatgtcgatttttaaatgcacctctgcatcgttcttcagtccccatatgctcactgagcaccactgcgactggcagacgggcacagggaggcgccacgaccagtcctggccttcaaggggcttgtggtctagtgggcccaatgctaggtggcgagtgctccaaagagtgtggtgcacgccttccgcttgaccgctctccagacgccacagggaggcacctcgcagctgaccacagatttctctctgtggagcagtgtcttcagagcggctgccatgccactgctgggcgagggtctgcgggcgggtagagccaggagcacctgtgaggaagtgcactgccattttcgtagctgcttcccgtgtgtctcagttacacacggctggcatgtgtgcactgatgagacgggaacgtgatggttgcttttcagcactgaaagggatactgctcagggggcgtgtttcaggatctggttagggaagaagcagcgagagcacagatggggccctgtgtggtaacaagaaaaaagtcctggttgacaacagtgccacgaagcgttagaacacatagggatgtttgtggagcatttgcatgtggaaagcagcaaaaacataatgggaacgggttcttttgttatgatttttaaaaatctcttttgtaacatccttcccgctgcgccgtttctgcatattcctttatgtagctttcaaactcctcttaggagttctggtccctacagggcgtgggagcccaggctttacgtagctttcaaactcctcttaggagttctggtccctacagggtgtgggagcccagggcctgtgccgagcagcctgcctccacgagctagacagaggaagggctggggttttgcctttttagtctcaaaattcgtactccagttgcttaggctctgactttccccacttggaaagtccctcacggccgagggtccctcccagccctgatttcacatcggcattttccccagtattagagccaaggccctccgcgggcaggtggggcagctgtgggagctggtgccagtctctgacctgcgtccctcctcccaggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgaggttgcatgtgggatggggatggagtgggaaagcctggaggtggagttgcctccgacttcccagcagattcgccagcagagcccagctcctccgctttaaagcagcaatgcctctggcccccaccccacccccgccacccaggcgcagcaggtgcttcccgtccccccagccctgacactcaggcacctgcttgctccttgcaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatgtatcctctctgggtccctggtgctggccccgtttcccttgtcaacaccgaggctcatgtttcatgataaggttttgaaacctaacctttgcaaaaaccccacagatgccagggtgacaggccctcagccccagggaagtaaaatgctgacaggggtacagaaaggagcacgtccagacatttgctgaccagggcctctcagaggggccggtgtatggcaggagggtcgcagctgaggggcctttctgtggagggcctgggtgaggggagcgagggtgggcggtggtctctgcagacgtcccgcccactcgcgggctctgtgtggctgggcttctcctgacactgcttctcattagctttggtcattgtgcctcgatcgccctctcggggaaaggcttaagtaaagatccagttcccacccccagatgctggctgccaggagtttccctttccacagcccttccccaagacagaccacaagagcctccaagcagcacagttgtcctggtgctgacagcacagccttgcccggcgtgcctggcacggctctgccctcactgcattggagcagggctagtggaggccagcggaagcaccggccaccagcgctgcacaggagccaggccaggtgagtgctgccgagtgggtgccctgcctgcagggcatccagccagccaagggttgcaggaatggaggtggaggcgctgatgcagctggaggcatccaggtggcccttccggggctctgctcgctctccaggctccctggacccctttgtagactgtttcaggagaggaactcccaggtgaggacagggaggcagcattcccctcatttgccggcctttttccttaactcctgcaccagcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatcagttgttgctgtgtgaggcttgctttgcttcatgagaacctagaccttgctgagctggagtcttaggaagcagtctcctaagtgcttctccagcaggggcagaaactgtcccaccagctaacatctggcattatggagggtcccccaggcagctgccagcagggacaggccccgtgttttctgtagccagggatgaggaagtggccccagggcatgggcctggctgggtgcttctgcaagggccttcccaaaccacagtacaggtggtcttcctgccctgcagatgggagctgtgggagctgctggagctgctggagccttcatggtcaagtgacatcataagcttatatgacatacacaagcctcaggacttggcccatggcactgaagcaggtcatcaggcccagcacagagactagagctgtgttctcacagggcccaccacccttccacctccttggccattgacacctgcgtccctggcccagctgctcccaggtaacccccaaagcagctggcacatcccacctctggtgtggccggggctgctgtgtgtccgcagggcctgccccgtctattctagcttgtttgtcctgtctgaaccagcgcctactccaagaagcctctgctcagcccagcggggatgcttctaagctccggacgagcctctcggaagccttggtgattggtggtgtagtcatcttgggatgcagatgtcttaccaacctgcaagaacaaaaaccctgtggcttcctctggtgcagggtatttagtcaatgtttgctgaggtcccgtctggttctggctaattggcaggggtcgtccacccattctttccctgctctgctgtctgtgccaggagagacgggggccagtcggccaaggggccagctcctgctgcctgctcctcttgggcacgtgcgggggccccctttctctgagcagggatagggatcagtctgccggagggatgtggtggacaggcctaaagcatttggggcggggcatgccacttgagctccctaaatctgtctcctcataggtgacaccgctccagggccccccagtggcctctcctttcagagctacctaaattctggtcacttcagagaaatggagcacccccttctccctggtccaggtgtggacagcctggcacactgagcacacctggcatggctggtaatttcagaaagaagaggggccggggtccagtgggaagcagcggtgaacccctcgtgagtgggctttgcagtccctccccatgccacggcagagctgccctcaacacagccttcctcttcctcatcggagagcacaccctgtccccttgccgagctgtgccctgtgccttcggtggtatttgattttggctgctactggctttgttgggatctggaagtcgcttcccctgcgtggtgcgtggagcactgtaagtcagatgagggaagtagccagggtgaggtgagtaccgggtggagccgccactgaagggactgggtaggggggccttgcctctacatgatgtgacacagccaaccgaggacagaggaagccccgttcctgggggtgtggggtgcacccctcagggaagcctgcagtggggcctgaggaaaggcatcctccgcgagcccacgagtctggtccatgagcaccgtgacagtgtctgtgggtagaggtggacccggccttgtgtcatcaccaggacctcttttgggaaaccatgtggacatcgcttgcgggtcccccaggctctgcagccccagcagcctggctgccttttgggcaagtggcttgagccacagaggacccagtcctgttgcagccacatcctctgggggggcccgccagtgtggccggctttctccaccctacaccaggcctccaggtgtcctggtcgggggtgtctgggccctgggtgggccctgtggacctgtgaggtcagggtcagggcatcactggaggcagagggctgaagttgtgggtctgggttccccttgtgtgcacaggcccctgccctccatgcttggtcaggcagctacccccaaaactgctaggacaggctggtcctgaggtggatcctggcccctgtaccctctggacagcccacccgcccaaccttctaccctgccccagcggcggcagtgttggccacatccttcccctcctggccccaattgctctggggaagtccaggctccggagcctgcccaggggccccccgtgatttgggcccaggactccacgtggttctctgccttcacccaagccctgaactcctcagctgccaaatccccacccatctgcacaggctgtgctcaccactgctgctcctggaaggtgcccctcagtgggacgcccacctcctctctgggcttctgtgtttgggagccctgctgcccccacccttggtcagtccccatgtcctgctggcctgtcaggcagggcagaaaatccacccagaaatgctgagcaggatgagagtctagttgggcccagcctcattatttagaagggatggaggcctagggagcatgcttctagcctgagcccagcagggccccgcccatgtcccaggtctgcaccagggacagctcctgccgaggcctgacctgccccttctccctcaggtgctgctggttgaccagcctctggccctaggagaccccgtagcgactgagggtcccagcaggccatgcagctttgccaaggtacgagcccctccccagcaggggacagatgtggggaccctcccaggcaggagcagctgggtgcctggtgctgccatctgctgcctgcctggttcttgtcctcacattggaggtcagtgtgagggctctgcctcgggaaaggccatggagcttgccctgtccagggcctcccatgtgcactgagcctgggaagagagggttggagttgagccttttaccctgggaatgctgcctggaggatggtgcgggtgtggggtggcaccctgccaggcagggccctgcctccctgcgcccactggaactcgggcaggcaggggtgtaggtgcctcc

SEQ ID NO: 2 - DNA, 13481 bp, Homo sapiens
  source 1..13481: mol_type = genomic DNA
gctgccgggacgggtccaagatggacggccgctcaggttctgcttttacctgcggcccagagccccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagcagcagcagcagcagcagcagcagcaacagccgccaccgccgccgccgccgccgccgcctcctcagcttcctcagccgccgccgcaggcacagccgctgctgcctcagccgcagccgcccccgccgccgcccccgccgccacccggcccggctgtggctgaggagccgctgcaccgaccaaagaaagaactttcagctaccaagaaagaccgtgtgaatcattgtctgacaatatgtgaaaacatagtggcacagtctgtcagaaattctccagaatttcagaaacttctgggcatcgctatggaactttttctgctgtgcagtgatgacgcagagtcagatgtcaggatggtggctgacgaatgcctcaacaaagttatcaaagctttgatggattctaatcttccaaggttacagctcgagctctataaggaaattaaaaagaatggtgcccctcggagtttgcgtgctgccctgtggaggtttgctgagctggctcacctggttcggcctcagaaatgcaggccttacctggtgaaccttctgccgtgcctgactcgaacaagcaagagacccgaagaatcagtccaggagaccttggctgcagctgttcccaaaattatggcttcttttggcaattttgcaaatgacaatgaaattaaggttttgttaaaggccttcatagcgaacctgaagtcaagctcccccaccattcggcggacagcggctggatcagcagtgagcatctgccagcactcaagaaggacacaatatttctatagttggctactaaatgtgctcttaggcttactcgttcctgtcgaggatgaacactccactctgctgattcttggcgtgctgctcaccctgaggtatttggtgcccttgctgcagcagcaggtcaaggacacaagcctgaaaggcagcttcggagtgacaaggaaagaaatggaagtctctccttctgcagagcagcttgtccaggtttatgaactgacgttacatcatacacagcaccaagaccacaatgttgtgaccggagccctggagctgttgcagcagctcttcagaacgcctccacccgagcttctgcaaaccctgaccgcagtcgggggcattgggcagctcaccgctgctaaggaggagtctggtggccgaagccgtagtgggagtattgtggaacttatagctggagggggttcctcatgcagccctgtcctttcaagaaaacaaaaaggcaaagtgctcttaggagaagaagaagccttggaggatgactctgaatcgagatcggatgtcagcagctctgccttaacagcctcagtgaaggatgagatcagtggagagctggctgcttcttcaggggtttccactccagggtcagcaggtcatgacatcatcacagaacagccacggtcacagcacacactgcaggcggactcagtggatctggccagctgtgacttgacaagctctgccactgatggggatgaggaggatatcttgagccacagctccagccaggtcagcgccgtcccatctgaccctgccatggacctgaatgatgggacccaggcctcgtcgcccatcagcgacagctcccagaccaccaccgaagggcctgattcagctgttaccccttcagacagttctgaaattgtgttagacggtaccgacaaccagtatttgggcctgcagattggacagccccaggatgaagatgaggaagccacaggtattcttcctgatgaagcctcggaggccttcaggaactcttccatggcccttcaacaggcacatttattgaaaaacatgagtcactgcaggcagccttctgacagcagtgttgataaatttgtgttgagagatgaagctactgaaccgggtgatcaagaaaacaagccttgccgcatcaaaggtgacattggacagtccactgatgatgactctgcacctcttgtccattgtgtccgccttttatctgcttcgtttttgctaacagggggaaaaaatgtgctggttccggacagggatgtgagggtcagcgtgaaggccctggccctcagctgtgtgggagcagctgtggccctccacccggaatctttcttcagcaaactctataaagttcctcttgacaccacggaataccctgaggaacagtatgtctcagacatcttgaactacatcgatcatggagacccacaggttcgaggagccactgccattctctgtgggaccctcatctgctccatcctcagcaggtcccgcttccacgtgggagattggatgggcaccattagaaccctcacaggaaatacattttctttggcggattgcattcctttgctgcggaaaacactgaaggatgagtcttctgttacttgcaagttagcttgtacagctgtgaggaactgtgtcatgagtctctgcagcagcagctacagtgagttaggactgcagctgatcatcgatgtgctgactctgaggaacagttcctattggctggtgaggacagagcttctggaaacccttgcagagattgacttcaggctggtgagctttttggaggcaaaagcagaaaacttacacagaggggctcatcattatacagggcttttaaaactgcaagaacgagtgctcaataatgttgtcatccatttgcttggagatgaagaccccagggtgcgacatgttgccgcagcatcactaattaggcttgtcccaaagctgttttataaatgtgaccaaggacaagctgatccagtagtggccgtggcaagagatcaaagcagtgtttacctgaaacttctcatgcatgagacgcagcctccatctcatttctccgtcagcacaataaccagaatatatagaggctataacctactaccaagcataacagacgtcactatggaaaataacctttcaagagttattgcagcagtttctcatgaactaatcacatcaaccaccagagcactcacatttggatgctgtgaagctttgtgtcttctttccactgccttcccagtttgcatttggagtttaggttggcactgtggagtgcctccactgagtgcctcagatgagtctaggaagagctgtaccgttgggatggccacaatgattctgaccctgctctcgtcagcttggttcccattggatctctcagcccatcaagatgctttgattttggccggaaacttgcttgcagccagtgctcccaaatctctgagaagttcatgggcctctgaagaagaagccaacccagcagccaccaagcaagaggaggtctggccagccctgggggaccgggccctggtgcccatggtggagcagctcttctctcacctgctgaaggtgattaacatttgtgcccacgtcctggatgacgtggctcctggacccgcaataaaggcagccttgccttctctaacaaaccccccttctctaagtcccatccgacgaaaggggaaggagaaagaaccaggagaacaagcatctgtaccgttgagtcccaagaaaggcagtgaggccagtgcagcttctagacaatctgatacctcaggtcctgttacaacaagtaaatcctcatcactggggagtttctatcatcttccttcatacctcaaactgcatgatgtcctgaaagctacacacgctaactacaaggtcacgctggatcttcagaacagcacggaaaagtttggagggtttctccgctcagccttggatgttctttctcagatactagagctggccacactgcaggacattgggaagtgtgttgaagagatcctaggatacctgaaatcctgctttagtcgagaaccaatgatggcaactgtttgtgttcaacaattgttgaagactctctttggcacaaacttggcctcccagtttgatggcttatcttccaaccccagcaagtcacaaggccgagcacagcgccttggctcctccagtgtgaggccaggcttgtaccactactgcttcatggccccgtacacccacttcacccaggccctcgctgacgccagcctgaggaacatggtgcaggcggagcaggagaacgacacctcgggatggtttgatgtcctccagaaagtgtctacccagttgaagacaaacctcacgagtgtcacaaagaaccgtgcagataagaatgctattcataatcacattcgtttgtttgaacctcttgttataaaagctttaaaacagtacacgactacaacatgtgtgcagttacagaagcaggttttagatttgctggcgcagctggttcagttacgggttaattactgtcttctggattcagatcaggtgtttattggctttgtattgaaacagtttgaatacattgaagtgggccagttcagggaatcagaggcaatcattccaaacatctttttcttcttggtattactatcttatgaacgctatcattcaaaacagatcattggaattcctaaaatcattcagctctgtgatggcatcatggccagtggaaggaaggctgtgacacatgccataccggctctgcagcccatagtccacgacctctttgtattaagaggaacaaataaagctgatgcaggaaaagagcttgaaacccaaaaagaggtggtggtgtcaatgttactgagactcatccagtaccatcaggtgttggagatgttcattcttgtcctgcagcagtgccacaaggagaatgaagacaagtggaagcgactgtctcgacagatagctgacatcatcctcccaatgttagccaaacagcagatgcacattgactctcatgaagcccttggagtgttaaatacattatttgagattttggccccttcctccctccgtccggtagacatgcttttacggagtatgttcgtcactccaaacacaatggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtttctattacaactggttggtattcttttagaagacattgttacaaaacagctgaaggtggaaatgagtgagcagcaacatactttctattgccaggaactaggcacactgctaatgtgtctgatccacatcttcaagtctggaatgttccggagaatcacagcagctgccactaggctgttccgcagtgatggctgtggcggcagtttctacaccctggacagcttgaacttgcgggctcgttccatgatcaccacccacccggccctggtgctgctctggtgtcagatactgctgcttgtcaaccacaccgactaccgctggtgggcagaagtgcagcagaccccgaaaagacacagtctgtccagcacaaagttacttagtccccagatgtctggagaagaggaggattctgacttggcagccaaacttggaatgtgcaatagagaaatagtacgaagaggggctctcattctcttctgtgattatgtctgtcagaacctccatgactccgagcacttaacgtggctcattgtaaatcacattcaagatctgatcagcctttcccacgagcctccagtacaggacttcatcagtgccgttcatcggaactctgctgccagcggcctgttcatccaggcaattcagtctcgttgtgaaaacctttcaactccaaccatgctgaagaaaactcttcagtgcttggaggggatccatctcagccagtcgggagctgtgctcacgctgtatgtggacaggcttctgtgcacccctttccgtgtgctggctcgcatggtcgacatccttgcttgtcgccgggtagaaatgcttctggctgcaaatttacagagcagcatggcccagttgccaatggaagaactcaacagaatccaggaataccttcagagcagcgggctcgctcagagacaccaaaggctctattccctgctggacaggtttcgtctctccaccatgcaagactcacttagtccctctcctccagtctcttcccacccgctggacggggatgggcacgtgtcactggaaacagtgagtccggacaaagactggtacgttcatcttgtcaaatcccagtgttggaccaggtcagattctgcactgctggaaggtgcagagctggtgaatcggattcctgctgaagatatgaatgccttcatgatgaactcggagttcaacctaagcctgctagctccatgcttaagcctagggatgagtgaaatttctggtggccagaagagtgccctttttgaagcagcccgtgaggtgactctggcccgtgtgagcggcaccgtgcagcagctccctgctgtccatcatgtcttccagcccgagctgcctgcagagccggcggcctactggagcaagttgaatgatctgtttggggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgaggccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccgttgcagtgcagcctggagagcagcttcttagtccagaaagaaggacaaataccccaaaagccatcagcgaggaggaggaggaagtagatccaaacacacagaatcctaagtatatcactgcagcctgtgagatggtggcagaaatggtggagtctctgcagtcggtgttggccttgggtcataaaaggaatagcggcgtgccggcgtttctcacgccattgctaaggaacatcatcatcagcctggcccgcctgccccttgtcaacagctacacacgtgtgcccccactggtgtggaagcttggatggtcacccaaaccgggaggggattttggcacagcattccctgagatccccgtggagttcctccaggaaaaggaagtctttaaggagttcatctaccgcatcaacacactaggctggaccagtcgtactcagtttgaagaaacttgggccaccctccttggtgtcctggtgacgcagcccctcgtgatggagcaggaggagagcccaccagaagaagacacagagaggacccagatcaacgtcctggccgtgcaggccatcacctcactggtgctcagtgcaatgactgtgcctgtggccggcaacccagctgtaagctgcttggagcagcagccccggaacaagcctctgaaagctctcgacaccaggtttgggaggaagctgagcattatcagagggattgtggagcaagagattcaagcaatggtttcaaagagagagaatattgccacccatcatttatatcaggcatgggatcctgtcccttctctgtctccggctactacaggtgccctcatcagccacgagaagctgctgctacagatcaaccccgagcgggagctggggagcatgagctacaaactcggccaggtgtccatacactccgtgtggctggggaacagcatcacacccctgagggaggaggaatgggacgaggaagaggaggaggaggccgacgcccctgcaccttcgtcaccacccacgtctccagtcaactccaggaaacaccgggctggagttgacatccactcctgttcgcagtttttgcttgagttgtacagccgctggatcctgccgtccagctcagccaggaggaccccggccatcctgatcagtgaggtggtcagatcccttctagtggtctcagacttgttcaccgagcgcaaccagtttgagctgatgtatgtgacgctgacagaactgcgaagggtgcacccttcagaagacgagatcctcgctcagtacctggtgcctgccacctgcaaggcagctgccgtccttgggatggacaaggccgtggcggagcctgtcagccgcctgctggagagcacgctcaggagcagccacctgcccagcagggttggagccctgcacggcgtcctctatgtgctggagtgcgacctgctggacgacactgccaagcagctcatcccggtcatcagcgactatctcctctccaacctgaaagggatcgcccactgcgtgaacattcacagccagcagcacgtactggtcatgtgtgccactgcgttttacctcattgagaactatcctctggacgtagggccggaattttcagcatcaataatacagatgtgtggggtgatgctgtctggaagtgaggagtccaccccctccatcatttaccactgtgccctcagaggcctggagcgcctcctgctctctgagcagctctcccgcctggatgcagaatcgctggtcaagctgagtgtggacagagtgaacgtgcacagcccgcaccgggccatggcggctctgggcctgatgctcacctgcatgtacacaggaaaggagaaagtcagtccgggtagaacttcagaccctaatcctgcagcccccgacagcgagtcagtgattgttgctatggagcgggtatctgttctttttgataggatcaggaaaggctttccttgtgaagccagagtggtggccaggatcctgccccagtttctagacgacttcttcccaccccaggacatcatgaacaaagtcatcggagagtttctgtccaaccagcagccatacccccagttcatggccaccgtggtgtataaggtgtttcagactctgcacagcaccgggcagtcgtccatggtccgggactgggtcatgctgtccctctccaacttcacgcagagggccccggtcgccatggccacgtggagcctctcctgcttctttgtcagcgcgtccaccagcccgtgggtcgcggcgatcctcccacatgtcatcagcaggatgggcaagctggagcaggtggacgtgaaccttttctgcctggtcgccacagacttctacagacaccagatagaggaggagctcgaccgcagggccttccagtctgtgcttgaggtggttgcagccccaggaagcccatatcaccggctgctgacttgtttacgaaatgtccacaaggtcaccacctgctgagcgccatggtgggagagactgtgaggcggcagctggggccggagcctttggaagtctgcgcccttgtgccctgcctccaccgagccagcttggtccctatgggcttccgcacatgccgcgggcggccaggcaacgtgcgtgtctctgccatgtggcagaagtgctctttgtggcagtggccaggcagggagtgtctgcagtcctggtggggctgagcctgaggccttccagaaagcaggagcagctgtgctgcaccccatgtgggtgaccaggtcctttctcctgatagtcacctgctggttgttgccaggttgcagctgctcttgcatctgggccagaagtcctccctcctgcaggctggctgttggcccctctgctgtcctgcagtagaaggtgccgtgagcaggctttgggaacactggcctgggtctccctggtggggtgtgcatgccacgccccgtgtctggatgcacagatgccatggcctgtgctgggccagtggctgggggtgctagacacccggcaccattctcccttctctcttttcttctcaggatttaaaatttaattatatcagtaaagagattaattttaacgtaactctttctatgcccgtgtaaagtatgtgaatcgcaaggcctgtgctgcatgcgacagcgtccggggtggtggacagggcccccggccacgctccctctcctgtagccactggcatagccctcctgagcacccgctgacatttccgttgtacatgttcctgtttatgcattcacaaggtgactgggatgtagagaggcgttagtgggcaggtggccacagcaggactgaggacaggcccccattatcctaggggtgcgctcacctgcagcccctcctcctcgggcacagacgactgtcgttctccacccaccagtcagggacagcagcctccctgtcactcagctgagaaggccagccctccctggctgtgagcagcctccactgtgtccagagacatgggcctcccactcctgttccttgctagccctggggtggcgtctgcctaggagctggctggcaggtgttgggacctgctgctccatggatgcatgccctaagagtgtcactgagctgtgttttgtctgagcctctctcggtcaacagcaaagcttggtgtcttggcactgttagtgacagagcccagcatcccttctgcccccgttccagctgacatcttgcacggtgaccccttttagtcaggagagtgcagatctgtgctcatcggagactgccccacggccctgtcagagccgccactcctatccccaggccaggtccctggaccagcctcctgtttgcaggcccagaggagccaagtcattaaaatggaagtggattctggatggccgggctgctgctgatgtaggagctggatttgggagctctgcttgccgactggctgtgagacgaggcaggggctctgcttcctcagccctagaggcgagccaggcaaggttggcgactgtcatgtggcttggtttggtcatgcccgtcgatgttttgggtattgaatgtggtaagtggaggaaatgttggaactctgtgcaggtgctgccttgagacccccaagcttccacctgtccctctcctatgtggcagctggggagcagctgagatgtggacttgtatgctgcccacatacgtgagggggagctgaaagggagcccctcctctgagcagcctctgccaggcctgtatgaggcttttcccaccagctcccaacagaggcctcccccagccaggaccacctcgtcctcgtggcggggcagcaggagcggtagaaaggggtccgatgtttgaggaggcccttaagggaagctactgaattataacacgtaagaaaatcaccattccgtattggttgggggctcctgtttctcatcctagctttttcctggaaagcccgctagaaggtttgggaacgaggggaaagttctcagaactgttggctgctccccacccgcctcccgcctcccccgcaggttatgtcagcagctctgagacagcagtatcacaggccagatgttgttcctggctagatgtttacatttgtaagaaataacactgtgaatgtaaaacagagccattcccttggaatgcatatcgctgggctcaacatagagtttgtcttcctcttgtttacgacgtgatctaaaccagtccttagcaaggggctcagaacaccccgctctggcagtaggtgtcccccacccccaaagacctgcctgtgtgctccggagatgaatatgagctcattagtaaaaatgacttcacccacgcatatacataaagtatccatgcatgtgcatatagacacatctataattttacacacacacctctcaagacggagatgcatggcctctaagagtgcccgtgtcggttcttcctggaagttgactttccttagacccgccaggtcaagttagccgcgtgacggacatccaggcgtgggacgtggtcagggcagggctcattcattgcccactaggatcccactggcgaagatggtctccatatcagctctctgcagaagggaggaagactttatcatgttcctaaaaatctgtggcaagcacccatcgtattatccaaattttgttgcaaatgtgattaatttggttgtcaagttttgggggtgggctgtggggagattgcttttgttttcctgctggtaatatcgggaaagattttaatgaaaccagggtagaattgtttggcaatgcactgaagcgtgtttctttcccaaaatgtgcctcccttccgctgcgggcccagctgagtctatgtaggtgatgtttccagctgccaagtgctctttgttactgtccaccctcatttctgccagcgcatgtgtcctttcaaggggaaaatgtgaagctgaaccccctccagacacccagaatgtagcatctgagaaggccctgtgccctaaaggacacccctcgcccccatcttcatggagggggtcatttcagagccctcggagccaatgaacagctcctcctcttggagctgagatgagccccacgtggagctcgggacggatagtagacagcaataactcggtgtgtggccgcctggcaggtggaacttcctcccgttgcggggtggagtgaggttagttctgtgtgtctggtgggtggagtcaggcttctcttgctacctgtgagcatccttcccagcagacatcctcatcgggctttgtccctcccccgcttcctccctctgcggggaggacccgggaccacagctgctggccagggtagacttggagctgtcctccagaggggtcacgtgtaggagtgagaagaaggaagatcttgagagctgctgagggaccttggagagctcaggatggctcagacgaggacactcgcttgccgggcctgggcctcctgggaaggagggagctgctcagaatgccgcatgacaactgaaggcaacctggaaggttcaggggccgctcttcccccatgtgcctgtcacgctctggtgcagtcaaaggaacgccttcccctcagttgtttctaagagcagagtctcccgctgcaatctgggtggtaactgccagccttggaggatcgtggccaacgtggacctgcctacggagggtgggctctgacccaagtggggcctccttgtccaggtctcactgctttgcaccgtggtcagagggactgtcagctgagcttgagctcccctggagccagcagggctgtgatgggcgagtcccggagccccacccagacctgaatgcttctgagagcaaagggaaggactgacgagagatgtatatttaattttttaactgctgcaaacattgtacatccaaattaaaggaaaaaaatggaaaccatca

SEQ ID NO: 3 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 4 - DNA, 20 bp, synthetic construct
  source 1..20: mol_type = other DNA
ctcagtaacattgacaccac